(19) United States
(12) Patent Application Publication
(10) Pub. No.: US 2024/0327364 A1
(43) Pub. Date: Oct. 3, 2024

(54) THERAPEUTIC TARGETING OF GPR68 TO INDUCE FERROPTOSIS

(71) Applicant: University Of Maryland, Baltimore, Baltimore, MD (US)

(72) Inventors: Charles C. HONG, Baltimore, MD (US); Charles H. WILLIAMS, Odenton, MD (US); Samantha Rea, Baltimore, MD (US); Leif Neitzel, Baltimore, MD (US); Jessica Cornell, Columbia, MD (US)

(21) Appl. No.: 18/173,962

(22) Filed: Feb. 24, 2023

(51) Int. Cl. C07D 285/14; A61K 31/351; A61K 31/4985; A61P 35/00; G01N 33/50

(52) U.S. Cl. CPC C07D 285/14; A61K 31/351; A61K 31/4985; A61P 35/00; G01N 33/5011

(57) ABSTRACT

The invention relates to small molecule inhibitors of GPR68, useful as therapeutic agents in conditions benefitted from inhibition of GPR68, such as cancers and acute lung injuries. Selective inhibition of GPR68 caused robust cell death in glioblastoma cell lines, without toxicity, as well as death of other cancer cells, such as lung and pancreatic cancers. Synergistic benefits are achieved using combination treatment of GPR68 inhibitor compounds with radiation therapy or traditional chemotherapies for cancers. Thus, GPR68 inhibition enhances the therapeutic efficacy of ionizing radiation and chemotherapies. GPR68 inhibition is a therapeutic approach to induce ferroptosis in glioblastoma multiforme and other cancers, in a synergistic manner with ionizing radiation. Since ferroptosis is a form of immunogenic cell death, GPR68 inhibition represents an attractive approach to enhance cancer immunotherapies, including check-point inhibitors and cancer vaccines. The treatment methods also show beneficial results in acute lung injury such as acute respiratory distress syndrome.

### GOVERNMENT FUNDING SUPPORT

This invention was made with government support under grant no. GM 118557 awarded by the National Institutes of Health. The government has certain rights in the invention.

### REFERENCE TO SEQUENCE LISTING SUBMITTED VIA EFS-WEB

The application contains a Sequence Listing which has been submitted electronically in .XML format and is hereby incorporated by reference in its entirety. Said .XML copy, created on Jun. 26, 2023, is named “15024-380US0.xml” and is 39,373 bytes in size. The sequence listing contained in this .XML file is part of the specification and is hereby incorporated by reference herein in its entirety.

### BACKGROUND OF THE INVENTION

### 1. Field of the Invention

The invention relates to the general field of medicine and in particular to the use of a novel class of small molecule GPR68 inhibitors for treating diseases and conditions benefited by such inhibition. Conditions for which the methods are useful include cancer (such as pancreatic cancer and glioblastoma) and acute lung injury such as acute respiratory distress syndrome. Important GPR68 inhibitors include compounds of the 1,2-dihydro-3′H-spiro[indole-3,2′-(1,3,4}thiadiazole]-2-one class. The methods include inducing ferroptosis, a type of programmed cell death dependent on iron and characterized by the accumulation of lipid peroxides and disruption of mitochondria for cancer and acute lung injury; and methods using a combination of GPR68 inhibition along with ionizing radiation or traditional chemotherapeutic agents, such as temozolomide, for cancer.

### 2. Background of the Invention

The Warburg effect, a phenomenon in which cancer cells produce energy via aerobic glycolysis, is a hallmark of cancer and the basis for cancer and metastasis detection by positron emission tomography (PET) scan. A key physiological consequence of the Warburg effect is lactate secretion, which acidifies the tumor milieu (acidic tumor microenvironment, pH about 6.4). Acidic tumor milieu, which thought to confer tumor resistance to chemotherapy and radiotherapy, occurs in many cancers, for example, in glioblastoma. The effect of an acidic environment on cancer progression can be categorized into effects on tumor cell survival, tumor metastasis, inflammatory response, and blood vessels. Yet the underlying signaling and cell biological underpinnings of these phenomena are not well understood. Acidic TME promotes pro-oncogenic programs such as tumor survival, metastasis, therapeutic resistance, and escape from anti-tumor immune response. Proton-sensing G protein-coupled receptor (GPCR), GPR68, is a key mediator of the pro-oncogenic program activated by the acidic TME.

Highly malignant, invasive, and metastatic cancers have markedly elevated glycolytic activity, producing an oncologically favorable acidotic extracellular environment; a phenomenon called the Warburg effect (Vander Heiden et al., 2009). This acidification (pH <7.4) of the environment is marked by increases in efflux mechanisms H+ATPases and Na+-H+ exchangers (Martinez-Zaguilan et al., 1993; Martinez-Zaguilin et al., 1998; McLean et al., 2000; Sennoune et al., 2004). Acidification promotes tumor malignancy, including metabolic reprogramming and invasiveness. Investigations have shown that in numerous animal models of solid tumors, small molecule inhibition of NHE1 can have an effect on tumor growth and metastasis (Matthews et al., 2011), however the cellular mechanisms triggered by the acidification of the tumor environment are still not wholly understood.

Glioblastoma multiforme (GBM) is the most common and most fatal primary brain tumor in adults. Despite aggressive standard management, which includes maximal surgical resection, followed by radiation and chemotherapy with the frontline agent temozolomide (TMZ), median survival for individuals with GBM is only 14 months, and recurrence is the rule. The poor prognosis of GBM is attributed to therapeutic resistance and molecular heterogeneity. Resistance to the alkylating agent TMZ commonly involves induction of the DNA repair enzyme O(6)-methylguanine-DNA methyltransferase, encoded by the MGMT gene. Through this mechanism, TMZ resistance is almost universal in recurrent GBM.

In addition, GBM tumors are notorious for their high molecular heterogeneity. For example, although amplifications of EGFR (epidermal growth factor) and PDGFR (platelet-derived growth factor) are common in GBM, they tend to be unevenly distributed within individual tumors, often rendering receptor tyrosine kinase inhibitors that target them ineffective. Moreover, analysis of TCGA (The Cancer Genome Atlas) reveals significant heterogeneity among patients, and single-cell RNA-seq of primary GBM tumors demonstrate significant intratumoral heterogeneity, marked by at least four distinct malignant cell states. Given such cellular plasticity and heterogeneity, it remains unclear whether a single therapeutic target can lead to an efficacious “universal” treatment for GBM.

Asthma exacerbation is often triggered by airway acidification. This can be caused by exogenous factors such as air pollution as well as by endogenous factors such as gastroesophageal reflux disease (GERD). There are major processes in asthma pathogenesis: (1) mucous hyperproduction, (2) bronchoconstriction, and (3) inflammation. The inflammation is highly characteristic of an abnormal immune response, highly skewed toward the arm of the immune system commonly called “Th2” or “type 2” immunity. There are particular cell types involved in the immune response including innate immune cells called “eosinophils” and antigen presenting cells called “dendritic cells” or “DCs” as well as particular cytokines that include, but are not limited to, IL-4, IL-5, and IL-13 (1). Asthma severity has been shown to correlate with the pH of induced sputum samples. In other words, the less well-controlled the asthma, the lower the sputum pH. Furthermore, poor asthma control also is correlated with an increased percentage of eosinophils in sputum samples. The proton-sensing molecule, GPR68, is implicated in all cardinal pathogenic processes in asthma.

Aspiration pneumonia is a leading cause of pneumonia in the intensive care unit and is one of the leading risk factors for acute lung injury (ALI) and acute respiratory distress syndrome (ARDS). Patients placed on a ventilator are particularly susceptible to aspiration pneumonia. The mechanisms underlying the impact of acid aspiration on lung inflammation are poorly understood. The main pathologic characteristics of acid aspiration-induced lung injury include increased permeability of the alveolus-capillary interface, interstitial inflammation, and edema that eventually fills the alveolar air sacs, which are also consistent with the pathology of ARDS. Hypoxia is a hallmark of lung injury and hypoxia and ultimately leads to activation of hypoxia-inducible factor (HIF)-1 alpha (7). HIF-1alpha plays a central role in the inflammation caused by acid aspiration characterized by an accumulation alveolar macrophages, neutrophils, and increased permeability via type II alveolar epithelial cells (i.e. the cells that produce surfactant).

Hypoxia and inflammation are interconnected and linked in multiple ways and may induce and influence each other. Inflammation may be hypoxia-driven or hypoxia may be induced by inflammation (inflammatory hypoxia). Hypoxia not only maintains or aggravates inflammation via stabilization of HIF-1alpha but can also lower the local tissue pH. This acidic environment is not only the result of inflammation, but also affects the degree and outcome of inflammation. Inflammation has been attributed to an increase in local proton concentration and lactate production and has also been linked to subsequent proinflammatory cytokine production, including TNF-alpha. IL-1 beta, IL-6, and interferon-gamma. Thus, hypoxia, inflammation, and low pH can create a pathological feedforward loop.

Hypoxia also positively regulates the expression of GPR68, as demonstrated in surgical resection specimens from subjects with inflammatory bowel disease (IBD). In particular, GPR68 is directly upregulated on intestinal submucosal macrophages in IBD compared to normal control in specimens incubated in a hypobaric chamber. In addition, HIF-1 alpha binds to the GPR68 promoter under the hypoxic conditions. Thus, it appears that hypoxia leads to a local acidic microenvironment and causes up-regulation of GPR68 via HIF-1 alpha-induced transcription, which in turn drives mucosal inflammation.

Currently, many cancers and acute lung injury are difficult to treat.

### SUMMARY OF THE INVENTION

Therefore, there is a need in the art for treatments for various cancers such as glioblastoma multiforme and pancreatic cancer, as well as acute respiratory distress.

The invention relates to a class of small molecule inhibitors of GPR68/OGR1, a proton-sensing/stretch-sensing/sheer-stress-sending G-protein coupled receptor, and related receptors GPR4 and GPR65. These inhibitors are useful as a therapeutic for glioblastoma and other neoplasms, as a monotherapy or adjuvant. Additionally, the inhibitors can be used as a treatment for other conditions, such as osteoporosis, inflammatory bowel diseases, autoimmune and chronic inflammatory diseases, such as multiple sclerosis, and inflammatory pain syndromes, asthma, chronic obstructive pulmonary disease, aspiration pneumonia, viral pneumonia, coronavirus pneumonia and lung injury, acute lung injury (ALI), acute respiratory distress syndrome (ARDS), diabetes type 1, osteoporosis, inflammatory bowel disease, chronic inflammatory disease, atherosclerosis, cardiovascular disease, multiple sclerosis, inflammatory pain syndrome, and autoimmune disease.

In particular embodiments, the present invention relates to a method of inducing ferroptosis for treatment in a subject in need thereof, comprising administering to the subject a therapeutic GPR68 inhibiting 1,2-dihydro-3′H-spiro[indole-3,2′-[1,3,4]thiadiazole]-2-one agent of Formula I or a salt thereof:

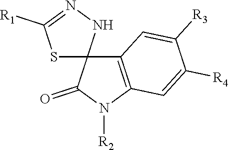

wherein R₁ is an optionally substituted

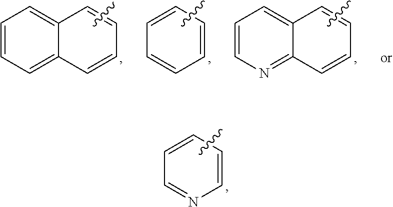

wherein the substitution is selected from —H, —CH₃, —CH₂CH₃, —OCH₃, —Br, —F, and —CF₃; wherein R₂ is —H or —CH₃; and wherein R₃ and R₄ independently are —H, —CH₃, —CH₂CH₃, —OCH₃, —CN, —F, —COOCH₃, —COOH, —SO₂NH₂.

In certain embodiments, the subject is suffering from cancer or an acute lung injury.

In certain embodiments, the subject is suffering from cancer. In these embodiments, the cancer preferably is a lymphoma, a leukemia, a germ cell tumor, a blastoma, a sarcoma, a blood cancer, a skin cancer, a breast cancer, a cervical cancer, an ovarian cancer, a breast cancer, a prostate cancer, a kidney cancer, a lung cancer, a pancreatic cancer, a liver cancer, a colon or colorectal cancer, and a brain cancer. More preferably, the cancer is glioblastoma multiforme, medulloblastoma, fibrosarcoma, monocytic leukemia, B-cell lymphoma, chronic myelogenous leukemia, neuroendocrine prostate cancer, lung, colon, breast, pancreatic, and melanoma.

In certain embodiments, the methods further comprise administering a cancer chemotherapeutic agent to the subject. This cancer chemotherapeutic agent can be temozolomide or doxorubicin. Preferably, co-administration of the therapeutic GPR68 inhibiting 1,2-dihydro-3′H-spiro[indole-3,2′-[1,3,4]thiadiazole]-2-one agent of Formula I and a cancer chemotherapeutic agent synergistically induces ferroptosis, immunogenic cell death, or both in cancer cells in the context of acidic tumor microenvironment.

In other embodiments, the methods further comprise administering radiation therapy to the subject. Preferably, co-administration of the therapeutic GPR68 inhibiting 1,2-dihydro-3′H-spiro[indole-3,2′-[1,3,4]thiadiazole]-2-one agent of Formula I and radiation therapy to the subject synergistically induces ferroptosis, immunogenic cell death, or both in cancer cells in the context of acidic tumor microenvironment.

In certain embodiments, the methods further comprise administering an ATF4 activating agent to the subject to overcome therapeutic resistance.

In certain embodiments, the methods further comprise administering a cancer immunotherapy agent to the subject. Cancer immunotherapy agents preferably are selected from the group consisting of ipilimumab, pembrolizumab, nivolumab, and atezolizumab.

More preferably, co-administration of the therapeutic GPR68 inhibiting 1,2-dihydro-3′H-spiro[indole-3,2′-[1,3,4]thiadiazole]-2-one agent of Formula I and a cancer immunotherapy agent to the subject promotes anti-cancer immunity.

In certain embodiments of the invention, the subject is suffering from an acute lung injury. This acute lung injury can be caused by bacterial infection, viral infection, inhalation injury, trauma, or mechanical ventilation-induced barotrauma. In particular embodiments of the invention, the subject is suffering from acute respiratory distress syndrome or is at risk of developing acute respiratory distress syndrome.

In preferred embodiments, the therapeutic GPR68 inhibiting 1,2-dihydro-3′H-spiro[indole-3,2′-[1,3,4]thiadiazole]-2-one agent of Formula I or a salt thereof is OGM2, OGM17, OGM24, or OGM74.

In certain embodiments, the invention comprises a method of inducing ferroptosis for treatment in a subject in need thereof, comprising administering to the subject a therapeutic amount of a therapeutic DNA or RNA molecule, wherein the DNA or RNA molecule comprises a sequence that silences, degrades or modulates GPR68-coding RNA. In further embodiments, the invention comprises such methods wherein the administering the DNA or RNA molecule comprises a method selected from the group consisting of; (a) administering the DNA or RNA molecule in a lipid nanoparticle formulation by intravenous injection; (b) administering the DNA or RNA molecule in a polymeric nanoparticle formulation by inhalation; (c) administering the DNA or RNA molecule in a viral vector formula by intramuscular injection; (d) administering the DNA or RNA molecule in a conjugate formulation by topical application; (e) administering the DNA or RNA molecule in a prodrug formulation by oral administration; and (f) administering the DNA or RNA molecule in a nanoparticle formulation comprising a targeting ligand by subcutaneous injection. In preferred embodiments the DNA or RNA molecule is an siRNA or a microRNA.

In certain embodiments, the invention comprises a method of predicting the sensitivity of individual cancers to killing by GPR68 inhibition, comprising the steps of: (a) obtaining a fresh or frozen tumor sample from a patient at the time of diagnostic tissue biopsy, surgical excision, bone marrow biopsy or peripheral blood draw; (b) isolating mRNA from the tumor sample; (c) generating cDNAs from the mRNA; (d) determining the normalized expressions of GPR68 and GPR4 in the tumor sample by performing real-time qPCR; (e) determining the ratio of the normalized expression of GPR68 relative to the normalized expression of GPR4 in the tumor sample, wherein a ratio of 2:1 GPR68:GPR4 or higher in an individual tumor sample indicates increased sensitivity of the tumor to killing by GPR68 inhibition, and wherein a ratio of less than 2:1 a ratio of 2:1 GPR68:GPR4 in an individual tumor sample indicates a lack of increased sensitivity of the tumor to killing by GPR68 inhibition. In some embodiments, a ratio of 2:1 GPR68:GPR4 or higher in the individual tumor sample predicts therapeutic efficacy of the therapeutic agents.

In certain embodiments, the invention comprises a method of enhancing the therapeutic efficacy of cancer immunotherapy in a subject in need thereof, comprising the steps of: (a) administering to the subject a therapeutic amount of a therapeutic DNA or RNA molecule, wherein the DNA or RNA molecule comprises a sequence that silences, degrades or modulates GPR68-coding RNA; and (b) initiating cancer immunotherapy for the subject after or during the performance of (a). In certain embodiments, the cancer immunotherapy comprises administration of a checkpoint inhibitor or a tumor cell-killing chimeric antigen receptor T cells.

In certain embodiments, the invention comprises a method of enhancing immunological memory against tumors, comprising the steps of: (a) administering to the subject a therapeutic amount of a therapeutic DNA or RNA molecule, wherein the DNA or RNA molecule comprises a sequence that silences, degrades or modulates GPR68-coding RNA; and (b) administering to the subject a tumor vaccine.

### BRIEF SUMMARY OF THE DRAWINGS

Certain embodiments are illustrated by way of example, and not by way of limitation, in the figures of the accompanying drawings.

FIG. 1A through FIG. 1D. GPR68 variant Rs61745752 associated with oncological signals including metastasis. FIG. 1A shows the number of heterozygous and homozygous synonymous ‘silent’ mutations and stop gain variant carriers in the imputed UK Biobank dataset. FIG. 1B are the results of functional outcome algorithm for Rs61745752 showing 4/5 predicting a deleterious or pathogenic consequence. FIG. 1C shows highlighted neoplasm ICD10 codes. FIG. 1D shows increased association of Stop gain variant rs61745752 with both benign and malignant neoplasm ICD10 codes, while silent mutations do not share the same associations.

FIG. 2A through FIG. 2C. Discovery of the ogremorphin (OGM) class of compounds. FIG. 2A provides the structure of Ogremorphin-1 (OGM1; 5-ethyl-5′-naphthalen-1-ylspiro[1H-indole-3,2′-3H-1,3,4-thiadiazole]-2-one. FIG. 2B shows a dorsal view of a DMSO vehicle control-treated zebrafish embryo at 48 hours post fertilization (hpf).

FIG. 2C shows a dorsal view of an OGM1 (10 μM) treated zebrafish embryo at 48 hours pf.

FIG. 3A and FIG. 3B: OGM1 affects neural crest cell derived pigment development in zebrafish embryo. FIG. 3A presents a temporal phenotypic analysis. FIG. 3B shows a lateral view of zebrafish at the 10-somite stage, control and after in situ hybridization of FOXD3, a neural crest marker, indicates the OGM1 treatment does not grossly perturb neural crest cell differentiation.

FIG. 4 shows the results of a Millipore™ GPCRome screen. OGM1 only inhibited 2 GPCRs in a screen of 158 GPCRs. OGM1 is a reversible inhibitor of GPR68.

FIG. 5A through FIG. 5H show that that loss of LPAR1 activity did not correlate with loss of the zebrafish phenotype. GPR68 Knockdown recapitulated OGM2 induced zebrafish phenotype. FIG. 5A is a set of photographs of a morpholino knock down experiment, showing that knock down of GPR68 in zebrafish recapitulate phenotypes of GPR68 inhibition by OGM molecules. FIG. 5A: top=control; middle=OGM treatment; Bottom=morphant. FIG. 5B, FIG. 5C, FIG. 5D, and FIG. 5E: consistent with ogremorphin treatment, Morpholino and Cas9 knockdown of GPR68 resulted in craniofacial dysmorphogenesis, disrupted pigmentation, and a wavy notochord (Red arrow) as shown in FIG. [[5A]]5E. Cas9 targeting of GPR68 generated the same phenotype as both OGM treatment and GPR68 knockdown with a shortened body axis, abnormal pigmentation, and defects in notochord integrity. Cas9^{GPR68}=GPR68 targeted sgRNA and Cas9; CTL=control; MoGPR68=GPR68 morpholino treatment. FIG. 5F, FIG. 5G, and FIG. 5H are bar graphs showing quantitation of the phenotypes present in percentage of treated embryos.

FIG. 6A and FIG. 6B. HNMR and LCMS of resynthesis of OGM2. FIG. 6A is a graph showing HNMR spectra confirming resynthesis of OGM2. FIG. 6B is a graph showing LCMS spectra confirming resynthesis of OGM2.

FIG. 7A and FIG. 7B are tables that show the degree of homology (FIG. 7A) and the degree of homology (FIG. 7B) for the similarity of GPCR family members.

FIG. 8 shows representative dose response assay of OGM2 (OGM8345), and an OGM1 analog.

FIG. 9A and FIG. 9B. Gene profiling datasets. FIG. 9A is a graph showing overall survival of glioblastoma over time. FIG. 9B is a graph presenting data inhibition of calcium flux response to acidification by OGM2 in U87 human glioblastoma cells.

FIG. 10A and FIG. 10B. OGM2 inhibits human melanoma migration. FIG. 10A shows that OGM2 (also referred to as OGM8345) and EIPA both prevent wound closure compared to DMSO (CTL). FIG. 10B is a bar graph showing results from quantitation of inhibition of scratch assay across three melanoma lines.

FIG. 11: RT PCR of proton-sensing GPCRs in human melanoma cell lines. FIG. 11 is a photograph of a gel showing WM115, MeWo, and A2058, as indicated.

FIG. 12A through FIG. 12G. FIG. 12A is a schematic of an agarose drop assay. FIG. 12B, FIG. 12C, FIG. 12D are photographs of agarose gel plates. FIG. 12E is a bar graph that presents data from quantitation of the cells that escaped from an average of 5 gel drop replicates.

FIG. 13A through FIG. 13D. Variant Rs61745752 is a functional c-terminal truncation. FIG. 13A is a phosphocode prediction of beta-arrestin binding sites downstream of amino acid 335. The figure shows SEQ ID NO:1 (rhodopsin), SEQ ID NO:2 (vasopressin 2), SEQ ID NO:3 (GPR68) and SEQ ID NO:4 (rs61745752). FIG. 13B is a set of photographs of GPR68-GFP and 336X-GFP transfected HEK293 cells. FIG. 13C is a bar graph presenting data from quantitation of the number of puncta in each cell. FIG. 13D shows calcium flux assay of truncation variant in transfected HEK293 cells showing elevated calcium levels.

FIG. 14: E336X-GFP plasmid is still active in SRE-luciferase assay. FIG. 14 is a bar graph presenting a composite of results from a serum responsive element (SRE-) luciferase assay.

FIG. 15: OGM2 still able to inhibit GPR68 activation in truncation variant. FIG. 15 is a bar graph.

FIG. 16A through FIG. 16C: Mutations in GPR68 correlate with poor prognosis in breast cancer. FIG. 16A, FIG. 16B, and FIG. 16C are Kaplan-Mier plots generated in cBioportal of the breast cancer cohort carrying alterations in GPR68 (FIG. 16A), and alterations in members of the same protein family, alterations in GPR4 (FIG. 16B), and alterations in GPR65 (FIG. 16C).

FIG. 17A through FIG. 17D. GPR68 activity destabilized MCF7 spheroids. FIG. 17A is a set of photographs of MCF7 cells transfected with GFP, GPR68-GFP, and 336X-GFP, seeded in Ultra low attachment plates to form spheroids. FIG. 17B presents data from quantitation of the number of spheroids without intact outer rings. FIG. 17C presents data from quantitation of the size of the outgrowth from spheroids. FIG. 17D is a set of photographs allowing visualization of GFP+ cells in spheroids.

FIG. 18A and FIG. 18B Relative matrix invasion of U87 (glioblastoma) cells in Matrigel™. FIG. 18A is a set of representative photographs of control and OGM2-treated cells. FIG. 18B shows quantitation of these data in graph form.

FIG. 19A and FIG. 19B. GPR68 inhibition of Glioblastoma growth in monolayer culture. FIG. 19A and FIG. 19B are graphs showing growth attenuation in TMZ sensitive (FIG. 19A) and insensitive (FIG. 19B) glioblastoma models.

FIG. 20A and FIG. 20B. GPR68 inhibition of Glioblastoma growth in spheroid culture. FIG. 20A and FIG. 20B are graphs showing growth attenuation in both TMZ sensitive (FIG. 20A) and insensitive (FIG. 20B) glioblastoma models.

FIG. 21A and FIG. 21B. Ogerin GPR68 Positive Allosteric Modulator (PAM). FIG. 21A is a photograph of tumor spheroids treated with DMSO, or OGM2. FIG. 21B is a graph showing growth stimulation in 3D tumoroid culture in glioblastoma models over time when treated with DMSO, Ogerin or OGM2.

FIG. 22A and FIG. 22B. GPR68 inhibition increases cell death. FIG. 22A is a set of photographs showing 2D cancer cell assays. FIG. 22B is a set of photographs showing 3D tumor spheroid assays.

FIG. 23A and FIG. 23B. pH dependent OGM inhibition of tumoroid growth. FIG. 23A is a set of photographs showing treated tumor spheroids. FIG. 23B is a bar graph showing data for tumor spheroids treated at the indicated pH.

FIG. 24: OGM2 reduces clonogenicity of U87 glioblastoma cells. FIG. 24 is a photograph of a six-well plate containing U87 cells treated with increasing concentrations of OGM2.

FIG. 25: HCT116 colon cancer treatment. FIG. 25 is a graph showing cells treated with OGM2 at the indicated concentrations.

FIG. 26A through FIG. 26B. Studies of PAN02 pancreatic cancer cells. FIG. 26A is a graph showing the effect of high doses of OGM2 on PAN02 pancreatic cancer cells. FIG. 26B is a set of photographs showing reduction of migration by OGM2 in PAN02 pancreatic cancer cells. FIG. 26C is a photograph of a six-well plate containing PANC02 cells treated with increasing concentrations of OGM2.

FIG. 27A and FIG. 27B. Studies of A549 lung cancer cells. FIG. 27A is a graph showing the effect of high doses of OGM on A549 lung cancer cells. FIG. 27B is a photograph of a six-well plate containing A549 cells treated increasing concentrations of OGM2.

FIG. 28A and FIG. 28B. Viability of cancer cells. FIG. 28A is a graph showing that lower GPR68 expression correlates to better prognosis of multiple cancer cell types. FIG. 28B is a graph showing that OGM2 reduces the viability of Prostate and breast cancer cells.

FIG. 29A and FIG. 29B. OGM2 synergizes with TMZ and radiation. FIG. 29A is a graph showing data for viability of U87 cells treated with increasing concentrations of TMZ, with or without OGM2. FIG. 29B is a set of photographs of wells containing irradiated PANC02 cells treated with OGM2.

FIG. 30A through FIG. 30F. OGM2 inhibits acid induced Mucin production. FIG. 30A and FIG. 30B are dot blots and corresponding western blots to detect Mucin-5AC and alpha tubulin, respectively. FIG. 30A shows that acidification increases Mucin production.

FIG. 30B shows that OGM2 inhibits Mucin production. FIG. 30C and FIG. 30D are photomicrographs showing A549 cells in culture stained to detect mucins. FIG. 30E is a schematic of cas9 mediated endogenous tagging of MU5AC genetic locus with nano-luciferase in A549 cells. FIG. 30F is a bar graph showing data for Muc5ac-Luc cell lysates assayed for luciferase activity.

FIG. 31: Schematic of GPR68 activation effects in the lungs. FIG. 31 is a drawing showing negative effects of GPR68 activation in certain pulmonary disease conditions.

FIG. 32 shows that OGM2 is a direct inhibitor of GPR68-mediated mechanosensing signaling, blocking calcium response induced by laminar flow.

FIG. 33A through FIG. 33D: Cyclic stretch-triggered endothelial cell (EC) activation is abolished by OGM2 and OGM1. FIG. 33A: In human pulmonary artery endothelial cells (HPAECs), pretreatment with OGMs (OGM2 at 5 μM and OGM1 at 3 μM) abolished Rho activation by 18% cyclic stretch (18% CS), as determined by Rho-GTP pulldown assay. ST, static condition. FIG. 33B: Treatment with OGM2 or OGM1 blocked CS-triggered activation of myosin phosphatase target (MYPT) and myosin light chain (MLC) in ECs, as determined by their phosphorylation MYPT and MLC. FIG. 33C: OGM2 or OGM1 blocked CS-induced TNF-α mRNA expression in ECs. n=3, ^{#}P<0.05 versus ST, *P<0.05 versus 18% CS. Error bars, standard deviation. FIG. 33D: OGM2 and OGM1 blocked CS-induced VCAM-1 expression in ECs. Here, OGM=OGM2.

FIG. 34A through FIG. 34C: GPR68 plays a central role in acidification-induced endothelial cell (EC) dysfunction in vitro, and OGM2 abrogates EC dysfunction. FIG. 34A and FIG. 34B show that acidic media (pH 6.5) induces inflammatory mediators TNFα (tumor necrosis factor-alpha), VCAM1 (vascular cell adhesion molecule-1) and ICAM1 (intercellular adhesion molecule-1) mRNA (FIG. 34A) and protein (FIG. 34B) expression. These effects were abolished by OGM2 (OGM) at 3 uM and augmented by GPR68 positive allosteric modulator Ogerin (Og) at 2 uM. *P<0.05; Mann-Whitney test, n=5. FIG. 34C: Medium acidification reduced transendothelial resistance (TER), indicative of disruption of EC barrier function. OGM2, but not GPR4 inhibitor (GPR4i, 5 uM) abolished TER decline caused by low pH (6.5).

FIG. 35A through FIG. 35F: Medium acidification has additive effects with bacterium and LPS (lipopolysaccharide) in causing EC dysfunction, all of which are abolished by OGM2. FIG. 35A: mRNA expression of inflammatory markers TNFα, VCAM1, IL-8, IL-1β are elevated in HPAECs (human pulmonary artery endothelial cells) in response to low pH (6.5) and HKSA (heat-killed staph aureus), and in combination. *p <0.05; Mann-Whitney test, n=3. FIG. 35B: LPS (50 ng/ml)-induced decline in TER was further augmented by lowering pH. FIG. 35C: OGM2 protected against HKSA-induced permeability as monitored by XPerT visualization assay; n=4. FIG. 35D: Surfact biotinylation assay reveals that HKSA decreased VE-cadherin expression at the endothelial cell surface, this effect was attenuated by OGM. FIG. 35E: OGM2 treatment up to 2 hours after LPS exposure reduces LPS-induced EC inflammation. FIG. 35F: OGM2 treatment 6 hours after LPS exposure reduces LPS-induced EC hyper-permeability.

FIG. 36A through FIG. 36H: Additive effects of cyclic strain (CS) with LPS and HKSA on EC GPR68 signaling and EC dysfunction are abolished by OGM2. FIG. 36A: In GPR68 activation-Tango luciferase assay, GPR68 activation by LPS and HKSA in HPAEC under static conditions was augmented by cell exposure to 18% CS (4 hr) *p<0.05 vs Veh; **p<0.05 vs static; n=3. FIG. 36B: VCAM1 expression stimulated by 18% CS (6 hours) was inhibited by OGM2 (5 μM). FIG. 36C: LPS-induced VCAM1 expression was augmented by 18% CS; the CS effect was inhibited by OGM2 (5 μM) or OGM1 (3 μM); n=3. FIG. 36D: increased VCAM1 expression due to a submaximal dose (2 μM) of Ogerin, a positive allosteric modulator of GPR68, was further enhanced by 18% CS. FIG. 36E: in Co-immunoprecipitation assay with a GPR68 antibody, 18% CS (30 minutes) induced GPR68 association with TLR2; n=3. FIG. 36F: 18% CS-induced VCAM1 protein expression (24 hrs) was augmented by low pH and attenuated by OGM2 (5 μM), but not by GPR4i (5 μM). FIG. 36G: HKSA-induced mRNA expression of inflammatory markers in stretched ECs was suppressed by OGM but not by GPR4i; *p<0.05 vs HKSA alone; n=4. FIG. 36H: Low pH augmented EC gap formation (arrows) caused by 18% CS; this effect was attenuated by OGM2. Immunofluorescence staining for VE-cadherin (green) and F-actin (red). Here, OGM=OGM2.

FIG. 37A through FIG. 37D: OGM2 attenuates HKSA- and LPS-induced acute lung injury (ALI) in vivo. FIG. 37A: GPR68 mRNA is markedly elevated in lungs, heart and brain of HKSA-challenged (5×10⁷/kg, i/n, 18 hr) C57Bl mice). FIG. 37B: LPS (0.7 mg/kg intratracheal) and HTV-induced ALI in mice markedly increases mRNA expression of GPR68, relative to GPR4, GPR65, and GPR132; *p<0.01 vs. vehicle; n=3. FIG. 37C: in C57Bl mice, HKSA-induced ALI, characterized by lung vascular leak monitored by extravasation of Evans blue-labeled albumin into the lung tissue, was significantly attenuated by OGM2 (40 mg/kg, Up) at 3 hr post OGM-administration. FIG. 37D: in the setting of HKSA-induced ALI, OGM2 treatment (40 mg/kg, i.p.) significantly attenuated induction of inflammatory cytokine (TNFα, IL-6, KC—keratinocyte chemoattractant, IL-1β) transcripts in the lungs; *p<0.05 vs HKSA alone; n=5.

FIG. 38A through FIG. 38D: High-tidal volume (HTV) mechanical ventilation causes ALI and exacerbates ALI caused by LPS (“two-hit”) in C57Bl mice, and OGM2 attenuates these effects. FIG. 38A: C57Bl mice exposed to HTV (30 ml/kg, 4 hr) with or without LPS pre-treatment (0.8 mg/kg, i/t, 16 hour) exhibit greater Evans blue assay of lung vascular leak that mice exposed to spontaneous ventilation or LPS alone. Treatment with OGM2 (40 mg/kg ip) 15 minutes before HTV with or without LPS exposure, significantly attenuated lung vascular leak. Lungs from mice treated with OGM2 are marked with “+”. FIG. 38B: OGM2 significantly attenuated the rise in bronchoalveolar lavage (BAL) cell count in the setting of ALI induced by HTV, LPS and both (HTV/LPS). FIG. 38C: OGM2 significantly attenuated the rise in bronchoalveolar lavage (BAL) protein concentration in the setting of ALI induced by HTV, LPS and both (HTV/LPS). *p<0.05 vs. control; **p<0.05 OGM-8345 vs. vehicle; n=5. FIG. 38D: OGM2 blunted the rise in inflammatory cytokine mRNA expression in the ALI triggered by LPS, HTV or both. *p<0.05 OGM vs. vehicle: n=4. Here, OGM and OGM8345=OGM2.

FIG. 39A, FIG. 39B, and FIG. 39C show that OGM17, a highly potent analog of OGM2, blocked LPS-induced ALI in vivo. In mice exposure to LPS (0.7 mg/kg, intratracheally), OGM2 and OGM17 delivered intraperitoneally, reduced inflammatory cell infiltrates in BAL. PMN (polymorphonuclear neutrophils).

FIG. 40 presents a model by which the OGM class of compounds can serve as pharmacological countermeasures to prevent or treat acute lung injury (ALI) triggered by variety of causes, including infection, toxin and chemical exposure, ventilator injury (ventilator-induced barotrauma) and trauma. By blunting lung vascular leakage and inflammation, the OGM class of compounds will prevent and reverse ALI-induced respiratory failure acutely and prevent chronic sequela such as lung fibrosis and alveolar destruction.

FIG. 41 shows the structure activity relationship (SAR) analysis and identification of OGM2 (also known as OGM8345), which is a highly selective inhibitor of GPR68.

FIG. 42A through FIG. 42C: Quantification of GPR68 knockdown and inhibition phenotypes. Morpholino-injected embryos exhibited dose-dependent increases in phenotype prevalence with a shortened body axis (FIG. 42A), abnormal pigmentation (FIG. 42B), and defects in notochord (FIG. 42C) at 1.5 ng and 3 ng. The mismatched morpholino (3 ng) had minimal effects. OGM2 had increasing effects on these phenotypes at 10 and 20 μM. Cas9 targeting of GPR68 generated the same phenotype as both OGM2 treatment and GPR68 knockdown with a shortened body axis, abnormal pigmentation, and defects in notochord integrity. MM=mismatch morpholino; MO=morpholino.

FIG. 43 shows the target sequence of the zebrafish GPR68 morpholino (SEQ ID NO:5; ZDB-GENE-100419-2; GeneBank:CF347559).

FIG. 44 shows the target sequence of the zebrafish gpr68 sgRNA (ZDB-GENE-100419-2; GeneBank:CF347559 (SEQ ID NO:6 and SEQ ID NO:7).

FIG. 45A shows that acidic stimulation of GPR68 expressed in HEK 293 elicits a calcium response that is inhibited by OGM2. FIG. 45B shows that a serum-responsive element-luciferase (SRE-luc) reporter by itself had low basal activity in 293T cells. Upon co-transfection with GPR4, luciferase activity increased with acidification but was not inhibited by OGM2 at 1, 10, or 100 μM. By contrast, when GPR68 was co-transfected with SRE-luc and stimulated by acidification, 10 μM OGM2 completely inhibited the signal. (n=4, ****P <0.0001).

FIG. 46A and FIG. 46B: Glioblastoma cells activate GPR68 by acidifying their extracellular milieu. FIG. 46A provides a schematic representation of the extracellular pH reporter GPI-anchored pHluorin2 (pHluorin2-GPI), which increases in fluorescence intensity upon acidification. FIG. 46B is a graph showing a strong correlation between fluorescence intensity and extracellular pH in cells stably expressing pHluorin2-GPI, imaged at 469 nm excitation/525 nm emission.

FIG. 47A, FIG. 47B, and FIG. 47C show that pHluorin2-GPI fluorescence was quenched by the vital dye trypan blue, which is excluded from live cells, confirming that the visualized acidic microdomains are extracellular. FIG. 47A: confocal images; FIG. 47B: pHluorin2-GPI fluorescence; FIG. 47C: trypan blue quenching.

FIG. 48A and FIG. 48B show that U87 glioma cells expressing pHluorin2-GPI reporter exhibited higher-intensity fluorescence particularly at cellular protrusions in neutral pH media. Fluorescence was markedly attenuated within 20 seconds of buffering to pH 8.4 (After), confirming the correlation of fluorescence intensity with low extracellular pH.

FIG. 48C shows the quantitation of fluorescence intensity along the red line in FIG. 48A and FIG. 48C, and confirmed a drastic reduction at pH 8.4.

FIG. 49A and FIG. 49B: Glioma spheroids form organized highly acidotic cores not dependent on size. FIG. 49A shows representative images of spheroids made with U87 cells expressing pHluorin2-GPI, acquired over time from different seeding densities. FIG. 49B presents data on the quantitation of mean intensity of spheroids shown in FIG. 49A with 95% CI. The extracellular, environment of glioblastoma spheroids show progressive acidification detected via an increase in 469ex/525em of extracellular pH indicator pHluorin2-GPI (n=12 spheroids per condition).

FIG. 50A shows that the overall cellular intensity of the pHluorin2-GPI signal was reduced upon addition of alkaline buffer (P<0.05, n=6). FIG. 50B shows that when grown in spheroids, the extracellular acidification increases over time and becomes more organized.

FIG. 50C and FIG. 50D show a Kymograph of U87 cells (FIG. 50C) responding to acidification (stimulation) with calcium release. OGM2 (FIG. 50D) greatly attenuated acid-induced calcium release, in contrast to DMSO vehicle control.

FIG. 51 reveals that the peak calcium responses of U87 cells to acid stimulation in the presence of the GPR68 inhibitor OGM2, the GPR4 inhibitor NE52-QQ or the PLC inhibitor U73122 are mediated specifically by the GPR68-PLC pathway, but not by GPR4 (N=6 OGM2, P<0.0001; NE52QQ, not significant; U73122, P<0.0001).

FIG. 52A through FIG. 52D: GPR68 regulates cell survival in Glioblastoma. FIG. 52A shows that OGM2 is a more potent inhibitor of U87 cell growth in 2D cell assay than Temozolomide (TMZ). FIG. 52B shows that OGM2 is a more potent inhibitor of U87 spheroid growth than TMZ. FIG. 52C shows that OGM2, but not temozolomide (TMZ), caused dose-dependent inhibition of U138 cell growth in 2D culture. FIG. 52D shows that OGM2, but not TMZ, significantly decreased the growth of U138 3D spheroids.

FIG. 53A through FIG. 53C: OGM2 synergizes with the frontline therapeutic Temozolomide. FIG. 53A shows the dose response of OGM2 on PDX 08-387 glioblastoma cell survival. FIG. 53B shows data on concurrent treatment of PDX with OGM2 and TMZ at increasing concentrations of TMZ. FIG. 53C shows the calculated coefficient of drug interaction (CDI) of <1, indicated strong synergistic killing by OGM2 and TMZ.

FIG. 54A and FIG. 54B show that siRNA targeting GPR68 in U87 cells reduced GPR68 expression and reduced cell survival whereas control siRNA had no effect on either.

FIG. 54C and FIG. 54D show that siRNA targeting GPR68 in U138 cells reduced GPR68 expression and reduced cell survival while control siRNA had no effect. FIG. 54E, FIG. 54F, and FIG. 54G show that CRISPRi targeting GPR68 in U87 cells reduced both survival and expression of GPR68, while sgRNA alone and dCas9 alone have no effect on survival or expression. FIG. 54H, FIG. 54I, and FIG. 54J show that OGM2 reduced survival of U138s. U87 and U138 CRISPRi, n=3.

FIG. 55A through FIG. 55F: show that the OGM class of compounds reduces viability of Human Glioblastoma lines through GPR68. CRISPRi targeting GPR68 in U87 cells, with additional guides against GPR68 reduced both survival and expression of GPR68, while sgRNA alone and dCas9 alone have no effect on survival or expression (FIG. 55A, FIG. 55B, and FIG. 55C). CRISPRi targeting GPR68 in U138 cells, with additional guides against GPR68 reduced both survival and expression of GPR68, while sgRNA alone and dCas9 alone have no effect on survival or expression (FIG. 55D, FIG. 55E, and FIG. 55F).

FIG. 56A, FIG. 56B, FIG. 56C, and FIG. 56D show that OGM2 reduced survival of 4 different PDX lines in 2D cell survival assays.

FIG. 57A and FIG. 57B show that OGM2 reduced the viability of 2 PDX lines in 2D cell survival assays. FIG. 57C and FIG. 57D show that OGM2 reduced the viability of 2 PDX lines in 3D tumor spheroid assays. FIG. 57E shows that OGM2 reduced the viability of mouse GBM line GL261.

FIG. 58 provides a full list of GBM cell lines sensitive to killing by OGM class of compounds with LC₅₀ (Lethal concentration-50; concentration causing 59% lethality) for OGM2.

FIG. 59A through FIG. 59D: OGM2 causes specific cell death in glioblastoma cells. FIG. 59A shows that OGM2 reduced U87 viability, but not HEK293 cell viability. FIG. 59B shows the quantitation of the cell survival assay in FIG. 59A. FIG. 59C shows acridine orange stain of zebrafish embryos treated with OGM2 to visualize cell death. FIG. 59D shows that quantitation of results showed OGM2 did not cause significant excess death in zebrafish embryos.

FIG. 60A through FIG. 60C: Acid induced GPR68 activation promotes cell survival in glioblastoma. FIG. 60A shows that acidic media (pH 6.2) promoted U87 spheroid growth at 3 days, in comparison to the basic media (pH 8.0; P<0.0001). The enhanced growth in acidic media was blocked by OGM2 (P<0.0001) (N=8 for each condition). FIG. 60B shows the quantitation of data from FIG. 62A (N=8 for each condition). FIG. 60C shows the growth trends of U87 spheroids treated with Ogerin (OGRN), OGM2 and DMSO vehicle. OGRN promoted U87 spheroids growth rate, whereas OGM2 impaired spheroid growth rate. (N=8) for each condition).

FIG. 61 through FIG. 71A and FIG. 71B: RNA-seq of PDX GBM indicates OGM2 induces ferroptosis.

FIG. 61 is a heatmap of gene expression changes shows broad changes in transcriptomes in PDX cells after OGM2 treatment. Here, OGM, OGM1, and OGM2 all indicate replicates of OGM2 treatment.

FIG. 62 is a PCA comparison of transcriptomes demonstrates that differences across cell types are greater than differences induced by OGM2 treatment. This difference is also shown through hierarchical clustering in FIG. 64.

FIG. 63 is a chart showing that glioblastoma cancer lines, which are highly dissimilar at the transcriptional level, respond to OGM2 treatment with comparatively small transcriptional changes, including induction of ferroptosis-associated genes.

FIG. 64A, FIG. 64B, and FIG. 64C show that the number of significantly differentially expressed (SDE) genes from 913, 08-387, and Mayo PDX cells after OGM2 treatment were 837, 687, and 309, respectively (FDR <0.01).

FIG. 65 shows gene set enrichment analysis of shared pathways altered by OGM2 treatment.

FIG. 66 is a table that shows data on seven Shared differentially expressed genes.

FIG. 67A is a comparison of SDE genes, of which only 7 common genes were altered in all three OGM2-treatment groups. FIG. 67B is a string analysis for 7 common genes altered by OGM2 treatment.

FIG. 68 is a subset of Gene set enrichment analysis of terms implicate ferroptosis as a mechanism of cell death. FIG. 65 provides a full analysis for the data presented in FIG. 75.

FIG. 69A through FIG. 69E: OGM2 induces hallmarks of ferroptosis. Known markers and mediators of ferroptosis were increased in PDX cells treated with OGM2: FIG. 69A, ASNS; FIG. 69B, FTH1; FIG. 69C, FTL; FIG. 69D, HMOX1; and FIG. 69E, SLC3A2.

FIG. 70A through FIG. 70C: Known suppressors of ferroptosis were decreased in PDX cells treated with OGM2. FIG. 70A, CA9; FIG. 70B, FADS2; and FIG. 70C, SREBF1.

FIG. 71A (ATF4) and FIG. 71B (CHAC1) show involvement in both ferroptosis and ER stress response, were increased in OGM2 treatment groups.

FIG. 72A through FIG. 72E: OGM2 induces ferroptosis but not apoptosis. FIG. 72A is a western blot which shows that OGM2 increased protein expression of TFRC, a marker of ferroptosis. OGM2 also increased protein expression of HO-1, a marker of oxidative stress.

FIG. 72B shows that OGM2 did not increase cleaved caspase 3 (apoptosis maker) level, whereas doxorubicin, a positive control apoptosis inducer, did. FIG. 72C shows the quantitation of 3 biological replicates of western blots for TFRC shown in FIG. 72A. FIG. 72D shows the quantitation of 3 biological replicates of western blots for HO-1 as shown in FIG. 72A. FIG. 72E shows the quantitation of 3 biological replicates of western blots for cleaved Caspase 3 as shown in FIG. 72B.

FIG. 73 is a graph showing that OGM2 decreased GSH levels in U87 cells.

FIG. 74A through FIG. 74B show that OGM2 significantly increased lipid peroxidation, the canonical marker of ferroptosis, in U87 and U138 human glioblastoma cells. Erastin, a cystine-glutamate antiporter system X_{c} inhibitor, is a positive control ferroptosis inducer. Chi square >4 is considered significantly different with p<0.01. FIG. 74A shows OGM2 induces ferroptosis in U87 cells based on Liperfluo™ lipid peroxidation assay. FIG. 74B shows OGM2 induces ferroptosis in U138 cells.

FIG. 75A shows that erastin, a nonselective ferroptosis inducer, but not OGM2, increased lipid peroxidation in HEK293. FIG. 75B shows that erastin, but not OGM, dramatically reduced HEK293 cell survival. Thus, in contrast to erastin, OGM2 induces ferroptosis selectively in cancer cells.

FIG. 76A and FIG. 76B show that OGM2 disrupted mitochondria structure (Mitotracker) in U87, but not lysosome structure (lysotracker).

FIG. 77 is a graph showing that OGM2 disrupted mitochondria function in U87 glioblastoma cells, as assessed by TMRM (tetramethylrhodamine methyl ester perchlorate), a mitochondrial membrane potential indicator.

FIG. 78 shows the TEM ultrastructure of U87 cells treated with OGM2 show disrupted mitochondria (*) with normal ER (▴), providing further evidence that OGM2 induces ferroptosis in cancer cells.

FIG. 79A through FIG. 79F: OGM2 synergizes with ionizing radiation to induce ferroptotic cell death in glioblastoma cells. FIG. 79A through FIG. 79F show that OGM2 synergizes with radiation in glioblastomas. FIG. 79A, FIG. 79B, and FIG. 79C provide data showing that OGM2 and 2Gy ionizing radiation demonstrated very strong synergy (CDI <0.06) for inducing lipid peroxidation in U87 cells. All treatments were highly significant with Chi-squared >4 indicating a significant difference with p<0.01. FIG. 79D, FIG. 79E, and FIG. 79F provide data showing that OGM2 and 2Gy ionizing radiation demonstrated exceptionally strong synergy (CDI <0.006) for inducing lipid peroxidation in U138 cells. All treatments were highly significant with Chi-squared >4 indicating a significant difference with p<0.01.

FIG. 80A through FIG. 80D: Loss of GPR68 causes ferroptosis in GBM. FIG. 80A, FIG. 80B, FIG. 80C, and FIG. 80D show that siRNA knock-down of GPR68 increased expression of the ferroptosis markers TFRC. ATF4, and CHAC1, and the oxidative stress marker HMOX1 in U87 cells, like the OGM2 treatment, whereas control siRNA had no effect.

FIG. 81A, FIG. 81B, FIG. 81C, and FIG. 81D show that CRISPRi knock-down of GPR68 increased expression of TFRC, ATF4, CHAC1, and HMOX1 in U87 cells, while guide RNAs or dCas9 alone had no effect.

FIG. 82A and FIG. 82B: Knockdown of GPR68 induces ATF4 transcriptional target SLC7A11. FIG. 82A and FIG. 82B show that siRNA knock down of GPR68 increased expression of SLC7A11 in U87 and U138 cells.

FIG. 83A, FIG. 83B, FIG. 83C, and FIG. 83D show that siRNA knock down of GPR68 increased expression of TFRC, ATF4, CHAC1, and HMOX1 in U138 cells.

FIG. 84A, FIG. 84B, FIG. 84C, and FIG. 84D show that CRISPRi knock-down of GPR68 increased expression of TFRC, ATF4. CHAC1, and HMOX1 in U138 cells.

FIG. 85A and FIG. 85B show that CRISPRi knock-down of GPR68 increased expression of SLC7A11, while guide RNAs or dCas9 alone had no effect, in U87 and U138 cells.

FIG. 86A through FIG. 86B show data indicating that OGM2 induces ferroptosis in cancer cells through upregulation of ATF4. FIG. 86A: CRISPRi knock-down of ATF4 prevented OGM2-induced cell death in U87 cells, while guide RNAs or dCas9 alone had no effect on survival. FIG. 86B: Knock-down of ATF4 prevented OGM2-induced cell death in U138 cells. CRISPRi successfully reduced ATF4 expression even in the setting of OGM-induced expression in U87 cells and in U138 cells.

FIG. 87A through FIG. 87F show data indicating that OGM2 induces ferroptosis through upregulation of ATF4. CRISPRi knock-down prevented OGM2-induced expression of ferroptosis marker TFRC in U87 cells (FIG. 87A) and in U138 cells (FIG. 87B). CRISPRi knock-down of ATF4 prevented OGM2 induced expression of direct ATF4 target CHAC1 in U87 cells (FIG. 87C) and in U138 cells (FIG. 87D). CRISPRi knockdown of ATF4 prevented OGM2 induced increase in oxidative stress response marker HMOX1 in U87 cells (FIG. 87E) and in U138 cells (FIG. 87F).

FIG. 88A through FIG. 88B: ATF4 knockdown prevents induction of Ferroptosis indicators by OGM2. These results indicate that ATF4 loss is a potential mechanism for tumor resistance to GPR68 inhibition and that expression of ATF4 will overcome potential resistance of cancer cells, acquired or otherwise, to ferroptosis induction. FIG. 88A and FIG. 88B show that CRISPRi knock-down prevented OGM2-induced expression of ferroptosis marker TFRC in U87 cells (FIG. 88A) and in U138 cells (FIG. 88B).

FIG. 89A and FIG. 89B show that CRISPRi knock-down of ATF4 prevented OGM2 induced expression of direct ATF4 target CHAC1 in U87 cells (FIG. 89A) and in U138 cells (FIG. 89B).

FIG. 90A and FIG. 90B show that CRISPRi knockdown of ATF4 prevented OGM2 induced expression of direct ATF4 target SLC7A11 in U87 cells (FIG. 90A) and in U138 cells (FIG. 90B).

FIG. 91A and FIG. 91B show that CRISPRi knockdown of ATF4 prevented OGM2-induced increase in oxidative stress response marker HMOX1 in U87 cells (FIG. 91A) and in U138 cell (FIG. 91B).

FIG. 92A through FIG. 92H show that ATF4 targeting guides alone and Cas9 alone have no effect on Ferroptosis markers. Controls for ATF4 CRISPRi knock-down had no effect on expression of ferroptosis marker TFRC in U87 cells (FIG. 92A) and in U138 cells (FIG. 92B). Controls for ATF4 CRISPRi knock-down had no effect on expression of direct ATF4 target CHAC1 in U87 cells (FIG. 92C) and in U138 cells (FIG. 92D). Controls for ATF4 CRISPRi knock-down had no effect on expression of direct ATF4 target SLC7A11 in U87 cells (FIG. 92E) and in U138 cells (FIG. 92F). Controls for ATF4 CRISPRi knock-down had no effect on expression of oxidative stress response marker HMOX1 in U87 cells (FIG. 92G) and in U138 cells (FIG. 92H).

FIG. 93 presents a model depicting GPR68 mediated inhibition of ferroptosis.

FIG. 94A shows that OGM2 treatment increases immunogenic cell death markers in U87 glioblastoma cells, including the release of ATP into the extracellular environment (FIG. 94A) at 6 hours post-treatment, similar to ferroptosis inducer erastin and higher than the apoptosis inducer Doxyrubicin (DOX). FIG. 94B and FIG. 94C: OGM2 treatment increases calreticulin (CRT) externalization, another marker of immunogenic cell death, in cancer cells at 3 and 6 hours post-treatment.

FIG. 95 shows that OGM2 reverses lactate-mediated immune-suppression, as assessed by NF-kB p65 activation in bone-marrow derived macrophages (BMMPs). BMMP cells were treated with soluble lactic acid (sLA) or nanoparticles composed of PLA-PEMA (PLA), which breakdown into lactic acid, or polystyrene-COOH (PS). The addition of OGM2, a GPR68 inhibitor, reverses the inhibition of p65 activation seen with PLA following particle incubation and 1-hr LPS stimulation. Samples were immunoblotted for phosphor-NF-κB p65 (Ser536), total NF-κB p65, phosphor-IκB (Ser32), total IκB, phosphor-p38 (Thr180/Tyr182), total p38, and β-actin.

FIG. 96A through FIG. 96D present data showing that GPR68 inhibition synergizes with radiation to induce more G2/M arrest in cancer cells, increasing their sensitivity to ionizing radiation. Cell cycle analysis of OGM2 treatment and irradiation in PANC02 pancreatic cancer cells (FIG. 96A). Quantification of % change in G2 phase fraction from (A) shows synergy between OGM2 and 3Gy radiation (FIG. 96B) with CDI (coefficient of drug interaction)<1 indicates synergy. FIG. % C and FIG. 96D show additive PANC02 cell killing by combination of OGM2 and ionizing radiation. FIG. 96E and FIG. 96F show additive A549 lung cancer cell killing by combination of OGM2 and ionizing radiation.

FIG. 97A through FIG. 97F show that OGM2 synergizes with ionizing radiation in inducing ferroptosis in lung and pancreatic cancer. FIG. 97A, FIG. 97B, and FIG. 97C provide data showing that OGM2 and ionizing radiation demonstrated very strong synergy (CDI <0.232) for inducing lipid peroxidation (ferroptosis) in mouse Panc02 cells. All treatments were highly significant with Chi-squared >4, which indicates a p<0.01. FIG. 97D, FIG. 97E, and FIG. 97F provide data showing that OGM2 and ionizing radiation demonstrated very strong synergy (CDI <0.232) for inducing lipid peroxidation (ferroptosis) in A549 cells. All treatments were highly significant with Chi-squared squared >4 which indicates a p<0.01.

FIG. 98A through FIG. 98E: OGM2 induces key ferroptosis markers in patient derived GBM lines. OGM2 induced key markers of ferroptosis ATF4 (FIG. 98A), CHAC1 (FIG. 98B), HMOX1 (FIG. 98C), TFRC (FIG. 98D), and SLC7A11 (FIG. 98E), confirmed via qPCR.

FIG. 99A through FIG. 99B: OGM2 induces Ferroptosis through upregulation of ATF4. FIG. 99A shows that CRISPRi knockdown of ATF4, with additional sgRNAs, prevented OGM2-induced cell death in U87 cells, while guide RNAs or dCas9 alone had no effect on survival. FIG. 99B shows that knock-down of ATF4 prevented OGM2-induced cell death in U138 cells. Additional ATF4 targeting CRISPRi successfully reduced ATF4 expression even in the setting of OGM2-induced expression in U87 cells and in U138 cells.

FIG. 100A through FIG. 100D show that OGM2 significantly increased lipid peroxidation, the canonical marker of ferroptosis, and reduce viability in DU145 and PC3 human prostate cancer cells. OGM2 induces ferroptosis in DU145 cells (FIG. 100A) and PC3 cells (FIG. 100C) based on Liperfluor™ lipid peroxidation assay. Erastin, a cystine-glutamate antiporter system X_{c}⁻ inhibitor, is a positive control ferroptosis inducer. Chi square >4 is considered significantly different with p<0.01. OGM2 reduces viability of DU145 cells (FIG. 100B) and PC3 cells (FIG. 100D) based on CellTiter-Glo assay2.

FIG. 101A and FIG. 101B show data related to the externalization of calreticulin (CRT), a damage-associated molecular pattern (DAMP).

FIG. 102 is a bar graph relating to ATP secretion. N=4 for each condition. Additional ATF4 targeting CRISPRi successfully reduced ATF4 expression even in the setting of OGM-induced expression in U87 cells in U138 cells.

### DETAILED DESCRIPTION OF EMBODIMENTS OF THE INVENTION

### 1. Overview

A hallmark of glioblastoma is its acidic extracellular tumor microenvironment (TME), which drives cancer progression by promoting malignant clonal selection, metastasis, and immune escape. For example, extracellular acidification can trigger significant pro-oncogenic transcriptional responses that provide growth advantage to the tumor. However, the mechanism by which these extracellular pH changes are sensed by the cancer are generally not fully understood. In medulloblastoma cells, extracellular acidification triggers calcium (Ca²⁺) fluxes in a phospholipase C (PLC)-dependent manner, implicating the involvement of a GPCR. GPR68, also known as ovarian cancer G-coupled protein receptor 1 (OGR-1), is a member of the proton sensing GPCR family which is activated in response to subtle extracellular acidification (inactive at pH 7.4 and fully active at pH 6.4). The receptor is known to couple to Gq/11, which leads to activation of the phospholipase C/Ca²⁺ pathway, as well as Gs, leading to activation of the adenylyl cyclase/cAMP pathway, and G13, which activates Rho. An increasing body of evidence points to a potential role for acid-sensing GPCRs in the progression of a variety of cancers. A novel class of small molecule GPR68/OGR-1 inhibitors named ogremorphins demonstrate that genetic and pharmacological disruption of GPR68 signaling in glioblastoma cells results in ferroptosis, an iron mediated cell death program, in a ATF4-dependent manner.

Acute respiratory distress syndrome (ARDS) is a serious and potentially life-threatening condition that affects the respiratory system. It is characterized by inflammation, vascular leakage and fibrosis of the lungs, which can lead to respiratory failure and dangerous low oxygen levels in the blood. ARDS can be caused by a variety of factors, including infection, such as pneumonia and tuberculosis, sepsis, trauma, toxins, inhaling toxic substances, such as smoke or chemical fumes aspirating vomit or stomach contents. An increasing body of evidence points to ventilation (Ventilator induced lung injury, VILI) and acid (from aspiration subsequent to long term intubation) being two mechanisms that can cause ARDS. GPR68 has been described as sensing both of these stimuli and have expression in various relevant cell types in the lungs. A novel class of small molecule GPR68/OGR-1 inhibitors named ogremorphins demonstrate that genetic and pharmacological disruption of GPR68 signaling in the mouse preclinical model of ARDS can attenuate the vascular leakage and inflammation, reversing or preventing acute lung injury/ARDS, which can be life-threatening on its own but can also lead to chronic sequela such as lung fibrosis and chronic lung disease.

Disruption of the positive feed forward pathologic loop that results from gastric acid aspiration and that subsequently leads to ARDS, by inhibiting GPR68, is an attractive and logical therapeutic strategy. Thus, an inhibitor or antagonist of GPR68 could be administered to a mammal, including a human subject with COPD, especially with aspiration, in an amount effective to prevent or to attenuate ALI/ARDS, including chronic sequelae such as lung fibrosis.

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the term “subject” or “patient” refers to any mammal (preferably a human) which is diagnosed with a malignancy, an acute lung injury, or an autoimmune/inflammatory condition, is suspected of suffering from a malignancy, an acute lung injury, or an autoimmune/inflammatory condition, or is at risk for developing a malignancy, an acute lung injury, or an autoimmune/inflammatory condition.

As used herein, the term “mammal” refers to any mammalian species, including humans, laboratory animals, zoo animals, farm animals, companion animals, service animals, and the like. In particular, humans, and animals such as apes, monkeys, rodents, bovines, equines, caprines, canines, felines are included in the definition of “mammal” as known in the art of biology.

As used herein, the terms “treating,” “treatment.” and their cognates, refers to an action taken to obtain a desired pharmacologic and/or physiologic therapeutic effect. “Treatment,” therefore, includes: (a) preventing the condition or disease or symptom thereof from occurring in a subject which may be predisposed to the condition or disease but has not yet been diagnosed as having it; (b) inhibiting the condition or disease or symptom thereof, such as, arresting its development: (c) relieving, alleviating or ameliorating the condition or disease or symptom thereof, such as, for example, causing regression of the condition or disease or symptom thereof, and (d) reducing the amount or frequency of standard drugs needed to treat the condition on a continuing basis.

As used herein, the terms “prevent,” “prevention,” and their cognates, refers to complete prevention of a disease or condition such that the disease or condition does not occur, and also includes decreasing the likelihood of a subject contracting or developing the disease or condition, causing the disease or condition to occur less frequently or with less severity in a subject or in a population, and shortening the duration of a disease or condition in a subject or a population.

As used herein, the terms “malignancy.” “malignant,” and “cancer” refer to a hyperproliferative disorder, disease, or condition with the presence of cancerous cells. Malignancies or cancer generally are characterized by anaplasia, invasiveness, and/or metastasis, but these do not occur in all cases. Examples of diseases and conditions falling under the definition of cancer include, but are not limited to, carcinomas, sarcomas, lymphomas and leukemias (e.g., acute monocytic leukemia, B-cell lymphoma, chronic myelogeous leukemia), germ cell tumors, and blastomas (e.g., glioblastoma, medulloblastoma). Specifically included are cancers of the blood, kidney, lung (e.g., lung adenocarcinoma, lung large cell cancer, lung small cell cancer, lung squamous cell), skin (e.g., melanoma), pancreas (e.g., pancreatic adenocarcinoma), brain, breast (e.g., breast adenocarcinoma), cervix, prostate (e.g., prostate adenocarcinoma), ovary, liver (e.g., hepatocellular), glioblastoma, medulloblastoma, and the like, such as neuroendocrine prostate cancer, and melanoma. In particular, glioblastoma, prostate, colon or rectal (e.g., colon adenocarcinoma, colorectal adenocarcinoma), breast, lung, and pancreatic cancer are included in this definition.

As used herein, the term “autoimmune/inflammatory condition” refers to any disease or condition known as autoimmune diseases in the art and as inflammatory diseases in the art. These two groups of conditions overlap to a degree and are grouped together here. In general, an “autoimmune disease” is a condition arising from an abnormal immune response of the body which attacks itself. Major autoimmune diseases include, celiac disease, diabetes type 1, Graves' disease, inflammatory bowel disease, multiple sclerosis, psoriasis, rheumatoid arthritis, and systemic lupus erythematosus.

Inflammatory disorders and conditions broadly are those involving inflammation and are often mediated by the immune system. Such disorders include cancer, ischemic heart disease, atherosclerosis, asthma, chronic obstructive pulmonary disease (COPD), osteoporosis, chronic inflammatory disease, inflammatory pain syndrome, celiac disease, colitis, diverticulitis, inflammatory bowel disease, hypersensitivities, rheumatic fever, rheumatoid arthritis, sarcoidosis, vasculitis, and the like. Thus, any of these type of diseases and conditions are contemplated for treatment and prevention by embodiments of this invention. In particular, autoimmune diseases such as type 1 diabetes (T1D), rheumatoid arthritis (RA) and multiple sclerosis (MS), irritable bowel disease (IBS), are part of this invention, as well as allergic conditions such as seasonal pollen allergy, pet dander or mold, or infectious or hyperproliferative disorders. For asthma, an inhibitor or antagonist of GPR68 could be administered to a mammal, including a human subject with controlled asthma, in an amount effective to reduce the likelihood of exacerbations and to reduce the amount of standard of care intranasal corticosteroids necessary for asthma control. Conditions and diseases of this type that are amenable to treatment by the invention which define an appropriate subject or patient will be discerned easily by the person of skill in the art based on the disclosures herein.

As used herein, the term “acute lung injury” refers to any known disease or condition regardless of cause that has a sudden or severe damage to the alveoli, or air sacs, or the lungs. Acute lung injury may be caused by a variety of factors, including toxic exposure, respiratory infections, and physical trauma. This damage results can cause any or all of the following features, inflammation, vascular leakage and fibrosis causing difficulty breathing and oxygen deprivation to the body's tissues. In particular, the term “acute respiratory distress” refers to conditions (also known as acute respiratory failure or acute respiratory distress syndrome (ARDS)). It is a severe respiratory condition characterized by rapid onset and severe difficulty breathing. It is often caused by a severe infection, injury, or inflammation in the lungs, leading to fluid accumulation and inflammation in the alveoli (air sacs) and impaired oxygen exchange in the blood. Symptoms may include shortness of breath, rapid breathing, chest pain, and a bluish color to the skin due to low oxygen levels. Once initiated, ARDS is fatal in 36% of cases though it can be higher, as seen in the early days of COVID19 pandemic. Treatment may include oxygen therapy, mechanical ventilation, and medications to reduce inflammation and fluid accumulation in the lungs. Examples of conditions that may lead to acute respiratory distress include pneumonia, sepsis, trauma, and inhalation injury.

As used herein, the term “anticancer agent” refers to a therapeutic agent that has the effect of killing, decreasing the size of, preventing metastasis of, reducing metastasis of, halting the growth of, reducing the speed of growth of, or ameliorating a symptom of cancer.

As used herein, the term “anti-inflammatory agent” refers to a therapeutic agent that has the effect of preventing, ameliorating, reducing the severity of, reducing the likelihood of, or ameliorating a symptom of an autoimmune/inflammatory condition as discussed here, particularly relating to inflammation of/injury to the lung, and various types of pneumonia.

As used herein, the terms “administer,” “administration,” and their cognates, refers to contacting a subject with a therapeutic compound or composition. As used herein, “administering” and its cognates refers to introducing an agent to a subject, and can be performed using any of the various methods or delivery systems for administering agents and pharmaceutical compositions known to those skilled in the art. Modes of administering include, but are not limited to oral administration or intravenous, subcutaneous, intramuscular or intraperitoneal injections, rectal administration by way of suppositories or enema, or local administration directly into or onto a target tissue (such as the pancreas or skin), or administration by any route or method that delivers a therapeutically effective amount of the drug or composition to the cells or tissue to which it is targeted. The method of administration is selected by the practitioner based on the disease to be treated, the target tissue or cell type, and the desired pharmacokinetic and pharmacodynamic properties. The targeting ligand is selected by the practitioner based on its affinity for a receptor or antigen expressed on the target tissue or cell type. Prodrug formulations can be designed to release the active RNA molecule in response to a specific physiological condition, such as a change in pH or an enzyme activity. Conjugate formulations can comprise a carrier molecule that enhances the stability or cellular uptake of the RNA molecule.

As used herein, the term “therapeutic agent” refers to any compound that exerts a “therapeutic effect” (or pharmaceutical composition that contains such a compound). A “therapeutic effect” is a pharmacological or physiological effect of preventing, curing, delaying, ameliorating, improving, shortening the duration of, decreasing the likelihood of, decreasing the symptoms of, and the like, a disease or condition in a subject.

### 3. Overview

Embodiments of this invention relate to Ogremorphin, a first-in-class inhibitor of proton-sensing GPCR GPR68. Discovered from a chemical genetic zebrafish screen, Ogremorphin perturbs neural crest migration. Furthermore, Ogremorphin inhibits migration in a number of human melanoma lines which derive from the neural crest. Phenome-wide association study identified a natural variant that is ectopically active and is associated with metastasis of common cancers. The GPR68 activity is associated with an increased ability for cancer cell migration and contributes to metastasis, making the compounds according to this invention suitable for treatment of hyperproliferative and autoimmune or chronic inflammatory conditions, and acute lung injury.

The diseases contemplated for treatment using this invention include acute lung injury and symptomology of acute respiratory distress syndrome as a result of bacterial infection, viral infection, inhalation injury, trauma, or mechanical ventilation-induced barotrauma; cancer treatment as a monotherapy for a lymphoma, a leukemia (including but not limited to monocytic leukemia, B-cell lymphoma, chronic myelogeous leukemia), a germ cell tumor, a blastoma, a sarcoma, a blood cancer, a skin cancer (including but not limited to melanoma and basal cell carcinoma), a breast cancer (including but not limited to her2⁺, ER⁺ or triple negative subtypes), a cervical cancer, an ovarian cancer, a prostate cancer (including neuroendocrine prostate cancer), a kidney cancer, a lung cancer (including but not limited non-small cell lung cancer, small cell lung cancer and mesothelioma), a pancreatic cancer, a liver cancer, a colon or colorectal cancer, and a brain cancer (including but not limited to gliomas such as glioblastoma multiforme, medulloblastoma, ependymoma, and chordoma). This treatment can also be co-administered (the same day or preceding up to a week before treatment) with other chemotherapeutics (including but not limited to temozolomide and doxorubicin), or with radiation, or with immunotherapy agents including but not limited to (ipilimumab, pembrolizumab, nivolumab, and atezolizumab).

### 4. Embodiments of the Invention

A class of small molecules, Ogremorphins (OGMs), were found to inhibit GPR68, causing robust and selective cancer cell death in a dose-dependent manner. Acidic TME activates the extracellular proton-sensing receptor GPR68, which activates anti-cancer pathways such as ferroptosis. Small molecule GPR68 inhibitors induce ferroptosis, an iron-dependent programed cell death pathway, selectively in cancer cells. Moreover, small molecule GPR68 inhibitor and ionizing radiation therapy induces ferroptosis in cancer cells in a synergistic manner. Additionally, since ferroptosis is a form of immunogenic cell death, blocking GPR68 signaling can promote anti-tumor immune response. Thus, acidic TME and GPR68 is a promising therapeutic target for GBM and other cancers, and compound and genetic strategies to block GPR68 signaling are valuable as cancer therapeutics, in conjunction with cancer immunotherapies and traditional therapies such as ionizing radiation.

Inhibiting or knocking out/down GPR68 is an effective method to selectively kill GBM cells. In TMZ-sensitive glioblastoma, lower doses can be used due to synergistic effects allowing for greater tolerance and reduced side effects. TMZ is the only FDA approved drug for GBM. In addition, GPR68 represents an attractive target for therapeutic intervention for GBM and other cancers. Pharmacological blockade of GPR68 with OGMs or genetic ablation of GPR68 may be singularly effective as treatment for both treatment-naïve and treatment-resistant glioblastomas, in conjunction with the standard therapy. TMZ, with radiation, or with cancer immunotherapies, including check point inhibitors (e.g., PD-1/PD-L1 and CTLA-4/B7-1/B7-2). Moreover, since ferroptosis is a form of immunogenic cell death, blocking GPR68 signaling can promote anti-tumor immune response. In summary, since acidic tumor microenvironment is a general mechanism that promotes cancer progression and therapeutic resistance, pharmacological blockade of GPR68 with OGMs, or genetic ablation or gene knockdown of GPR68 can be effective as anti-cancer agent for wide variety of cancers, in conjunction with traditional chemotherapies and novel cancer immunotherapies. This specification describes a method to directly kill cancer cells as well as to target the acidic tumor microenvironment to induce programed cell death by ferroptosis. The method also is useful for treatment of acute lung injury.

An inhibitor of the proton-sensing GPCR (G protein-coupled receptor), GPR68, which modulates migratory behavior of melanoma cells in vitro and in vivo was identified. Targeting proton dynamics, and the dysregulation of pH has emerged as a possible therapeutic avenue for cancer and GPR68 has been implicated in the regulation of cancer in diverse and even divergent ways. GPR68, when overexpressed in PC3 prostate cancer cells has significantly reduced metastasis. Furthermore, in ovarian cancer cells HEY1, overexpression of GPR68 reduced cell migration and increased cell adhesion. However, in medulloblastoma GPR68 activation increases TRPC4 activity which enhances tumor cell migration. GPR68 has been identified as a regulator of cancer-associated fibroblasts in colon cancer and pancreatic cancer and may play a critical role in regulating cancer progression.

The results presented here demonstrate that Ogremorphin-1 (OGM1) is a reversible GPR68 inhibitor, a first-in-class negative regulator of this molecule. Additionally, embodiments of this invention relate to a role for GPR68 in zebrafish development. Previous studies have shown that GPR68 is expressed during the early development of zebrafish and responds to acidification like its mammalian homolog. Inhibition of GPR68 during neural crest migration affects the development of pigmentation and craniofacial cartilage formation is demonstrated here. In Xenopus, v-ATPase regulates pH in a regional manner to affect craniofacial morphogenesis; recent studies of GPR68 have shown that the receptor senses flow and stretch. However, both of these stimuli are dependent on mildly acidic conditions. While it is still not clear if GPR68 is sensing protons in neural crest cells or the surrounding tissues, this is the first data that suggests that there could be proton mediated signaling events that are critical for normal development in zebrafish. Notably, treatment of zebrafish with some proton efflux machinery phenocopies OGM2 treatment while others do not.

Recent studies have focused on the mechanisms through which the acidification and pH modulation affects cancer cell behavior through proton-sensing receptors such as GPR4, GPR65, and GPR68. In both in vitro and in vivo models, acidification promotes melanoma cell migration. Consistent with this observation, the studies presented here with OGM2 suggest that melanoma cells sense acidification through GPR68 to modulate their migration. Furthermore, using a functional genomics approach, the data presented here show that rs61745752, which results in a truncation of the c-terminal tail of GPR68 upstream of a putative O-arrestin binding domain, causes a loss of receptor internalization and an aberrant receptor activation, and is associated with increased risk of cancer.

Preferred compounds identified here as GPCR68 inhibitors include, but are not limited to:

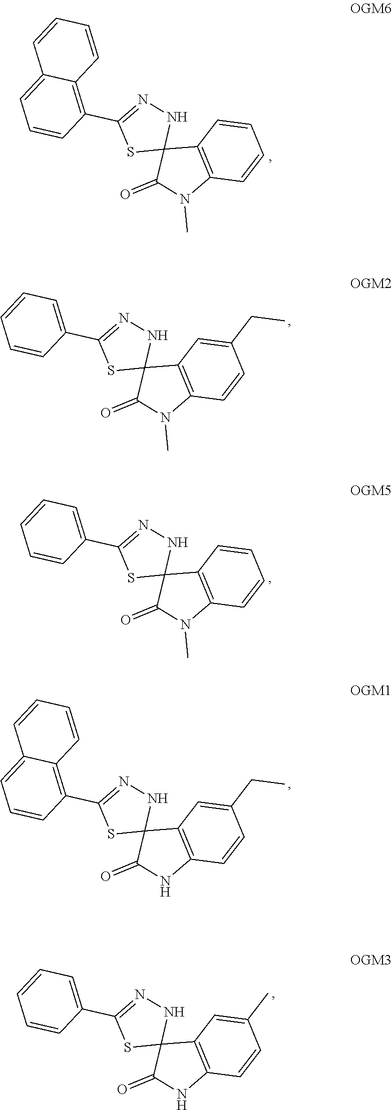 [SMILES: CN1C(=O)C2(NN=C(c3cccc4ccccc34)S2)c2ccccc21 ; CCc1ccc2c(c1)C1(NN=C(c3ccccc3)S1)C(=O)N2C ; CN1C(=O)C2(NN=C(c3ccccc3)S2)c2ccccc21 ; CCc1ccc2c(c1)C1(NN=C(c3cccc4ccccc34)S1)C(=O)N2 ; Cc1ccc2c(c1)C1(NN=C(c3ccccc3)S1)C(=O)N2]

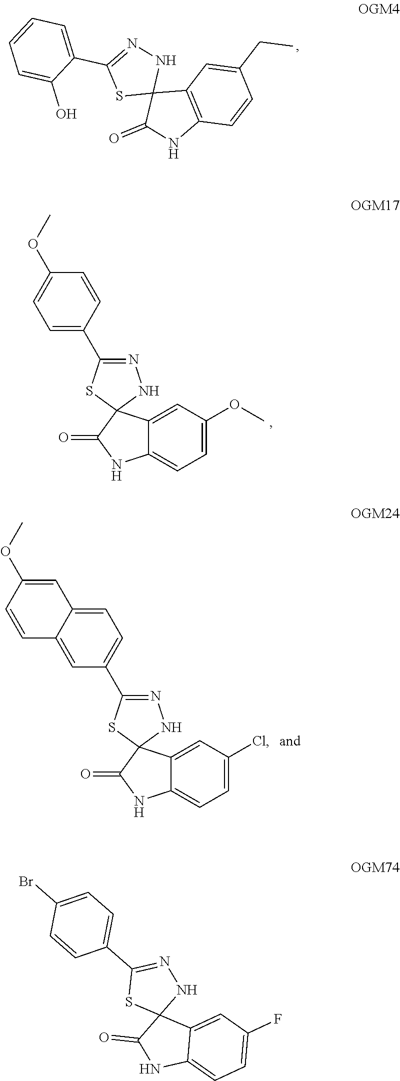 [SMILES: CCc1ccc2c(c1)C1(NN=C(c3ccccc3O)S1)C(=O)N2 ; COc1ccc(C2=NNC3(S2)C(=O)Nc2ccc(OC)cc23)cc1 ; COc1ccc2cc(C3=NNC4(S3)C(=O)Nc3ccc(Cl)cc34)ccc2c1 ; "and" ; O=C1Nc2ccc(F)cc2C12NN=C(c1ccc(Br)cc1)S2]

Other compounds suitable for use with embodiments of the invention are described below. Embodiments of the invention include the compounds disclosed here.

Suitable compounds for use in the inventive methods include, but are not limited to OGM1, OGM2, OGM17, OGM22, and OGM74. Preferred compounds are OGM17 and OGM74.

The compounds of the invention include the base, and any pharmaceutically acceptable hydrate, solvate, acid or salt, and can be amorphous or in any crystalline form, or as an oil or wax. Any pharmaceutically acceptable salt can be used, as may be convenient.

Generally, these salts are derived from pharmaceutically and biologically acceptable inorganic or organic acids and bases or metals. Examples of such salts include, but are not limited to: acetate, adipate, alginate, ammonium, aspartate, benzoate, benzenesulfonate (besylate), bicarbonate, bisulfate, butyrate, citrate, camphorate, camphorsulfonate, carbonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, magnesium, maleate, malonate, methanesulfonate (mesylate), 2-naphthalenesulfonate, nicotinate, nitrate, oxalate, palmoate, pectinate, persulfate, 3-phenylpropionate, phosphate, picrate, pivalate, potassium, propionate, salicylate, sodium, succinate, sulfate, tartrate, thiocyanate, toluenesulfonate (tosylate) and undecanoate salts.

The compounds also include any or all stereochemical forms of the therapeutic agents (i.e., the R and/or S configurations for each asymmetric center). Therefore, single enantiomers, racemic mixtures, and diastereomers of the therapeutic agents are within the scope of the invention. Also within the scope of the invention are steric isomers and positional isomers of the therapeutic agents. The therapeutic agents of some embodiments are also meant to include compounds which differ only in the presence of one or more isotopically enriched atoms. For example, therapeutic agents in which one or more atom is replaced by, for example, deuterium, tritium, ¹³C, ¹⁴C (or any isotopic labels as commonly used in the art such as phosphorus, calcium, iodine, chlorine, bromine, or any other convenient element for isotopic labeling) are within the scope of this invention.

Further embodiments of the invention include pharmaceutical compounds that include one or more compounds according to the invention, combined with one or more pharmaceutical carriers or vehicles as known in the art. Also, in preferred method embodiments, the compounds described herein are formulated and are administered as a pharmaceutical composition that includes a pharmaceutically acceptable carrier and one or more pharmaceutical or therapeutic agent, including one or more of the inventive compounds described herein, and optionally including one or more additional agents.

A pharmaceutically acceptable carrier refers to any convenient compound or group of compounds that is not toxic and that does not destroy or significantly diminish the pharmacological activity of the therapeutic agent with which it is formulated. Such pharmaceutically acceptable carriers or vehicles encompass any of the standard pharmaceutically accepted solid, liquid, or gaseous carriers known in the art, such as those discussed in the art. A suitable carrier depends on the route of administration contemplated for the pharmaceutical composition. Routes of administration are determined by the person of skill according to convenience, the health and condition of the subject to be treated, and the location and stage of the condition to be treated.

Such routes can be any route which the practitioner deems to be most effective or convenient using considerations such as the patient, the patient's general condition, and the specific condition to be treated. For example, routes of administration can include, but are not limited to: local or parenteral, including oral, intravenous, intraarterial, intrathecal, subcutaneous, intradermal, intraperitoneal, rectal, vaginal, topical, nasal, local injection, buccal, transdermal, sublingual, inhalation, transmucosal, wound covering, direct injection into a tumor or the area surrounding a tumor, and the like. The administration can be given by transfusion or infusion, and can be administered by an implant, an implanted pump, or an external pump, a nebulizer, an inhaler, or any device known in the art. Preferably, the administration according to embodiments of the invention is oral, inhalation, intramuscular, topical, intratumoral, or intravenous. Preferred means of administration include:
(a) administering the DNA or RNA molecule in a lipid nanoparticle formulation via intravenous injection;
(b) administering the DNA or RNA molecule in a polymeric nanoparticle formulation via inhalation;
(c) administering the DNA or RNA molecule in a viral vector formulation via intramuscular injection;
(d) administering the DNA or RNA molecule in a conjugate formulation via topical application;
(e) administering the DNA or RNA molecule in a prodrug formulation via oral ingestion; and
(f) administering the DNA or RNA molecule in a nanoparticle formulation comprising a targeting ligand via subcutaneous injection.

The RNA molecule may be a small interfering RNA (siRNA), a microRNA (miRNA), or any other RNA or DNA molecule that is capable of silencing, degrading or otherwise modulating GPR68 coding RNA. The delivery method may be selected based on the disease to be treated, the target tissue or cell type, and the desired pharmacokinetic and pharmacodynamic properties. The targeting ligand may be selected based on its affinity for a receptor or antigen expressed on the target tissue or cell type. The prodrug formulation may be designed to release the active RNA molecule in response to a specific physiological condition, such as a change in pH or an enzyme activity. The conjugate formulation may comprise a carrier molecule that enhances the stability or cellular uptake of the RNA molecule.

Therefore, the forms which the pharmaceutical composition can take will include, but are not limited to tablets, capsules, caplets, lozenges, dragees, pills, granules, oral solutions, powders for dilution, powders or fluids for inhalation, vapors, gases, sterile solutions or other liquids for injection or infusion, transdermal patches, buccal patches, inserts and implants, rectal or vaginal suppositories, creams, lotions, oils, ointments, topical coverings (e.g., wound coverings and bandages), suspensions, emulsions, lipid vesicles, and the like. Preferred compositions take the form of tablets, capsules, and preparations for injection or inhalation.

Treatment regimens include a single administration or a course of administrations lasting two or more days, including a week, two weeks, several weeks, a month, two months, several months, a year, or more, including administration for the remainder of the subject's life. The regimen can include multiple doses per day, one dose per day or per week, for example, or a long infusion administration lasting for an hour, multiple hours, a full day, or longer. A dose of about 0.01 mg/kg to about 200 mg/kg per dose is suitable. The dose preferably is about 0.1 mg/kg to about 50 mg/kg, more preferably about 0.1 mg/kg to about 10 mg/kg, and most preferably about 0.2 mg/kg to about 5 mg/kg.

Dosage amounts per administration include any amount determined by the practitioner and will depend on the size of the subject to be treated, the state of the health of the subject, the route of administration, the condition to be treated or prevented, and the like. In general, it is contemplated that for the majority of subjects, a dose in the range of about 0.01 mg/kg to about 200 mg/kg is suitable, preferably about 0.1 mg/kg to about 50 mg/kg, more preferably about 0.1 mg/kg to about 10 mg/kg, and most preferably about 0.2 mg/kg to about 5 mg/kg are useful. This dose can be administered weekly, daily, or multiple times per day. A dose of about 0.1 mg, 0.2 mg, 0.25 mg, 0.5 mg, 1 mg, 5 mg, 10 mg, 20 mg, 40 mg, 80 mg, 100 mg, 250 mg, 500 mg, or 1000 mg can be administered.

A549 lung cancer cells produce the predominant airway and lung mucin, Mucin5AC, at low pH. Inhibition of GPR68 inhibits Mucin 5AC in a dose-dependent manner.

In certain embodiments, the invention is contemplated for use in malignancies (cancer), as discussed. The methods of these embodiments include treatment and/or prevention of cancer, such as glioblastoma multiforme, pancreatic cancer, lung cancer, medulloblastoma, prostate cancer (e.g., neuroendocrine prostate cancer), skin cancer (e.g., melanoma), breast cancer, ovarian cancer, cervical cancer, colon or colorectal cancer, kidney cancer, fibrosarcoma, hepatocellular cancer, acute monocytic leukemia, B-cell lymphoma, and chronic myelogenous leukemia.

GPR68 is expressed on bronchial airway epithelial cells, on bronchial airway smooth muscle cells, and on bronchial airway dendritic cells. Therefore, inhibitors or antagonists of GPR68 can be useful in asthma, in either a prophylactic context (control) or the treatment of acute symptoms (rescue) of the disease. Furthermore, GPR inhibitors can target all three cardinal pathogenic processes in asthma. No current standard of care medication can address all three cardinal processes in asthma. In some aspects, embodiments of the invention include treatment of a subject with poorly controlled asthma during an exacerbation. The inhibitor or antagonist can be administered in combination with standard of care beta adrenergic agonists, anticholinergic compounds, leukotriene modulators, and/or systemic corticosteroids to improve pulmonary function and end the exacerbation.

Therefore, in certain other embodiments, the invention is contemplated for use in autoimmune/inflammatory disease and conditions, as discussed. Preferably, the methods of these embodiments include treatment and/or prevention of conditions such as asthma, chronic obstructive pulmonary disease (COPD), chronic bronchitis, aspiration pneumonia, viral pneumonia, coronavirus pneumonia and lung injury (e.g., COVID-19), acute lung injury (ALI), acute respiratory distress syndrome (ARDS), cystic fibrosis, osteoporosis, diabetes type 1, inflammatory bowel disease, atherosclerosis and other inflammatory cardiovascular conditions, multiple sclerosis, inflammatory pain syndrome, chronic inflammatory disease, or other autoimmune or inflammatory diseases.

One well-established experimental model of ALI/ARDS is acid (HCl) instillation into animal's lungs. In this model chemical injury of lung tissue caused by HCl incites the disease process. This model was used to interrogate the pulmonary pathogenesis of the SARS virus (SARS CoV). The SARS CoV receptor is shared by SARS CoV-2 (the etiologic agent of COVID-19), namely angiotensin converting enzyme-2 (ACE2). Recombinant SARS CoV spike protein was administered to animals with acid induced ARDS. Spike-Fc protein localized to bronchial epithelial cells, inflammatory exudates and alveolar pneumocytes.

As for treatment or prevention of ALI, the OGM series of compounds may be administered by inhalation or via systemic administration in the setting of first clinical recognition of ALI or as a prophylaxis or medical countermeasures in the setting of exposures, infectious or otherwise, that can precipitate ALI and ARDS.

OGM compounds for the work in all experiments in the Examples have used the same synthesis for OGM2 or have been repeated with the same synthesis of OGM2. Key findings have been replicated with OGM17 using similar methods as OGM2, which was synthesized as previously described (Williams et al., 2019). Temozolomide was purchased from TOCRIS bioscience (Cat No. 2706). NE 52-QQ57 was purchased from Sellckchem™. NFkB inhibitor BAY 11-7082 was purchased from Calbiochem/EMD Biosciences™. All cell culture media and reagents were obtained from Gibco™.

By timelapse microscopy of an extracellular pH-indicator, pHluorin2-GPI, the establishment of the acidic extracellular microenvironment during formation of GBM spheroids in vitro has been visualized for the first time. Moreover. GBM cells responded to acidification of media with a calcium response, which was sensitive to ogremorphin (OGM) and PLC inhibition, indicating a role for GPR68/Gq signaling in sensing low extracellular pH in these cells. Others have shown that medulloblastoma cells also elicit a calcium ion flux upon extracellular acidification, suggesting a shared mechanism for these two CNS tumor types, in response to acidic tumor microenvironment.

GBM is universally fatal because of its extreme treatment resistance, which has been attributed to extensive heterogeneity and therapy-induced cellular plasticity. Inhibition of GPR68 signaling dramatically reduces the survival of both TMZ-sensitive and insensitive GBM cell lines U87 and U138, without inducing nonselective toxicity in cells of developing zebrafish embryos or in HEK293 cells. Moreover, OGM2 reduced survival of all 12 GBM lines tested, regardless of molecular subtype or species. These studies raise the possibility that GBM utilizes GPR68 proton-sensing signaling as a shared survival mechanism activated by the acidic tumor microenvironment, and therefore represents an attractive therapeutic target for many GBM subtypes regardless of their molecular heterogeneity.

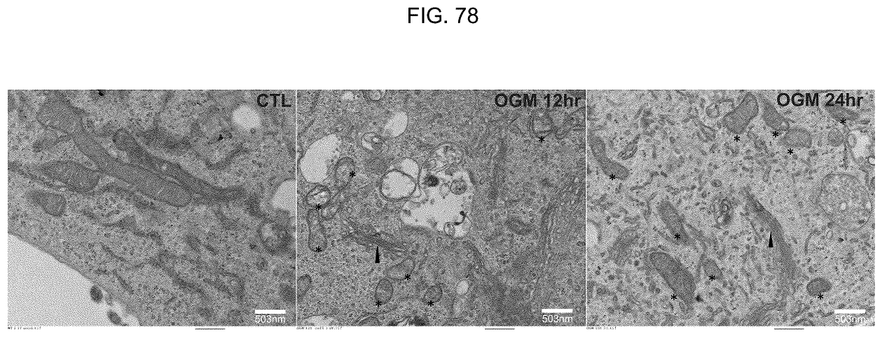

GPR68 inhibition induced ferroptosis through the up-regulation of ATF4 and its downstream targets. One such direct transcriptional target of ATF4 is CHAC1, which induces oxidative stress by degrading glutathione (GSH), resulting in two hallmarks of ferroptosis: increased lipid peroxidation and mitochondria disintegration. While the RNA-seq results here support that ferroptosis and UPR/ER stress response, share overlapping mechanisms, the electron microscopy studies showed no evidence of ER stress response (see FIG. 78). For example, ASNS (asparagine synthetase), upregulated in certain tumor microenvironments to promote cell proliferation, chemoresistance, and metastatic behavior, is known to be activated by UPR and amino acid depletion, the latter of which triggers ferroptosis. Importantly. ASNS is a transcriptional target of ATF4, alongside CHAC1 and SLC7A11. ATF4 has been implicated in ferroptotic neuronal death in response to oxidative stress during stroke. Additionally, GPR68 has been found to protect neurons from death in the context of oxidative stress during stroke. Therefore, this neuroprotective mechanism can be weaponized by GBM, so that the acidic tumor microenvironment and/or Warburg effect activates GPR68-dependent pro-survival pathways including repression of ATF4.

Many of the current anti-cancer therapies aim to activate apoptosis, which triggers a cascade that concludes with caspase-3 activation. However, the “Achilles heel” of apoptosis is its dependence on the p53 tumor suppressor, which is dysregulated in 84% of GBM patients. In this regard, ferroptosis represents an attractive alternative cell death pathway.

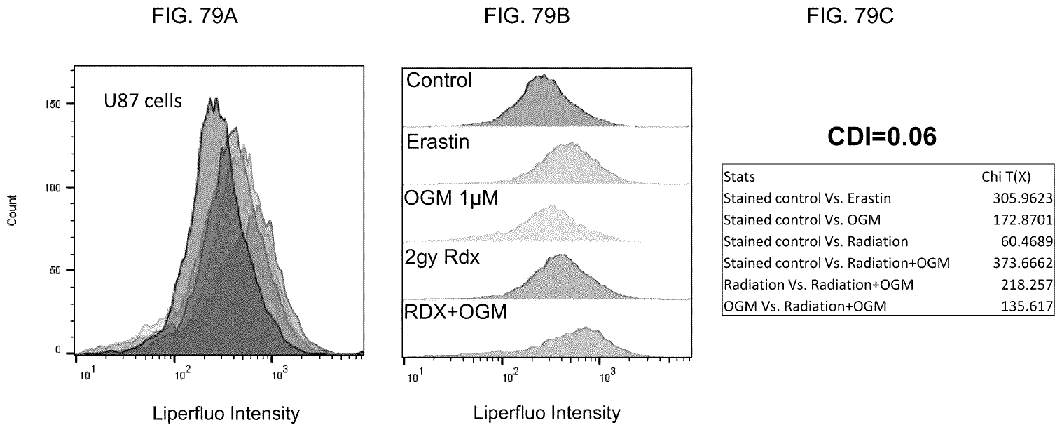

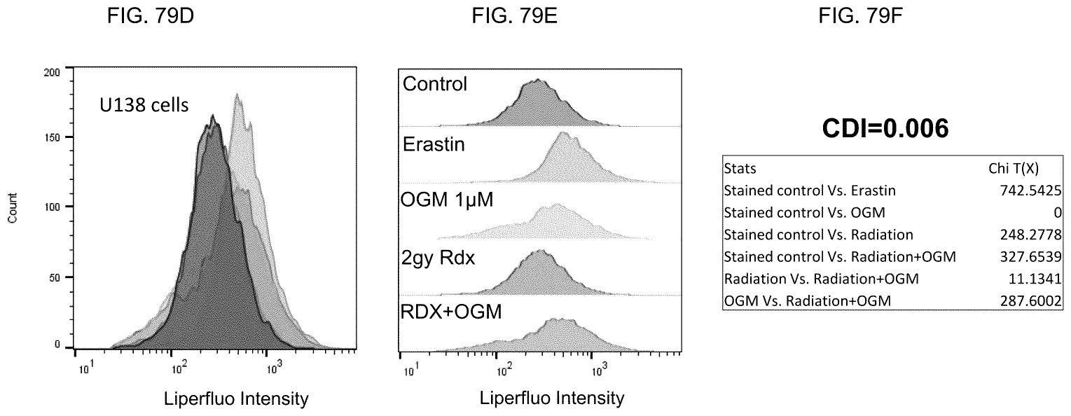

Furthermore, OGM2 could also boost the efficacy of ionizing radiation since low extracellular pH confers radio-resistance to human glial cells and GPR68 is known to be upregulated in radioresistant GBM cell lines. Consistent with this view, GPR68 inhibition with OGM2 and ionizing radiation demonstrated a dramatic synergistic induction of ferroptosis in U87 and U138 cells (see FIG. 79A through FIG. 79F). Interestingly, OGM2 also demonstrated synergistic GBM cell killing with TMZ. In summary, the results indicate that acidic extracellular milieu, similar to that which is found in tumor microenvironments possibly due to activation of Warburg effects, activates a key survival pathway conserved in GBM. Thus. GPR68 inhibitors like ogremorphins represent an attractive therapeutic class to selectively induce ferroptosis in GBM, especially in conjunction with the frontline therapies TMZ and ionizing radiation. Thus. GPR68 inhibitors represent an attractive approach to enhance therapeutic efficacy of traditional chemotherapeutic agents and radiation therapy by sensitizing glioblastoma to these frontline therapies. Finally, because GPR68 inhibitors do not indiscriminately induce ferroptosis in non-cancerous tissues and cells, they are safe and effective way to induce cell killing selectively in cancer cells.

Consistent with the notion that Warburg effect/acidic tumor microenvironment is a hallmark of many cancers, our evidence suggest that GPR68 inhibitors are efficacious against variety of cancers, including pancreatic cancer, prostate cancers, and lung cancers and they exhibit synergistic cell killing in conjunction with the frontline therapies such as TMZ and ionizing radiation. Moreover, GPR68 inhibitors like OGM2 induce ferroptosis in pancreatic and lung cancers in a synergistic manner with ionizing radiation. Thus, GPR68 inhibitors like the ogremorphin class represent an attractive approach to selectively kill brain, pancreatic and lung cancers, especially in conjunction with the frontline therapies of ionizing radiation and traditional chemotherapies, and to enhance therapeutic efficacy of traditional chemotherapeutic agents and radiation therapy by sensitizing brain, pancreatic, lung and other cancers to these frontline therapeutic modalities.

The results also indicate that ATF4 loss, either due to mutation or gene expression regulation, is a potential mechanism for resistance to tumor killing by GPR68 inhibition and that expression of ATF4, either through gene expression regulation, gene therapy or other methods of forced expression, will overcome this potential resistance. Therefore, further administration of an ATF4 activating agent can be used in treatment of cancers.

Finally, consistent with the fact that ferroptosis is a form of immunogenic cell death. GPR68 inhibitors induce hallmarks of immunogenic cell death such as extracellular ATP release and calreticulin (CRT) externalization in cancer cells. Moreover, GPR68 inhibitor blocks macrophage immunosuppression induced by extracellular lactate. Therefore, GPR68 inhibitors represent an attractive approach to enhance cancer immunotherapies, including check-point inhibitors, cancer vaccines and CAR-T therapies.

Cancer cell killing by GPR68 inhibition results in enhanced immunological memory against the treated tumors. This can result in lasting protection against residual cancers or cancer recurrence in patients treated with GPR68 inhibitors. Moreover, therapeutic agents to block GPR68 can be used in conjunction with tumor vaccine treatments to boost their therapeutic efficacy. Specifically, GPR68 inhibitors may be administered prior to the initiation of the tumor vaccination to “prime” the anti-cancer immune cells and/or delivered after vaccination for a period of time, for example a month, to boost anti-cancer immune response.

### 5. Examples

This invention is not limited to the particular processes, compositions, or methodologies described, as these may vary. The terminology used in the description is for the purpose of describing the particular versions or embodiments only and is not intended to limit the scope of the present invention which will be limited only by the appended claims.

Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: General Methods

### A. Chemical Screening

A chemical screen for small molecules that perturb embryonic development (dorsoventral axis) in zebrafish was performed as previously described (see Chen et al., 2009; Hao et al., 2013; Williams et al., 2015; Yu et al., 2008)). Briefly, pairs of WT zebrafish were mated, and fertilized eggs were arrayed in 96-well microtiter plates (5 embryos/well) containing 1001 E3 water. At 4 hours post fertilization (pf), a small molecule library from Vanderbilt High Throughput Screening Facility was added to each well to a final concentration of 10 μM. Embryos were incubated at 28.5 C until 24 and 48 hours pf, when they were examined for gross morphologic changes indicative of reproducible embryonic defects (dorsalization of the embryonic axis). A total of 30,000 compounds were screened.

### B. Alcian Blue Staining

Staged embryos and larvae were anesthetized with Tricaine and killed by immersion in 4% formaldehyde (prepared from paraformaldehyde, and buffered to pH 7 in phosphate-buffered saline (PBS)). The fixed animals were rinsed in acid-alcohol (0.37% HCl, 70% EtOH), and stained overnight in Alcian blue (Schilling et al., 1996a). After destaining in several changes of acid-alcohol, the preparations were rehydrated. Following rinsing and clearing in a solution of 50% glycerol and 0.25% KOH, the cartilages were visualized under a stereo microscope.

### C. Whole-Mount Zebrafish In Situ Hybridization

In situ hybridization was performed as previously described (Westerfield, 2000). Zebrafish foxD3 probes were synthesized as previously described (Stewart et al., 2006).

### D. Target Profiling Assays for GPCR

GPCR profiling assays were performed by Millipore (St. Louis, MO) using in cells expressing Gα15, a promiscuous G protein that enhances GPCR coupling to downstream Ca2+ signaling pathways. KinomeScan was conducted by DiscoveRx (Fremont, CA).

### E. Zebrafish Injections

OGR1 morpholino 5′-TTTTCCAACCACATGTTCAGAGTC-3′ (SEQ ID NO:8) and Mismatch morpholino 5′-CCTCTTACCTCAGTTACAATTTATA-3 (SEQ ID NO:9) was synthesized by Genetools™. Morpholino and mRNA was injected as previously described (Westerfield, 2000).

### F. Real-Time PCR

Melanoma cell lines were collected and total RNA was isolated with the RNeasy kit (Qiagen™). After subsequent cDNA amplification using Superscript III (Invitrogen™, Carlsbad, CA), samples were visualized in an agarose gel. See Table 1 for primer sequences.

TABLE 1
GPR Primers.

| Human Gene | Forward Primer | Reverse Primer | SEQ ID NO |
|---|---|---|---|
| GPR4 | CCCTCCTGTCATAATTCCATCC | TGGTCTACAGGGAAGAGATGAG | 10, 11 |
| GPR65 | TGGCTGTTGTCTACCCTTTG | CCACAACATGACAGCATTGAAG | 12, 13 |
| GPR68 | GTTTGAAGGCGGCAGAAATG | GTGGAATGAGGAGGCATGAA | 14, 15 |
| GPR132 | TTCAGGAGCATCAAGCAGAG | CGAAGCAGACTAGGAAGATGAC | 16, 17 |
| GAPDH | GTTTGAAGGCGGCAGAAATG | GTGGAATGAGGAGGCATGAA | 18, 19 |

cDNAs were generated using High-Capacity cDNA Reverse Transcription Kit with RNase Inhibitor (ThermoFisher, 4374%6). Samples were run on a Quant Studio 5 real-time PCR system (Applied Biosystems™) with TaqMan™ Universal Master Mix II, with UNG (ThermoFisher, 4440042). Primers used were GAPDH: Hs02786624g1, GPR68: Hs00268858s1, ATF4: Hs00909569_g1, CHAC1: Hs00225520_m1, SLC7A11: Hs00921938_m1, HMOX1: Hs01110250_m1, TFRC: Hs00951083_m1.

### G. Cell Culture and Transfection

HCT116, U87, U138, HEK293, HEK293T, MeWo, A2058 and MCF7 cells were cultured in DMEM supplemented with 10% FBS (GIBCO™) and 1% penicillin-streptomycin (Cellgro™). Human temozolomide sensitive glioblastoma cell line (U87) was a kind gift from Rebecca Ihrie (Vanderbilt University, Nashville, TN). The human temozolomide-insensitive glioblastoma cell line (U138) was purchased from ATCC (HTB-16). WM115 cells were cultured in DMEM/F12 supplemented with 10% FBS (GIBCO™) and 1% penicillin-streptomycin (Cellgro™).

### H. Luciferase Assay Transfection

12-well plates of cells were transfected with 1 μg DNA using Lipofectamine 3000 with sre:luciferase and either the vector backbone, GFP, GPR68, GPR68-GFP, GPR4, or 336X-GFP. After 3 days, these cells were then lysed, and cell extracts were subjected to Steady-Glo luciferase assay (Promega™) according to the manufacturer's instructions. The results were normalized to cell titer, as determined using Cell Titer-Glo luminescence assay (Promega™). For some procedure, the cells were treated in serum-free medium, and were lysed on the third day.

### I. 336X-GFP Plasmid Generation

A deletion between amino acid 336 and the beginning of GFP was generated from the MCSV:GPR68-GFP plasmid using the Q5 mutagenesis kit (NEB).

### J. Agarose Drop Assay

WM115 cells were trypsinized and resuspended at 100,000 cells per mL in low melt agarose. A 10 μl drop of the cell mixture was added to 12-well plate. After solidifying Normal culture media was added with either DMSO 0.5% or OGM2 5 μM. The area around the agarose drop was visualized manually with light microscopy. The cells were incubated for 5 days and visualized. Cells outside of the agarose drop were visualized and quantitated.

### K. Scratch Assay

transfected using Lipofectamine 3000 with pTol2 (Ubi:pHluorin2-GPI), and pCMV-Tol2 (Addgene:31823). Three weeks post-transfection, cells were flow-sorted for pHluorin2-GPI expression and clonally selected.

### N. Alkalization Assay

pHluorin1-GPI cells in HEPES-buffered FluoroBrite medium (ThermoFisher™) were imaged using 487 nm excitation/525 nm emission using the Lionheart Imager (BioTek™). Alkaline medium was added to the well using the automated injection system to adjust the pH of the well to pH 8.4 and imaged with the same settings after 20 seconds.

### O. In Vitro Cell Viability Assays

GBM neurospheres and low passage PDX models were plated in 96-well plates at 10,000 cells per well in 50 μL of neural stem cell media. The next day, the cells were treated with OGM2 compounds at the indicated concentrations, in triplicates, by adding an equal volume of medium containing 2× the final concentration of compound. Following 72 hours incubation under standard cell culture conditions, relative cell number was assessed using Cell Titer-Glo Luminescent Cell Viability Assay (Promega™) following the manufacturer's instructions. Luminescence was determined using a Cytation 5 reader and Gen5 software package (BioTek™). For U87 and U138 cell lines, one thousand cells were plated per well in a standard 96-well plate and allowed to attach for 24 hours before exposure to concentrations of vehicle, OGM2, or TMZ. Cells were treated for 72 hours and then stained with DAPI. A 10× magnification lens on a LionheartFX (BioTek™) was used to image the wells, and images were stitched together with Gen5 software (BioTek™). Automated nuclei counting was also done using the Gen5 software. Results reported as percent response relative to DMSO control. IC₅₀ (the concentration causing 50% inhibition) was determined by GraphPad Prism™ version 6.07.

### P. GBM Spheroid Assay

One thousand cells per well were plated in an ultra-low attachment, round-bottomed, 96-well plate, and spheroids were allowed to form for 3 days. Wells were then exposed to concentrations of vehicle, OGM2 or TMZ or a combination for 3 days. Spheroids were imaged in brightfield at 10× using z-stacks that were collapsed into z-projections in the Gen5 software using the LionheartFx (BioTek™). Automated measurements of the spheroid area were obtained using Gen5 software.

Q. GBM neurospheres and PDX Propagation

The GBM neurospheres and PDX models we used are considered to be genetically and pathologically superior models as they stably maintain the genomic changes of primary tumors, allow maintenance and expansion of glioma stem cells (GSC), and provide clinically relevant GBM models in mice. The neurospheres were provided as a generous gift. The PDX models were acquired from the PDX National Resource at the Mayo Clinic. All neurosphere lines and PDX models have were tested for mycoplasma contamination and identified by STR analysis before the beginning of the study.

### R. Western Blot Analysis

Using Pierce™ BCA Protein Assay Kit (ThermoFisher Scientific™), protein concentrations were determined for each sample following manufacturing protocol. Gel electrophoresis was conducted on NuPAGE™ 4-12% Bis-Tris gels (Invitrogen™) using 20 ug of total protein per sample. Proteins were transferred to PVDF membranes by semi-dry transfer. Membranes were blocked using Intercept® (PBS) Blocking Buffer (LI-COR™) for one hour at room temperature on a tilting shaker. Primary antibodies in 5% non-fat dry milk were added to the membranes for overnight incubation at 4° C. on a rotating shaker. Primary antibodies included transferrin receptor (TFRC), heme-oxygenase 1 (HO-1), and cleaved caspase 3 with GAPDH and α-tubulin used as normalization controls. The next day, membranes were washed in three, five-minute intervals with PBST (Tween 0.5%). Corresponding secondary HRP-conjugated antibodies in 5% non-fat dry milk were added to incubate at room temperature for one hour on a tilting shaker. The membranes were washed with PBST in four, five-minute intervals before protein visualization using Radiance Q (Azure Biosystems™) on Bio-Rad™ ChemiDoc™ MP Imaging System. Protein quantification was completed in triplicate using Fiji software package.

S. GPR68 Knockdown with siRNA

siRNAs for knockdown were obtained from Dharmacon™. GPR68-siRNA1 and GPR68-siRNA2. For controls we used Dharmacon™ siGENOME Non-Targeting siRNA. Control Pool standard non targeting siRNA was used. Cells were reverse transfected 10 ng of siRNA with lipofectamine RNAiMAX (ThermoFisher™) in a 12-well plate (CELLTREAT Scientific Products™) in DMEM with high glucose, GlutaMAX, HEPES. Penicillin-Streptomycin and 10% FBS. Three days post transfection cells were lysed 1× Passive lysis buffer (Promega™) and assessed with CellTiter-Glo (Promega™) or stained with ReadyProbes Cell Viability Imaging Kit (ThermoFisher™). Alternatively, total RNA was collected 24 hours post transfection of siRNA for qRT-PCR.

T. GPR68 Knockdown with CRISPRi

Cells were reverse transfected on a 12-well cell culture dish in DMEM with high glucose, GlutaMAX, HEPES, Penicillin-Streptomycin and 10% FBS with 2.5 μgs dCas9 per well. The next day, fresh media was added to the wells and the cells were transfected with 12 pmol sgRNA using lipofectamine RNAiMAX. Three days later, cell survival was assessed by lysing the cells with 1× Passive lysis buffer and quantification with Cell titer glow. Alternatively, total RNA was collected after three days for cDNA generation and qRT-PCR. Alt-R modified sgRNAs were obtained from IDT targeting sequences were:

GPR68 sgRNA1:
(SEQ ID NO: 20)
5′-ACCGCCAUCCUGUUUAUAGA-3′,
and
GPR68 sgRNA2:
(SEQ ID NO: 21)
5′-GAAGGGGCCACACUCCUCAU-3′;
GPR68 sgRNA3:
(SEQ ID NO: 22)
5′-CCAUACCAUCCACCAGACGC-3′,
and
GPR68 sgRNA4:
(SEQ ID NO: 23)
5′-GCCCCUUCAGGCCCAAAGA7U-3′.

### U. RNA-seq

One Million 913 and 08-387 cells were treated with 0.5 μM OGM2, Mayo6 and Mayo39 cells were treated with 2 μM for 72 hours. Cells were trypsinized and flash frozen. Cell pellets were sent to Azenta™ for RNA-isolation, all RNA samples had RIN between 7.7 and 10.0, and sequencing, 20-30 Million reads on Illumina™ HiSeq, PE 2×150 bp. Read counts were normalized with and differential expression was determined using DESeq2. Gene Set Enrichment Analysis was done on DAVID.

### V. Glutathione Assay

GSH-Glo™ Glutathione Assay (Promega™) was performed according to manufacturer protocol. Briefly, U87 cells were plated at 10,000 cells per well in a 96-well plate. The following day cells were treated with DMSO control vehicle or 2 μM OGM2 for 24 hours. Medium was removed from wells and 100 μl GSH-Glo™ Reagent was added to each well and incubated on a plate shaker for 30 minutes. One hundred microliters Luciferin Detection Reagent was added to each well and mixed briefly. After 15 minutes incubation, luminescence was detected on Promega™ GloMax luminometer.

### W. Liperfluo™

Cells were plated on a 100 mm cell culture dish (CELLTREAT Scientific Products) in 10 mls of DMEM with high glucose, GlutaMAX, HEPES, Penicillin-Streptomycin (ThermoFisher™) and 10% FBS. Cells were incubated overnight at 37° C. in 5% CO₂. Media were then replaced with 30 mL fresh media with DMSO, OGM2, or Erastin (APExBIO Technology™) and the cells incubated for 3 days. On the third day, 3 mL fresh media with 2.5 μM Liperfluo™ (Dojindo Molecular Technologies™, Inc.) resuspended in DMSO was added and cells were incubated at 37° C. in 5% CO₂ for 1 hour. Cells were subsequently trypsinized for 5 minutes, pelleted by centrifugation, and resuspended in cell sorting media (1% BSA and 1 mM EDTA in PBS pH 7.4). Ten thousand events were recorded on a BD LSR II and the data processed using the FlowJo™ software.

### X. ATF4 Knock Down

Cells were reverse transfected on a 12-well cell culture dish (CELLTREAT Scientific Products™) in DMEM with high glucose, GlutaMAX, HEPES, Penicillin-Streptomycin and 10% FBS with 2.5 μgs dCas9 per well. The next day, fresh medium with or without 2 μM OGM2 was added to the wells and the cells were transfected with 12.5 pmol sgRNA using lipofectamine RNAiMAX. Three days later, cell survival was assessed by lysing the cells with 1× Passive lysis buffer and quantitation with Cell titer glow. Alternatively, total RNA was collected after three days for cDNA generation and qRT-PCR. Alt-R modified sgRNAs were obtained from IDT targeting sequences:

ATF4 sgRNA1:
(SEQ ID NO: 24)
5′-GAUGUCCCCCUUCGACCAGU-3′,
ATF4 sgRNA2:
(SEQ ID NO: 25)
5′-GCGGUGCUUUGCUGGAAUCG-3′,
ATF4 sgRNA3:
(SEQ ID NO: 26)
5′-CCACCAACACCUCGCUGCUC-3′,
ATF4 sgRNA4:
(SEQ ID NO: 27)
5′-AGCUCAUUUCGGUCAUGUUG-3′,
and
ATF4 sgRNA5:
(SEQ ID NO: 28)
5′-AAUGAGCUUCCUGAGCAGCG-3′. Y.

### Transmission Electron Microscopy (TEM)

U87 cells were treated with DMSO or 2 μM of OGM2 for 12 or 24 hours, then fixed with 2.5% glutaraldehyde. The samples were prepared for TEM imaging after fixation. Samples were imaged on FEI™ Tecnai T12.

### Z. Statistics

Where appropriate, ANOVA, or student's T-test were conducted in PRISM. Chi squared T(X) was used for Liperfluo analysis. A value T(X)>4 implies that the two distributions are different with a p<0.01 (99% confidence). For drug interaction and therapeutic interaction, the coefficient of drug interaction (CDI) was calculated as follows: CDI=AB/(A×B). According to the impact of each group, AB is the value of combined treatment and A or B is the value of the single agent group to control group. Thus, CDI <1, =1 or >1 indicates that the drugs are synergistic, additive, or antagonistic, respectively. CDI <0.7 indicates that the drug is significantly synergistic.

### Example 2: Chemical Schemes and Structures

### A. General Synthetic Scheme

Below is the general scheme for synthesis of compounds according to embodiments of the invention. The scheme refers to the following reagents and conditions: i) dry THF, 0° C.-room temperature; ii) chloroacetic acid, NaHCO₃·H₂O, 1 hour, room temperature; iii) IN NaOH, NH₂NH₂·H₂O, 1 hour, room temperature; iv) EtOH, 40° C. R₁ is —H, —CH₃, —Cl, —F, or —OCH₃; R₂ is —H, —C₂H₅, —Cl, —F, or —OCH₃.

### B. Example Procedure for the Synthesis of 5-fluoro-5′-(quinolin-6-yl)-1,2-dihydro-3′H-spiro[indole-3,2′-[1,3,4]thiadiazol]-2-one; OGM45

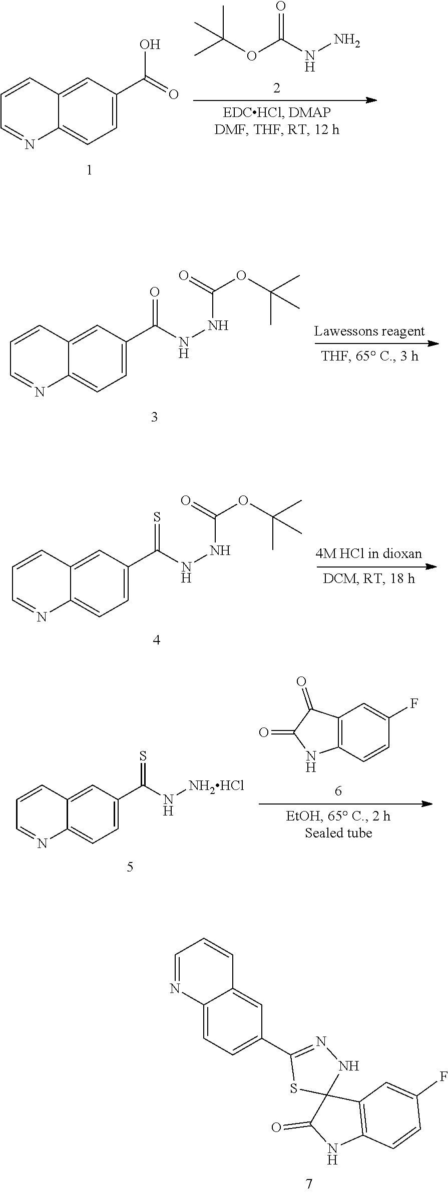

### Synthesis of tert-butyl 2-(quinoline-4-carbonyl)hydrazine-1-carboxylate (3)

Procedure: To a stirred solution of quinoline-4-carboxylic acid (2.00 g, 11.5 mmol, 1.0 eq) in 30 mL of a mixture of DMF and THF (1:1) were added (tert-butoxy)carbohydrazide (1.68 g, 12.7 mmol, 1.1 eq), EDC·HCl (2.66 g, 13.9 mmol, 1.2 eq) and 4-(dimethylamino)pyridin-1-ium (0.021 g, 0.173 mmol, 0.015 eq). After 10 minutes, the mixture became homogeneous and stirring was continued for 3 hours. The reaction was monitored by TLC. The reaction mixture was poured into ice and extracted with ethyl acetate (3×30 mL). The combined organic layer was washed with water (20 mL), brine (20 mL), dried over anhydrous sodium sulfate and concentrated under reduced pressure to give crude product. The crude product was purified by flash column chromatography over silica gel by using a Combiflash™ purifier with 5% methanol in DCM as eluent to give tert-butyl 2-(quinoline-4-carbonyl)hydrazine-1-carboxylate (1.8 g, 54%) as white solid. LCMS: m/z=288.2 [M+H]+.

### Synthesis of Tert-butyl 2-(quinoline-6-carbonothioyl)hydrazine-1-carboxylate (4)

Procedure: To a stirred solution of N′-[(tert-butoxy)carbonyl]quinoline-6-carbohydrazide (1.3 g, 4.52 mmol, 1.0 eq) in THF (20 mL) was added Lawesson's Reagent (1.83 g, 4.52 mmol, 1.0 eq) at RT. The reaction mixture was heated to 65° C. and stirred for 3 h. Reaction was monitored by TLC and LCMS. The reaction mixture was cooled to RT and quenched with saturated sodium bicarbonate (20 mL) and extracted with ethyl acetate (2×10 mL). The combined organic layer was washed with water (10 mL), brine (10 mL), dried over anhydrous sodium sulphate and evaporated under reduced pressure to give crude product. The crude product was purified by flash column chromatography over silica gel by using a combiflash purifier with 6% MeOH in DCM as eluent to give tert-butyl 2-(quinoline-6-carbonothioyl)hydrazine-1-carboxylate (1.0 g, 73%) as yellow solid compound. LCMS: m/z=304.1 [M+H]+.

### Synthesis of quinoline-6-carbothiohydrazide hydrochloride (5)

Procedure; To a stirred solution of tert-butyl 2-(quinoline-6-carbonothioyl)hydrazine-1-carboxylate (1.0 g, 3.30 mmol, 1.0 eq) in DCM (10 mL) was added 4M HCl in dioxane (10 mL) at 0° C. and stirred at room temperature for 18 hours. Progress of the reaction was monitored by TLC (MeOH/DCM=5:95). After 12 hours the reaction was complete and the reaction mixture was concentrated to give residue. It was washed with diethyl ether (30 mL) and dried to give quinoline-6-carbothiohydrazide hydrochloride (0.7 g, 89%) as light yellow solid compound. LCMS: m/z=204.2 [M+H]+.

### Synthesis of 5-fluoro-5′-(quinolin-6-yl)-1,2-dihydro-3′H-spiro[indole-3,2′-[1,3,4]thiadiazol]-2-one (7) (OGM45)

Procedure: To a sealed tube vial, N-aminoquinoline-6-carbothioamide hydrochloride (0.1 g, 0.41 mmol, 1.0 eq), 5-fluoro-2,3-dihydro-1H-indole-2,3-dione (0.069 g, 0.417 mmol, 1.0 eq) and ethanol (5 mL) were added and heated to 65° C. for 2 hours. Reaction was monitored by TLC and LCMS. After completion of the starting material, reaction was cooled to RT and the solvent was concentrated to give the residue. It was diluted with EtOAc (10 mL) and washed with saturated bicarbonate (10 mL). Aqueous layer was extracted in to EtOAc (3×5 mL). Combined organics were washed with brine (10 mL), water (10 mL) and dried over Na₂SO₄, filtered and concentrated to give crude product. The crude product was purified by flash column chromatography by using combiflash purifier with 5-10% MeOH in DCM as eluent. Pure fractions were concentrated to give 5-fluoro-5′-(quinolin-6-yl)-1,2-dihydro-3′H-spiro[indole-3,2′-[1,3,4]thiadiazol]-2-one as brown solid (0.018 g, 12%). LCMS (ES) m/z=351.0 [M+H]+; 1H NMR (400 MHz, DMSO d6) δ=10.70 (s, 1H), 9.11 (s, 1H), 8.91 (s, 1H), 8.45 (d, J=8.0 Hz, 1H), 8.10-8.02 (m, 2H), 7.95 (s, 1H), 7.58-7.55 (m, 1H), 7.41-7.39 (m, 1H), 7.16 (t, J=8.8 Hz, 1H), 6.88-6.86 (m, 1H), HPLC purity: 97.11% at 240 nm.

### Example Compounds

See Table 2, below for example compounds synthesized using the general procedure outlined above for compound OGM45.

TABLE 2
Selected Example Compounds.

| Compound | | |
|---|---|---|
| Number | Compound Structure | Analytical Data |
| OGM37 | 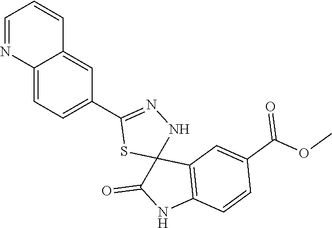 [SMILES: COC(=O)c1ccc2c(c1)C1(NN=C(c3ccc4ncccc4c3)S1)C(=O)N2] | LCMS (ES) m/z = 391.1 [M + H]+; 1H NMR (400 MHz, DMSO d6) δ = 11.00 (s, 1H), 9.11 (s, 1H), 8.91 (s, 1H), 8.46 (d, J = 8.4 Hz, 1H), 8.11- 8.09 (m, 2H), 8.05-7.95 (m, 3H), 7.59-7.56 (m, 1H), 7.00 (d, J = 8.4 Hz, 1H), 3.79 (s, 3H); HPLC purity: 99.59% at 254 nm |
| OGM38 | 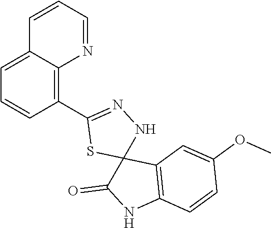 [SMILES: COc1ccc2c(c1)C1(NN=C(c3cccc4cccnc34)S1)C(=O)N2] | LCMS (ES) m/z = 363.2 [M + H]+; 1H NMR (400 MHz, DMSO d6) δ = 10.14 (s, 1H), 8.83-8.78 (m, 2H), 8.42 (d, J = 8.0 Hz, 1H), 8.37 (d, J = 7.6 Hz, 1H), 8.02 (d, J = 8.0 Hz, 1H), 7.67 (t, J = 8.0 Hz, 1H), 7.59-7.56 (m, 1H), 7.08 (s, 1H), 6.84 (d, J = 8.0 Hz, 1H), 6.75 (d, J = 8.4 Hz, 1H), 3.70 (s, 3H); HPLC purity: 97.87% at 254 nm. |
| OGM39 | 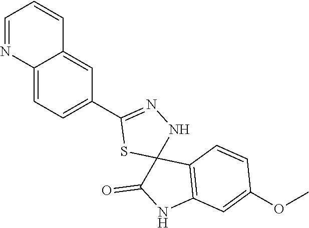 [SMILES: COc1ccc2c(c1)NC(=O)C21NN=C(c2ccc3ncccc3c2)S1] | LCMS (ES) m/z 363.1 [M + H]+; 1H NMR (400 MHz, DMSO d6) δ = 10.5 (s, 1H), 8.99 (s, 1H), 8.90 (s, 1H), 8.46-8.44 (m, 2H), 8.09-8.01 (m, 1H), 7.99 (m, 1H), 7.57-7.55 (m, 1H), 7.44-7.42 (m, 1H), 6.61- 6.59 (s, 1H), 6.40 (m, 1H), 3.89 (s, 3H); HPLC purity: 98.7% at 254 nm. |
| OGM40 | 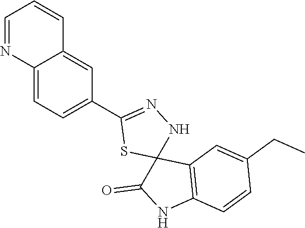 [SMILES: CCc1ccc2c(c1)C1(NN=C(c3ccc4ncccc4c3)S1)C(=O)N2] | LCMS (ES) m/z = 361.3 [M + H]+; 1H NMR (400 MHz, DMSO d6) δ = 10.42 (s, 1H), 9.09 (s, 1H), 8.94 (s, 1H), 8.53 (d, J = 7.6 Hz, 1H), 8.13- 8.11 (m, 1H), 8.06 (m, 1H), 8.04- 7.97 (m, 1H), 7.63-7.62 (m, 1H), 7.37 (s, 1H), 7.15-7.13 (m, 1H), 6.78-6.76 (m, 1H), 2.56-2.48 (m, 2H), 1.14-1.10 (m, 3H); HPLC purity: 99.56% at 254 nm. |
| OGM41 | 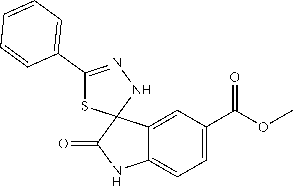 [SMILES: COC(=O)c1ccc2c(c1)C1(NN=C(c3ccccc3)S1)C(=O)N2] | LCMS (ES) m/z = 340.0 [M + H]+; 1H NMR (400 MHz, DMSO-d₆) 1H NMR (400 MHz, DMSO-d6) δ = 10.95 (s, 1H), 8.90 (s, 1H), 7.96- 7.93 (m, 2H), 7.56-7.55 (m, 2H), 7.45-7.44 (m, 3H), 6.98-6.96 (d, J = 8.4 Hz, 1H), 3.79 (s, 3H). HPLC purity: 96.50% at 254 nm. |
| OGM42 | 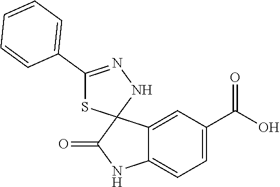 [SMILES: O=C(O)c1ccc2c(c1)C1(NN=C(c3ccccc3)S1)C(=O)N2] | LCMS (ES) m/z = 326.1 [M + H]+; 1H NMR (400 MHz, DMSO-d₆) δ 12.93 (s, 1H), 10.89 (s, 1H), 8.83 (s, 1H), 7.97-7.90 (m, 2H), 7.69 (d, J = 7.6 Hz, 1H), 7.55-7.44 (m, 3H), 7.22 (t, J = 7.2 Hz, 1H), 6.94 (d, J = 7.6 Hz, 1H). HPLC purity: 99.79% at 254 nm. |
| OGM43 | 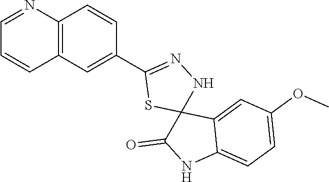 [SMILES: COc1ccc2c(c1)C1(NN=C(c3ccc4ncccc4c3)S1)C(=O)N2] | LCMS (ES) m/z = 363.1 [M + H]+; 1H NMR (400 MHz, DMSO-d₆) δ 10.37 (s, 1H), 9.10-9.08 (m, 2H), 8.50 (d, J = 8.0 Hz, 1H), 8.11 (d, J = 7.2 Hz, 1H), 8.04 (d, J = 7.2 Hz, 1H), 7.96 (s, 1H), 7.59-7.52 (m, 1H), 7.11 (s, 1H), 6.88 (d, J = 8.4 Hz, 1H), 6.78 (d, J = 7.2 Hz, 1H), 3.70 (s, 3H). HPLC purity: 98.04% at 254 nm. |
| OGM44 | 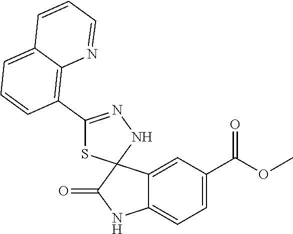 [SMILES: COC(=O)c1ccc2c(c1)C1(NN=C(c3cccc4cccnc34)S1)C(=O)N2] | LCMS (ES) m/z = 391.1 [M + H]+; 1H NMR (400 MHz, DMSO-d₆) δ 10.79 (s, 1H), 8.83 (s, 1H), 8.44 (d, J = 7.6 Hz, 1H), 8.38 (d, J = 7.6 Hz, 1H), 8.05-8.00 (m, 2H), 7.92 (d, J = 7.6 Hz, 1H), 7.69 (t, J = 8.0 Hz, 1H), 7.60-7.57 (m, 1H), 6.96 (d, J = 8.0 Hz, 1H), 3.79 (s, 3H). HPLC purity: 97.07% at 254 nm. |
| OGM46 | 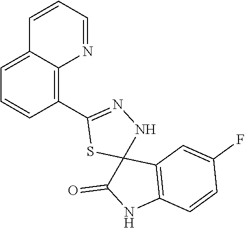 [SMILES: O=C1Nc2ccc(F)cc2C12NN=C(c1cccc3cccnc13)S2] | LCMS (ES) m/z = 351.1 [M + H]+; 1H NMR (400 MHz, DMSO d6) δ = 10.36 (s, 1H), 8.83 (s, 2H), 8.43 (d, J = 8.4 Hz, 1H), 8.37 (d, J = 6.8 Hz, 1H), 8.03 (d, J = 8.0 Hz, 1H), 7.68 (t, J = 7.6 Hz, 1H), 7.59-7.58 (m, 1H), 7.34-7.33 (m, 1H), 7.08 (t, J = 8.0 Hz, 1H), 6.83-6.82 (m, 1H); HPLC purity: 98.44% at 254 nm. |
| OGM47 | 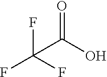 | LCMS (ES) m/z = 341.1 [M + H]+; 1H NMR (400 MHz, DMSO-d₆) δ 10.96 (s, 1H),9.11 (s, 1H), 8.76 (s, 1H), 8.62-8.61 (m, 1H), 7.98-7.94 (m, 1H), 7.53 (t, J = 7.2 Hz, 3H), |
| | 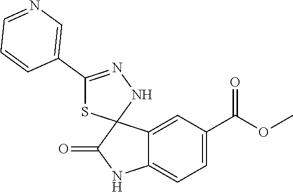 [SMILES: COC(=O)c1ccc2c(c1)C1(NN=C(c3cccnc3)S1)C(=O)N2] | 6.98 (d, J = 7.6 Hz, 1H), 3.79 (s, 3H). HPLC purity: 99.05% at 254 nm. |
| OGM48 | 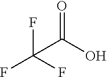 | LCMS (ES) m/z = 311.1 [M + H]+; 1H NMR (400 MHz, DMSO-d₆) δ 10.43 (s, 1H), 9.11 (bs, 1H), 8.74 (s, 1H), 8.60 (d, J = 4.0 Hz, 1H), 7.94 (d, J = 7.6 Hz, 1H), 7.51 (t, J = 5.2 |
| | 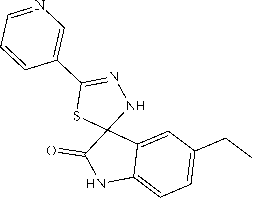 [SMILES: CCc1ccc2c(c1)C1(NN=C(c3cccnc3)S1)C(=O)N2] | Hz, 1H), 7.35 (s, 1H), 7.13 (s, 1H), 7.13 (d, J = 8.0 Hz, 1H), 6.76 (d, J = 8.0 Hz, 1H), 2.58-2.52 (m, 2H), 1.12 (t, J = 7.2 Hz, 3H). HPLC purity: 99.46% at 254 nm. |
| OGM50 | 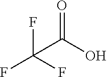 | LCMS (ES) m/z = 313.1 [M + H]+; 1H NMR (400 MHz, DMSO-d₆) δ 10.35 (s, 1H), 9.10 (s, 1H), 8.75 (s, 1H), 8.61 (d, J = 8.0 Hz, 1H), 7.97 (d, J = 7.2 Hz, 1H), 7.54 (t, J = 5.2 |
| | 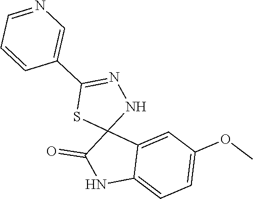 [SMILES: COc1ccc2c(c1)C1(NN=C(c3cccnc3)S1)C(=O)N2] | Hz, 1H), 7.08 (s, 1H), 6.88 (d, J = 8.4 Hz, 1H), 6.77 (d, J = 8.4 Hz, 1H), 3.69 (s, 3H). HPLC purity: 98.43% at 254 nm. |
| OGM52 | 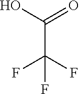 | LCMS (ES) m/z = 361.0 [M + H]+; 1H NMR (400 MHz, DMSO d6) δ = 10.89 (s, 1H), 8.94 (s, 1H), 7.90 (s, 1H), 7.74 (d, J = 8.0 Hz, 1H), 7.56- 7.54 (m, 2H), 7.45-7.43 (m, 3H), 7.28 (s, 2H), 6.98 (d, J = 8.4 Hz, |
| | 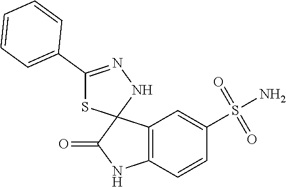 [SMILES: NS(=O)(=O)c1ccc2c(c1)C1(NN=C(c3ccccc3)S1)C(=O)N2] | 1H); HPLC purity: 99.29% at 254 nm. |
| OGM53 | 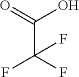 | LCMS (ES) m/z = 360.1 [M − H]+; 1H NMR (400 MHz, DMSO d6) δ = 10.93 (s, 1H), 9.16 (bs, 1H), 8.76 (s, 1H), 8.61-8.60 (m, 1H), 7.96 (d, J = 8.0 Hz, 1H), 7.91 (s, 1H), 7.76 (d, |
| | 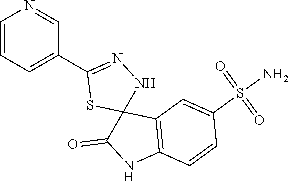 [SMILES: NS(=O)(=O)c1ccc2c(c1)C1(NN=C(c3cccnc3)S1)C(=O)N2] | J = 8.0 Hz, 1H);, 7.53-7.50 (m, 1H), 7.18 (s, 2H), 6.99 (d, J = 8.4 Hz, 1H); HPLC purity: 99.67% at 254 nm. |
| OGM54 | 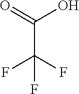 | LCMS (ES) m/z = 377.1 [M + H]+; 1H NMR (400 MHz, DMSO d6) δ = 12.8 (bs, 1H), 10.95 (s, 1H), 9.10 (s, 1H), 8.92 (s, 1H), 8.48 (d, J = 7.6 Hz, 1H), 8.11 (d, J = 8.8 Hz, 1H), 8.04 (d, J = 8.8 Hz, 1H), 7.98 (s, 2H), 7.92 |
| | 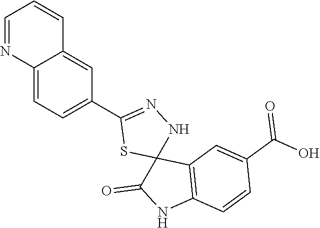 [SMILES: O=C(O)c1ccc2c(c1)C1(NN=C(c3ccc4ncccc4c3)S1)C(=O)N2] | (d, J = 8.4 Hz, 1H), 7.60-7.57 (m, 1H), 6.96 (d, J = 8.4 Hz, 1H); HPLC purity: 99.1% at 254 nm. |
| OGM55 | 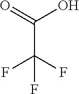 | LCMS (ES) m/z = 412.1 [M + H]+; 1H NMR (400 MHz, DMSO d6) δ = 10.95 (s, 1H), 9.18 (s, 1H), 8.95 (s, 1H), 8.54 (d, J = 7.6 Hz, 1H), 8.14 (d, J = 8.4 Hz, 2H), 8.08-8.01 (m, 1H), 7.94 (s, 1H), 7.76 (d, J = 8.0 Hz, |
| | 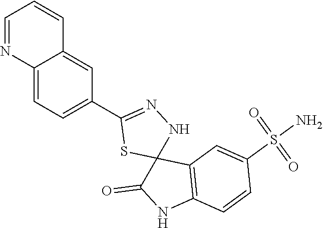 [SMILES: NS(=O)(=O)c1ccc2c(c1)C1(NN=C(c3ccc4ncccc4c3)S1)C(=O)N2] | 1H), 7.63 (s, 1H), 7.29 (s, 2H), 7.01 (d, J = 8.4 Hz, 1H); HPLC purity: 95.10% at 224 nm. |
| OGM56 | 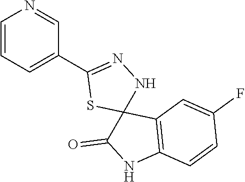 [SMILES: O=C1Nc2ccc(F)cc2C12NN=C(c1cccnc1)S2] | LCMS (ES) m/z = 301.0 [M + H]+; 1H NMR (400 MHz, DMSO d6) δ = 10.55 (s, 1H), 9.08 (s, 1H), 8.72 (s, 1H), 8.57 (s, 1H), 7.90 (d, J = 7.6 Hz, 1H), 7.48-7.45 (m, 1H), 7.37- 7.36 (m, 1H);, 7.16-7.12 (m, 1H), 6.86-6.84 (m, 1H); HPLC purity: 98.88% at 220 nm. |
| OGM58 | 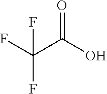 | LCMS (ES) m/z = 361.2 [M + H]+; 1H NMR (400 MHz, DMSO d6) δ = 10.36 (s, 1H), 8.83 (s, 2H), 8.43 (d, J = 8.4 Hz, 1H), 8.37 (d, J = 6.8 Hz, 1H), 8.03 (d, J = 8.0 Hz, 1H), 7.68 (m, 1H), 7.59-7.58 (m, 1H), 7.34- |
| | 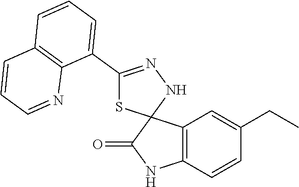 [SMILES: CCc1ccc2c(c1)C1(NN=C(c3cccc4cccnc34)S1)C(=O)N2] | 7.33 (m, 1H), 7.08 (s, 1H), 6.74- 6.72 (m, 1H), 2.58-2.56 (m, 2H), 1.15-1.11 (m, 3H); ; HPLC purity: 98.5% at 254 nm. |
| OGM61 | 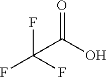 | LCMS (ES) m/z = 327.1 [M + H]+; 1H NMR (400 MHz, DMSO-d₆) δ 12.9 (s, 1H), 10.92 (s, 1H), 9.10 (s, 1H), 8.75 (s, 1H), 8.61 (s, 1H), 7.98- 7.91 (s, 3H), 7.54-7.52 (m, 1H), |
| | 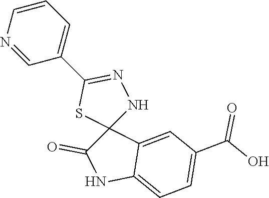 [SMILES: O=C(O)c1ccc2c(c1)C1(NN=C(c3cccnc3)S1)C(=O)N2] | 6.95 (d, J = 8.0 Hz, 1H). HPLC purity: 99.12% at 254 nm. |
| OGM17 | 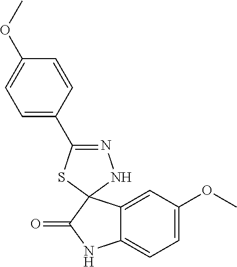 [SMILES: COc1ccc(C2=NNC3(S2)C(=O)Nc2ccc(OC)cc23)cc1] | ¹H NMR (400 MHz, DMSO-d₆): δ 3.71-3.85 (6H, 3.76 (s), 3.80 (s)), 6.44 (1H, dd, J = 2.8, 0.4 Hz), 6.63 (1H, dd, J = 8.8, 2.8 Hz), 6.97 (1H, dd, J = 8.8, 0.4 Hz), 7.25 (2H, ddd, J = 8.8, 1.1, 0.5 Hz), 7.38 (2H, ddd, J = 8.8, 1.8, 0.5 Hz). |
| OGM74 | 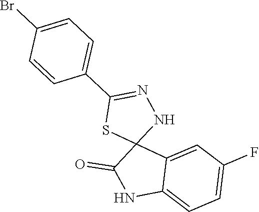 [SMILES: O=C1Nc2ccc(F)cc2C12NN=C(c1ccc(Br)cc1)S2] | ¹H NMR(400 MHz, DMSO-d₆): δ 6.82 (1H, dd, J = 8.7, 0.5 Hz), 6.99- 7.16 (2H, 7.05 (dd, J = 8.7, 1.8 Hz), 7.10 (dd, J = 1.8, 0.5 Hz)), 7.38-7.61 (4H, 7.45 (ddd, J = 8.7, 1.9, 0.5 Hz), 7.55 (ddd, J = 8.7, 1.6, 0.5 Hz)). |

### C Synthesis of methyl 2,3-dioxoindoline-5-carboxylate

Procedure: To a stirred solution of methyl 1H-indole-5-carboxylate (3.00 g, 17.1 mmol) in 50.0 mL of DMSO were added 1-iodopyrrolidine-2,5-dione (4.62 g, 1.2 eq., 20.5 mmol) and 1-hydroxy-1-oxo-3H-1λ⁵,2-benziodaoxol-3-one (14.4 g, 3 eq., 51.4 mmol) at 25° C. The reaction mixture was stirred for 3 hours at the same temperature, monitored by TLC. The reaction mixture was poured into ice and extracted with ethyl acetate (3×30 mL). The reaction was monitored by TLC. After completion of reaction, the reaction mixture was diluted with water (50 mL) and extracted with ethyl acetate (3×50 mL). The combined organic layer was washed with saturated Na₂S₂O₃ (30 mL), water (20 mL), brine (20 mL), dried over anhydrous sodium sulfate and concentrated under reduced pressure to give crude product. The crude product was purified by flash column chromatography over silica gel by using combiflash purifier with 70% ethyl acetate in heptane as eluent to give methyl 2,3-dioxoindoline-5-carboxylate as brown solid. (Yield: 2.2 g, 62%). m/z=204.0 [M+H]+.

### D. Synthesis of 5-ethyl-5′-(2-methoxypyridin-4-yl)-1,2-dihydro-3′H-spiro[indole-3,2′-[1,3,4]thiadiazol]-2-one: 2,2,2-trifluoroacetic acid (OGM49)

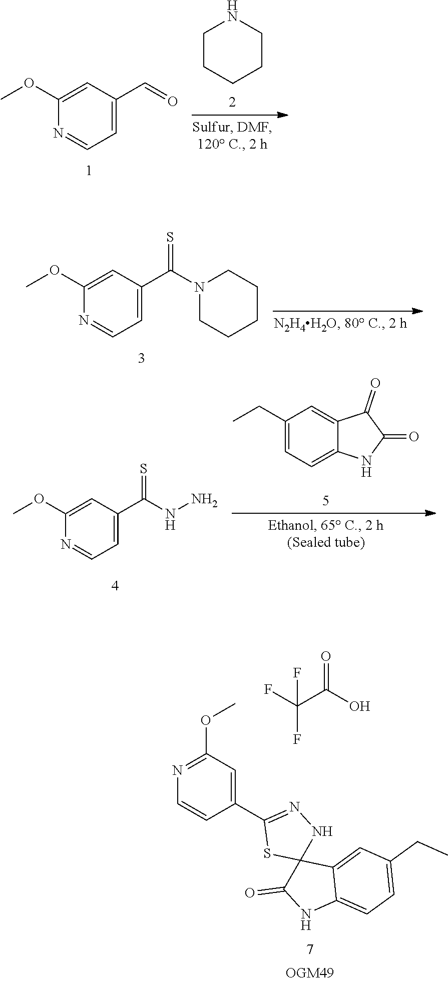

Procedure: to a stirred solution of 2-methoxypyridine-4-carbaldehyde (2.0 g, 14.6 mmol) in 10.0 mL of DMF were added piperidine (1.24 g, 14.6 mmol) and sulfur (1.87 g, 4 eq., 58.3 mmol) at room temperature. The reaction mixture was stirred for 3 h at 120° C. Reaction was monitored by TLC. The reaction mixture was poured into ice water (50 mL) and the precipitate was filtered and the solid washed with heptane and dried under vacuum to give crude (2-methoxypyridin-4-yl) (piperidin-1-yl)methanethione as yellow solid. (Yield:

$$3. g , 87 \% ) . m / z = 237.2 [ M + H ] + .$$

### F. Synthesis of N-amino-2-methoxypyridine-4-carbothioamide

A mixture of 2-methoxy-4-(piperidine-1-carbothioyl)pyridine (2.2 g, 9.31 mmol) and hydrazine monohydrate (10 mL) was stirred for 2 hours at 80° C. The reaction was monitored by TLC and LCMS, the reaction mixture was diluted with water (30 mL) and the solution carefully adjusted to pH-6 with acetic acid. The desired product was extracted with ethyl acetate (3×50 mL), washed with water (30 mL), brine (30 mL) and the combined organic layer was dried over sodium sulphate and evaporated under reduced pressure to give crude N-amino-2-methoxypyridine-4-carbothioamide as a brown solid. (Yield: 1.0 g, crude).

$$m / z = 184.1 [ M + H ] + .$$

### G. Synthesis of 5-ethyl-5′-(2-methoxypyridin-4-yl)-1,2-dihydro-3′H-spiro[indole-3,2′-[1,3,4]thiadiazol]-2-one: 2,2,2-trifluoroacetic acid (OGM49, UMMC01-JBL-55680-P-01)

Procedure: to a stirred solution of N-amino-2-methoxypyridine-4-carbothioamide (0.2 g, 1.09 mmol) in ethanol (5.00 mL), was added 5-ethyl-2,3-dihydro-1H-indole-2,3-dione (0.191 g, 1.09 mmol) at room temperature. The reaction mixture was stirred for 2 hours at 65° C. Reaction was monitored by TLC. The reaction mixture was poured into ice and extracted with ethyl acetate (3×30 mL). The reaction was monitored by TLC. After completion of reaction, the reaction mixture was diluted with water (50 mL) and extracted with ethyl acetate (3×50 mL). The combined organic layer was washed with water (20 mL), brine (20 mL), dried over anhydrous sodium sulfate and concentrated under reduced pressure to give crude product. The crude product was purified by flash column chromatography by using Combiflash™ purifier with 70% ethyl acetate in heptane as eluent. Further purification was on by Preparative HPLC using 0.1% TFA in water and acetonitrile. Column: X-BridgeC-18 (250 mm×4.6 mm×5mic) to give 5-ethyl-5′-(2-methoxypyridin-4-yl)-1,2-dihydro-3′H-spiro[indole-3,2′-[1,3,4]thiadiazol]-2-one: 2,2,2-trifluoroacetic acid as yellow solid. 1H NMR (400 MHz, DMSO-d6) δ 1H NMR (400 MHz, DMSO-d6) δ=10.43 (s, 1H), 9.21 (s, 1H), 8.17 (d, J=5.6 Hz, 1H), 7.31 (s, 1H), 7.13 (d, J=6 Hz, 2H), 6.77-6.72 (m, 2H), 3.85 (s, 3H), 2.56-2.48 (m, 2H), 1.10 (t, J=7.2 Hz, 3H). HPLC purity: 99.29% at 254 nm. m/z=341.2 [M+H]+(yield: 0.11 g. 29%).

### H. Example Compounds

See Table 3, below for example compounds synthesized using the general procedure outlined above for compound OGM49.

TABLE 3
Selected Example Compounds.

| Compound | | |
|---|---|---|
| Number | Compound Structure | Analytical Data |
| OGM51 | 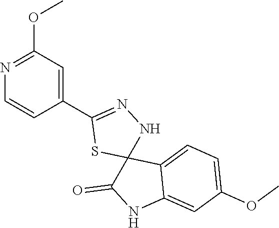 [SMILES: COc1ccc2c(c1)NC(=O)C21NN=C(c2ccnc(OC)c2)S1] | LCMS (ES) m/z = 343.2 [M + H]+; 1H NMR (400 MHz, DMSO-d₆) 1H NMR (400 MHz, DMSO-d6) δ = 10.34 (s, 1H), 9.25 (s, 1H), 8.18 (d, J = 4.4 Hz, 1H), 7.13- 7.07 (m, 2H), 6.88-6.86 (m, 1H), 6.77- 6.73 (m, 2H), 3.85 (s, 3H), 3.69 (s, 3H). HPLC purity: 98.47% at 254 nm. |
| OGM57 | 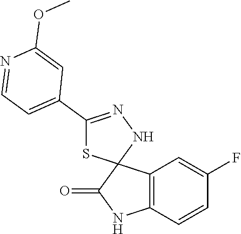 [SMILES: COc1cc(C2=NNC3(S2)C(=O)Nc2ccc(F)cc23)ccn1] | LCMS (ES) m/z = 331.2 [M + H]+; 1H NMR (400 MHz, DMSO d6) δ = 10.56 (s, 1H), 9.26 (s, 1H), 8.19-8.18 (m, 1H), 7.36 (d, J = 7.6 Hz, 1H), 7.16-7.12 (m, 2H), 6.80-6.70 (m, 1H), 6.37 (s, 1H), 3.86 (s, 3H); HPLC purity: 97.15% at 220 nm. |
| OGM59 | 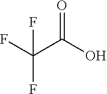 | LCMS (ES) m/z 313.1 [M + H]+; 1H NMR (400 MHz, DMSO d6) δ = 10.54 (s, 1H), 9.58 (s, 1H), 8.67 (d, J = 4.8 Hz, 2H), 7.63 (s, 2H), 7.41-7.38 (m, 1H), 6.60- 6.58 (m, 1H), 6.39 (m, 1H), 3.85 (s, 3H); HPLC purity: 99.07% at 254 nm. |
| | 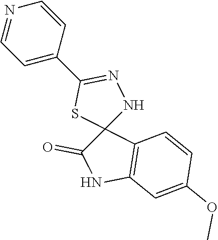 [SMILES: COc1ccc2c(c1)NC(=O)C21NN=C(c2ccncc2)S1] | |
| OGM60 | 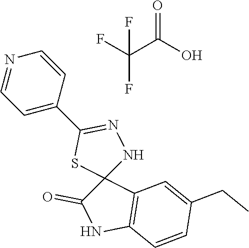 | LCMS (ES) m/z 311.2 [M + H]+; 1H NMR (400 MHz, DMSO d6) δ = 10.48 (s, 1H), 9.61 (s, 1H), 8.67 (d, J = 4.8 Hz, 2H), 7.62 (d, J = 5.2 Hz, 2H), 7.34 (s, 1H), 7.15 (d, J = 7.2 Hz, 1H), 6.76 (m, 1H), 2.57- 2.54 (m, 2H), 1.14-1.10 (m, 3H); HPLC purity: 95.5% at 254 nm. |
| OGM62 | 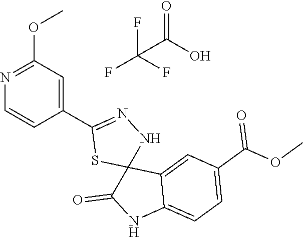 | LCMS (ES) m/z = 371.1 [M + H]+; 1H NMR (400 MHz, DMSO-d₆) δ 10.97 (s, 1H), 9.26 (s, 1H), 8.2 (d, J = 5.6 Hz, 1H), 7.94 (d, J = 8.0 Hz, 2H), 7.15 (d, J = 4.8 Hz, 1H), 6.97 (t, J = 8.4 Hz, 1H), 6.75 (s, 1H), 3.86 (s, 3H), 3.75 (s, 3H). HPLC purity: 99.15% at 254 nm. |
| OGM63 | 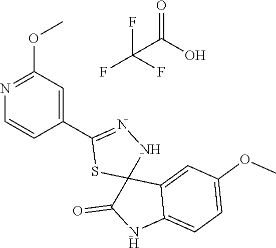 | LCMS (ES) m/z = 343.0 [M + H]+; 1H NMR (400 MHz, DMSO-d₆) δ 10.34 (s, 1H), 9.24 (s, 1H), 8.18 (d, J = 5.2 Hz, 1H), 7.13 (d, J = 5.2 Hz, 1H), 7.06 (s, 1H), 6.89-6.86 (m, 1H), 6.77-6.73 (m, 2H), 3.85 (s, 3H), 3.69 (s, 3H). HPLC purity: 99.19% at 254 nm. |
| OGM64 | 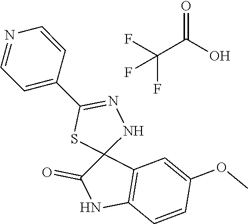 | LCMS (ES) m/z 313.1 [M + H]+; 1H NMR (400 MHz, DMSO d6) δ = 10.4 (s, 1H), 9.64 (s, 1H), 8.67 (d, J = 5.2, 2H). 7.63 (d, J = 5.2 Hz, 2H), 7.08 (s, 1H), 6.90-6.88 (m, 1H), 6.79-6.77 (m, 1H), 3.75- 3.69 (s, 3H); HPLC purity: 98.07% at 254 nm. |
| OGM65 | 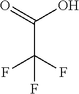 | LCMS (ES) m/z 341.0 [M + H]+; 1H NMR (400 MHz, DMSO d6) δ = 11.01 (s, 1H), 9.59 (s, 1H), 8.64 (d, J = 5.2 Hz, 2H), 7.96-7.94 (m, 2H), 7.62 (d, J = 5.2 Hz, 2H), 7.00-6.98 (m, 1H), 3.66 (s, 3H); HPLC purity: 99.87% at 254 nm. |
| | 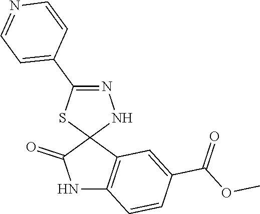 [SMILES: COC(=O)c1ccc2c(c1)C1(NN=C(c3ccncc3)S1)C(=O)N2] | |
| OGM66 | 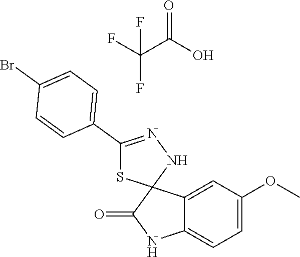 | LCMS (ES) m/z = 390.0 [M + H]+; 1H NMR (400 MHz, DMSO-d₆) δ 10.30 (s, 1H), 8.94 (s, 1H), 8.62 (d, J = 8.4 Hz, 2H), 7.46 (d, J = 8.4 Hz, 2H), 7.06 (s, 1H), 6.87-6.85 (m, 1H), 6.75 (s, 1H), 3.69 (s, 3H). HPLC purity: 99.95% at 254 nm. |
| OGM67 | 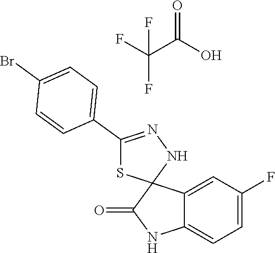 | LCMS (ES) m/z = 378.0 [M + H]+; 1H NMR (400 MHz, DMSO-d₆) δ 10.54 (s, 1H), 8.98 (s, 1H), 7.63 (d, J = 8.0 Hz, 2H), 7.46 (d, J = 8.4 Hz, 2H), 7.35 (d, J = 6.8 Hz, 1H), 7.13 (t, J = 7.6 Hz, 1H), 6.85-6.84 (m, 1H). HPLC purity: 99.89% at 254 nm. |
| OGM68 | 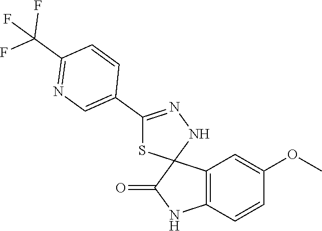 | LCMS (ES) m/z 351.0 [M + H]+; 1H NMR (400 MHz, DMSO d6) δ = 10.40 (s, 1H), 9.38 (s, 1H), 8.92 (s, 1H), 8.10 (d, J = 8.0 Hz, 1H), 7.94 (d, J = 8.0 Hz, 1H), 7.09 (s, 1H), 6.88 (d, J = 8.4 Hz, 1H), 6.77 (d, J = 8.4 Hz, 1H), 3.69 (s, 3H), HPLC purity: 99.66% at 254 nm |
| OGM69 | 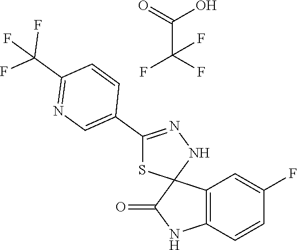 | LCMS (ES) m/z 369.2 [M + H]+; 1H NMR (400 MHz, DMSO d6) δ = 10.62 (s, 1H), 9.39 (s, 1H), 8.92 (s, 1H), 8.11 (d, J = 7.6 Hz, 1H), 7.95 (d, J = 8.0 Hz, 1H), 7.39 (d, J = 7.2 Hz, 1H), 7.15(t, J = 8.0 Hz, 1H), 6.87-6.84 (m, 1H), HPLC purity: 98.48% at 220 nm |
| OGM70 | 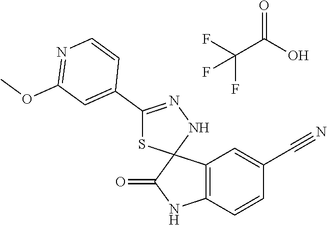 | LCMS (ES) m/z 338.3 [M + H]+; 1H NMR (400 MHz, DMSO d6) δ = 11.03 (s, 1H), 9.22 (s, 1H), 8.19 (d, J = 5.2 Hz, 1H), 7.95 (s, ,1H), 7.77 (d, J = 8.4 Hz, 1H), 7.14 (d, J = 4.8 Hz, 1H), 7.00 (d, J = 8.4 Hz, 1H), 6.75 (s, 1H), 3.86 (s, 3H); HPLC purity: 99.93% at 254 nm. |
| OGM71 | 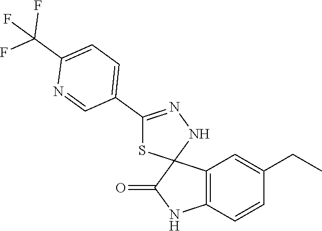 [SMILES: CCc1ccc2c(c1)C1(NN=C(c3ccc(C(F)(F)F)nc3)S1)C(=O)N2] | LCMS (ES) m/z 379.1 [M + H]+; 1H NMR (400 MHz, DMSO d6) δ = 10.49 (s, 1H), 9.35 (s, 1H), 8.91 (s, 1H), 8.09 (d, J = 8 Hz, 1H), 7.94 (d, J = 8.4 Hz, 1H), 7.36 (s, 1H), 7.14 (d, J = 8 Hz, 1H), 6.76 (d, J = 7.6 Hz, 1H), 2.64-2.55 (m, 2H), 1.13- 1.09 (m, 3H); HPLC purity: 99.61% at 254 nm. |
| OGM72 | 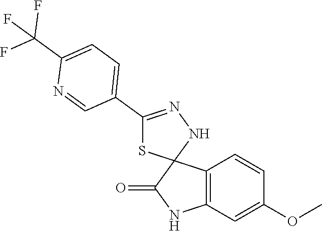 [SMILES: COc1ccc2c(c1)NC(=O)C21NN=C(c2ccc(C(F)(F)F)nc2)S1] | LCMS (ES) m/z 381.1 [M + H]+; 1H NMR (400 MHz, DMSO d6) δ = 10.53 (s, 1H), 9.29 (s, 1H), 8.90 (s, 1H), 8.08 (d, J = 7.6 Hz, 1H), 7.93 (d, J = 8.0 Hz, 1H), 7.41 (d, J = 8 Hz, 1H), 6.58 (d, J = 7.6 Hz, 1H), 6.38 (s, 1H) 3.78-3.74 (s, 3H); HPLC purity: 98.96% at 254 nm. |

### Example 3: UKbiobank has Both Synonymous Variants and a Rare Stopgain in GPR68

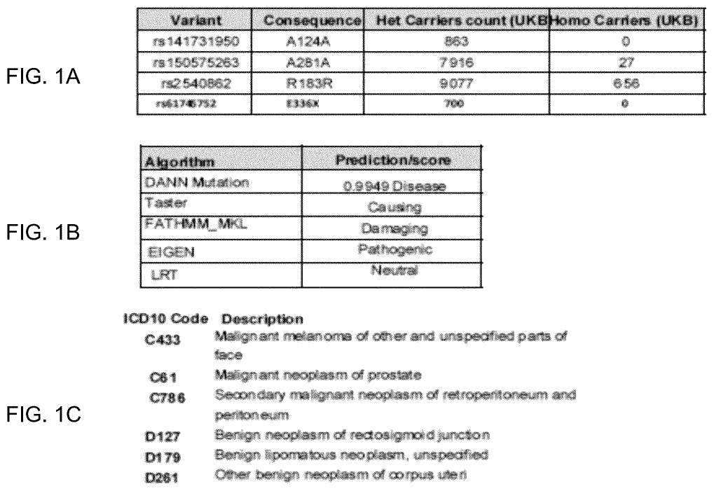

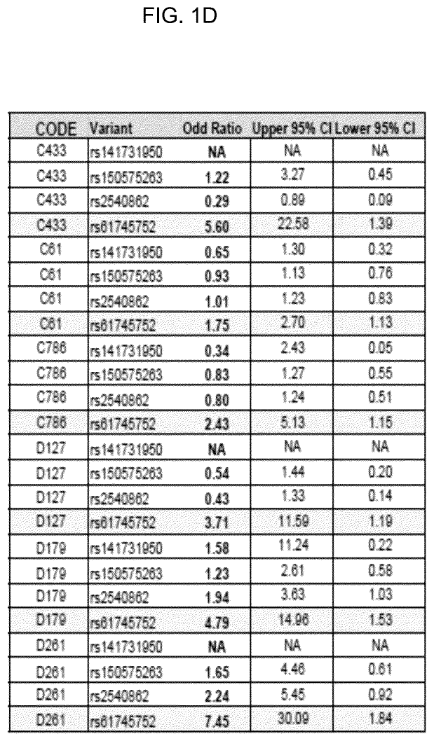

Of 500,000 individuals carriers of these variants were determined. The stop gain variant is predicted as impactful in multiple functional prediction algorithms. The stop gain variant (rs61745752, E336X) is associated with increased odds of malignant melanoma on the face, prostate cancer, secondary malignancy in the rectum and other benign neoplasms. See data in FIG. 1.

### Example 4: Initial Screenings to Discover the Active Compound OGM-1

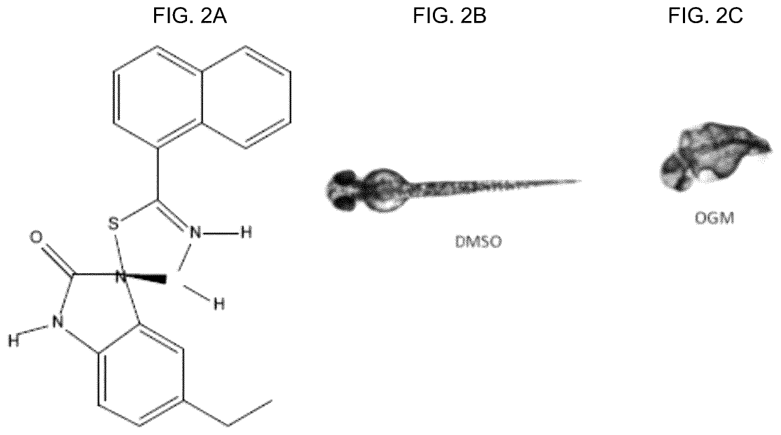

To discover novel small molecule modulators of embryonic development an unbiased screen of ˜30,000 small molecules was conducted, in which the compounds were assayed for their ability to induce phenotypic changes in the morphology of zebrafish. Further information on these assays can be found in Hao et al., 2013 and Williams et al., 2015. The compound 5-ethyl-5′-naphthalen-1-ylspiro[1H-indole-3,2′-3H-1,3,4-thiadiazole]-2-one (herein referred to as Ogremorphin-1 (OGM1) (see FIG. 2A) was identified. This compound was identified based on the phenotype of aberrant pigmentation. Compare FIG. 2B and FIG. 2C, which show a dorsal view of a representative DMSO vehicle control-treated zebrafish embryo at 48 hours pf and a dorsal view of a representative OGM1 (10 uM)-treated zebrafish embryo at 48 hours pf. In addition to pigmentation defects, Ogremorphin-1 reproducibly induced ventral curvature, wavy notochord, shortened body axis, craniofacial defects, and loss of retinal iridophores.

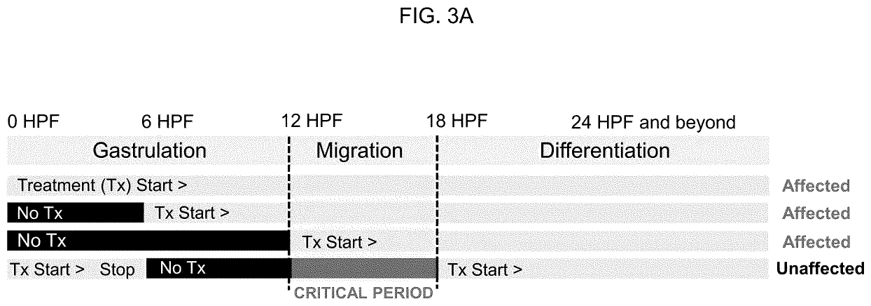

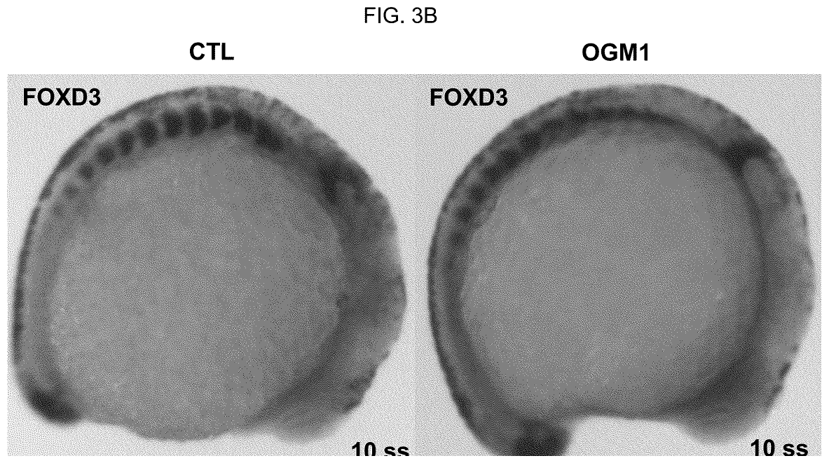

The loss of melanophores, iridophores and craniofacial cartilage are consistent with abrogation of neural crest development. Temporal phenotypic analysis was conducted. The results are shown in FIG. 3A. Pigmentation defect was dependent on treatment during the 12 hours pf-18 hours pf time window. Embryos treated throughout development (0-36 hours pf) had perturbed pigmentation. Embryos treated 6-36 hours pf and treated 12-36 hours pf also had perturbed pigmentation. Embryos treated 18-36 hours pf, however, had normal pigmentation, as well as embryos treated with a short pulse 0-6 hours pf. Thus, the time course analysis showed that pigmentation was subject to perturbation by OGM1 during a specific window of time correlated to neural crest migration (FIG. 3A).

To identify if neural crest progenitors were perturbed by OGM1, foxD3 staining was performed. This identified no difference. See FIG. 3B, which shows a lateral view of control and OGM1-treated zebrafish after in situ hybridization of FOXD3. The views on the right show a magnification of the indicated area, with arrows showing positive staining along the dorsal ridge of the embryo. This suggests that the target of OGM1 plays a role affecting pigment cells after the formation of neural crest progenitors.

### Example 5: Medicinal Chemistry

The compounds in Table 4, below, were tested for inhibition of GPR68/OGR1 using CHEM1-OGR1 cells. Briefly, the methods were: On the day before the assay 20,000 cells per well of CHEM1-OGR1 cells were plated in 10% DMEM containing 10% FBS and 1% Pen/Strep into a 96 well plate. After 24 hours media was removed and cells were stained with 80 μL Fluo-8 calcium indicator dye according to manufacturer's protocol. After staining cells were treated with 20 uL of 4× concentration of test compounds. Using kinetic imaging in the Lionheart HCS fluorescence intensity was continuously measure at 10 frames per second, for 5 seconds before 100 μL of acidified media was added to the well resulting in a pH of 6.4. A ratio of fluorescence intensities prior to addition and at peak were calculated and averaged. The resulting data were platted and using a 4 parameter logistic regression EC₅₀ (the concentration causing 50% effective inhibition) for each test compound was determined. The EC50 of each compound is provided in the table.

TABLE 4
Medicinal Chemistry Data.

| Compound Number | Compound Structure | EC₅₀ (μM) |
|---|---|---|
| OGM2 | 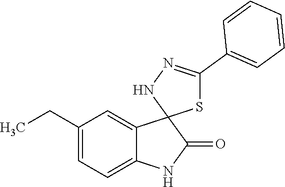 [SMILES: CCc1ccc2c(c1)C1(NN=C(c3ccccc3)S1)C(=O)N2] | 0.71 |
| OGM1 | 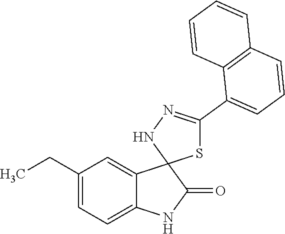 [SMILES: CCc1ccc2c(c1)C1(NN=C(c3cccc4ccccc34)S1)C(=O)N2] | 0.306 |
| OGM7 | 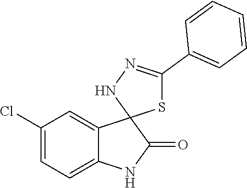 [SMILES: O=C1Nc2ccc(Cl)cc2C12NN=C(c1ccccc1)S2] | 1.17 |
| OGM8 | 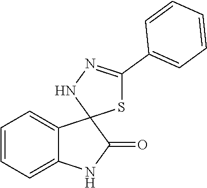 [SMILES: O=C1Nc2ccccc2C12NN=C(c1ccccc1)S2] | 2.744 |
| OGM9 | 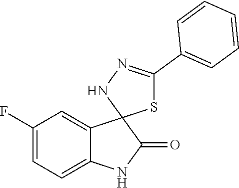 [SMILES: O=C1Nc2ccc(F)cc2C12NN=C(c1ccccc1)S2] | 2.839 |
| OGM10 | 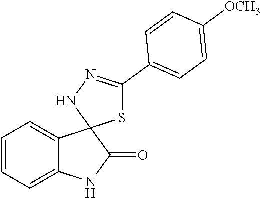 [SMILES: COc1ccc(C2=NNC3(S2)C(=O)Nc2ccccc23)cc1] | 1.44 |
| OGM11 | 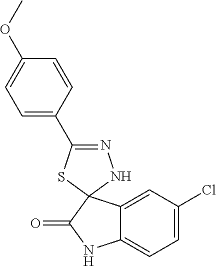 [SMILES: COc1ccc(C2=NNC3(S2)C(=O)Nc2ccc(Cl)cc23)cc1] | 4.392 |
| OGM12 | 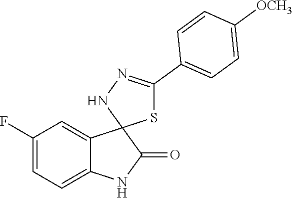 [SMILES: COc1ccc(C2=NNC3(S2)C(=O)Nc2ccc(F)cc23)cc1] | 0.756 |
| OGM13 | 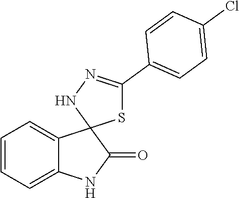 [SMILES: O=C1Nc2ccccc2C12NN=C(c1ccc(Cl)cc1)S2] | <0.826 |
| OGM14 | 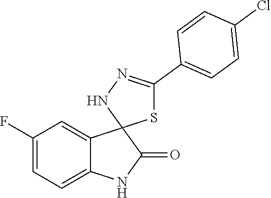 [SMILES: O=C1Nc2ccc(F)cc2C12NN=C(c1ccc(Cl)cc1)S2] | 1.831 |
| OGM15 | 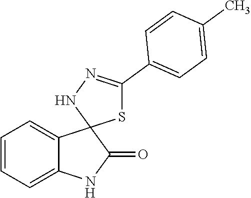 | 1.06 |
| OGM16 | 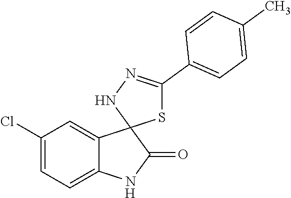 | 0.785 |
| OGM17 | 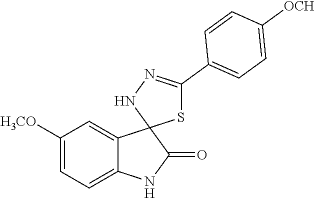 | 0.262 |
| OGM18 | 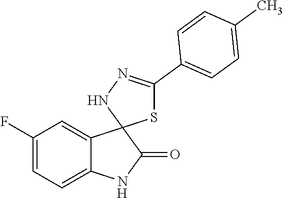 | 0.769 |
| OGM19 | 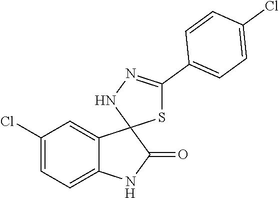 | 2.320 |
| OGM20 | 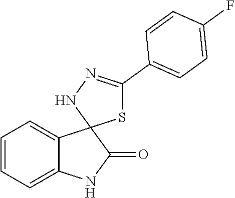 | 3.997 |
| OGM21 | 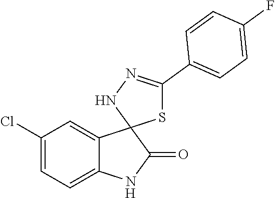 | 0.722 |
| OGM22 | 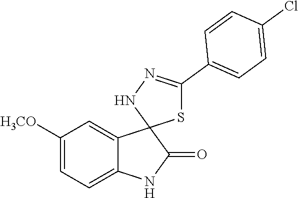 | 0.669 |
| OGM23 | 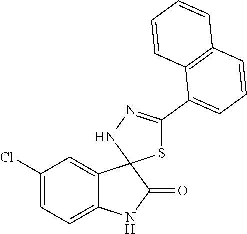 | 0.500 |
| OGM24 | 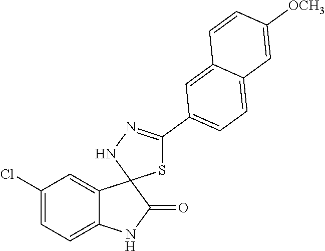 | 0.090 |
| OGM26 | 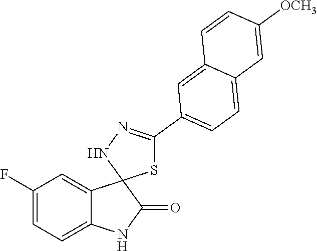 | 0.655 |
| OGM27 | 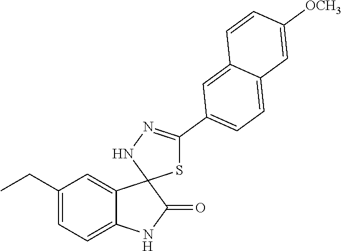 | >10 |
| OGM28 | 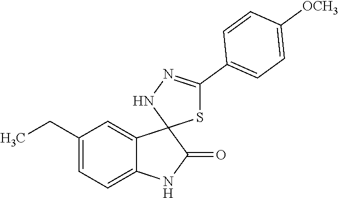 | >10 |
| OGM29 | 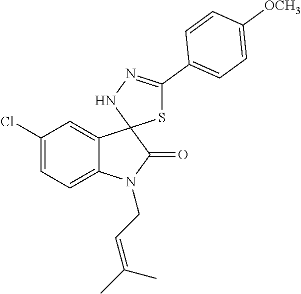 [SMILES: COc1ccc(C2=NNC3(S2)C(=O)N(CC=C(C)C)c2ccc(Cl)cc23)cc1] | >10 |
| OGM30 | 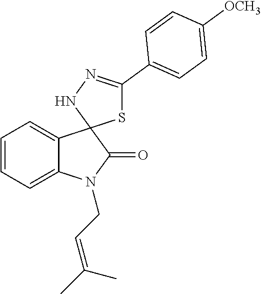 [SMILES: COc1ccc(C2=NNC3(S2)C(=O)N(CC=C(C)C)c2ccccc23)cc1] | >10 |
| OGM31 | 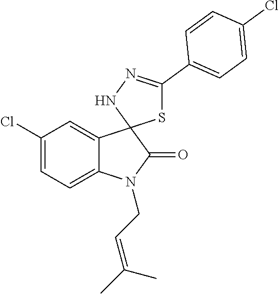 [SMILES: CC(C)=CCN1C(=O)C2(NN=C(c3ccc(Cl)cc3)S2)c2cc(Cl)ccc21] | 9.082 |
| OGM32 | 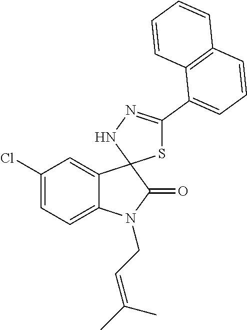 [SMILES: CC(C)=CCN1C(=O)C2(NN=C(c3cccc4ccccc34)S2)c2cc(Cl)ccc21] | >10 |
| OGM33 | 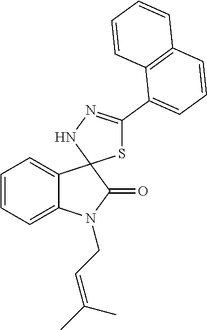 [SMILES: CC(C)=CCN1C(=O)C2(NN=C(c3cccc4ccccc34)S2)c2ccccc21] | >10 |
| OGM34 | 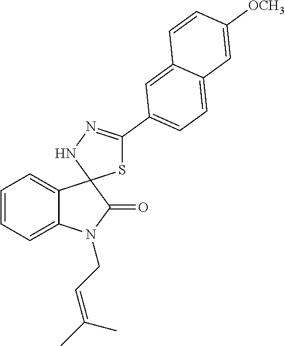 [SMILES: COc1ccc2cc(C3=NNC4(S3)C(=O)N(CC=C(C)C)c3ccccc34)ccc2c1] | >10 |
| OGM35 | 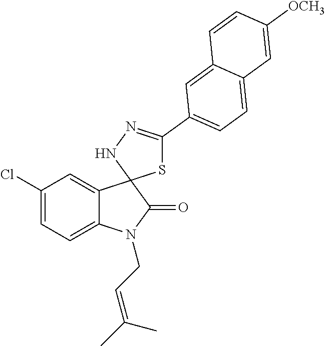 [SMILES: COc1ccc2cc(C3=NNC4(S3)C(=O)N(CC=C(C)C)c3ccc(Cl)cc34)ccc2c1] | >10 |
| OGM36 | 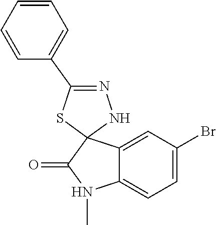 [SMILES: CN1C(=O)C2(NN=C(c3ccccc3)S2)c2cc(Br)ccc21] | 5.60 |
| OGM37 | 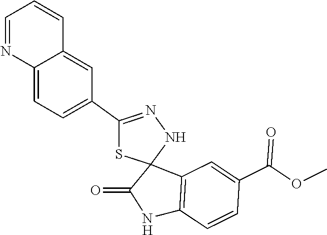 | 3.69 |
| OGM38 | 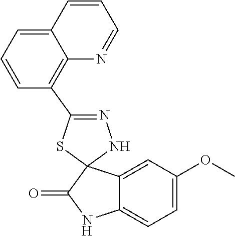 | 2.617 |
| OGM39 | 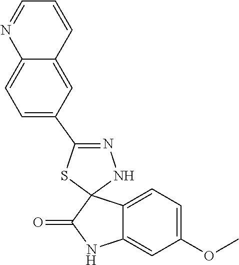 | 1.173 |
| OGM40 | 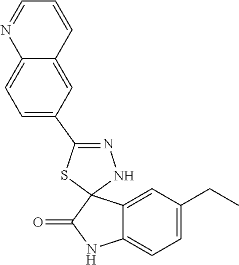 | 1.185 |
| OGM41 | 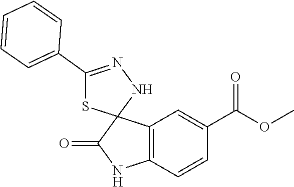 | 2.263 |
| OGM42 | 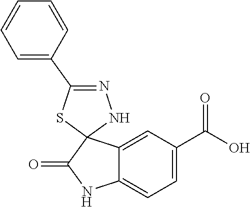 | 1.884 |
| OGM43 | 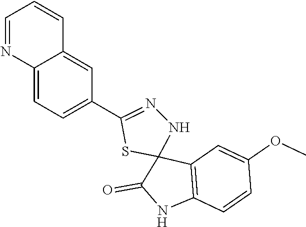 | >10 |
| OGM44 | 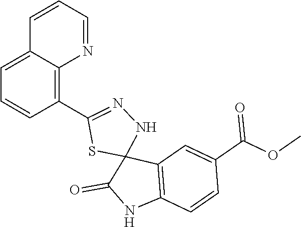 | 4.053 |
| OGM45 | 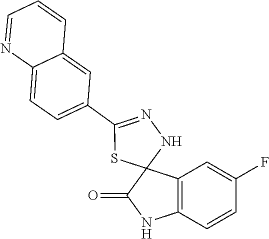 | 10 |
| OGM46 | 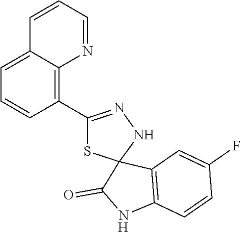 | 1.466 |
| OGM47 | 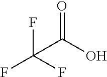 | >10 |
| | 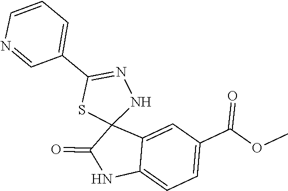 [SMILES: COC(=O)c1ccc2c(c1)C1(NN=C(c3cccnc3)S1)C(=O)N2] | |
| OGM48 | 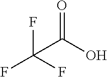 | >10 |
| | 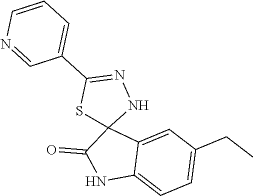 [SMILES: CCc1ccc2c(c1)C1(NN=C(c3cccnc3)S1)C(=O)N2] | |
| OGM49 | 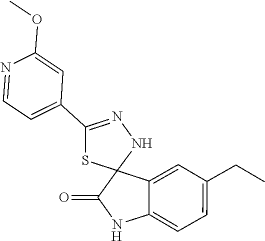 | 1.421 |
| OGM50 | 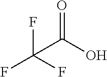 | 10.5 |
| | 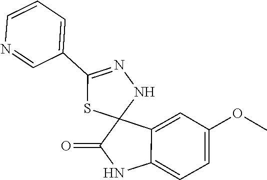 [SMILES: COc1ccc2c(c1)C1(NN=C(c3cccnc3)S1)C(=O)N2] | |
| OGM51 | 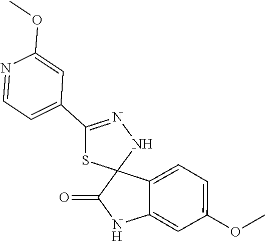 | 3.568 |
| OGM52 | 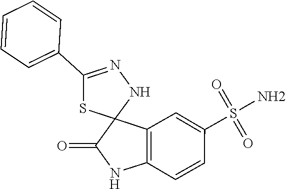 | 3.732 |
| | 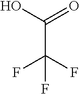 | |
| OGM53 | 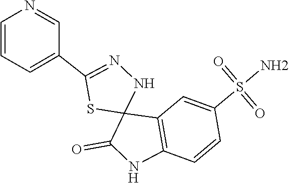 | 10.5 |
| | 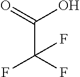 | |
| OGM54 | 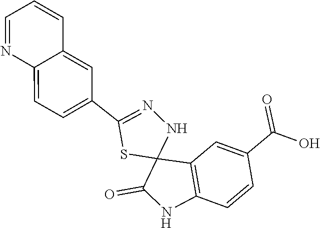 | 4.77 |
| | 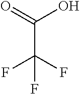 | |
| OGM55 | 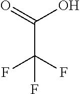 | 3.934 |
| | 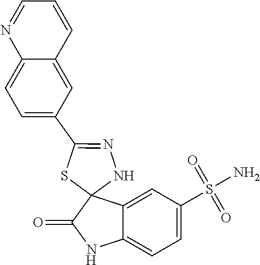 | |
| OGM56 | 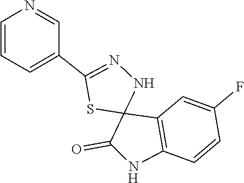 | 3.582 |
| OGM57 | 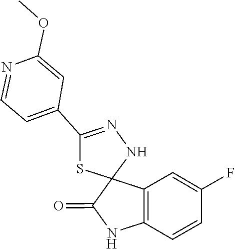 | 10.5 |
| OGM58 | 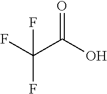 | 5.476 |
| | 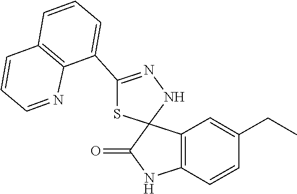 | |
| OGM59 | 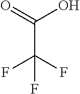 | 0.832 |
| | 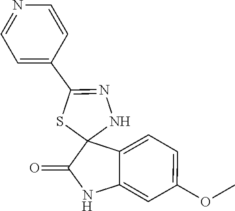 | |
| OGM60 | 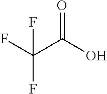 | 10.5 |
| | 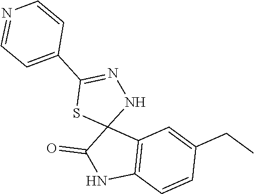 [SMILES: CCc1ccc2c(c1)C1(NN=C(c3ccncc3)S1)C(=O)N2] | |
| OGM61 | 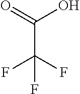 | 2.189 |
| | 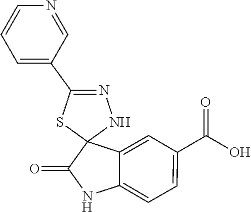 | |
| OGM62 | 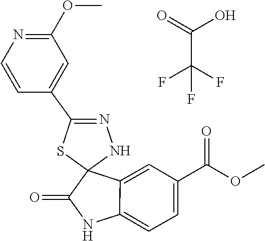 | 10.5 |
| OGM63 | 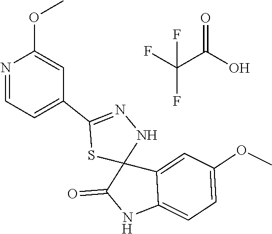 | 3.52 |
| OGM68 | 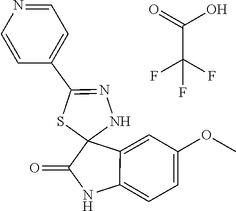 | 10.5 |
| OGM71 | 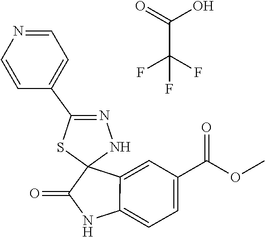 | 10.5 |
| OGM73 | 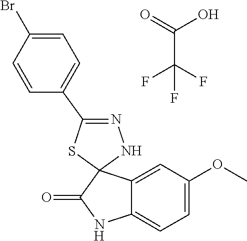 | >10 |
| OGM74 | 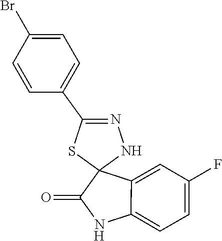 | 0.296 |
| OGM75 | 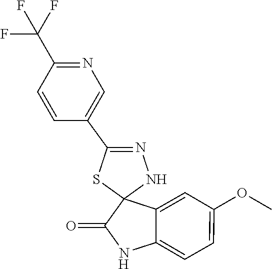 | 1.589 |
| OGM76 | 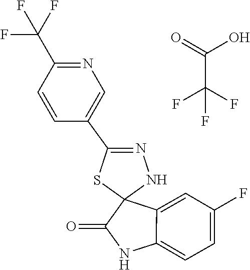 | 5.762 |
| OGM77 | 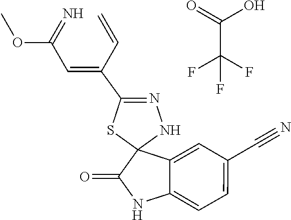 | ND |
| OGM78 | 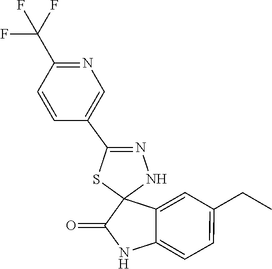 | ND |
| OGM79 | 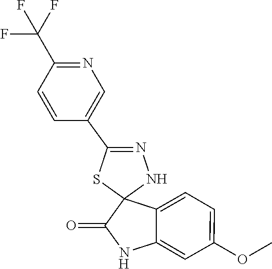 | ND |

### Example 6: GPCR Activity Based on Chemical Structure

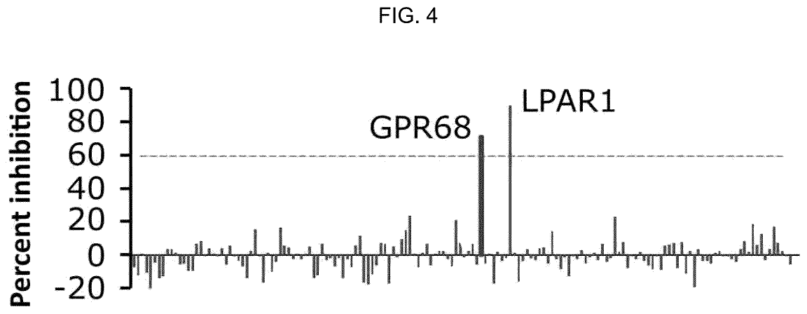

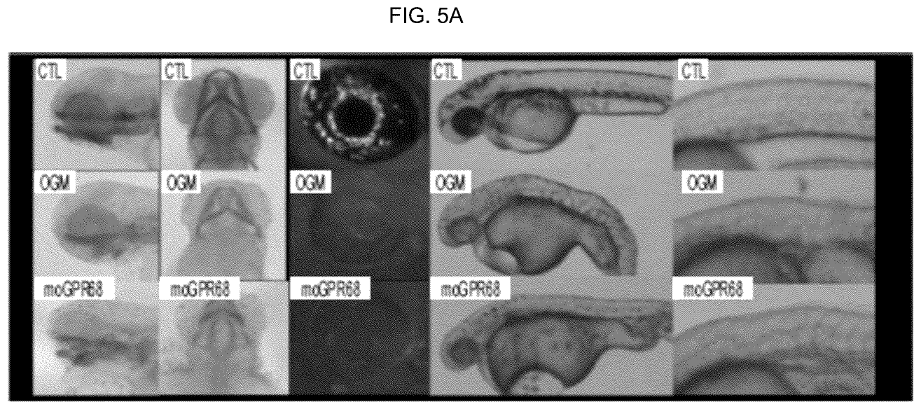

Since GPCRs represent >30% of targets for FDA-approved small molecules and are known to modulate cell migration, activity against 158 GPCRs was assessed based on their chemical structures. GPCRs (158) were tested for agonist and antagonist activity at 10 μM. For OGM, only GPR68 and LPA1 showed significant activity. UT2R also had an abnormal response and was also further investigated for thoroughness See FIG. 4. Chemical segregation analysis of zebrafish phenotype was conducted to further identify the inhibitory activity responsible for the zebrafish phenotype. Commercially available inhibitors of LPAR1 (Ki16425) and GPR14 (SB657510) were both assayed and failed to induce a noticeable phenotype at concentrations up to 200× the IC₅₀. A small-scale structure-activity relationship study around the core pharmacophore of OGM (see structure Formula A, below) showing the core scaffold for OGM derivatives) generated molecules that were similar to OGM but did not have LPAR1 activity. The identities of the R groups in Formula A are shown below in Table 5.

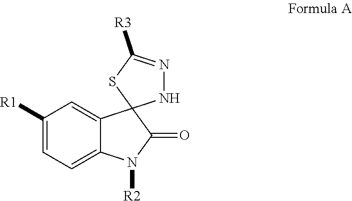

TABLE 5
Selected Compounds of interest.

| | | | | | GPR68 | Lpa1 |
|---|---|---|---|---|---|---|
| ID | R1 | R2 | R3 | Phenotype | (μM) | (μM) |
| OGM5 | H | Methyl | Benzyl | Y | 0.81 | — |
| OGM3 | Methyl | H | Benzyl | Y | 0.76 | 6.4 |
| OGM2 | Ethyl | H | Benzyl | Y | 0.71 | — |
| OGM4 | Ethyl | H | 2-phenyl | Y | 3.4 | 1.4 |
| OGM6 | H | Methyl | Naphthyl | Y | 1.5 | — |
| OGMI | Ethyl | H | Naphthyl | Y | 0.16 | 8.7 |
| LPAi | — | — | — | N | — | 0.25 |
| UT2i | — | — | — | N | — | — |

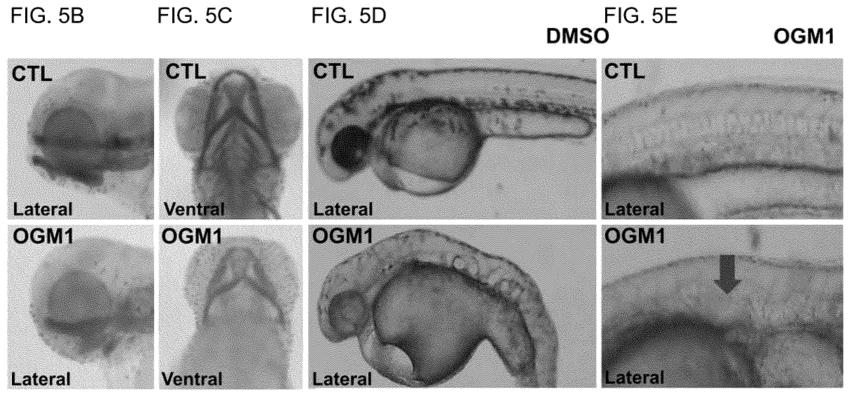

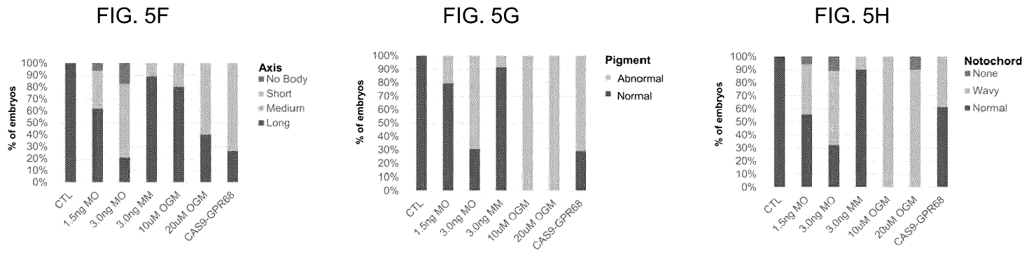

Chemical Linkage analysis was used to show that loss of LPA activity did not correlate with loss of phenotype in zebrafish. Commercial inhibitor of LPA1R (Ki16425, Sigma™) did not recapitulate phenotype. See FIG. 4, which shows the results of the Millipore GPCR screen. Chem1-GPR68 reporter cells were treated with 20 uM OM8345 for 2 hours and then rinsed with HBSS. These cells were then assayed for GPR68 at different increments of time, revealing the reversible inhibition of GPR68. The GPR68 inhibitory activity of OGM analogs segregated with the ability of the molecule to induce the phenotype in zebrafish. See FIG. 5A through FIG. 5E.

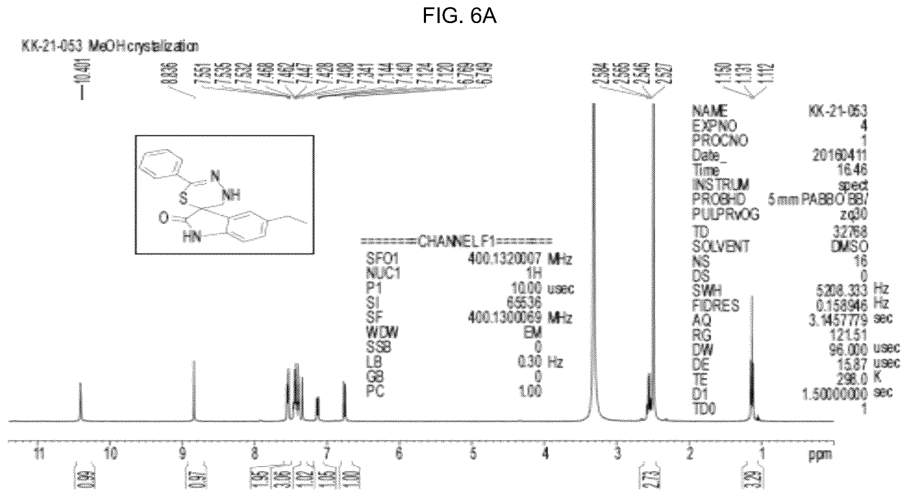

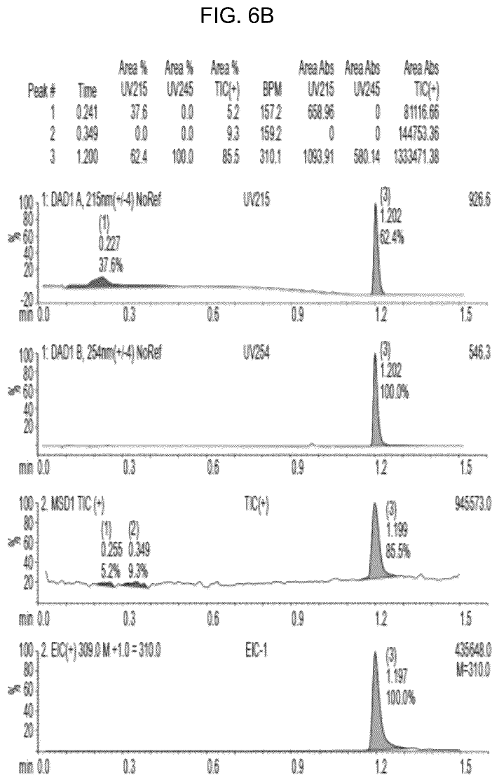

Additional experiments, whereby OGM2 was resynthesized and used (due to its submicromolar potency and GPR68 specificity) were performed. See FIG. 6A (HNMR of resynthesis of OGM2) and FIG. 6B (LCMS of resynthesis of OGM2). To test the possibility of OGM2 being an irreversible inhibitor, Chem1-OGR1 cells (Millipore™) were incubated with 10 μM OGM2 for 4 hours, and after removing the compound stimulated with pH 6.4 medium. GPR68 activity is restored rapidly after washout of inhibitor. Time till 50% max ˜52 minutes. The signal returned back towards baseline uninhibited levels in a rapid and time-dependent manner. Taken together, these data suggest that OGM2 is a reversible and highly specific inhibitor of GPR68.

### Example 7. Identification of a Highly Specific GPR68 Inhibitor

Key regulators of embryonic development are known to play critical roles in cancer. Therefore, an unbiased chemical genetic screen of ˜30,000 compounds was conducted to identify small molecules that selectively perturb zebrafish development. This screen identified 5-ethyl-5′-(1-naphthyl)-3′H-spiro [indole-3,2′-[1,3,4]thiadiazole]-2-one, herein named ogremorphin-1 (OGM1) (see FIG. 5). OGM1-associated phenotypes included a wavy notochord, abnormal pigmentation, craniofacial defects, ventral curvature, and a shortened body axis (see FIG. 5).

The disruption of melanophore and craniofacial cartilage development are consistent with defects in neural crest development. Expression of foxD3, an early marker of the pre-migratory neural crest, was not perturbed, indicating that OGM1 did not affect the formation of neural crest progenitors (see FIG. 3A).

However, the developmental window for perturbation of pigmentation by OGM1 coincides with the timing of neural crest migration (see FIG. 3B). FIG. 3B shows that embryos treated with OGM1 throughout development (from 0 to 36 hours post fertilization, hpf), from 6 to 36 hpf, or from 12 to 36 hpf had perturbed pigmentation. In contrast, embryos treated from 18 to 36 hpf had normal pigmentation. Embryos treated with a short pulse of OGM1 from 0 to 6 hpf also had normal pigmentation. Thus, the critical time window for pigment perturbation by OGM1 was between 12 and 18 hpf. DMSO=dimethylsulfoxide vehicle; Tx, treatment. Thus, OGM1 likely disrupts neural crest migration and/or subsequent differentiation.

### Example 8: GPR68 Knockdown Recapitulated OGM2-Induced Zebrafish Phenotype

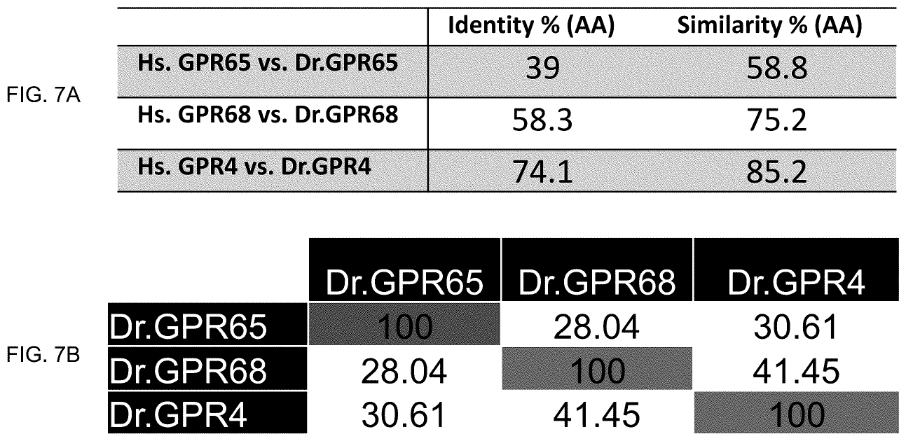

Zebrafish GPR68 and human GPR68 have a high degree of homology, with 58.3% identity and 75.2% similarity (see FIG. 7A), which shows the identity and similarity between human and zebrafish orthologs). Compared to this, in zebrafish the proton-sensing GPCR members GPR4 and GPR65 are 41.45% and 28.04% similar, respectively (see FIG. 7B), which shows similarity of acid-sensing GRCRs in zebrafish). To determine if the loss of GPR68 activity causes neural crest defects in zebrafish we knocked down GPR68 using morpholino oligonucleotides. The neural crest-specific phenotypes of iridophores, melanocyte, and craniofacial cartilage were all disrupted in a dose-dependent manner using 1.5 ng and 3 ng morpholino, the same amount of mismatch morpholino did not recapitulate the result. Additionally, the wavy notochord which is not known to be a neural crest-related phenotype was recapitulated in morphants as well. Taken together, the data suggest that the phenotypes seen in OGM2 and OGM1 treatment are due to loss of GPR68 activity and include causes defective neural crest development and wavy notochord formation. See FIG. 7A (degree of homology) and FIG. 7B (similarity of GPCR family members).

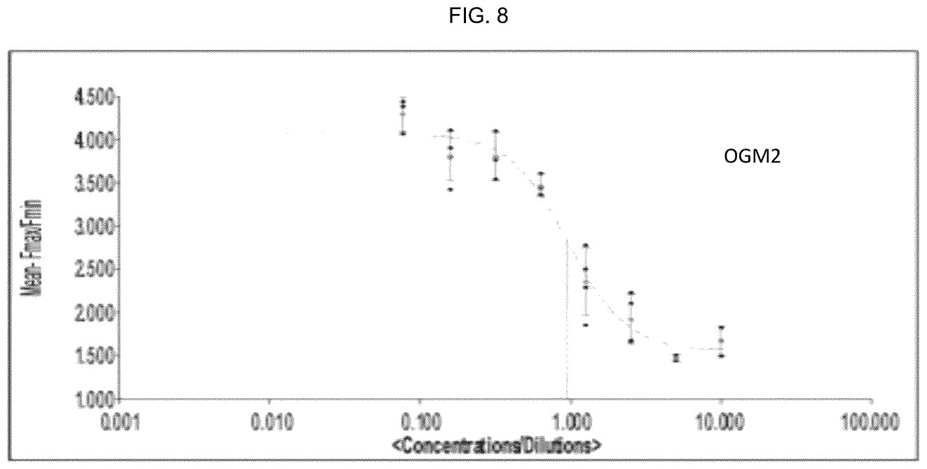

FIG. 8 shows data from a sample dose-response assay of OGM2 (Chem1-GPR68 assay) and FIG. 5 shows that morpholino knockdown of GPR68 induces craniofacial dysmorphogenesis, loss of iridophores, disrupted pigmentation, and wavy notochord. An alcian blue stain of craniofacial cartilage seen laterally and dorsally show abnormalities: loss of iridophores (left), abnormal pigmentation (middle), and undulating/kinked notochord (right). See FIG. 5. Doses of 3 ng Morpholino injections into zebrafish induced more embryos with phenotype than 1.5 ng. The 3 ng dose of mismatch morpholino had little effect. These phenotypes are consistent with 10 μM and 20 μM OGM treatments. See FIG. 5, which shows that the genetic loss of GPR68 activity phenocopies the phenotype generated by OGM2 treatment. The phenotype is the consequence of loss of GPR68 activity in vivo.

### Example 9: Gene Profiling Datasets Show that GPR68 Expression Correlates with Worse Prognosis and U87 Cells Respond to Acid Through GPR68

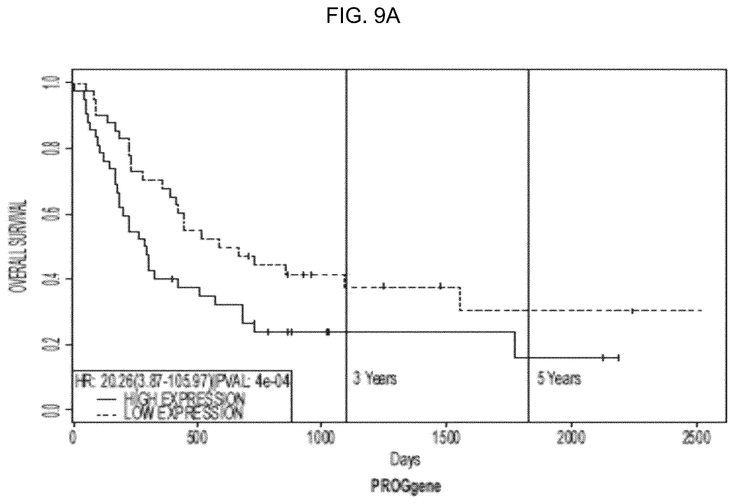

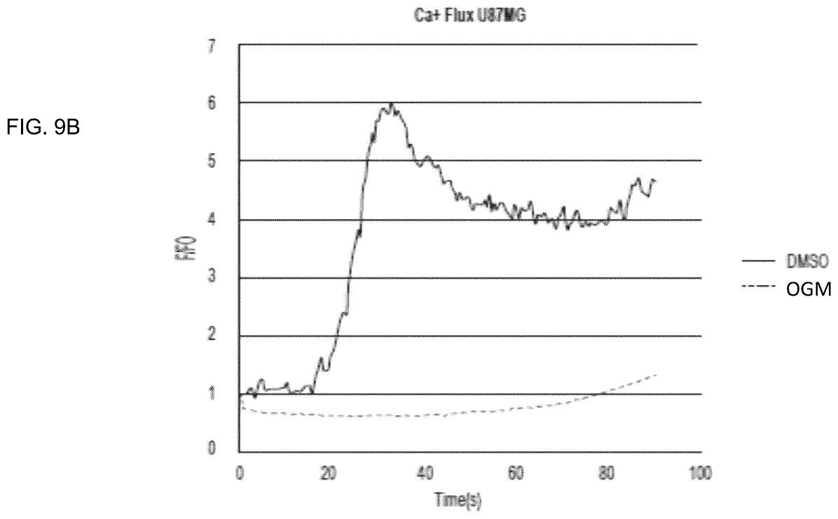

Transcriptional profiling has been applied to 85 gliomas from 74 patients to study glioma biology, prognosticate survival, and define tumor sub-classes. Patients undergoing surgical treatment at the University of California. Los Angeles for primary brain cancers between 1996 and 2003 were invited to participate in this Institutional Review Board approved study. Seventy-four of the patients participating in this broad protocol were analyzed as part of this study if their initial tumor was diagnosed as a grade III (n=24) or IV (n=50) glioma of any histologic type on initial surgical treatment and fresh frozen material was obtained. Grade III and IV gliomas were included in this study—the distinction between these grades is subtle and prone to misclassification. The time in days elapsed from resection to the day of death, or if the patient has remained alive, to the current day, was recorded for all samples studied. Patient ages at diagnosis vaned from 18 to 82 years. There were 46 females and 28 males. Probes were prepared using standard Affymetrix™ protocols, and hybridized to Affymetrix™ HG-U133A and HG-U133B arrays. The cohort was separated by High and low expression of GPR68 by above or below the median expression level. Survival of the cohort post-resection is plotted and found to be significantly different. See FIG. 9A.

GPR68 is known to signal through Gq which results in a calcium flux out of the Endoplasmic reticulum. U87 glioblastoma cells were stained, using a calcium-sensitive vital dye Fluo-8, and stimulated with acidification of the media to pH6.4. The cells respond to the acidification with a burst of calcium release, this is inhibited by OGM2. See FIG. 9B.

### Example 10: OGM Inhibits Human Melanoma Migration

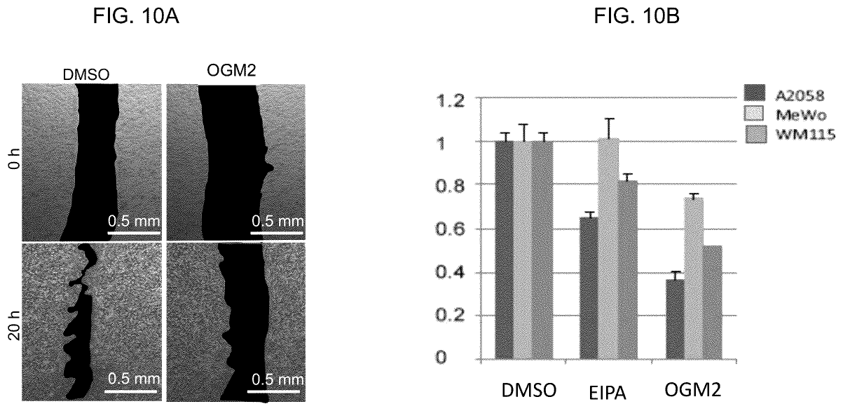

Since there are numerous parallels are found between neural crest cells and cancer during migration, we examined whether OGM could inhibit the migration of cells in human melanoma cell lines (Oppitz et al., 2007; Schriek et al., 2005, Sinnberg et al., 2018). In a scratch assay for migration of 3 human melanoma cell lines A2058, MeWo, and WM115, OGM2 (5 uM) treated cells migrated significantly less than the vehicle treated cells. A2058. MeWo and WM115 cells were grown to confluence and a stripe of cells were denuded with a p200 pipette tip “scratch” and rinsed with PBS. Cells were treated with OGM or DMSO and incubated in low serum media. Imaging was done at 30 minutes after “scratch” and then 20 hours afterwards. Areas were then measured and normalized to the initial area. FIG. 10A shows representative images of WM115 cells used in the scratch assay; OGM2 prevents wound closure compared to DMSO (CTL).

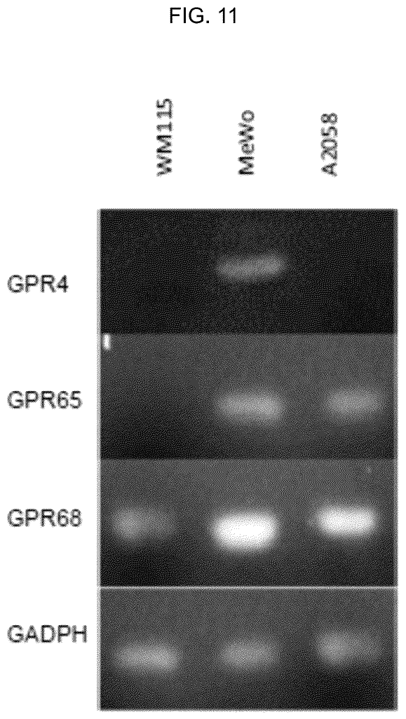

FIG. 10B shows quantification of inhibition in scratch assays across three melanoma lines. OGM2 inhibited migration in WM115, A2050 and MEWO cell lines. EIPA inhibited migration in WM115 and A2058 but not MeWo lines. Previous studies with the MV3 melanoma cell line have shown that disruption of proton efflux with EIPA inhibition of NHE1 can also reduce melanoma motility (Ludwig et al., 2013; Schneider et al., 2009; Stock et al., 2007, 2005; Stowe et al., 2007). Here, the data show that, although this effect is significant in A2058 and WM115, MeWo appears refractory to inhibition (FIG. 10B). Although the expression of GPR68 and other proton-sensing receptors have been studied in various skin cancers (Merkel cell carcinoma, dermatofibrosarcoma protuberans, atypical fibroxanthoma, and pleomorphic dermal sarcoma), expression in melanoma cell lines has not been (Nassios et al., 2019). All three of these melanoma cell lines express GPR68, but only WM115 cells did not express GPR4 or GPR65. See FIG. 11.

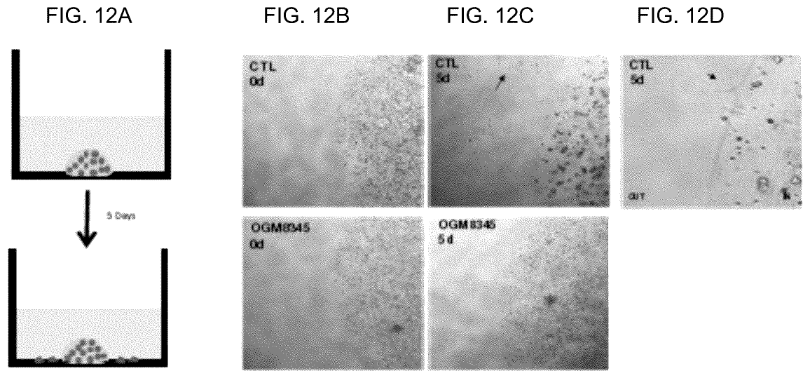

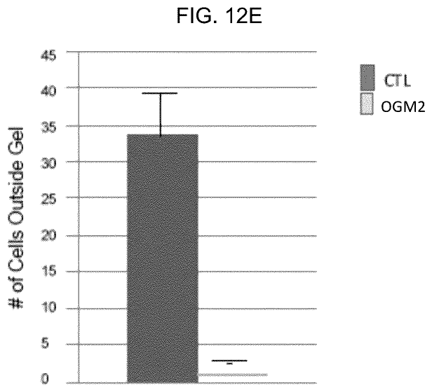

Furthermore, in a 3D model of melanoma extravasation, OGM2 treated WM115 cells migrated significantly less than the vehicle treated cells (see FIG. 12). FIG. 12A is a schematic of the agarose drop assay. WM115 cells were mixed into a low melt agarose solution and single 10 μL drops were seeded onto cell culture plates. After solidifying, media containing OGM2 (5 μM) or CTL (DMSO 0.05%) was added and wells were observed 5 days later. Immediately after media addition no cells are on the plate outside of the agarose gel in CTL or OGM2. Five days after seeding cells are observed outside of the gel (Black arrows) in CTL but not in OGM2. Close observation of CTL reveals cells crossing out of the gel drop (Black Arrowheads). Quantification on the number of cells that escaped from an average of 5 gel drop replicates. FIG. 12B through FIG. 12D show the cells after media addition. No cells are on the plate outside of the agarose gel in CTL or OGM2. Five days after seeding, cells are observed outside of the gel (arrows) in CTL but not in OGM2. Close observation of CTL reveals cells crossing out of the gel drop (arrowheads). FIG. 12E shows the quantification on the number of cells that escaped from an average of 5 gel drop replicates. Taken together this data suggests that GPR68 plays a critical role in melanoma migration in vitro.

### Example 11: Variant Rs61745752 is a Functional c-Terminal Truncation

Given the association of rs61745752 with cancer, we sought to determine the functional consequence of the variant which causes truncation after amino acid 335 (E336X). Mutations in the C-terminal tail of GPCRs can have a number of consequences, including loss of desensitization, and inactivation. GPCR desensitization is modulated through beta-arrestin binding to domains that are defined by GIRK phosphorylation.

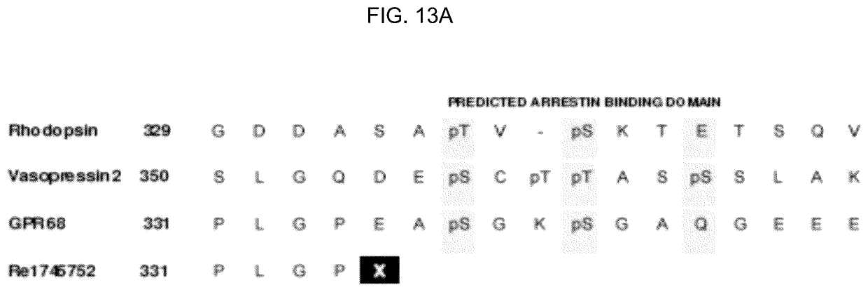

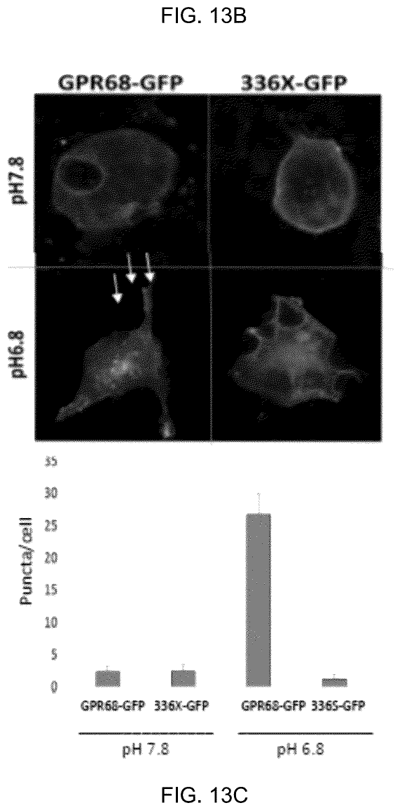

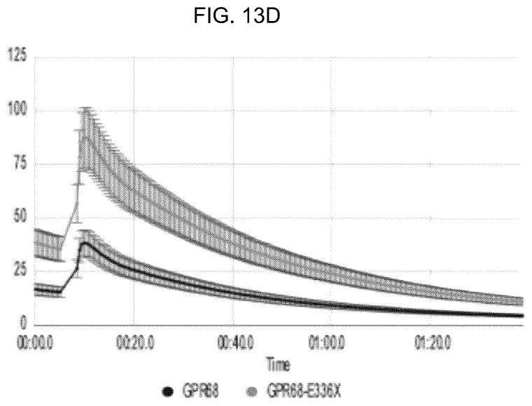

Prediction of putative binding sites for beta-arrestin binding identified a putative beta-arrestin binding site in the C-terminal cytosolic tail of the receptor, downstream of the early termination site (see FIG. 13A, a phosphocode prediction of beta-arrestin binding site downstream of amino acid 335). The sequence aligned with known beta-arrestin interaction domains of Rhodopsin and Vasopressin 2.

Transfection of native GFP-tagged (GPR68-GFP) and truncated GFP-tagged GPR68 (336X-GFP) in HEK293T revealed that the variant (335X-GFP) fails to internalize in response to stimulation, while the full-length (GPR68-GFP) internalizes in response to stimulation. See FIG. 13B and FIG. 13C. For the data shown in FIG. 13B. GPR68-GFP and 336X-GFP transfected HEK293 cells were stimulated with pH 6.8 media for 5 minutes. GPR68-GFP had puncta (white arrows) in the cell, but 336X-GFP did not form puncta under acidic conditions. FIG. 13C shows the quantitation of the number of puncta in each cell (n=20). GPR68-GFP and 336X-GFP transfected HEK293 cells have no significant receptor internalization under unstimulated conditions. Upon stimulation with pH 6.8 media for 5 minutes, GPR68-GFP had puncta (FIG. 13, white arrows) in the cell, 336X-GFP did not form puncta under acidic conditions.

293T cells were transfected with GPR68 and or E336X variant, 24 hours later cells were stained with fluo8 calcium indicator. Kinetic imaging was conducted with Lionheart HCS with a 5 second baseline reading prior to stimulation with acidification. See FIG. 13D. The variant has higher baseline levels of calcium in the cytosol which are increased upon stimulation.

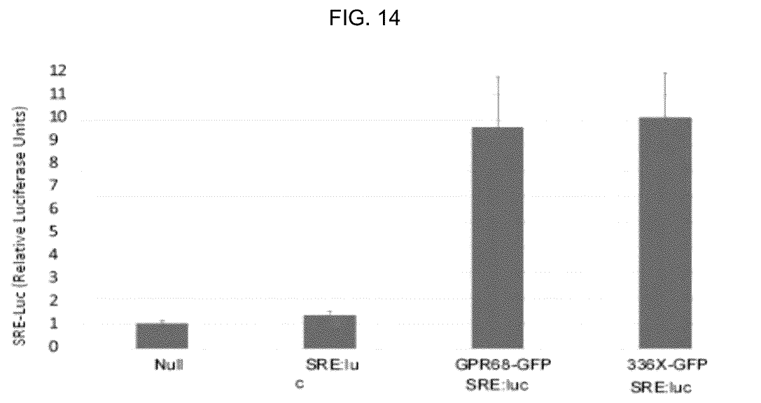

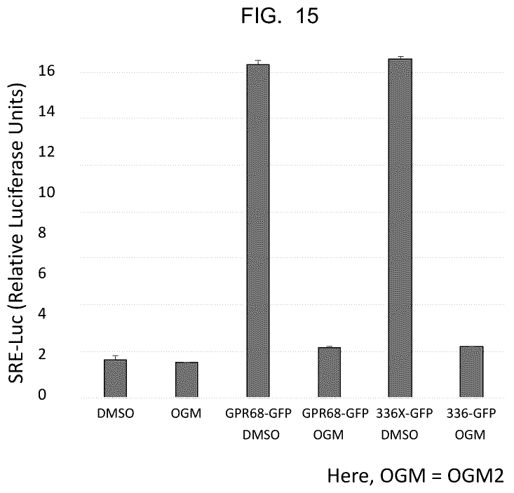

Loss of internalization can indicate loss of activity or loss of the ability to internalize through beta-arrestin. We assessed the activity of the truncation variant (336X-GFP) and found that the truncation variant still retained activity in a SRE (serum response element) responsive luciferase assay, which is activated by GPCR stimulation, particularly those coupled with Gi and Gq, through activation of the MAPK pathway. See FIG. 14, which shows that the E336X-GFP plasmid is still active in SRE-luciferase assay. The composite of results from the Serum responsive element (SRE-) luciferase assay showed low levels of activity in HEK293T cells. Compared to GFP controls, GPR68-GFP and 336X-GFP transfected cells had higher levels of activity. (n=3 biological replicates done in triplicate). Furthermore, the activity of the truncation variant was still inhibited by OGM. FIG. 15 shows representative results from a serum responsive element (SRE-) luciferase assay. The SRE shows low levels of activity in HEK293T cells. Compared to GFP controls, GPR68-GFP and 336X-GFP transfected cells had higher levels of activity. Incubation with OGM 10 μM reduced the level of luciferase activity in both GPR68-GFP and 336X-GFP conditions (n=3, technical replicates). Taken together the data suggests that rs61745752 is aberrantly active through the loss of desensitization.

### Example 12: GPR68 Activity Destabilized MCF7 Spheroids

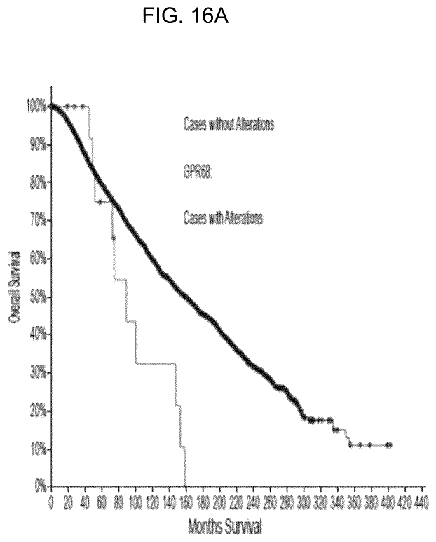

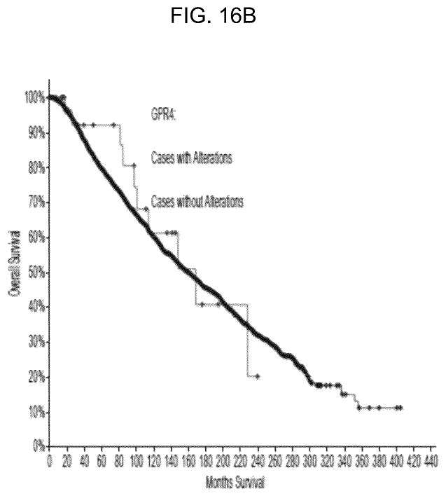

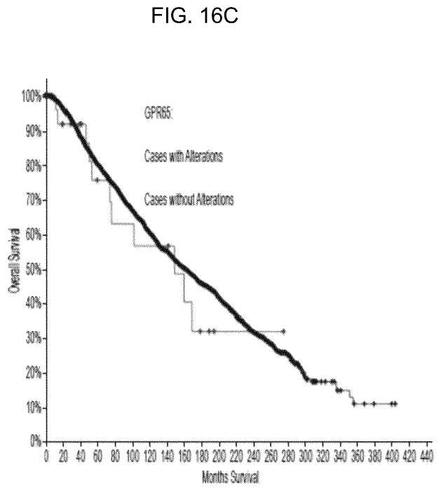

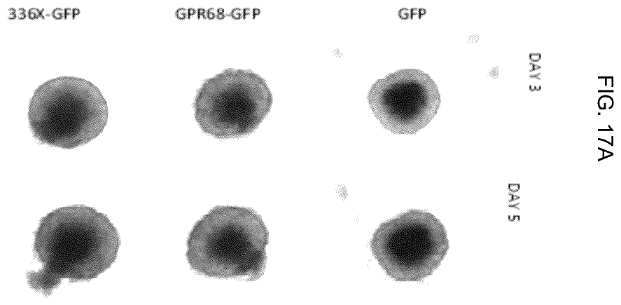

Finally, given the association of rs61745752 with cancer, we investigated mutational burden and survival in the cBioPortal cohort. To ascertain if GPR68 and its variant could modulate metastatic potential in breast cancer in vitro we utilized spheroid integrity and invasion assay as described in Gayan et al., 2017 and Jung et al., 2019. MCF7 cells transfected with GFP, GPR68-GFP, and 336X-GFP were seeded in Ultra low attachment plates to form spheroids. At 48 hours 1:4 geltrex matrix was added. The variant E336X-GFP expressing spheroids were less stable than those expressing the wildtype sequence of GPR68. Mutations in GPR68 correlated with poor prognosis in breast cancer. FIG. 16 provides Kaplan-Mier plots generated in cBioportal of Breast cancer cohort carrying alterations in GPR68 (p=0.013), alterations in GPR4 (p=0.785), and alterations in GPR65 (p=0.612). Breast cancer patients harboring mutations in GPR68 had worse overall survival than those without. By contrast, the closely related members of the proton-sensing GPCR family GPR4 and GPR65 did not have similar effects on overall survival. See also Tables 6-8, below.

TABLE 6
Breast Cancer Patients; alterations in GPR68.

| | Number of | Number of | Median Months |
|---|---|---|---|
| | Cases, Total | Cases, Deceased | Survival |
| Cases with Alterations | 18 | 10 | 89.96 |
| in GPR68 | | | |
| Cases without | 6447 | 1879 | 161.7 |
| Alterations in GPR68 | | | |

TABLE 7
Breast Cancer Patients; alterations in GPR4.

| | Number of | Number of | Median |
|---|---|---|---|
| | Cases, | Cases, | Months |
| | Total | Deceased | Survival |
| Cases with Alterations | 32 | 10 | 168.2 |
| in GPR4 | | | |
| Cases without | 6433 | 1879 | 160.3 |
| Alterations in GPR4 | | | |

TABLE 8
Breast Cancer Patients; alterations in GPR65.

| | Number of | Number of | Median |
|---|---|---|---|
| | Cases, | Cases, | Months |
| | Total | Deceased | Survival |
| Cases with Alterations | 27 | 11 | 148.1 |
| in GPR65 | | | |
| Cases without | 6438 | 1878 | 160.4 |
| Alterations in GPR65 | | | |

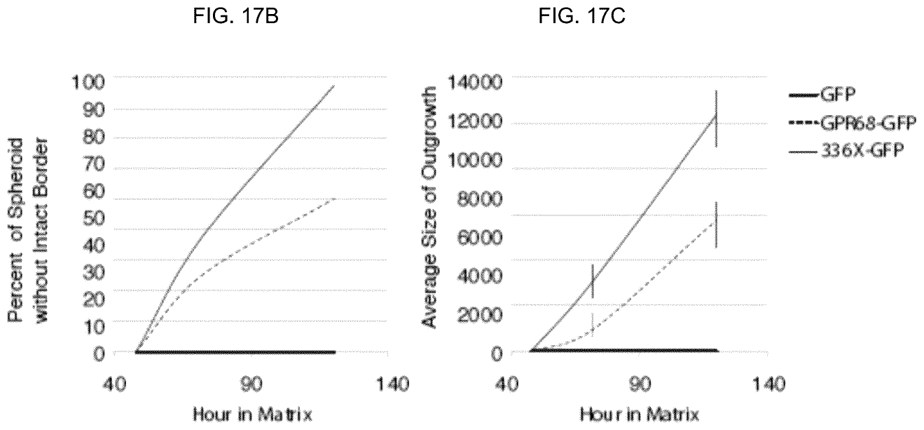

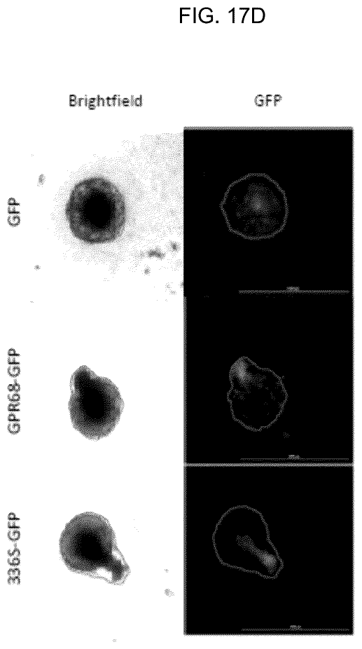

Transient transfection in MCF7 cells of GFP, GPR68-GFP, 336X-GFP revealed that, 3 days post matrix addition, the continuity of the outer ring of cells becomes disrupted by the inner core in GPR68-GFP and 336X-GFP transected spheroids. MCF7 cells transfected with GFP, GPR68-GFP, and 336X-GFP were seeded in Ultra low attachment plates to form spheroids. At 48 hours, 1:4 geltrex matrix was added. GFP transfected spheroids have the typical structure of outer proliferative ring with an inner quiescent core GPR68-GFP and 336X-GFP expressing spheroids have an irregular structure with the outer proliferative ring no longer intact on day 3. See FIG. 17A. On day 5 GPR68-GFP and 336X-GFP spheroids show outgrowths from where the outer ring is not intact. The cells of the inner core then extrude out into the matrix increasingly over 5-7 days post matrix addition. See FIG. 17A.

Furthermore, the degree of invasion in the 336X-GFP spheroids was greater than that of GPR68-GFP. See FIG. 17B and FIG. 17C, which show quantitation of the number of spheroids without intact outer rings (n=8) and quantitation of the size of the outgrowth from spheroids, respectively. Notably, the invasive cells from the inner core were primarily GFP+. See also FIG. 17D, which FIG. 17D presents an image of GFP+ cells in spheroids, revealing that GFP+ cells are the primary component of the spheroid outgrowths. Taken together, these data suggest that GPR68 and its variant rs61745752 are sufficient to induce a more invasive phenotype in noninvasive breast cancer MCF7 cells.

### Example 13: Detection of Rs61745752 for Diagnostic Purposes

Given the association of rs61745752 with cancer (FIG. 1) and the functional studies of increased metastatic ability in MCF7 cells expressing the truncation construct (FIG. 17), further applications of the invention include, the diagnostic testing for the SNP rs61745752 (In human genome assembly GRCh38.p12 this variant is on chromosome 14 at position 91234045 coding a change of the nucleotide Cytosine to Adenine) which results in an early truncation of the protein E336X, at the DNA, cDNA, or protein level to make medical care decisions in treatment of neoplasms.

### Example 14: Matrix Invasion in U87 Glioblastoma Spheroids

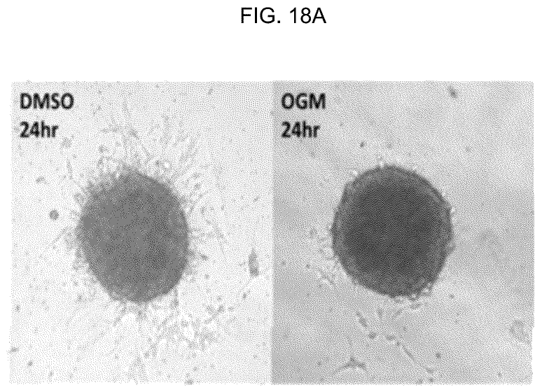

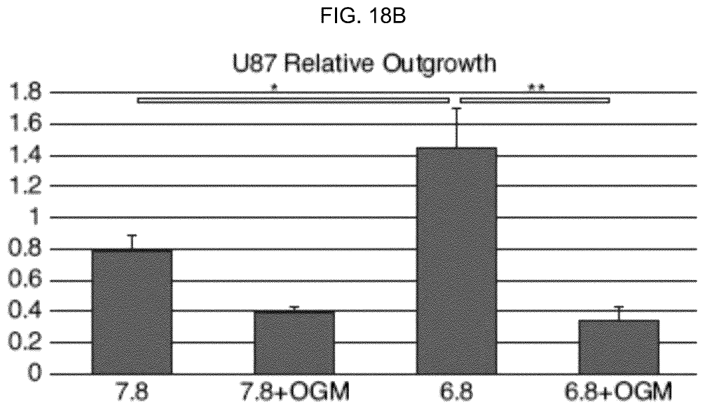

U87 glioblastoma spheroids were formed in low attachment round bottom plates for 3 days. Spheroids were then covered in Matrigel™, which was allowed to polymerize before treatment with acidified media with or without OGM2. Acidification increased the degree of matrix invasion which is attenuated by OGM2. See FIG. 18.

### Example 15: GPR68 Inhibition Attenuates Growth in 2D Cell Culture in Both TMZ Sensitive (U87) and Insensitive (U138) Glioblastoma Models

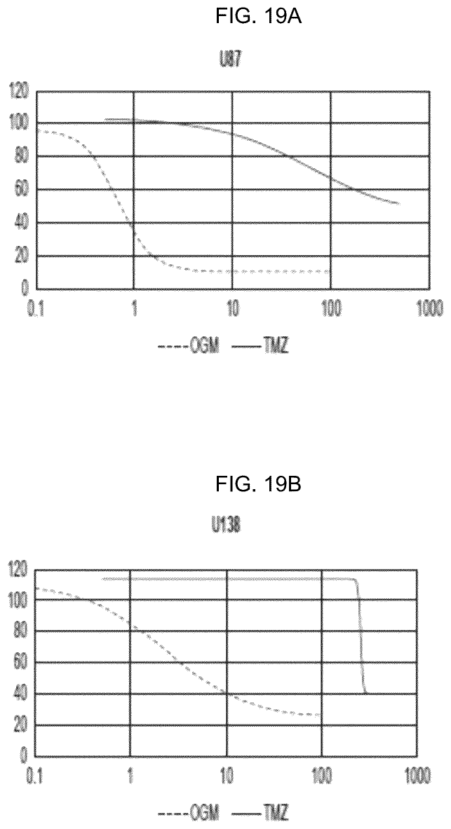

Temozolomide is the standard of care for glioblastoma but only works on 30% of patients. Temozolomide sensitive U87 (FIG. 19A) and Temozolomide insensitive U138 (FIG. 19B) glioblastoma cell lines were plated in 96-well plates and exposed to increasing doses of either Temozolomide or OGM2. The viability of cells was assayed using cell titer blue after 72 hours, and data was normalized to DMSO vehicle control. See results in FIG. 19. OGM2 was more effective than Temozolomide in this test.

### Example 16: GPR68 Inhibition Attenuates Growth in 3D Tumoroid Culture in Both TMZ Sensitive (U87) and Insensitive (U138) Glioblastoma Models

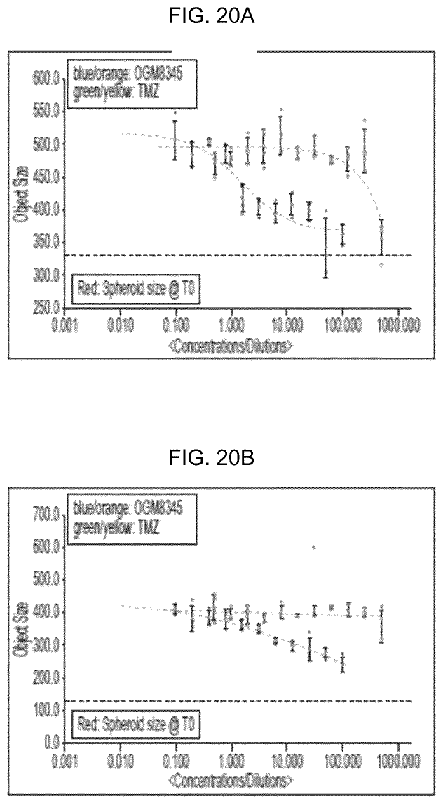

Tumor spheroid cultures are a more physiological model of tumor growth than typical 2D cell culture. Temozolomide sensitive U87 (FIG. 20A) and Temozolomide insensitive U138 (FIG. 20B) glioblastoma cell lines were plated in low attachment round bottom 96-well plates and allowed to form tumor spheroids for 3 days (T0). At T0, spheroids were imaged using Lionheart™ (HCS) High-Content Imaging Systems (Biotek™) and then were exposed to increasing doses of either Temozolomide or OGM2. After 5 days of treatment the spheroids were imaged again (T5). The size (area) of the spheroids after 5 days of treatment was quantified, and the average size of the spheroids at TO is depicted in red. OGM2 was more effective than Temozolomide in this test. See FIG. 20.

### Example 17: Ogerin GPR68 Positive Allosteric Modulator (PAM) Stimulates Growth in 3D Tumoroid Culture in Glioblastoma Models

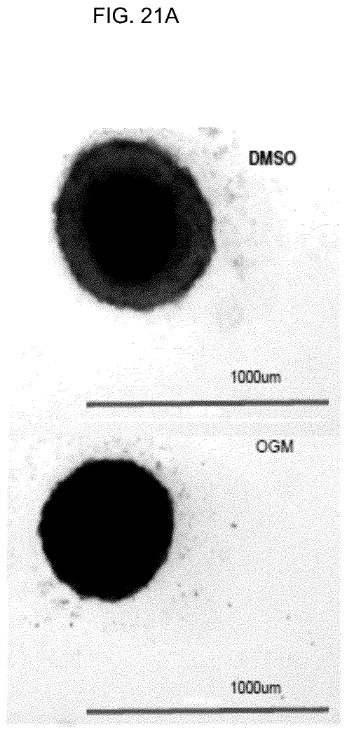

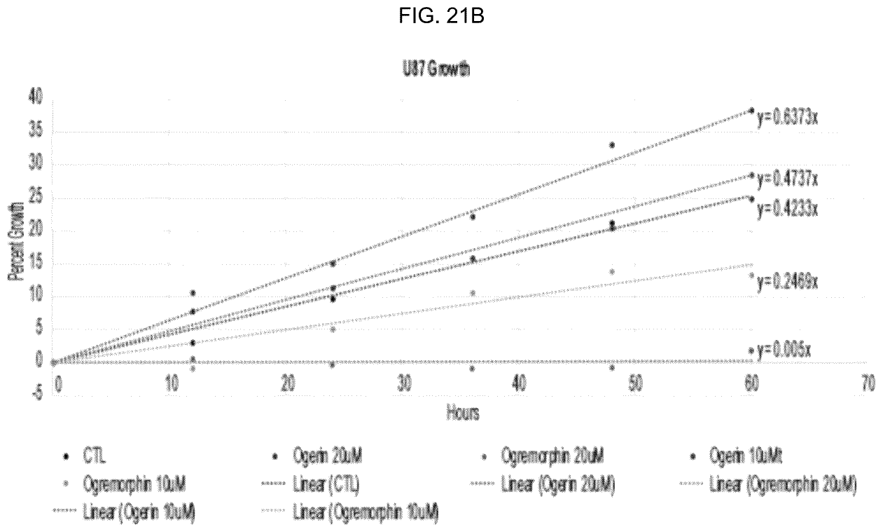

A GPR68-positive allosteric modulator, Ogerin, was tested. U87glioblastoma cells were plated in low attachment round bottom 96-well plates and allowed to form tumor spheroids for 3 days (TO). These spheroids were treated with Vehicle (DMSO), 10 μM/20 μM Ogerin or 10 μM/20 μM Ogremorphin-2 (OGM2). The spheroids were placed in the Lionheart™ HCS equipped with environmental control (5% CO₂ at 37° C.) and imaged every 12 hours. See FIG. 21A and FIG. 21B.

### Example 18: GPR68 Inhibition Increases Cell Death

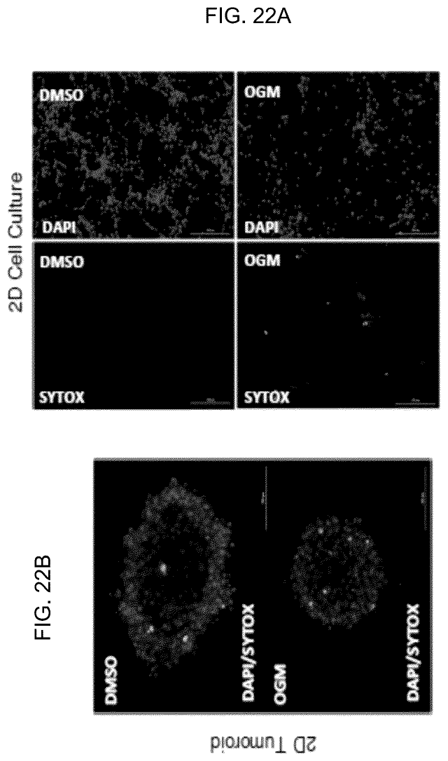

2D and 3D tumor spheroid assays were carried out as described herein with U87 cells using 10 μM OGM2 and stained with ReadyProbes™ Cell Viability Imaging Kit, Blue/Green (Thermo™). All cell nuclei labeled in blue, only nuclei with disrupted cell membranes are stained green, indicating cell death. See FIG. 22A and FIG. 22B.

### Example 19: OGM2 Inhibition of Tumoroid Growth is pH Dependent

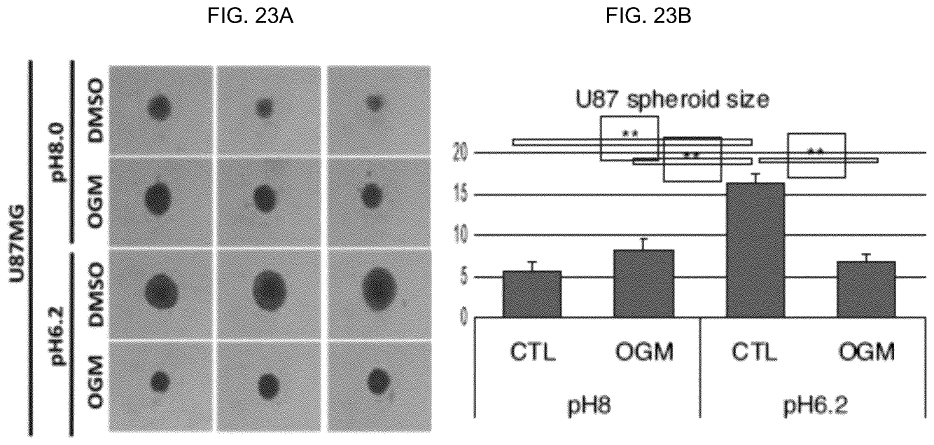

Spheroids were formed over 3 days using low attachment 96-well round bottom plates and then treated with DMSO or OGM2 in either pH 8 buffered media or pH 6.2 buffered media and grown for 3 days. The size (area) of spheroids was measured in relative area units. Spheroids grown in pH 6.2 were significantly larger than those grown at pH 8. Thus, GPR68 is inactive at pH 8 and is active at pH 6.2. This increase in growth was abrogated by OGM2. See FIG. 23.

### Example 20: OGM2 Reduces Clonogenicity of U87 Glioblastoma Cells

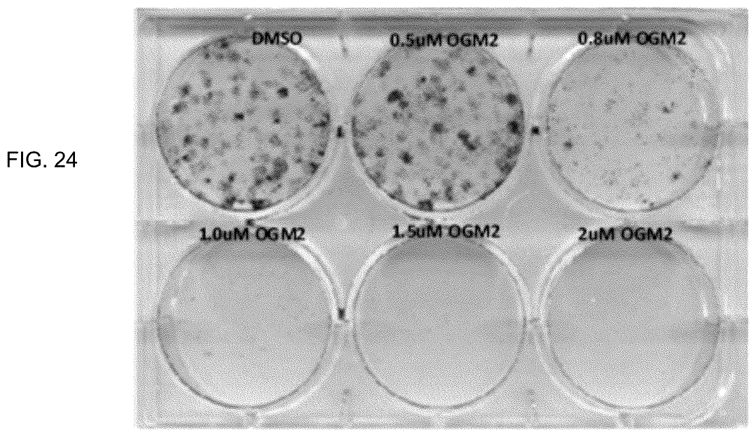

U87 cells were plated at 400 cells per well in a 6-well plate and on the next day were treated with increasing concentrations of OGM2 for 12 days. The doses were 0 uM (DMSO control) 0.1 μM 0.5 uM, 1 uM, 1.5 uM and 2 uM, as labelled. The plate was fixed with formalin and stained with crystal violet. See FIG. 24.

### Example 21: HCT116 Colon Cancer Effects

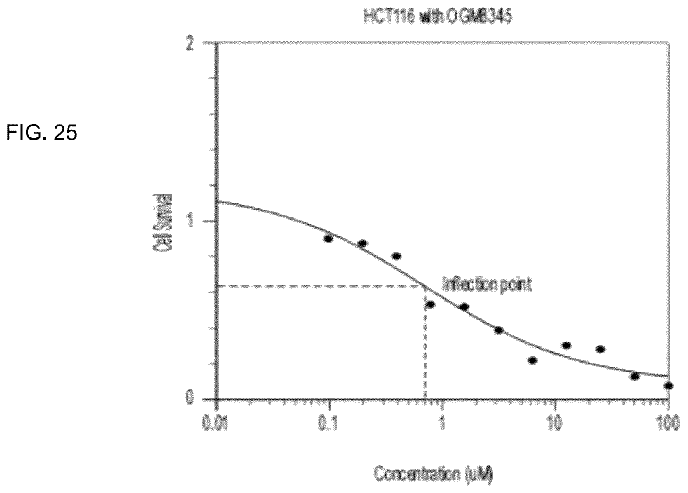

HCT116 colon cancer cells were treated with OGM2 at different concentrations for 48 hours. Cell viability was assessed using Cell titer blue. The results are shown in FIG. 25.

### Example 22: Effects on Pancreatic Cancer

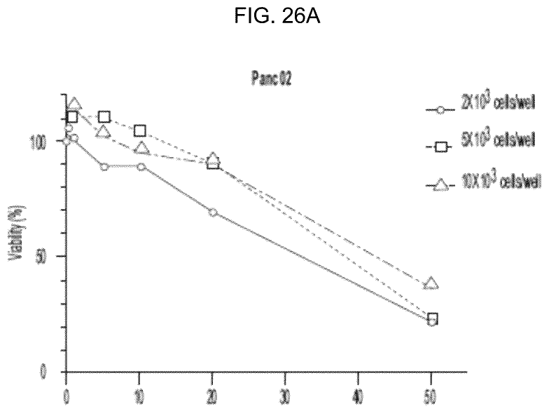

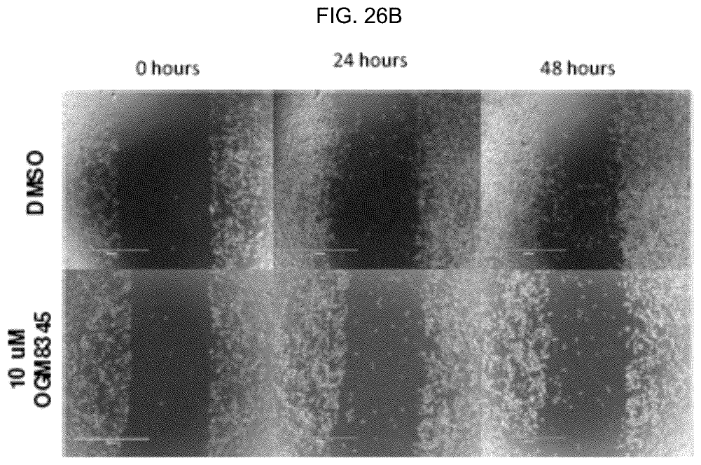

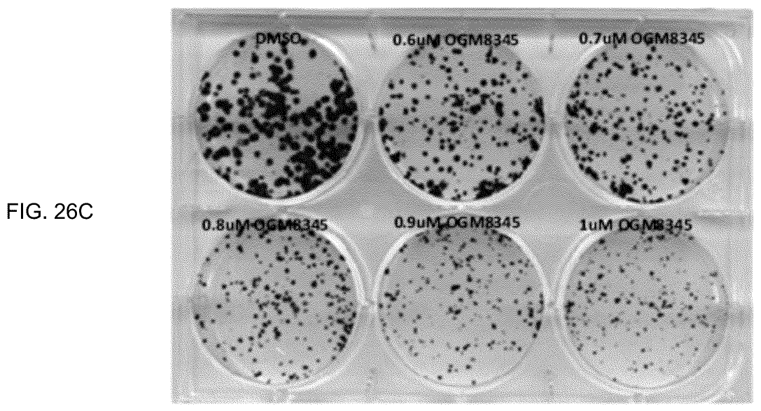

PANC02 pancreatic cells were plated at different concentrations of cell per well in a 96-well plate. On the following day, cells were treated with increasing doses of OGM2. Twenty-four hours later, the cell viability was assayed with crystal violet and normalized to untreated cells. See FIG. 26A.

In a second study. PANC02 pancreatic cells plated and grown to confluence. Using a P200 tip, cells were denuded and allowed to migrate for 24 hours in DMSO or 10 μM OGM2 and imaged. OGM2-treated wells had significantly reduced migration. See FIG. 26B.

In a third study, PANC02 pancreatic cells were plated at 400 cells per well in a 6-well plate and on the next day were treated with increasing concentrations of OGM2 for 7 days. The plate was fixed with formalin and stained with crystal violet. The results are shown in FIG. 26C for the indicated concentrations of OGM2, and demonstrate that OGM2 treated well reduced the clonogenicity of pancreatic cancer cells.

### Example 23: Effects on Lung Cancer

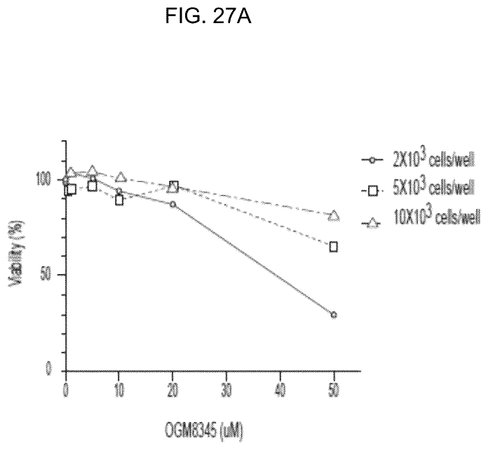

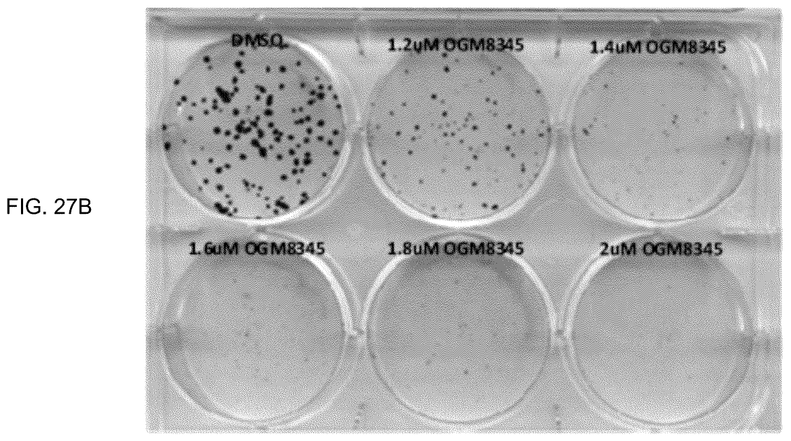

A549 lung cancer cells were plated at different concentrations of cell per well in a 96-well plate. On the following day cells were treated with increasing doses of OGM2. Twenty-four hours later cells viability was assayed with crystal violet and normalized to untreated. See FIG. 27A. These data show that relatively High doses of OGM2 are necessary for toxicity in A549 cells.

A549 lung cancer cells were plated at 300 cells per well in a 6-well plate and on the next day were treated with increasing concentrations of OGM2 for 8 days. The plate was fixed with formalin and stained with crystal violet. See FIG. 27B for results at the indicated concentrations. The results are shown in FIG. 27 for the indicated concentrations of OGM2, and demonstrate that OGM2 treated well reduced the clonogenicity of lung cancer cells.

### Example 24: Lower GPR68 Expression Correlates to Better Prognosis of Multiple Cancer Cell Types. OGM2 Reduces Viability of Prostate and Breast Cancer Cells

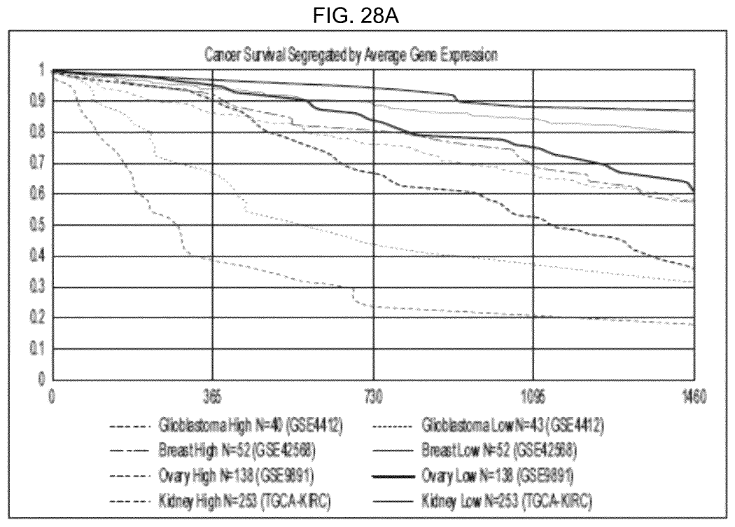

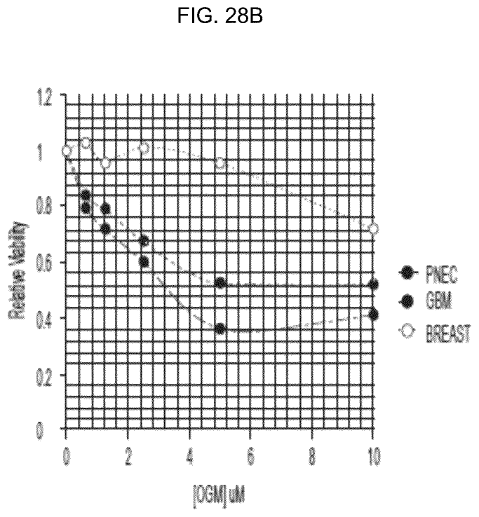

Multiple studies were conducted as discussed herein, but with different cancer type cohorts. Data was extracted from Gene expression omnibus using ProggeneV2. Briefly, probes were prepared using standard Affymetrix™ protocols, and hybridized to Affymetrix™ HG-U133A and HG-U133B arrays. The cohort was separated by High and low expression of GPR68 by above or below the median expression level. Survival of the cohorts was plotted in FIG. 28A. Survival of multiple cancer types including breast (MDA MB231), glioblastoma (U87), and Prostatic Neuroendocrine cancer (PNEC: PC3) is shown in FIG. 28B. Cells were plated in 96-well plates and then treated with increasing doses OGM2. Seventy-two hours later, survival was assayed with cell titer blue. Each cell line was normalized to DMSO treated control. See FIG. 28.

### Example 25: OGM2 Synergizes with TMZ and Radiation

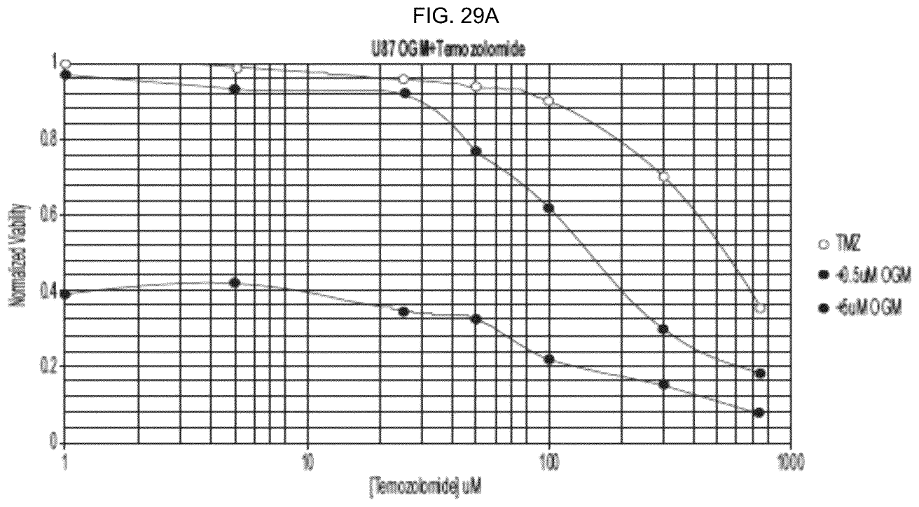

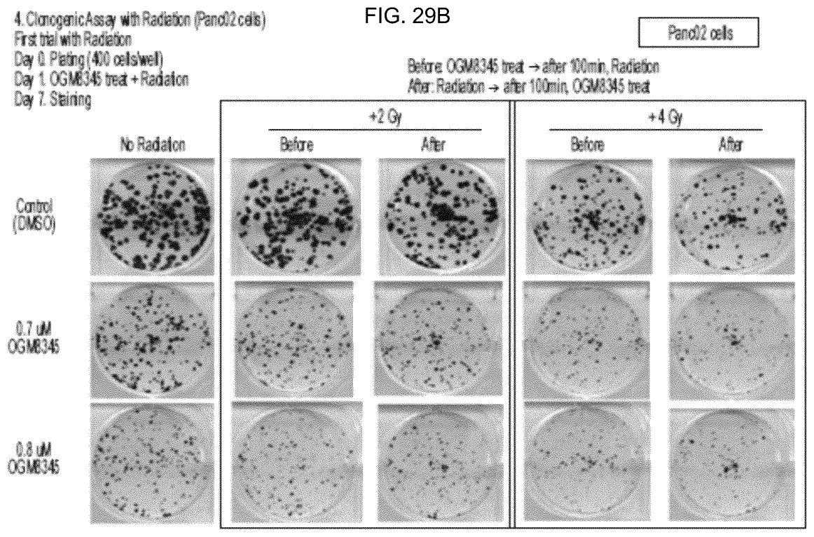

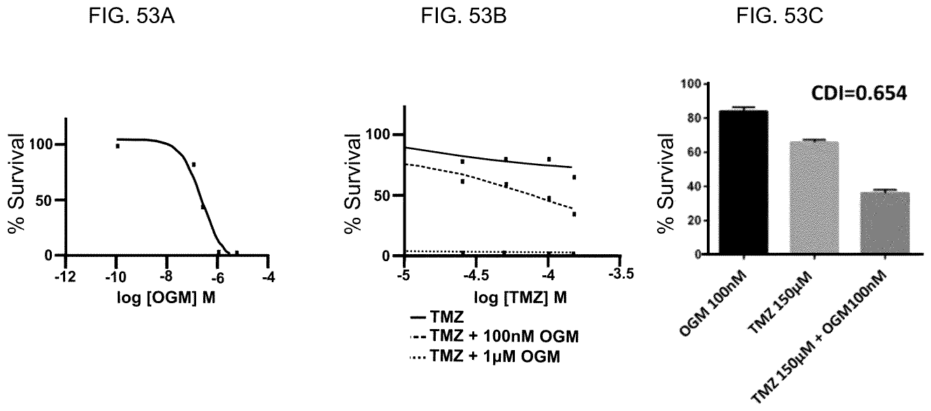

U87 cells were treated with increasing concentrations of temozolomide (TMZ) without OGM2 or with 0.5 μM or 5 μM OGM2. See results in FIG. 29A; see also FIG. 53A, showing a synergistic result for cell viability.

In a second study, four hundred PANC02 cells were plated per well in 6-well plates. The next day, cells were irradiated with either 2y of 4y of radiation, and also treated either 100 minutes before or 100 minutes after radiation with 0.7 μM or 0.8 μM OGM2. The results are presented in FIG. 29B.

### Example 26: OGM2 Inhibits Acid Induced Mucin Production

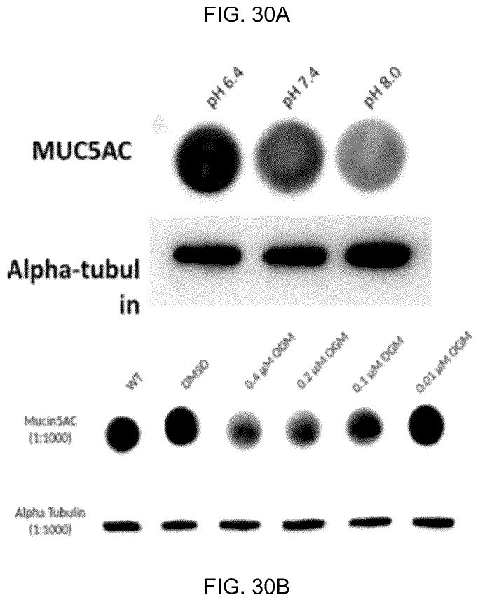

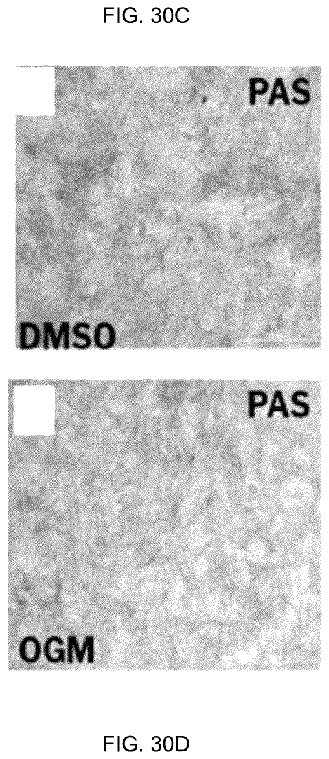

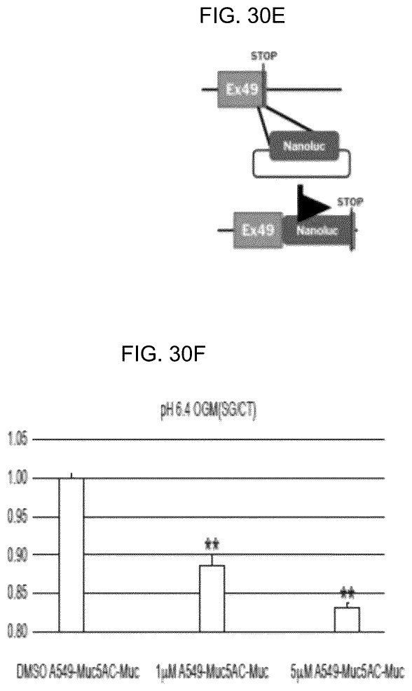

Lung (A549) cells were plated in 6-well plates. Cells were incubated in pH-buffered medium (at the pH indicated in FIG. 30A) for 24 hours. The cells then were rinsed and protein was isolated. Dot blots were made with anti Mucin-5AC antibodies, and an equivalent amount of protein was run on a western blot to detect alpha tubulin. See FIG. 30A. Lung (A549) cells were cultured as above, but in pH 6.4 buffered media with increasing concentrations of OGM2. Mucin5AC and alpha tubulin were detected in the same way. See FIG. 30B, which shows that inhibition of GPR68 inhibits Mucin 5AC in a dose-dependent manner.

In addition. A549 cells were cultured in pH 6.4 medium with or without OGM2 for 24 hours cells were then rinsed and then stained with Periodic Acid Staining solution which detects mucins. See FIG. 30C and FIG. 30D. FIG. 30E is a schematic of cas9 mediated endogenous tagging of MU5AC genetic locus with nano-luciferase in A549 cells. For FIG. 30F, Muc5ac-Luc cells were cultured in pH 6.4 buffered medium with and without OGM2 for 24 hours. Cell lysates were then assayed for luciferase activity and normalized to untreated control. See results in FIG. 30F.

### Example 27: Additional Pulmonary Indications

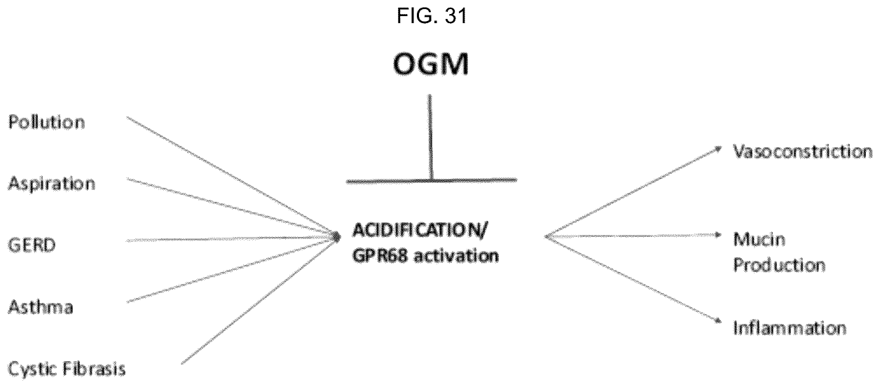

Further applications which are part of the invention and form embodiments of the invention include, but are not limited to, GERD, aspiration pneumonitis, COPD, ARDS, COVID-19, and the like. GPR68 is involved in sensing acidifications. In the airways this acidification is prevalent in asthmatic populations, those with GERD, some subjects exposed to certain pollutants, cystic fibrosis patients, and persons undergoing ventilation (where almost 90% of patients intubated for 4 or more days suffer from aspiration). Acidification of the mouse lung is a key model of ARDS, resulting in injury of the airway and alveolar epithelium, including type I alveolar epithelial cells, followed by a repair process that involves proliferation of alveolar type II cells, impairment in the alveolar epithelial fluid transport function, resulting in changes in alveolar fluid clearance independently of pulmonary blood flow or vascular filtration, and neutrophil infiltrations. The clinical development of ALI/ARDS typically involves a sudden, severe pulmonary inflammatory injury with the loss of integrity of alveolar-capillary permeability. Similarly, activation of GPR68 caused airway smooth muscles to contract using VASP in response to acidosis (pH 6.8). GPR68 was shown as critical for the inflammatory cascade, as well. See FIG. 31 for a diagram of these effects.

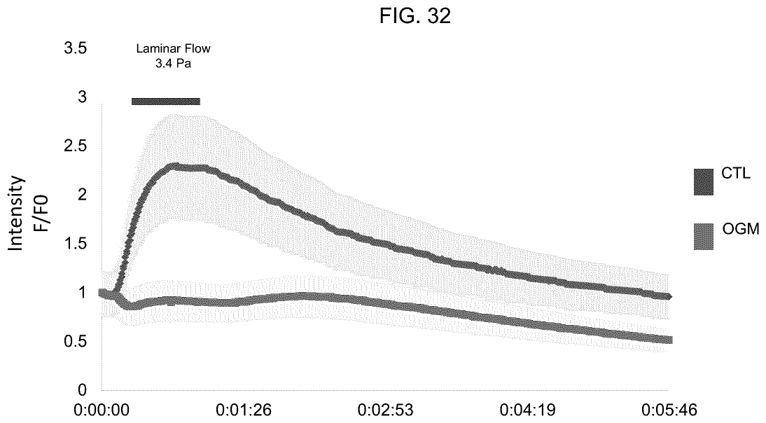

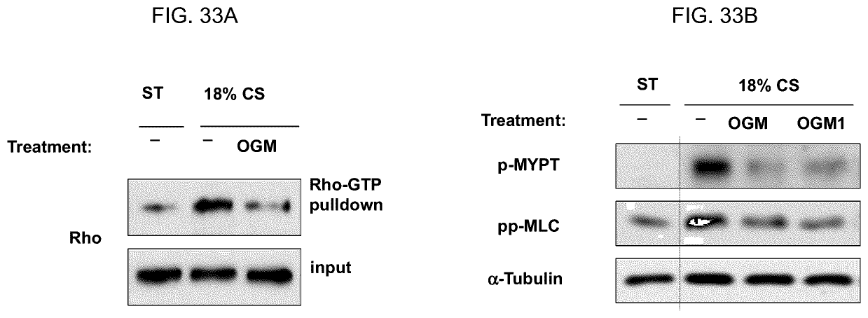

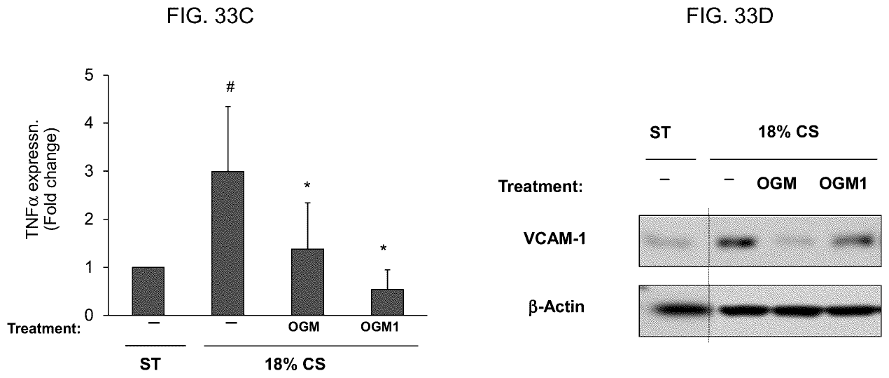

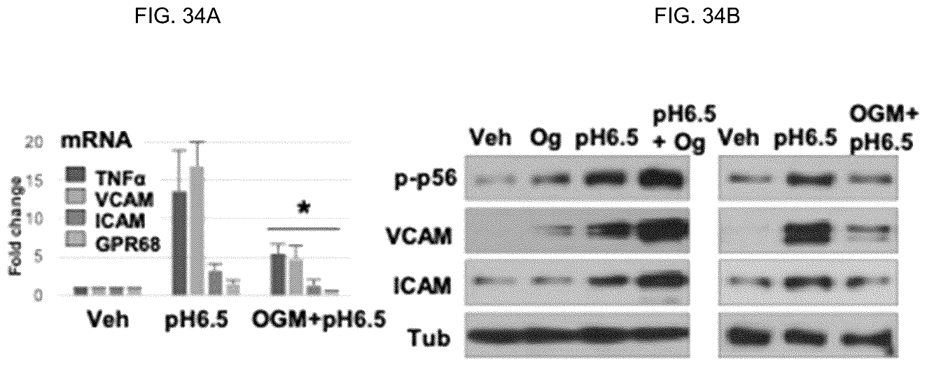

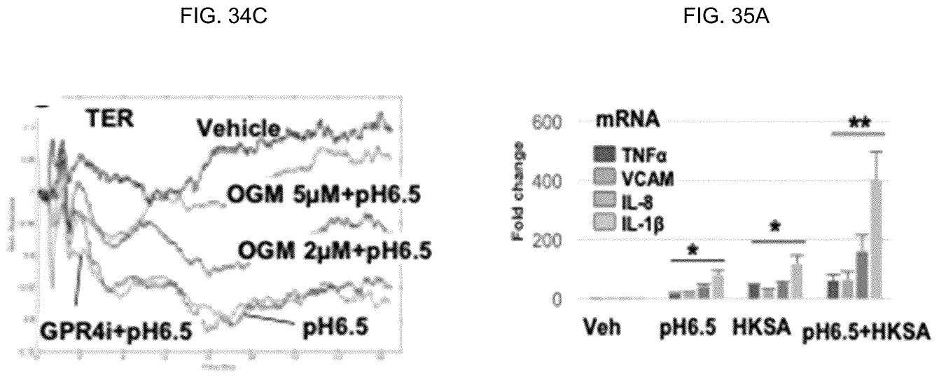

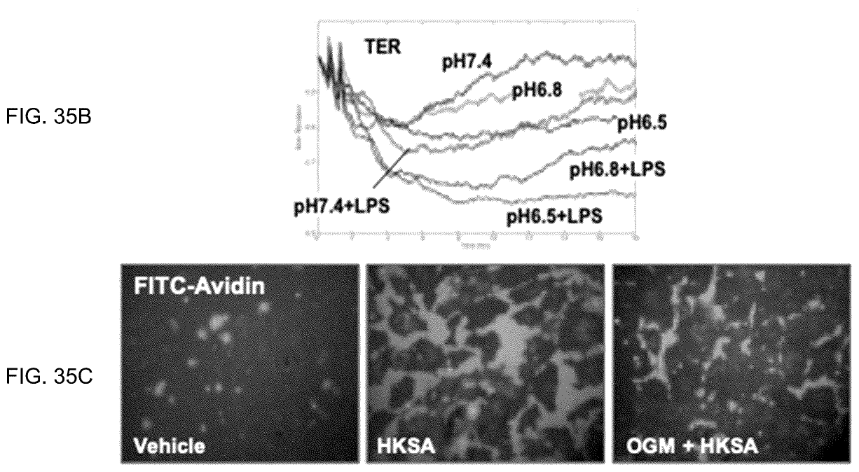

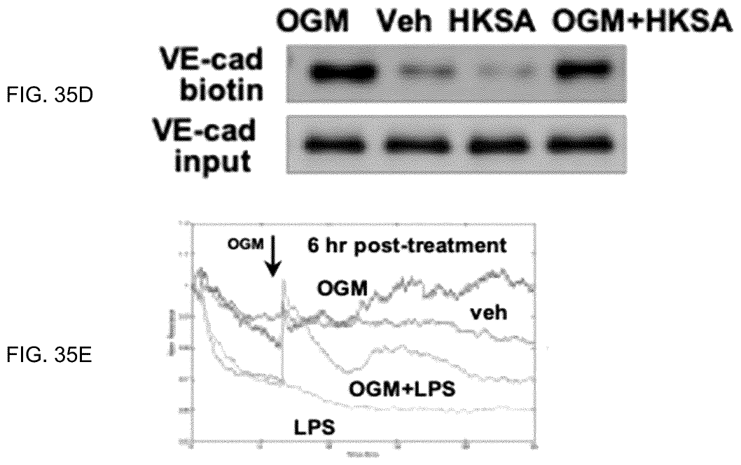

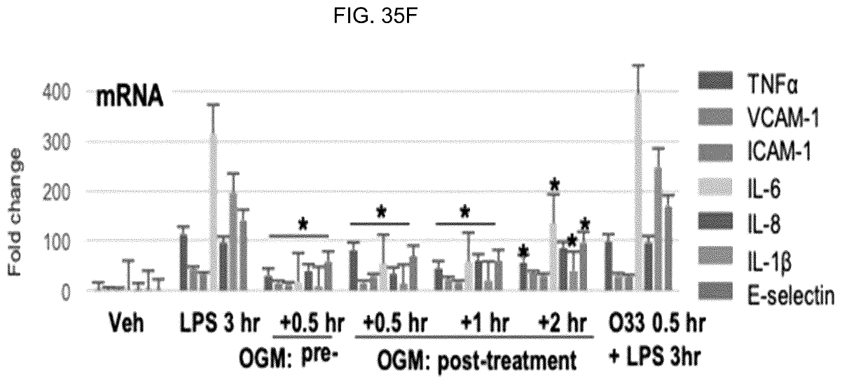

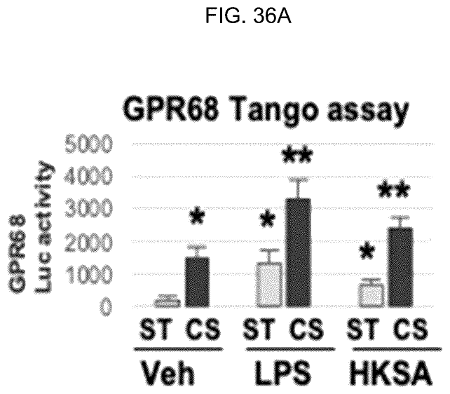

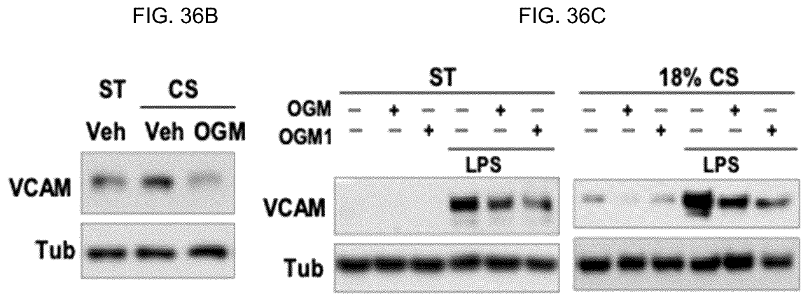

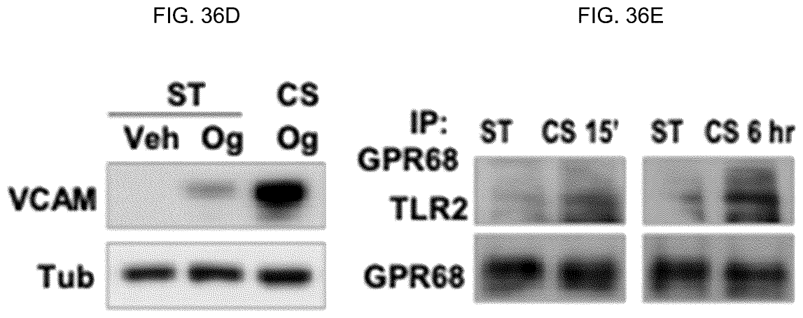

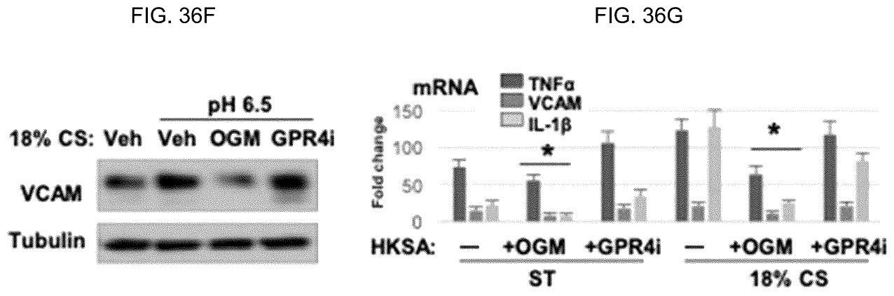

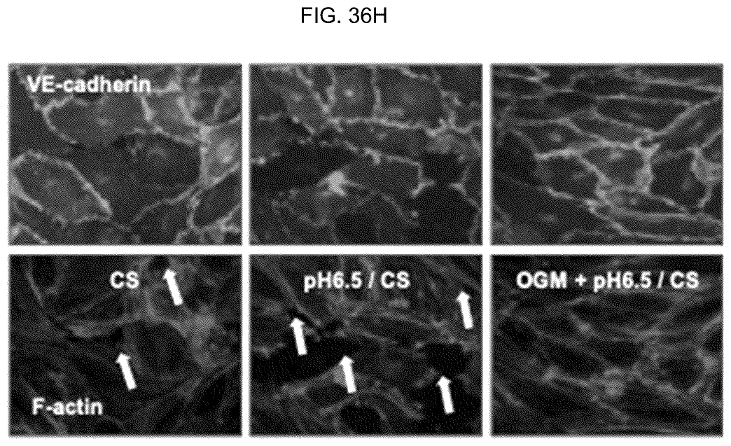

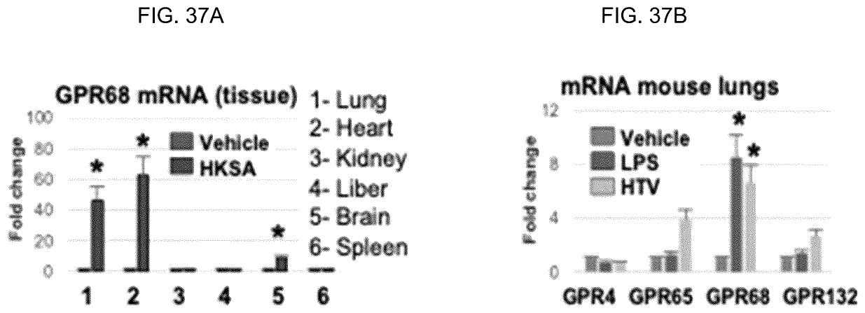

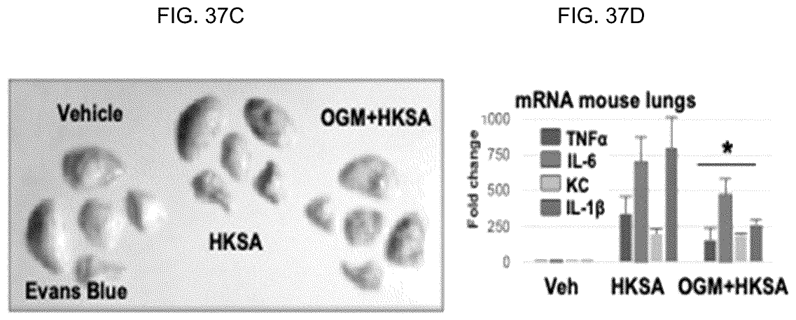

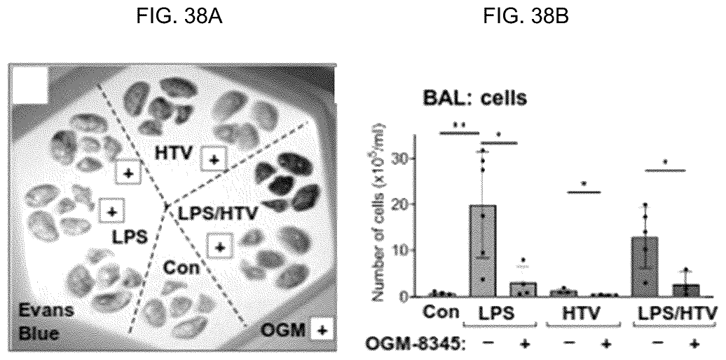

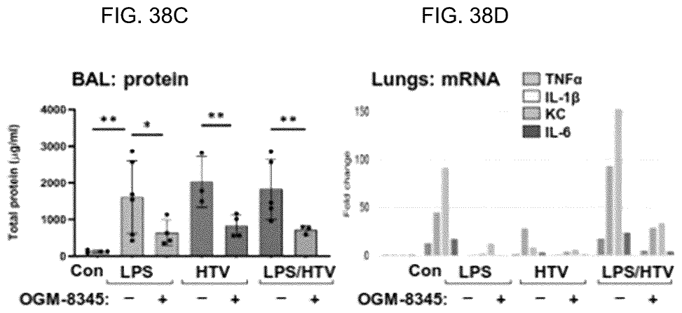

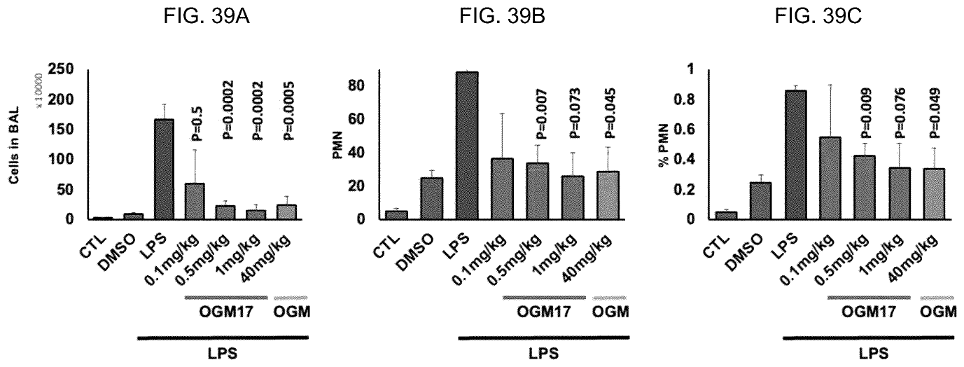

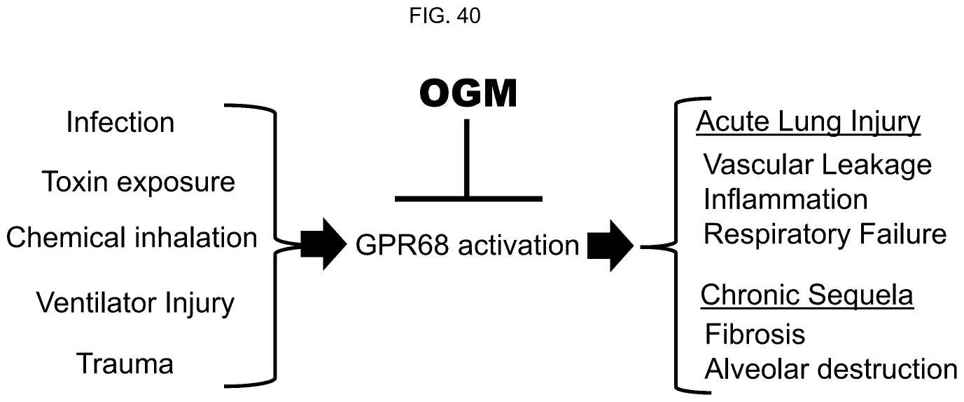

In addition to sensing extracellular acid, GPR68 is also a flow and stretch sensor, and OGM2 inhibits signaling due to laminar flow (FIG. 32) and cyclic stretch (FIG. 33). Consistent with critical role of GPR68 in inflammation induced endothelial cell (EC) dysfunction. OGM2 blocks acidification-induced endothelial dysfunction (FIG. 34). Moreover, extracellular acidification has additive effects with bacterium (HKSA) and LPS in causing EC dysfunction, all of which are abolished by OGM2 (FIG. 35). In addition, cyclic strain has additive effects with bacterium and LPS in causing EC dysfunction, all of which are abolished by OGM2 (FIG. 36). Importantly, OGM2 attenuates HKSA- and LPS-induced acute lung injury (ALI), as indicated by lung vascular leakage and increased inflammatory cytokines in lungs, in mice in vivo (FIG. 37). In addition, high-tidal-volume (HTV) ventilation exacerbates lung injury due to LPS, as indicted by lung vascular leakage, and cells and protein in broncheoalveolar lavage (BAL), and OGM2 attenuates the effects of HTV in vivo (FIG. 38). Finally, a more potent analog OGM17 is much more potent in reversing the markers of ALI due to LPS in vivo (FIG. 39). These results indicate that the OGM class of compounds are efficacious for blunting or preventing ALI due to variety of insults, as illustrated by FIG. 40.

### Example 28: Binding Panel

To identify the target of OGM1, the molecule was profiled for binding in a panel of 442 kinases (KinomeScan™, DiscoveRx™) and assessed its activity against 158 GPCRs in a single-point assay in Chem-1 cells that uses a promiscuous G_{α}15 protein to trigger calcium flux (see Table 9 and Table 10, below). In profiling studies, OGM1 did not physically interact with the kinase domain of any kinase, and OGM1 significantly inhibited only the lysophosphatidic acid receptor 1 (LPAR1), and extracellular proton-sensing GPR68/OGR1. See FIG. 4 and Table 9. Table 9. KinomeScan (DiscoverRx) profiling of OGM1.

| | % Control |
|---|---|
| | (OGM1 |
| Ambit Gene Symbol | [10 uM]) |
| AAK1 | 100 |
| ABL1(E255K)-phosphorylated | 77 |
| ABL1(F317I)-nonphosphorylated | 100 |
| ABL1(F317I)-phosphorylated | 82 |
| ABL1(F317L)-nonphosphorylated | 96 |
| ABL1(F317L)-phosphorylated | 92 |
| ABL1(H396P)-nonphosphorylated | 88 |
| ABL1(H396P)-phosphorylated | 83 |
| ABL1(M351T)-phosphorylated | 84 |
| ABL1(Q252H)-nonphosphorylated | 84 |
| ABL1(Q252H)-phosphorylated | 94 |
| ABL1(T315I)-nonphosphorylated | 73 |
| ABL1(T315I)-phosphorylated | 72 |
| ABL1(Y253F)-phosphorylated | 80 |
| ABL1-nonphosphorylated | 100 |
| ABL1-phosphorylated | 93 |
| ABL2 | 100 |
| ACVR1 | 100 |
| ACVR1B | 100 |
| ACVR2A | 100 |
| ACVR2B | 100 |
| ACVRLI | 100 |
| ADCK3 | 100 |
| ADCK4 | 70 |
| AKT1 | 78 |
| AKT2 | 93 |
| AKT3 | 100 |
| ALK | 100 |
| AMPK-alpha1 | 100 |
| AMPK-alpha2 | 100 |
| ANKK1 | 100 |
| ARK5 | 80 |
| ASK1 | 100 |
| ASK2 | 100 |
| AURKA | 100 |
| AURKB | 86 |
| AURKC | 100 |
| AXL | 100 |
| BIKE | 88 |
| BLK | 97 |
| BMPR1A | 100 |
| BMPR1B | 81 |
| BMPR2 | 92 |
| BMX | 100 |
| BRAF | 96 |
| BRAF(V600E) | 94 |
| BRK | 100 |
| BRSK1 | 100 |
| BRSK2 | 100 |
| BTK | 88 |
| CAMK1 | 50 |
| CAMK1D | 68 |
| CAMK1G | 80 |
| CAMK2A | 75 |
| CAMK2B | 60 |
| CAMK2D | 91 |
| CAMK2G | 85 |
| CAMK4 | 100 |
| CAMKK1 | 86 |
| CAMKK2 | 89 |
| CASK | 90 |
| CDC2L1 | 92 |
| CDC2L2 | 93 |
| CDC2L5 | 99 |
| CDK11 | 100 |
| CDK2 | 100 |
| CDK3 | 81 |
| CDK4-cyclinD1 | 69 |
| CDK4-cyclinD3 | 76 |
| CDK5 | 100 |
| CDK7 | 64 |
| CDK8 | 100 |
| CDK9 | 98 |
| CDKL1 | 88 |
| CDKL2 | 100 |
| CDKL3 | 100 |
| CDKL5 | 85 |
| CHEK1 | 100 |
| CHEK2 | 82 |
| CIT | 94 |
| CLK1 | 100 |
| CLK2 | 61 |
| CLK3 | 100 |
| CLK4 | 88 |
| CSF1R | 78 |
| CSK | 83 |
| CSNK1A1 | 75 |
| CSNK1A1L | 100 |
| CSNK1D | 100 |
| CSNK1E | 91 |
| CSNK1G1 | 82 |
| CSNK1G2 | 100 |
| CSNK1G3 | 100 |
| CSNK2A1 | 96 |
| CSNK2A2 | 93 |
| CTK | 100 |
| DAPK1 | 90 |
| DAPK2 | 89 |
| DAPK3 | 91 |
| DCAMKL1 | 72 |
| DCAMKL2 | 100 |
| DCAMKL3 | 100 |
| DDR1 | 100 |
| DDR2 | 86 |
| DLK | 40 |
| DMPK | 86 |
| DMPK2 | 93 |
| DRAK1 | 100 |
| DRAK2 | 100 |
| DYRK1A | 39 |
| DYRK1B | 58 |
| DYRK2 | 91 |
| EGFR | 100 |
| EGFR(E746-A750del) | 100 |
| EGFR(G719C) | 100 |
| EGFR(G719S) | 95 |
| EGFR(L747-E749del, A750P) | 91 |
| EGFR(L747-S752del, P753S) | 99 |
| EGFR(L747-T751del, Sins) | 100 |
| EGFR(L858R) | 100 |
| EGFR(L858R, T790M) | 89 |
| EGFR(L861Q) | 100 |
| EGFR(S752-1759del) | 91 |
| EGFR(T790M) | 74 |
| EIF2AK1 | 100 |
| EPHA1 | 92 |
| EPHA2 | 100 |
| EPHA3 | 78 |
| EPHA4 | 100 |
| EPHA5 | 97 |
| EPHA6 | 100 |
| EPHA7 | 100 |
| EPHA8 | 100 |
| EPHB1 | 100 |
| EPHB2 | 100 |
| EPHB3 | 100 |
| EPHB4 | 100 |
| EPHB6 | 76 |
| ERBB2 | 65 |
| ERBB3 | 84 |
| ERBB4 | 100 |
| ERK1 | 100 |
| ERK2 | 77 |
| ERK3 | 100 |
| ERK4 | 93 |
| ERK5 | 89 |
| ERK8 | 100 |
| ERN1 | 72 |
| FAK | 88 |
| FER | 100 |
| FES | 100 |
| FGFR1 | 100 |
| FGFR2 | 100 |
| FGFR3 | 100 |
| FGFR3(G697C) | 99 |
| FGFR4 | 100 |
| FGR | 100 |
| FLT1 | 100 |
| FLT3 | 79 |
| FLT3(D835H) | 100 |
| FLT3(D835Y) | 100 |
| FLT3(ITD) | 100 |
| FLT3(K663Q) | 100 |
| FLT3(N841I) | 91 |
| FLT3(R834Q) | 100 |
| FLT4 | 100 |
| FRK | 100 |
| FYN | 100 |
| GAK | 64 |
| GCN2(Kin.Dom.2, S808G) | 100 |
| GRK1 | 100 |
| GRK4 | 96 |
| GRK7 | 73 |
| GSK3A | 78 |
| GSK3B | 83 |
| HCK | 96 |
| HIPK1 | 99 |
| HIPK2 | 100 |
| HIPK3 | 67 |
| HIPK4 | 100 |
| HPK1 | 100 |
| HUNK | 83 |
| ICK | 81 |
| IGF1R | 84 |
| IKK-alpha | 83 |
| IKK-beta | 92 |
| IKK-epsilon | 100 |
| INSR | 80 |
| INSRR | 100 |
| IRAK1 | 100 |
| IRAK3 | 97 |
| IRAK4 | 88 |
| ITK | 100 |
| JAK1(JH1domain-catalytic) | 59 |
| JAK1(JH2domain-pseudokinase) | 100 |
| JAK2(JH1domain-catalytic) | 100 |
| JAK3(JH1domain-catalytic) | 84 |
| JNK1 | 63 |
| INK2 | 83 |
| JNK3 | 82 |
| KIT | 79 |
| KIT(A829P) | 72 |
| KIT(D816H) | 97 |
| KIT(D816V) | 100 |
| KIT(L576P) | 82 |
| KIT(V559D) | 71 |
| KIT(V559D, T670I) | 77 |
| KIT(V559D, V654A) | 100 |
| LATS1 | 100 |
| LATS2 | 100 |
| LCK | 100 |
| LIMK1 | 100 |
| LIMK2 | 100 |
| LKB1 | 78 |
| LOK | 100 |
| LRRK2 | 100 |
| LRRK2(G2019S) | 100 |
| LTK | 100 |
| LYN | 100 |
| LZK | 100 |
| MAK | 90 |
| MAP3K1 | 72 |
| MAP3K15 | 100 |
| MAP3K2 | 92 |
| MAP3K3 | 72 |
| MAP3K4 | 100 |
| MAP4K2 | 83 |
| MAP4K3 | 100 |
| MAP4K4 | 100 |
| MAP4K5 | 100 |
| MAPKAPK2 | 100 |
| MAPKAPK5 | 86 |
| MARK1 | 100 |
| MARK2 | 100 |
| MARK3 | 100 |
| MARK4 | 95 |
| MAST1 | 58 |
| MEK1 | 84 |
| MEK2 | 94 |
| MEK3 | 100 |
| MEK4 | 100 |
| MEK5 | 92 |
| MEK6 | 76 |
| MELK | 100 |
| MERTK | 80 |
| MET | 100 |
| MET(M1250T) | 68 |
| MET(Y1235D) | 100 |
| MINK | 97 |
| MKK7 | 99 |
| MKNK1 | 90 |
| MKNK2 | 91 |
| MLCK | 100 |
| MLK1 | 100 |
| MLK2 | 100 |
| MLK3 | 100 |
| MRCKA | 78 |
| MRCKB | 100 |
| MST1 | 100 |
| MST1R | 86 |
| MST2 | 69 |
| MST3 | 61 |
| MST4 | 81 |
| MTOR | 86 |
| MUSK | 90 |
| MYLK | 91 |
| MYLK2 | 100 |
| MYLK4 | 98 |
| MYO3A | 95 |
| MYO3B | 100 |
| NDR1 | 98 |
| NDR2 | 82 |
| NEK1 | 83 |
| NEK11 | 88 |
| NEK2 | 78 |
| NEK3 | 71 |
| NEK4 | 100 |
| NEK5 | 95 |
| NEK6 | 100 |
| NEK7 | 100 |
| NEK9 | 100 |
| NIM1 | 96 |
| NLK | 100 |
| OSR1 | 92 |
| p38-alpha | 54 |
| p38-beta | 100 |
| p38-delta | 81 |
| p38-gamma | 94 |
| PAK1 | 70 |
| PAK2 | 72 |
| PAK3 | 93 |
| PAK4 | 66 |
| PAK6 | 89 |
| PAK7 | 62 |
| PCTK1 | 77 |
| PCTK2 | 100 |
| PCTK3 | 99 |
| PDGFRA | 100 |
| PDGFRB | 72 |
| PDPK1 | 100 |
| PFCDPK1(P. falciparum) | 95 |
| PFPK5(P. falciparum) | 82 |
| PFTAIRE2 | 93 |
| PFTK1 | 92 |
| PHKG1 | 91 |
| PHKG2 | 71 |
| PIK3C2B | 95 |
| PIK3C2G | 50 |
| PIK3CA | 100 |
| PIK3CA(C420R) | 100 |
| PIK3CA(E542K) | 73 |
| PIK3CA(E545A) | 81 |
| PIK3CA(E545K) | 100 |
| PIK3CA(H1047L) | 100 |
| PIK3CA(H1047Y) | 59 |
| PIK3CA(I800L) | 100 |
| PIK3CA(M1043I) | 59 |
| PIK3CA(Q546K) | 95 |
| PIK3CB | 100 |
| PIK3CD | 55 |
| PIK3CG | 100 |
| PIK4CB | 71 |
| PIM1 | 56 |
| PIM2 | 84 |
| PIM3 | 73 |
| PIPSK1A | 100 |
| PIPSK1C | 97 |
| PIP5K2B | 100 |
| PIP5K2C | 100 |
| PKAC-alpha | 95 |
| PKAC-beta | 96 |
| PKMYT1 | 78 |
| PKN1 | 100 |
| PKN2 | 100 |
| PKNB(M. tuberculosis) | 79 |
| PLK1 | 100 |
| PLK2 | 100 |
| PLK3 | 100 |
| PLK4 | 71 |
| PRKCD | 100 |
| PRKCE | 68 |
| PRKCH | 100 |
| PRKCI | 94 |
| PRKCQ | 99 |
| PRKD1 | 100 |
| PRKD2 | 87 |
| PRKD3 | 100 |
| PRKG1 | 100 |
| PRKG2 | 91 |
| PRKR | 59 |
| PRKX | 98 |
| PRP4 | 100 |
| PYK2 | 100 |
| QSK | 100 |
| RAF1 | 100 |
| RET | 100 |
| RET(M918T) | 92 |
| RET(V804L) | 90 |
| RET(V804M) | 100 |
| RIOK1 | 100 |
| RIOK2 | 100 |
| RIOK3 | 100 |
| RIPK1 | 100 |
| RIPK2 | 100 |
| RIPK4 | 81 |
| RIPK5 | 56 |
| ROCK1 | 71 |
| ROCK2 | 73 |
| ROS1 | 100 |
| RPS6KA4(Kin.Dom.1-N-terminal) | 92 |
| RPS6KA4(Kin.Dom.2-C-terminal) | 74 |
| RPS6KA5(Kin.Dom.1-N-terminal) | 100 |
| RPS6KA5(Kin.Dom.2-C-terminal) | 91 |
| RSK1(Kin.Dom.1-N-terminal) | 78 |
| RSK1(Kin.Dom.2-C-terminal) | 73 |
| RSK2(Kin.Dom.1-N-terminal) | 95 |
| RSK3(Kin.Dom.1-N-terminal) | 100 |
| RSK3(Kin.Dom.2-C-terminal) | 54 |
| RSK4(Kin Dom.1-N-terminal) | 100 |
| RSK4(Kin.Dom.2-C-terminal) | 41 |
| S6K1 | 93 |
| SBK1 | 100 |
| SgK110 | 100 |
| SGK3 | 56 |
| SIK | 95 |
| SIK2 | 100 |
| SLK | 90 |
| SNARK | 100 |
| SNRK | 100 |
| SRC | 95 |
| SRMS | 80 |
| SRPK1 | 77 |
| SRPK2 | 100 |
| SRPK3 | 100 |
| STK16 | 88 |
| STK33 | 91 |
| STK35 | 100 |
| STK36 | 100 |
| STK39 | 59 |
| SYK | 98 |
| TAK1 | 72 |
| TAOK1 | 83 |
| TAOK2 | 81 |
| TAOK3 | 79 |
| TBK1 | 90 |
| TEC | 93 |
| TESK1 | 100 |
| TGFBR1 | 100 |
| TGFBR2 | 100 |
| TIE1 | 94 |
| TIE2 | 100 |
| TLK1 | 53 |
| TLK2 | 71 |
| TNIK | 71 |
| TNK1 | 100 |
| TNK2 | 100 |
| TNNI3K | 100 |
| TRKA | 81 |
| TRKB | 51 |
| TRKC | 62 |
| TRPM6 | 80 |
| TSSK1B | 79 |
| TTK | 75 |
| TXK | 99 |
| TYK2(JH1domain-catalytic) | 77 |
| TYK2(JH2domain-pseudokinase) | 100 |
| TYRO3 | 100 |
| ULK1 | 100 |
| ULK2 | 100 |
| ULK3 | 100 |
| VEGFR2 | 100 |
| VRK2 | 93 |
| WEE1 | 100 |
| WEE2 | 100 |
| YANK1 | 100 |
| YANK2 | 100 |
| YANK3 | 100 |
| YES | 100 |
| YSK1 | 80 |
| YSK4 | 100 |
| ZAK | 100 |
| ZAP70 | 73 |

TABLE 10
GPCR Profiler (Millipore) profiling of OGM1.

| | OGM1 | OGM1 |
|---|---|---|
| | (12.5 uM) | (10 uM) |
| Target | Agonism | Antagonism |
| 5-HT1A | −0.8 | −7.5 |
| 5-HT2A | 1.2 | −12.3 |
| 5-HT2B | −0.1 | 0.3 |
| 5-HT2C | 2.2 | −10.9 |
| 5-HT4B | 4.1 | −24.1 |
| 5-HT6 | −0.7 | −4.4 |
| A1 | 0.1 | −13.9 |
| A2B | 4.4 | −12.8 |
| A3 | −0.4 | 3.2 |
| ADRA1A | 1.9 | 3 |
| ADRA1B | 0.1 | 1 |
| ADRA1D | 0.8 | −5.7 |
| ADRA2A | 0.1 | −5.1 |
| ADRB1 | −1.3 | −9.6 |
| ADRB2 | −0.1 | −9.5 |
| ADRB3 | −0.2 | 605 |
| APJ | 0.9 | 7.7 |
| AT1 | −0.4 | −0.7 |
| BB1 | −0.2 | 3.5 |
| BB2 | −0.2 | 0 |
| BB3 | 1.3 | −0.7 |
| BDKR2 | −2.4 | 3.4 |
| BLY1 | 0.3 | −5.5 |
| C3aR | 0.2 | 5.4 |
| C5aR | 3.1 | −0.7 |
| CaS | −1.2 | −3.6 |
| CB1 | −0.1 | −6.8 |
| CB2 | −2.4 | −13.8 |
| CCK1 | 0.8 | 1.9 |
| CCK2 | −0.4 | 14.9 |
| CCR1 | −0.3 | −0.6 |
| CCR10 | −0.2 | −16.7 |
| CCR2B | 0.7 | 0.7 |
| CCR3 | 0.8 | −9.9 |
| CCR4 | −0.7 | −4.1 |
| CCR5 | 2.5 | 15.8 |
| CCR6 | 1.5 | 5.1 |
| CCR7 | 0 | 4 |
| CCR8 | 2 | −2.5 |
| CCR9 | 0.4 | 0.6 |
| CGRP1 | 0.5 | −2.3 |
| ChemR23 | 0 | 0.1 |
| CRF1 | −0.1 | 4.6 |
| CRF2 | 0.6 | −13.7 |
| CX3CR1 | 0.3 | −12.1 |
| CXCR1 | −0.7 | 6.5 |
| CXCR2 | 0.5 | −3.1 |
| CXCR3 | 0.1 | −2 |
| CXCR4 | 1.5 | −6.9 |
| CXCR5 | 0.9 | −2 |
| CXCR6 | 0 | −14 |
| CysLT1 | 0.8 | −2.4 |
| CysLT2 | −0.3 | −7.1 |
| D1 | 0 | 5 |
| D2 | −0.5 | 11.4 |
| D4 | 0.6 | −16.3 |
| D5 | −1.8 | −17.8 |
| DP | 0.8 | −11.7 |
| EP1 | 0.2 | −6 |
| EP2 | −0.2 | 7 |
| EP3 | 0.4 | 6.3 |
| EP4 | 0.9 | −17 |
| ETA | 0.1 | 4.7 |
| ETB | 9.1 | −1.6 |
| FP | 0.9 | 8.9 |
| FPR1 | −0.2 | 14.3 |
| FPR2 | −0.4 | 23 |
| GABAB1b | 0.6 | 0.1 |
| GAL1 | 1.6 | −7.3 |
| GAL2 | 0.4 | 1 |
| GCGR | −0.1 | 6.1 |
| Ghrelin | 0.3 | −6.1 |
| GIP | 1.5 | 0.2 |
| GLP-1 | −0.3 | 2.1 |
| GLP-2 | −0.2 | 2.1 |
| GnRH | 0.5 | −2.6 |
| GPR103 | −0.1 | −7 |
| GPR109A | −0.2 | 20.3 |
| GPR14 | 16.8* | — |
| GPR39 | 0 | 5.4 |
| GPR41 | 0.4 | 1.9 |
| GPR43 | −0.2 | 6.4 |
| GPR54 | −0.6 | −5.8 |
| GPR68 | −7.6 | 70.8 |
| GPR91 | 1.2 | −5.4 |
| GPR99 | 6.5 | 0.5 |
| H1 | 2.1 | −17.2 |
| H2 | 0.1 | 1.3 |
| H3 | 0.5 | −3.3 |
| IP1 | −0.5 | −2 |
| LPA1 | 0.5 | 89.5 |
| LPA3 | 0.3 | 1.1 |
| LPA5 | −0.1 | −16.2 |
| M1 | 0.8 | −3.3 |
| M2 | −0.1 | 3.3 |
| M3 | 0.7 | −1.9 |
| M4 | −0.6 | −2.8 |
| M5 | −0.6 | 3.4 |
| MC2 | 0.1 | 3.9 |
| MC4 | −0.3 | −5.4 |
| MC5 | 0.9 | 13.9 |
| MCHR1 | −0.4 | .2.3 |
| MCHR2 | 0.3 | −8.2 |
| mGlu2 | −0.4 | −1.2 |
| mGlu1 | 1.1 | −12.7 |
| Motilin | 1 | −0.7 |
| MrgD | −0.1 | −2.2 |
| MRGX1 | −0.1 | 2.3 |
| MRGX2 | −0.1 | 5.1 |
| NK1 | −1.5 | −1.4 |
| NK2 | 0 | 1 |
| NK3 | 0.5 | −3 |
| NMU1 | 1.8 | 6.2 |
| NMU2 | 0 | −4.2 |
| NOP | 0.2 | −1.9 |
| NPBW1 | 0.5 | 22.8 |
| NTR1 | 1 | 1.6 |
| OPRD1 | −0.1 | 7.4 |
| OPRK1 | 4.3 | −8.1 |
| OPRM1 | −0.7 | −0.7 |
| OT | 0.7 | −2.4 |
| OX1 | 1.4 | 1.6 |
| OX2 | 0.2 | −3.4 |
| P2Y1 | 1.7 | −6.3 |
| P2Y11 | −0.1 | −8.2 |
| P2Y12 | 1 | −0.3 |
| P2Y2 | 0.1 | −9 |
| P2Y4 | −0.8 | 5.4 |
| PAC1 | 0.9 | 5.8 |
| PAF | 0.4 | 6.6 |
| PK1 | 0.2 | −8 |
| PK2 | −0.1 | 7.2 |
| PRP | −0.3 | −11.1 |
| PTH1 | 0.7 | 2.2 |
| PTH2 | 1.1 | −19.2 |
| S1P1 | −0.4 | 3.1 |
| S1P2 | 0.5 | −3.3 |
| S1P3 | 0.2 | −3.7 |
| S1P4 | 0.3 | −5.2 |
| S1P5 | 1.8 | 0.9 |
| Secretin | 0.9 | 1.7 |
| sst2 | −0.2 | −1 |
| sst3 | 0 | 0 |
| sst4 | −1.1 | −2.6 |
| sst5 | −0.3 | −4 |
| Thrombin | 1 | 3.2 |
| Activated PARs | | |
| TP | 0.2 | 8.1 |
| TRH | 0.1 | 1.4 |
| Trypsin activated | 0 | 18.2 |
| PARS | | |
| TSH | −0.1 | 5.9 |
| V1A | 0.1 | 12.3 |
| V1B | −0.1 | −2.8 |
| V2 | 0.6 | 2.9 |
| VPAC1 | 0 | 16.6 |
| VPAC2 | 10.7 | 6.9 |
| XCR1 | 0.1 | 1.8 |
| Y2 | 2.5 | −0.6 |
| Y4 | −0.2 | −5.5 |

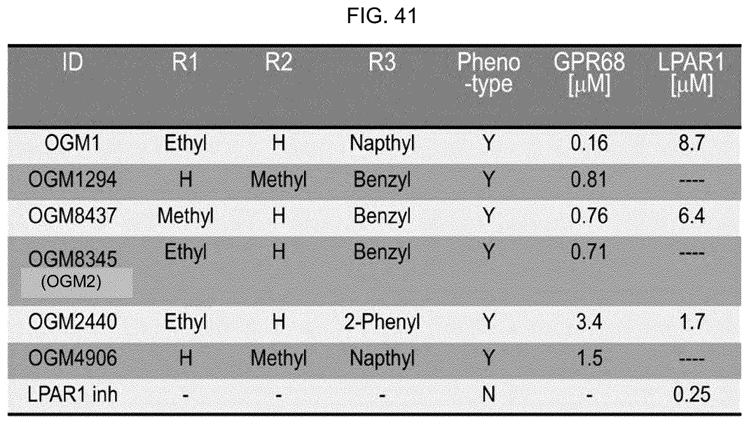

To distinguish which GPCR was involved in this phenotype, a chemical genetic segregation analysis was carried out in zebrafish embryos. A small-scale structure-activity relationship study around the core spiro[1H-indole-3,2′-3H-1,3,4-thiadiazole]-2-one pharmacophore generated 3 molecules that were similar to OGM1 but lacked LPAR1 activity. See FIG. 41. The GPR68 inhibitory activity of the analogs segregated with the ability to induce the zebrafish phenotype. Furthermore, commercially available inhibitors of LPAR1 (Ki16425) failed to induce the phenotype at concentrations up to 50 μM, ˜200× its IC₅₀. Given its sub-micromolar potency and GPR68 selectivity, one of the OGM1 analogs, OGM8345 (OGM2) was resynthesized and used for further experiments.

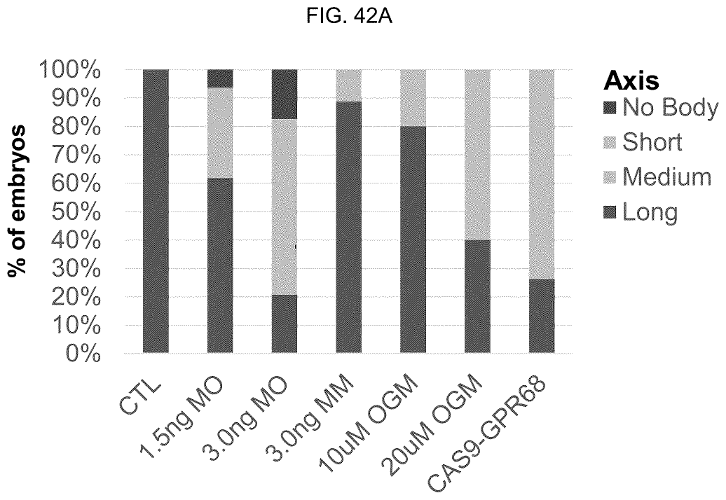

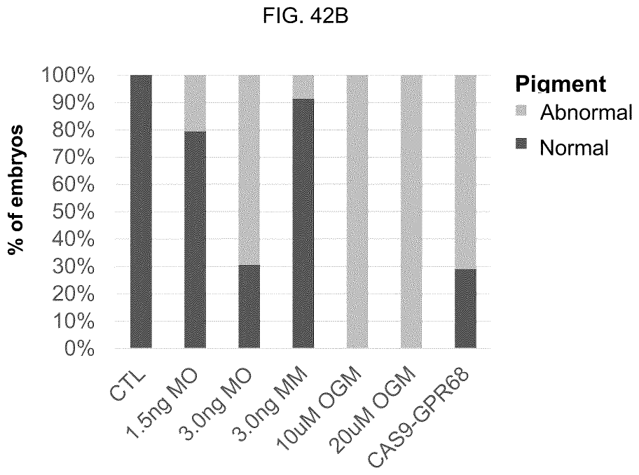

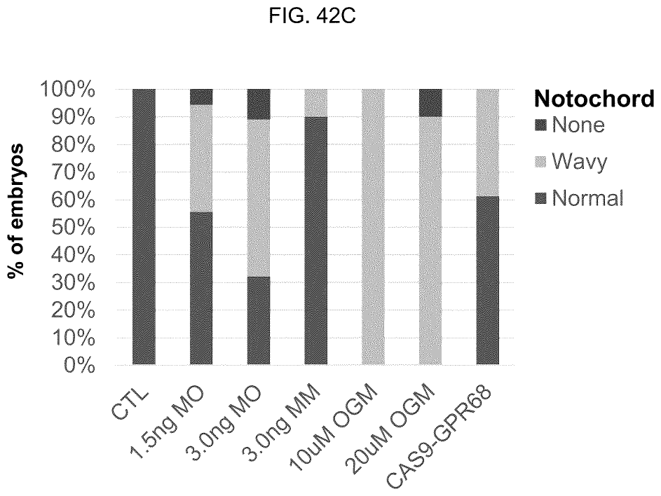

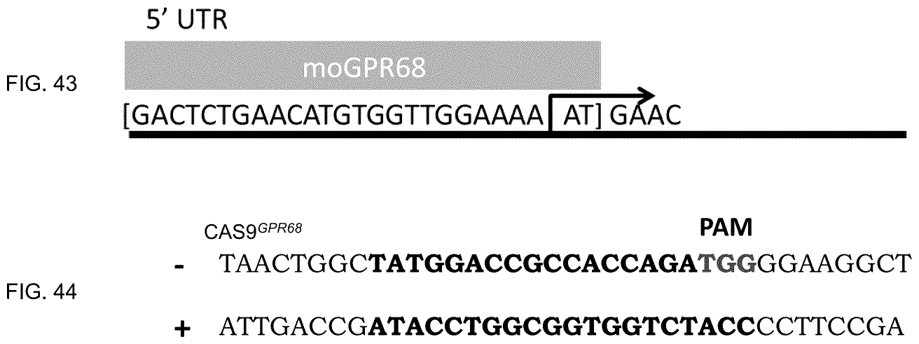

To confirm that the loss of GPR68 activity is sufficient to cause the phenotypes seen in OGM1-treated zebrafish, we used morpholino oligonucleotides to knock down GPR68 expression. Dose-dependent neural crest-specific phenotypes in melanocytes and craniofacial cartilage using 1.5 ng and 3 ng morpholino were observed, whereas the same amount of the mismatched morpholino did not recapitulate these phenotypes. See FIG. 5, FIG. 42, and FIG. 43. The same phenotype was observed in F0 embryos in which the GPR68 gene was targeted by CRISPR/Cas9. See FIG. 5, FIG. 42A, and FIG. 44. Finally, GPR68 knockdown/knockout recapitulated the OGM1-induced wavy notochord and short body-axis phenotypes. FIG. 5. These results demonstrate that the OGM1-induced phenotypes are specifically due to inhibition of GPR68.

### Example 29: Calcium Responses with Extracellular Acidification

To assess the specificity of the calcium response with extracellular acidification, we transfected GPR68 into human embryonic kidney (HEK293) cells, which normally do not express GPR68. The GPR68-transfected cells had a significantly greater calcium response than vector transfected control, which was inhibited by OGM2. Besides GPR68, the other members of the proton-sensing GPCR family are GPR4 and GPR65. Notably, the interspecies homology of orthologs hs.GPR68 and dr.GPR68, is significantly greater than that of human paralogs hs.GPR4 and hs.GPR65. See also FIG. 7.

Because GPR4, the paralog with highest homology with GPR68, was not covered in the initial GPCR profiling, whether OGM2 could inhibit GPR4 was tested by examining the effects of OGM2 on acid-induced serum responsive element (SRE) luciferase activity in HEK293 cells transfected with either GPR4 or GPR68. Mild acidification increased luciferase activity in GPR4 and GPR68 transfected cells above that of vector control.

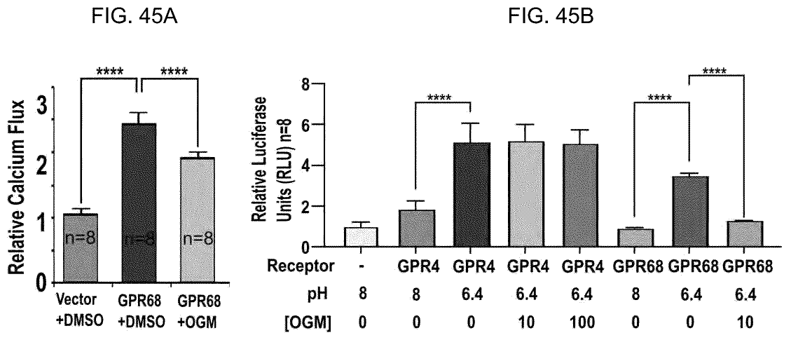

Commercial inhibitor (inh) of LPAR1 (Ki16425, Sigma) also failed to recapitulate the phenotype. Consistent with ogremorphin treatment, Morpholino and Cas9 knockdown of GPR68 resulted in craniofacial dysmorphogenesis, disrupted pigmentation, and a wavy notochord (Red arrow) as shown in FIG. 5. Cas9^{GPR68}=GPR68 targeted sgRNA and Cas9; CTL=control; MoGPR68=GPR68 morpholino treatment. Signaling was inhibited only in cells transfected with GPR68 (see FIG. 5, demonstrating that OGM2 is a selective, and specific inhibitor of GPR68. FIG. 45A shows HEK293 cells transfected with either GPR68 or empty vector control. These cells were stained with calcium indicator dye Calbryte520 AM and stimulated with acid to observe calcium response. These results show the specificity of the calcium response caused by GPR68 and OGM2's ability to inhibit this response. FIG. 45B show HEK293 cells transfected with either GPR4 with SRE (serum responsive element), luciferase or GPR68 and SRE:luciferase. These cells were exposed to acidic media and assayed for luciferase activity as a readout of the receptor activity. These results show that although GPR4 is similar to GPR68, OGM2 only inhibits GPR68 and does not inhibit GPR4.

### Example 30: Glioblastoma Senses Acidification Through GPR68

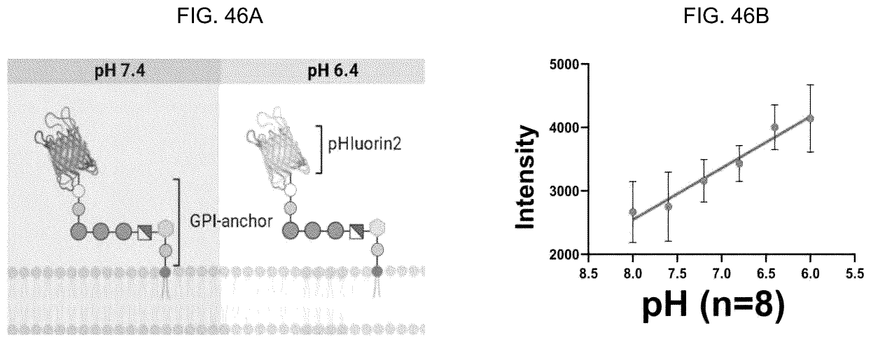

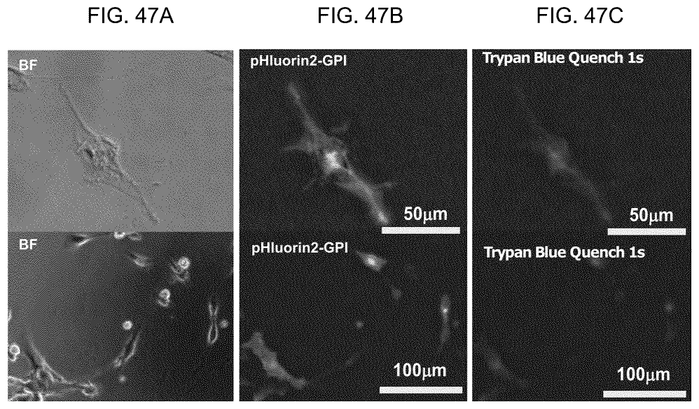

As in many solid cancers, the low extracellular pH of the tumor microenvironment of GBM promotes glioblastoma survival and chemoresistance. To visualize changes in the GBM acidic milieu, a Glycosylphosphatidylinositol (GPI) anchored pHluorin2 was generated. See FIG. 46A. The GPI anchor is a posttranslational modification to a protein that attaches it to the outer leaflet of the cell membrane, and pHluorin2 is a fluorescent protein which upon acidification, emits increased fluorescence following 469 nm excitation. See FIG. 46A and FIG. 46B. U87 glioblastoma cells, which highly express GPR68, were used to generate clones that stably expressed pHluorin2-GPI under a ubiquitous promoter. In these cells, the pHluorin2 fluorescence was quenched by Trypan blue. See FIG. 47A, FIG. 47B, and FIG. 47C. Since Trypan Blue is a vital stain which is excluded from entry into live cells, this result indicates that the acid-responsive fluorescence originates from the extracellular space.

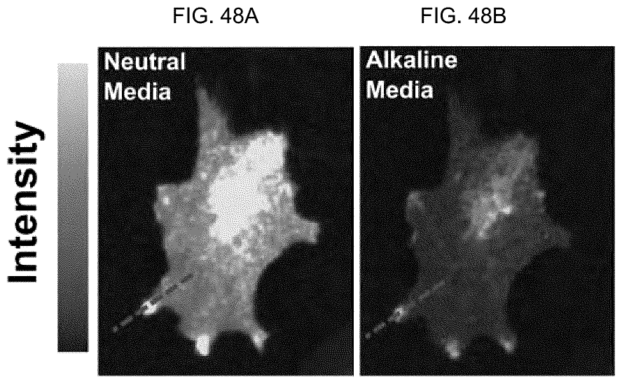

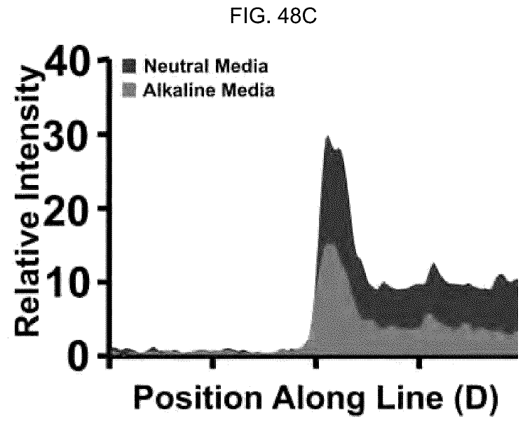

The pHluorin2-GPI-expressing U87 cells displayed foci of high-intensity fluorescence particularly in lamellipodia and filopodia, which were significantly attenuated in alkaline buffered media (pH 8.4). See FIG. 48A, FIG. 48B, and FIG. 48C. These results support the existence of extracellular zones of local acidification on the surface of cells cultured in globally neutral pH conditions.

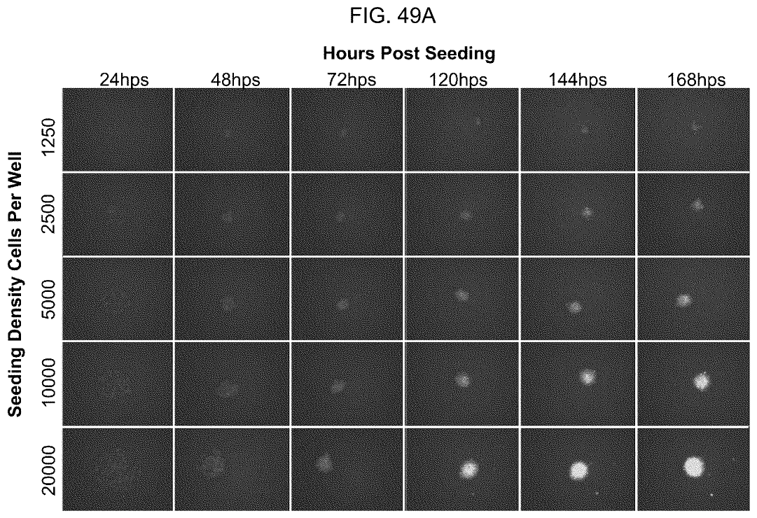

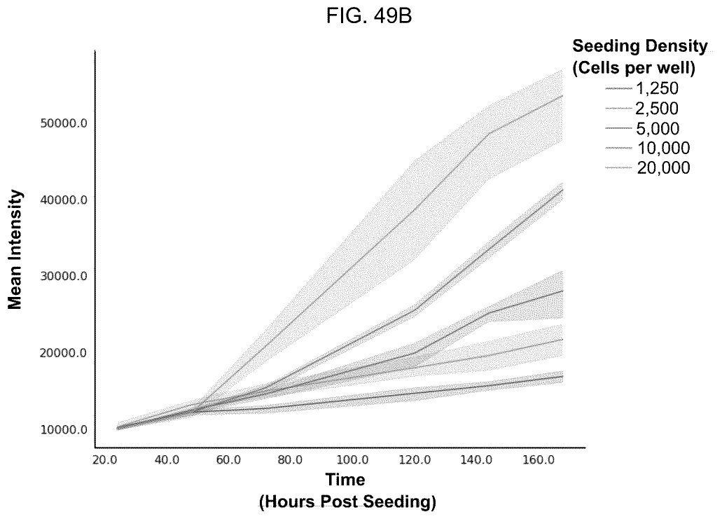

The in vitro 3D spheroid model, in which cancer cells are grown as aggregates, is designed to recapitulate many aspects of the tumor microenvironment (TME), including the nutrient, oxygen and pH gradients that exist in solid tumors in vivo. When 3D spheroids were generated from the U87 cells, extracellular acidification, as indicated by the pHluorin2-GPI fluorescence, was observed throughout the spheroids. See FIG. 48C, FIG. 49A, and FIG. 49B. After 72 hours of spheroid formation, acidic domains appeared to stabilize within the central core of growing tumor spheroids. This is consistent with previous findings that even well-oxygenated regions of tumors are acidic and that acidic regions of tumors extend beyond their hypoxic core. These results indicate that glioblastoma cells in culture acidify their own extracellular milieu.

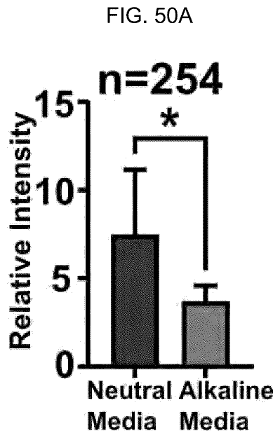

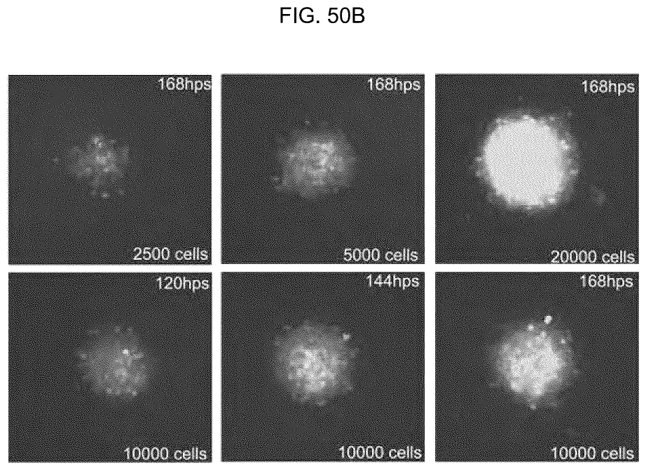

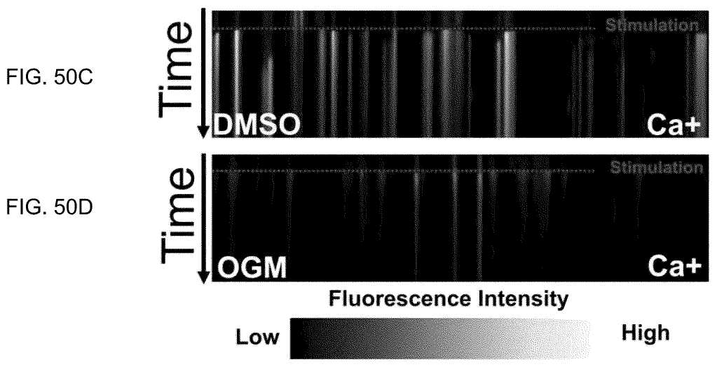

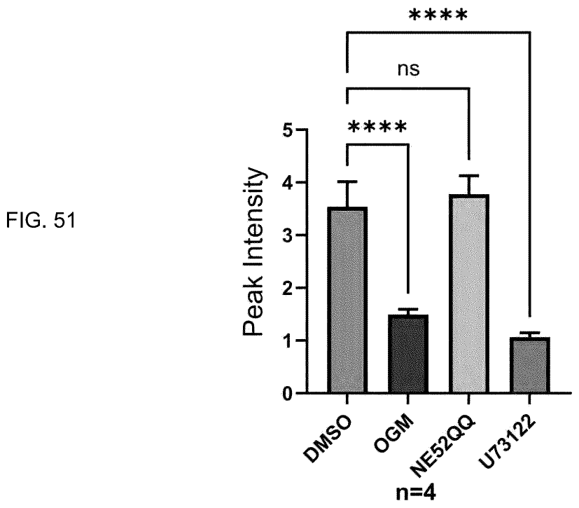

To determine if GBM cells respond to their own acidic extracellular milieu as a form of autocrine signaling, calcium release in response to acidification was measured. GPR68 is known to couple to the Gq subunit, which acts to release calcium from the ER in a PLC dependent pathway. The medium was shifted from pH 7.8 to 6.4, which triggered a rapid and robust calcium flux, as measured by the fluorescence intensity of calcium-sensitive dye Cal520-AM. See FIG. 50A and FIG. 50C. This calcium flux was blocked by OGM2 and PLC inhibitor U73122. FIG. 50A, FIG. 50B, FIG. 50C and FIG. 50D. FIG. 51 shows quantitation of calcium flux. These data suggest that acidification triggers GPR68 specific activation of the PLC pathway resulting in calcium flux.

### Example 31: Loss of GPR68 Activity Reduces GBM Survival

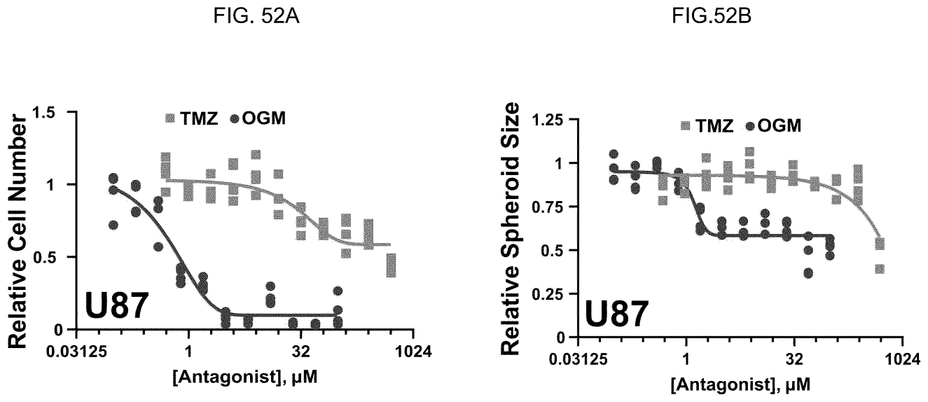

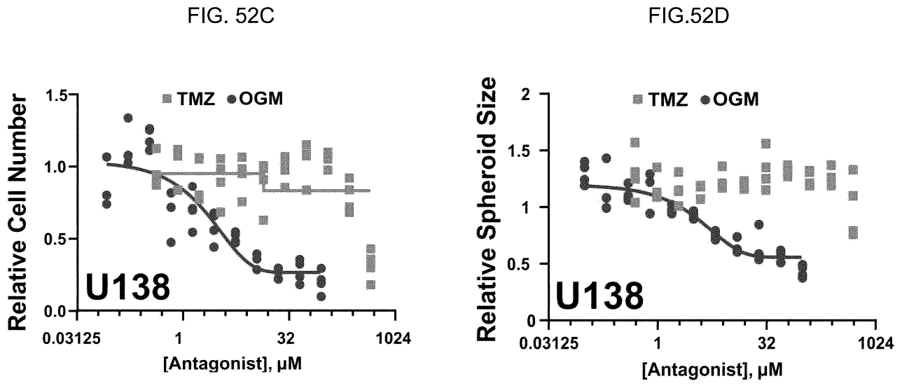

In GBM, it is thought that GPR68 mediates pro-survival mechanisms triggered by the acidic TME, since the acidic extracellular milieu is pro-oncogenic in various settings. Consistent with this, OGM2 treatment decreased viability of U87 cell more potently than temozolomide (TMZ), the first-line chemotherapy for GBM. See FIG. 52A. In the 3D spheroid model of U87 cells, the difference between the antitumor potencies of OGM2 and TMZ was even more marked than in the 2D monolayer. See FIG. 52B. Furthermore, OGM2 treatment decreased viability of U138 glioblastoma cells, which are resistant to TMZ, both in monolayer and 3D spheroid models. See FIG. 52C and FIG. 52D. Interestingly, OGM2 and TMZ demonstrated strong synergistic killing of PDX 08-387 cells with a coefficient of drug interaction (CDI)<0.7 (CDI <1 supports synergism (FIG. 53A, FIG. 53B, FIG. 53C). Taken together, these data suggest that OGM2 is more efficacious than TMZ at treating GBMs, but combinatorial therapy with TMZ may be better.

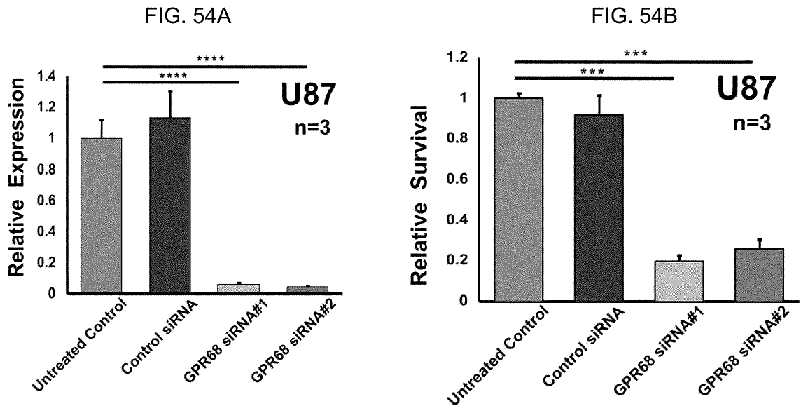

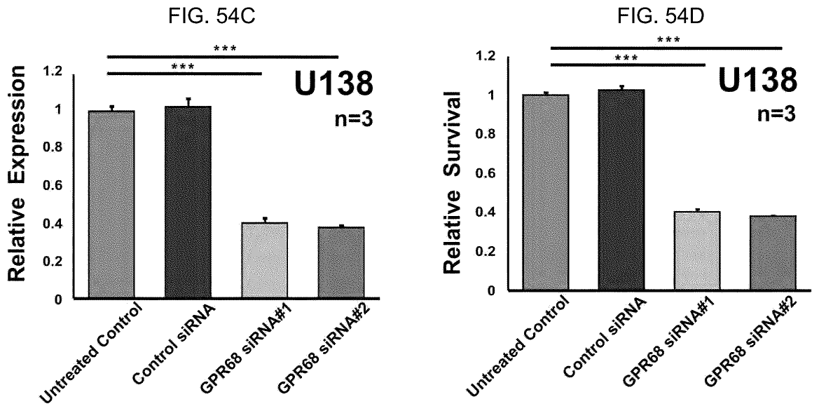

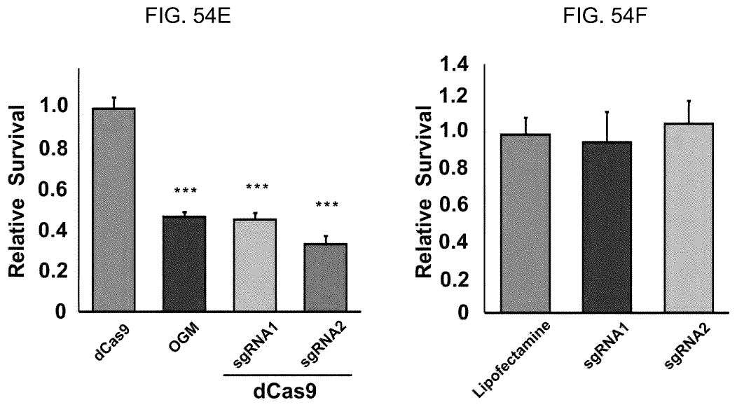

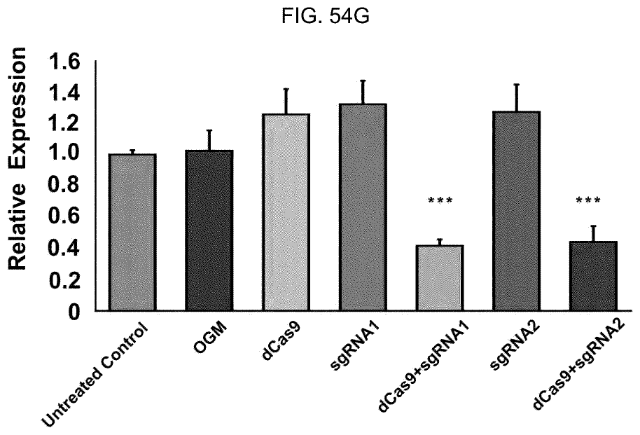

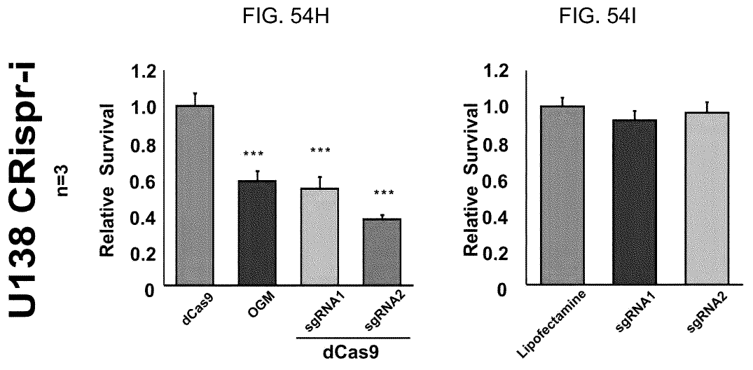

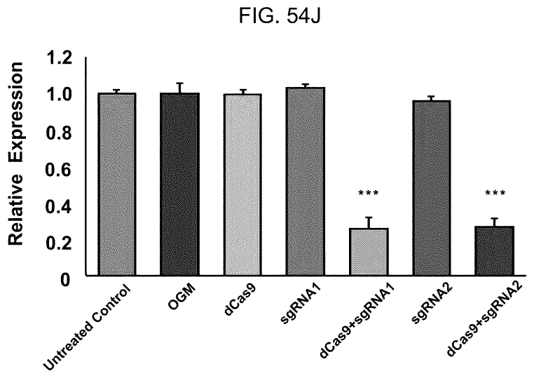

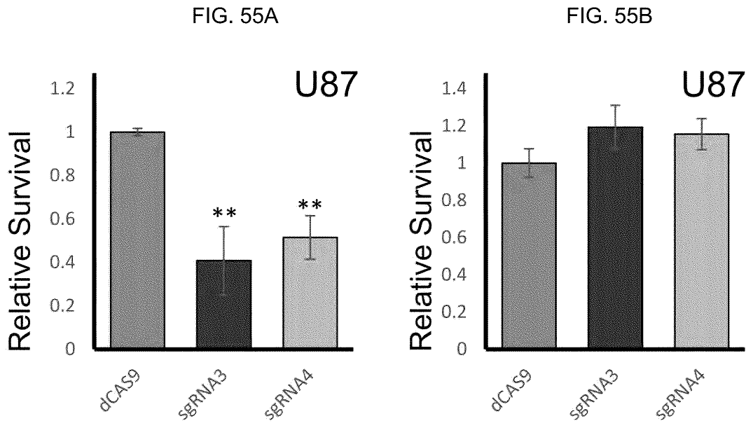

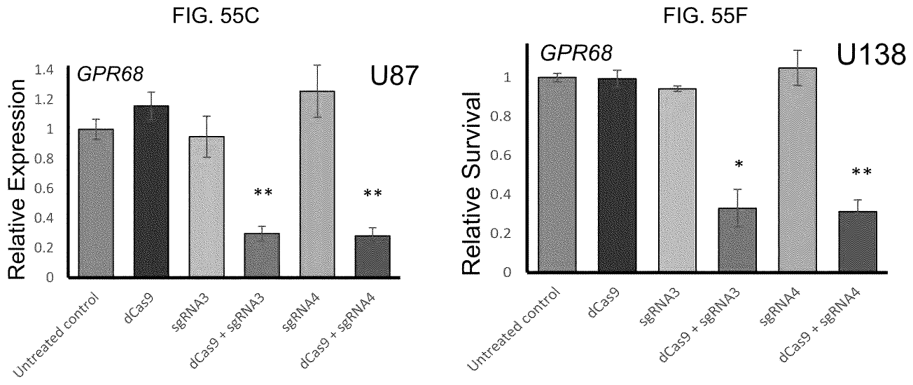

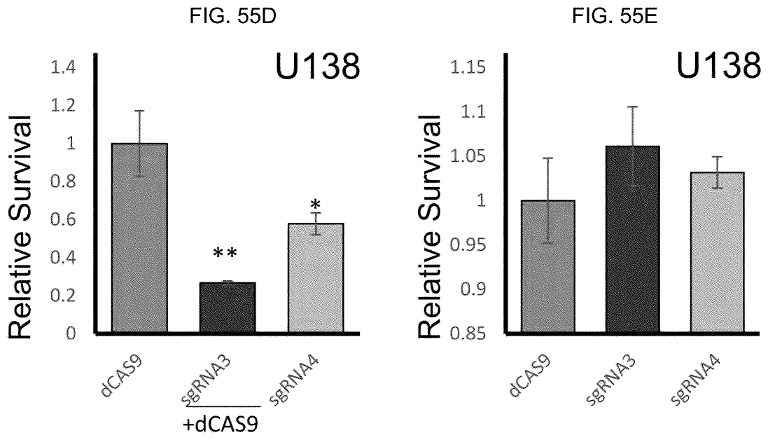

To confirm that the effect of OGM2 on glioma cells is due to GPR68 inhibition, GPR68 was knocked down using siRNA in U87 and U138 cells. Two GPR68-targeting siRNAs, which significantly decreased GPR68 transcript levels (see FIG. 54A), significantly decreased U87 viability, compared to the control siRNA (see FIG. 54B). Additionally, the GPR68-targeting siRNAs also significantly decreased U138 viability, compared to the control siRNA. FIG. 54C and FIG. 54D. Furthermore, knockdown of GPR68 using CRISPR interference (CRISPRi), reduced GPR68 expression and decreased cell viability in U87 and U138 cells, while neither the dCas9 alone nor the respective sgRNAs alone had any effect. See FIG. 54E, FIG. 54F. FIG. 54G, FIG. 54G. FIG. 54H, FIG. 54I, FIG. 54J, and FIG. 55. Therefore, the reduction in GBM viability by OGM2 mediated inhibition of GPR68 activity is recapitulated by siRNA and CRISPRi mediated knockdown of GRP68 expression.

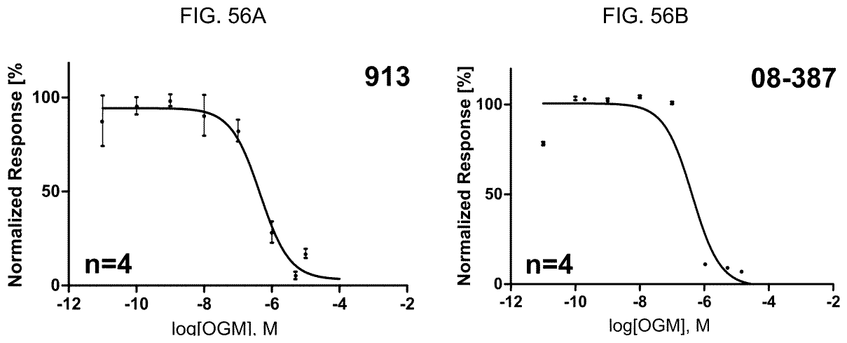

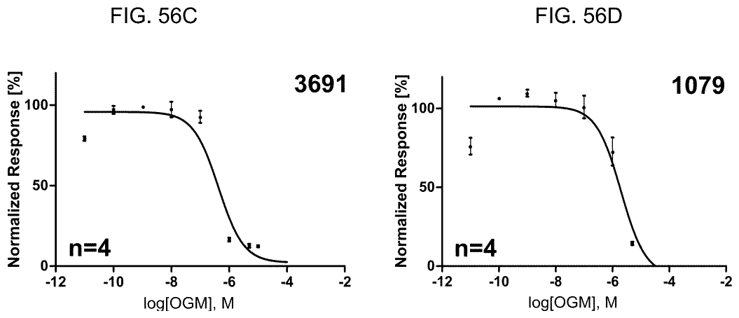

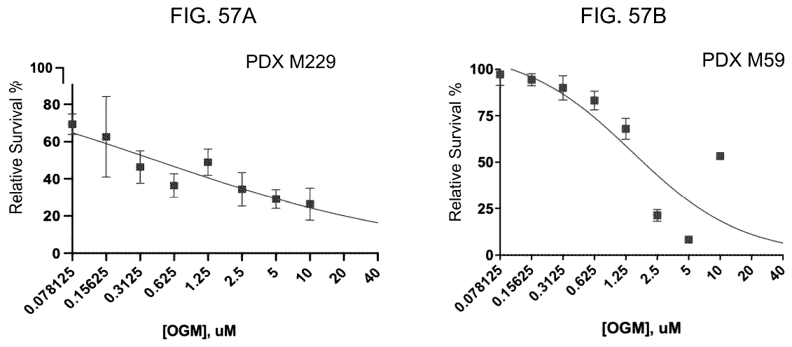

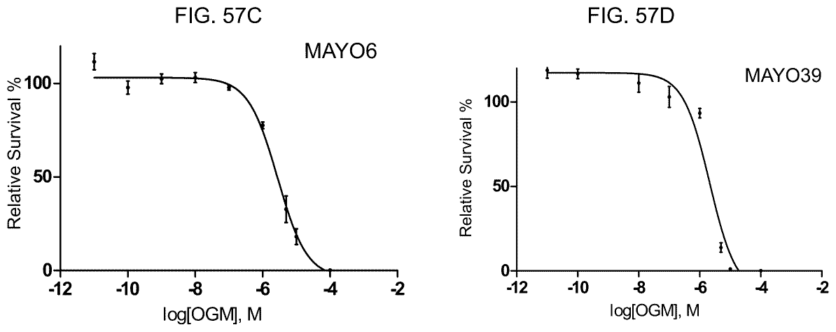

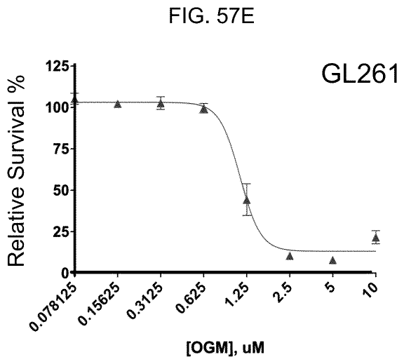

Because glioblastoma cells lines like U87 and U138 can lose some characteristics of the primary GBM tumors while in long-term culture, patient-derived xenograft (PDX) cell models are considered superior models that faithfully maintain the genomic and pathologic features found in the primary tumors. In 2D monolayer cultures, OGM2 treatment significantly reduced viability of each of the 6 independent patient derived lines (PDX and Neurospheres), with LC₅₀'s in the range of 0.42 to 2.7 μM. See FIG. 56, FIG. 55E, and FIG. 55F. In 3D spheroid models, OGM2 treatment significantly reduced viability of the PDX lines with a similar LC₅₀ range. See FIG. 57A and FIG. 57B. Similarly. OGM2 treatment significantly reduced viability of mouse glioblastoma line GL261. See FIG. 57C.

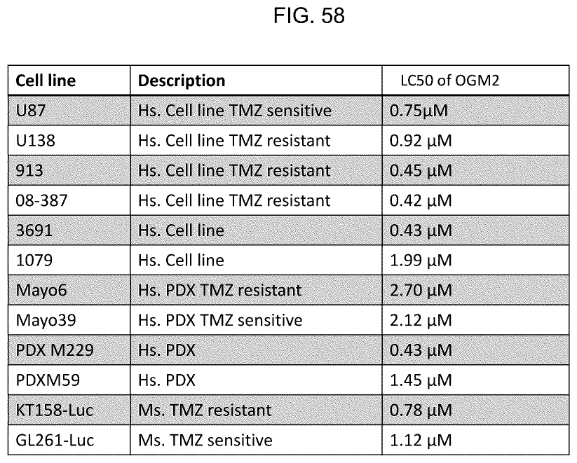

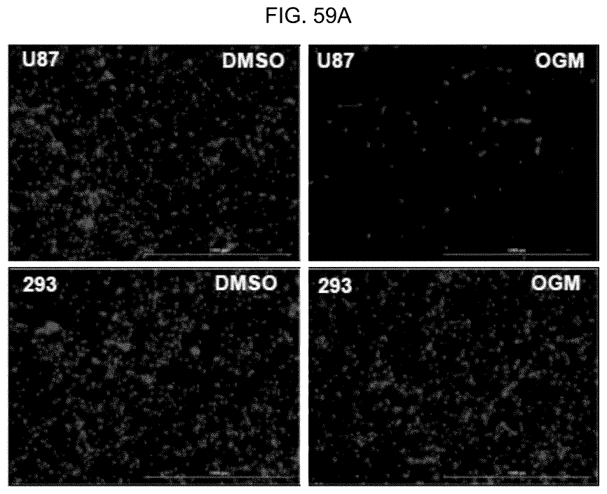

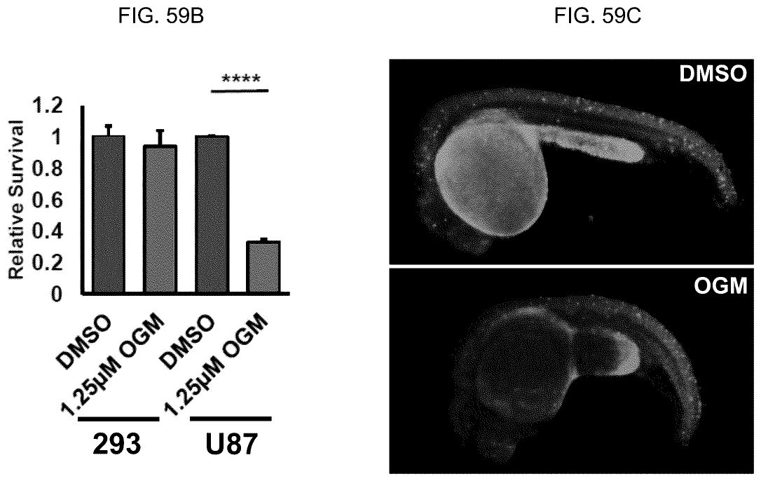

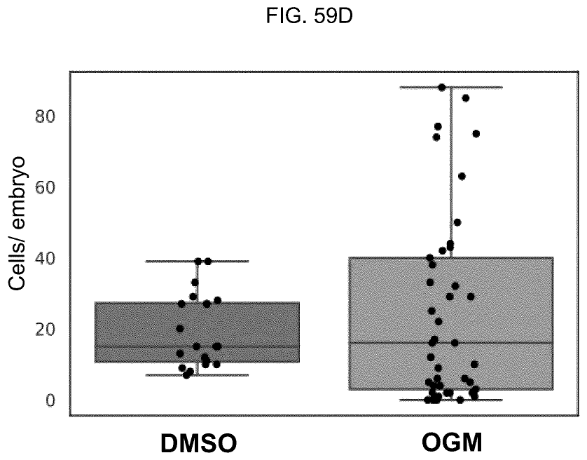

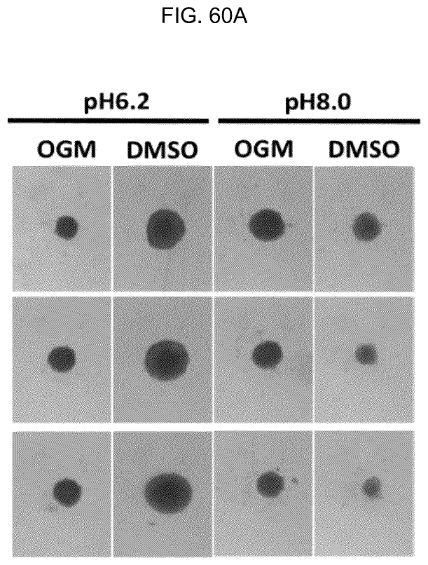

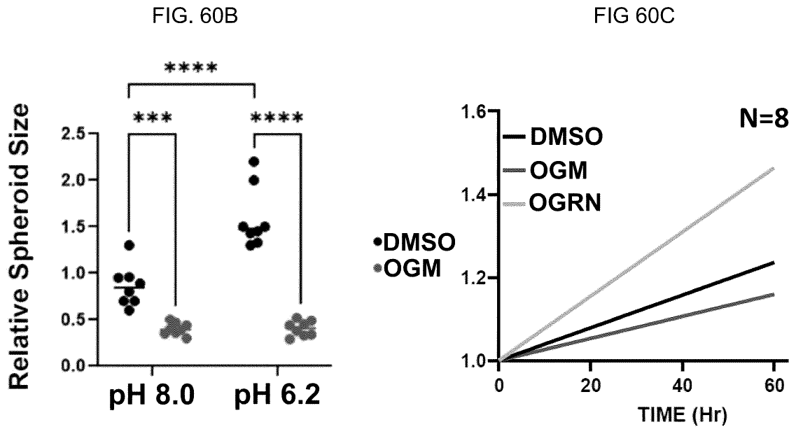

Overall, OGM2 treatment potently reduced viability of all 12 GBM cell lines tested. FIG. 57D and FIG. 57E. See also FIG. 58. By contrast, OGM2 had no effect on HEK293 cell viability, and did not induce excess cell death in zebrafish larvae. See FIG. 59, which rules out nonselective toxicity of OGM2. Additionally, spheroids grown in acidic media (pH 6.2) were larger and grew faster than those grown in basic media (pH 8.0). See FIG. 60A and FIG. 60B. Similarly, spheroids treated with Ogerin, a positive allosteric modulator of GPR68, grew faster than controls. See FIG. 60C. These results suggest that, in response to acidic extracellular milieu, GPR68 mediates both pro-survival and pro-growth pathways conserved in GBM cells, and that OGM2 selectively inhibits this pathway to kill GBM cells across species and subtypes.

### Example 32. OGM2 Triggers Ferroptosis in GBM Cells

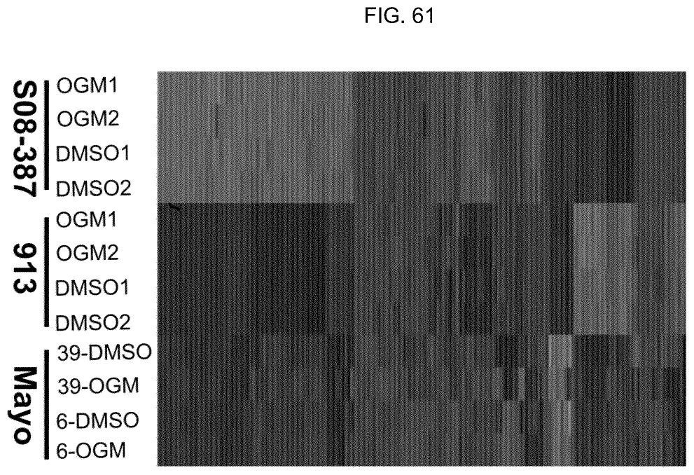

To understand the molecular mechanisms by which OGM causes GBM cell death, a global transcriptomic profiling (RNA-seq) was performed of four independent, molecularly heterogeneous, human GBM patient derived cell lines 913, 08-387. Mayo6 and Mayo39 treated with DMSO vehicle or OGM2 at respective LC₅₀'s for 72 hours. The differential gene expression analysis revealed significant transcriptomic changes with OGM2 in all lines. FIG. 61. In FIG. 61, “OGM,” “OGM1,” and “OMG2” all refer to OGM2 samples. See also Appendix A.

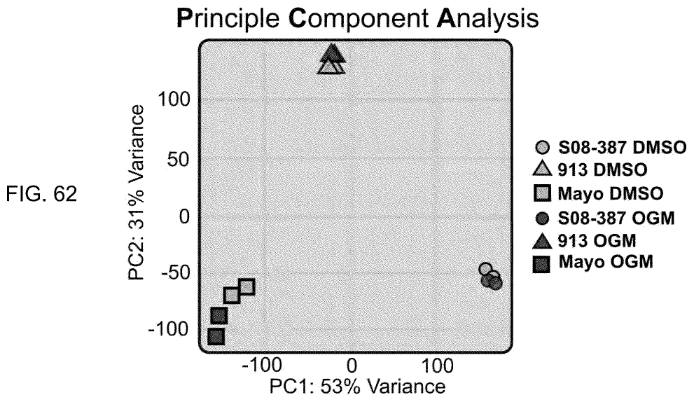

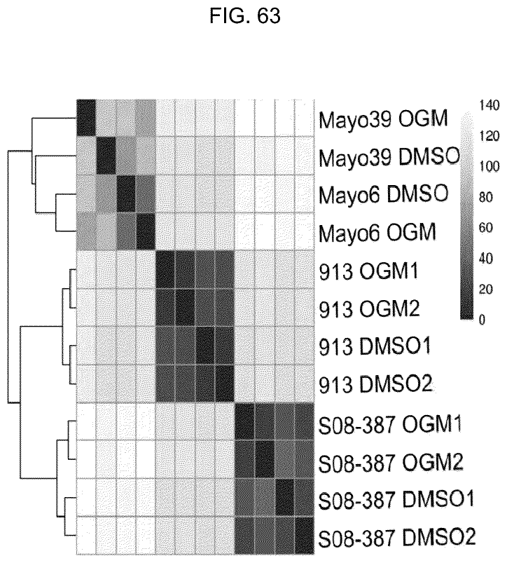

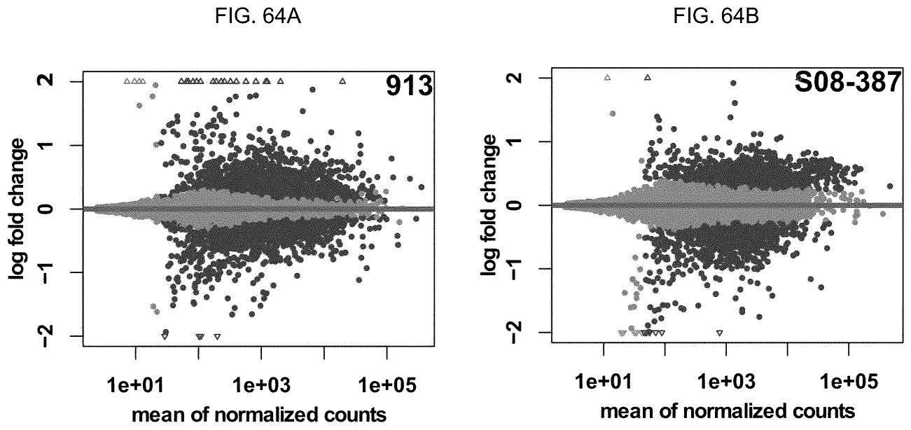

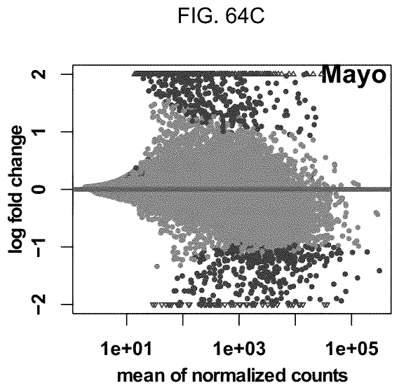

The principal component analysis (PCA) indicated that each GBM cell line were significantly different from each other (FIG. 62), consistent with known molecular heterogeneity of GBM cells. Moreover, each OGM2-treated cell line was transcriptionally most similar to its untreated counterpart, suggesting that the underlying difference between glioblastoma cell line is larger than the changes induced by the OGM2-treatment. See FIG. 62 and FIG. 63. In FIG. 63, “OGM,” “OGM1,” and “OGM2” all refer to different experimental runs with the same OGM2 samples. Next, we identified significantly differentially expressed (SDE) genes (ABS Log FC 0.585, False discovery rate (FDR)<0.01) induced by the OGM2-treatment for each GBM cell line. See FIG. 64A, FIG. 64B, FIG. 64C, and Appendices B, C, and D,

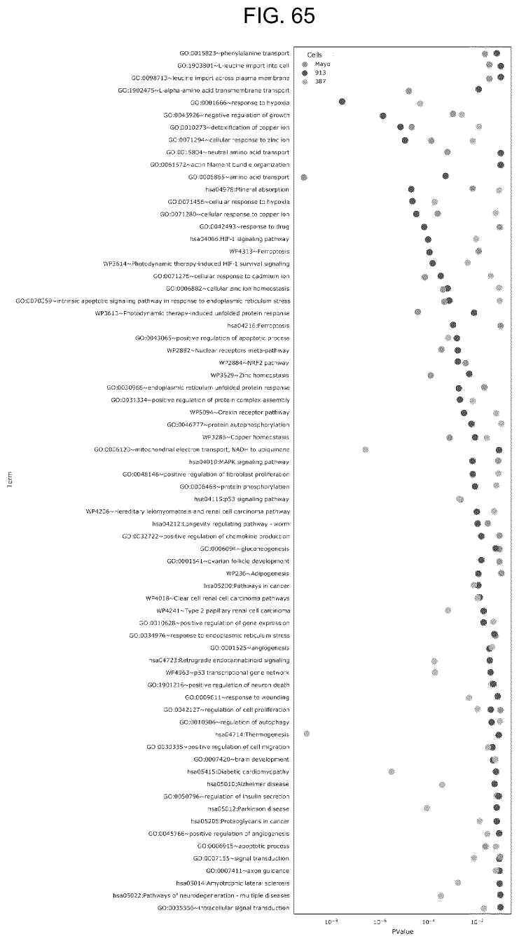

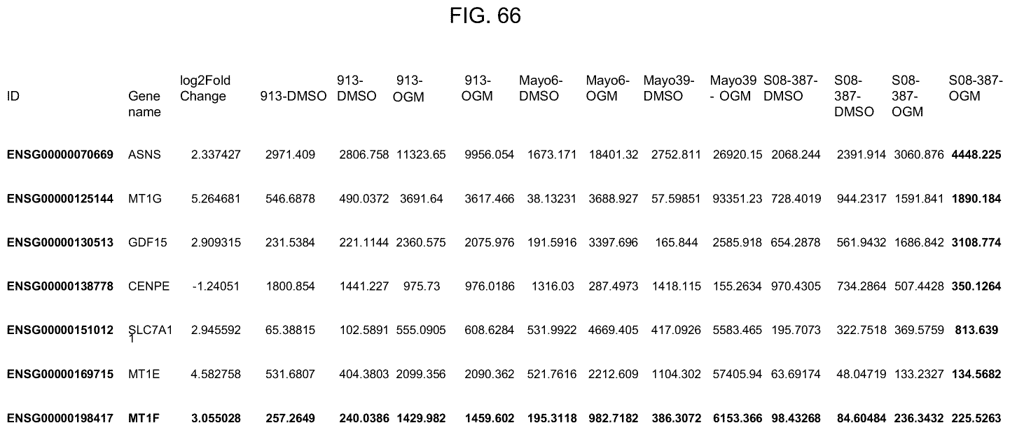

A Venn diagram highlights the 7 SDE genes that were consistently differentially expressed in OGM2 treatment across the different GBM types. See FIG. 65 and FIG. 66.

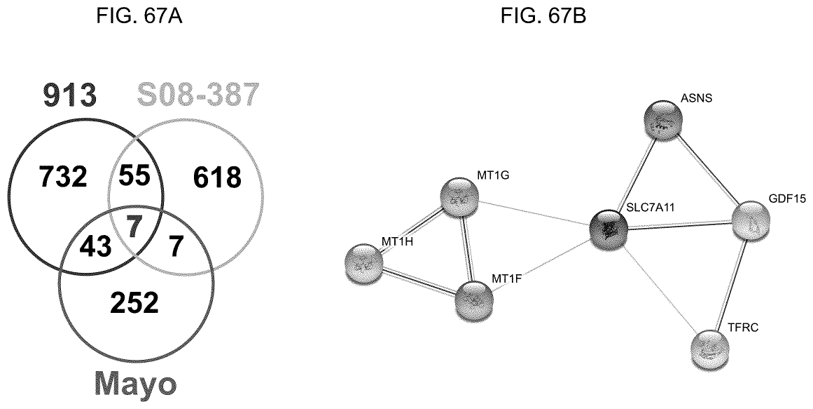

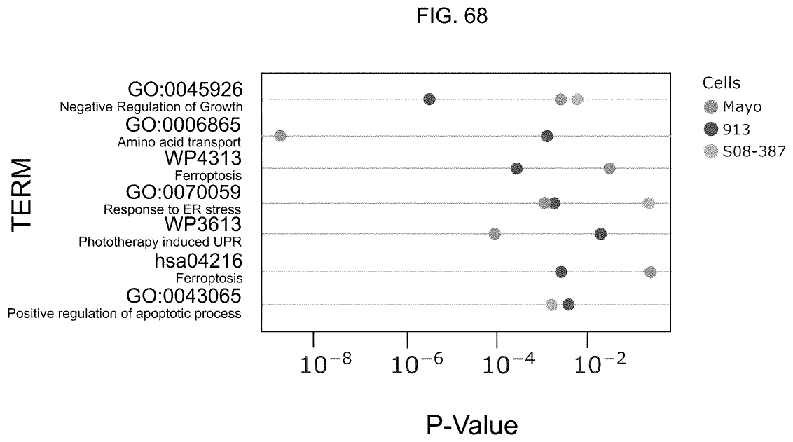

Given the substantial differences in the baseline transcriptomic landscape across different GBM types, and the relatively small differences between treatment and control groups for each line, identification of the dysregulated pathways that were shared was investigated. When SDE genes in each group were subjected to GO, KEGG and WIKIPATHWAY gene set enrichment analysis (FIG. 67 and Appendix E), it revealed “Negative Regulation of Growth” (GO:0045926), “Amino Acid Transport” (GO:0006865), “Ferroptosis” (WP4313, has:4216), “Unfolded Protein Response (UPR)/Endoplasmic Reticulum (ER) Stress” (GO:0070059, WP3613), and “Positive Regulation of Apoptotic Process” (GO:0043065) as shared enriched terms. See FIG. 68. Notably, 3 of the 7 SDE genes induced by the OGM2-treatment, ASNS. GDF15, and SLC7A11, are each annotated as a marker of ferroptosis In the FerrDB database. GO, KEGG and WIKIPATHWAYS are public informational databases with gene and functional information Statistical over representation of the number genes for each functional category was assessed to determine if certain “pathways” and molecular functions were disrupted.

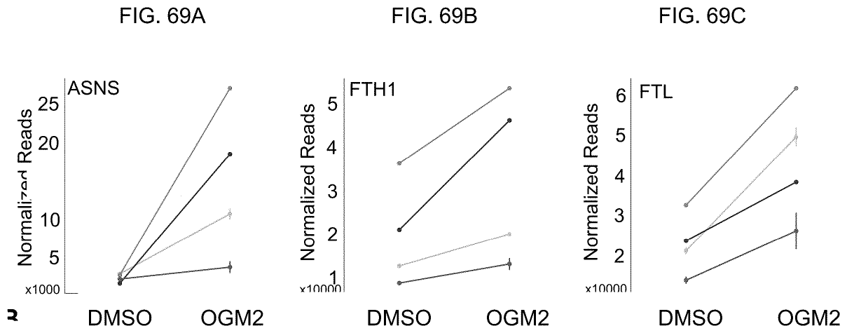

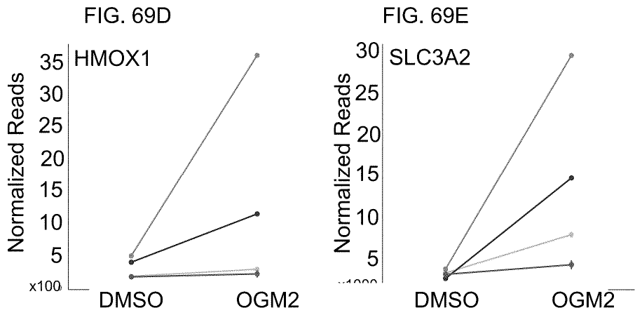

Ferroptosis is an iron-dependent programmed cell death pathway characterized by accumulation of lipid peroxides that is genetically and biochemically distinct from other programmed cell death mechanisms. Consistent with the induction of ferroptotic cell death in GBM cells, OGM2 treatment significantly altered expression of 3 of the genes encoding metallothioneins (MTs), which directly bind iron to protect cells from oxidative damage. See FIG. 64B. A closer examination of the RNA-seq data revealed that OGM2-treatment induced the expression of several classic ferroptosis markers, specifically TFRC ASNS, F7H1, F7L, HMOX1 and SLC3A2. See FIG. 69.

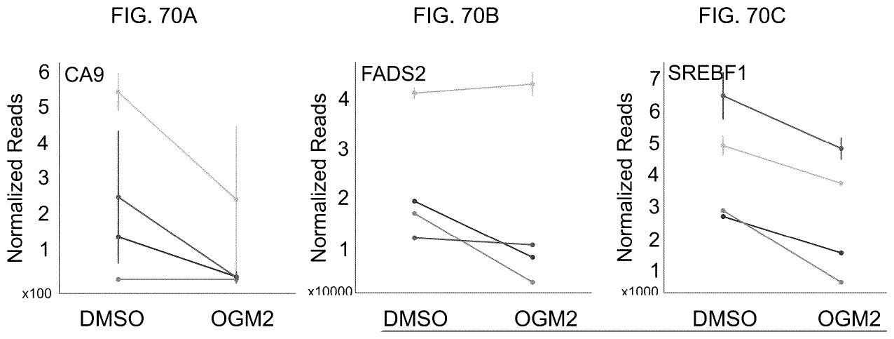

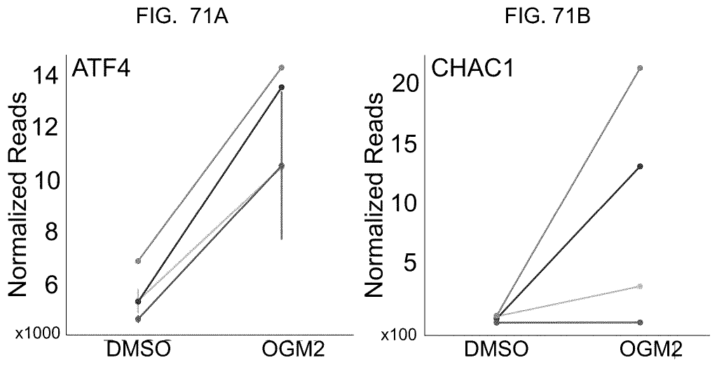

Moreover, OGM2 significantly reduced the expression of known ferroptosis suppressors C49, FADS2, and SREBF7 (FIG. 70). Additionally, OGM2-treatment induced expression of ATF4 and CHAC1, the core mediators of both ferroptosis and ER stress (Appendix A). Although commonly associated with ER stress, ATF4 can also induce ferroptosis as a key transcription factor that induces expression of CHAC1. See FIG. 71 CHAC1 encodes ChaC (also Glutathione Specific Gamma-Glutamylcyclotransferase-1) which degrades glutathione (GSH), the main antioxidant mechanism in cells, resulting in accumulation of toxic lipids. These results suggest that OGM2 treatment causes cell death in GBM cells through ferroptosis.

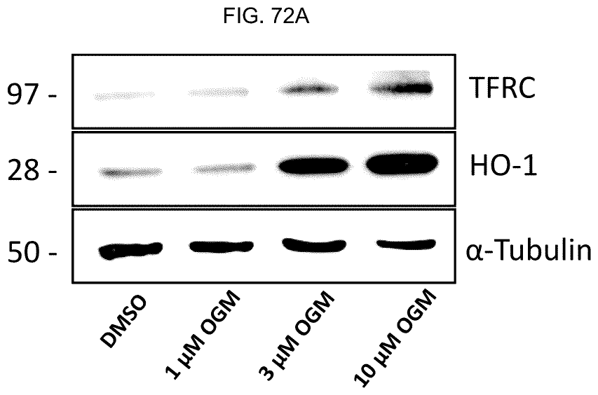

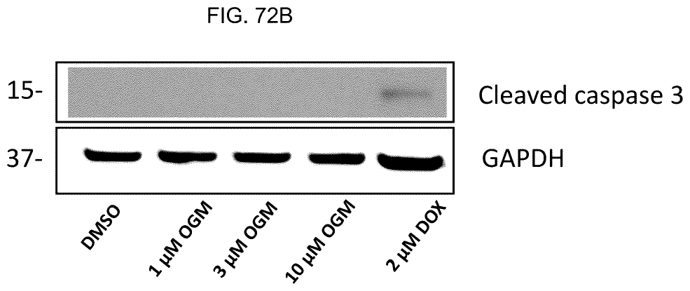

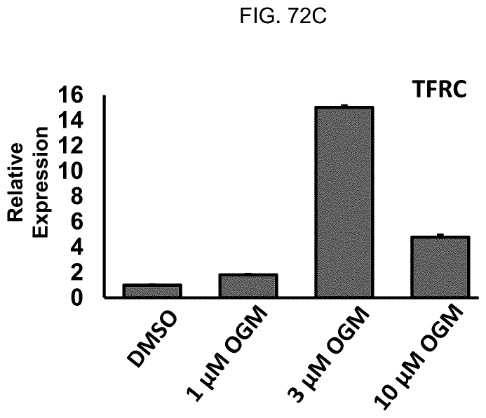

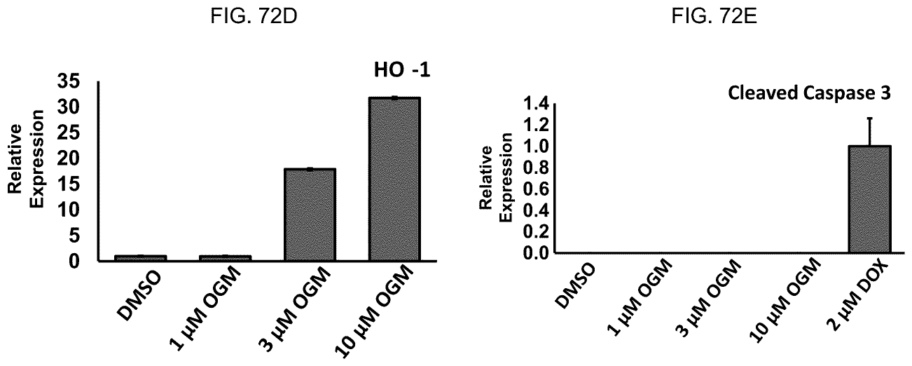

Consistent with the induction of ferroptosis genes observed in the RNA-seq analysis of OGM2-treated PDX models, OGM2-treatment of U87 cells significantly increased the protein levels of TFRC (Transferrin Receptor) and HO-1 (Heme Oxigenase-1), the established indicators of ferroptosis and ROS, respectively (FIG. 72A and FIG. 72B). FIG. 72C, FIG. 72D, and FIG. 72E are quantification of 3 or more biological replicates of the western blots shown in FIG. 72A and FIG. 72B.

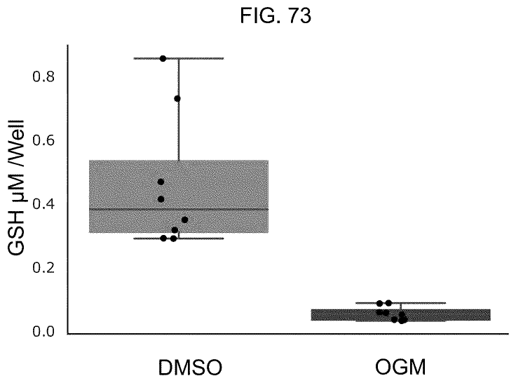

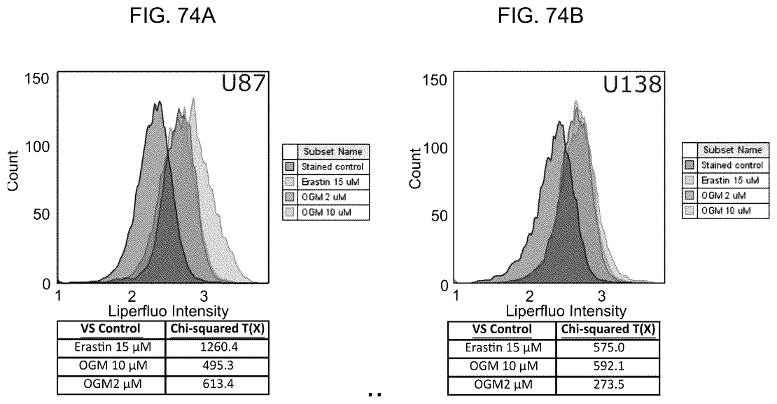

As expected with the established CHAC1 induction, OGM2 treatment dramatically reduced glutathione levels (FIG. 73) and significantly increased lipid peroxidation in U87 and U138 cells (U87 Chi-squared T(X)=625,495 for 2 μM and 10 μM, respectively; U138 Chi-squared T(X)=273, 592 for 2 μM and 10 μM, respectively; Chi-squared T(X)>4 is equal to p<0.01). See FIG. 74A and FIG. 74B.

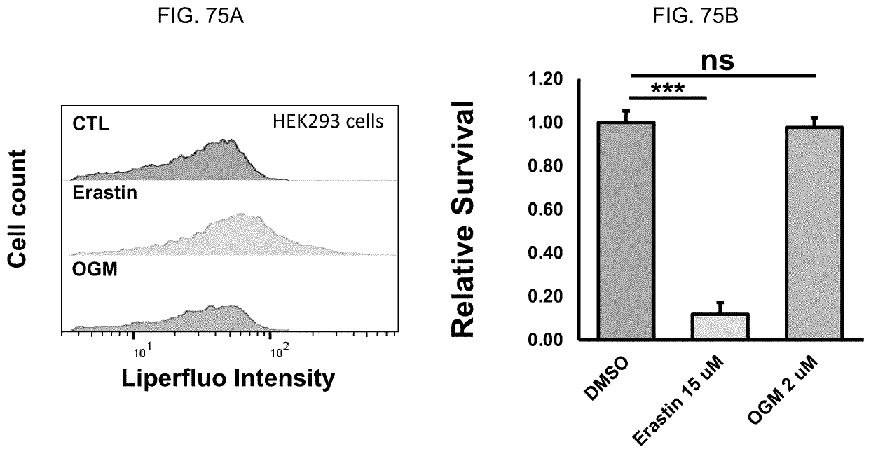

However, consistent with having no effect on HEK293 survival, OGM2 treatment did not induce lipid peroxidation in HEK293 cells (FIG. 75). The small molecule Erastin was used as a control because it triggers ferroptosis by inhibiting the cystine-glutamate antiporter system Xc. Thus, our results indicate that OGM2 selectively induces ferroptosis in GBM cells by blocking GBM's cellular glutathione (GSH)-dependent antioxidant defenses.

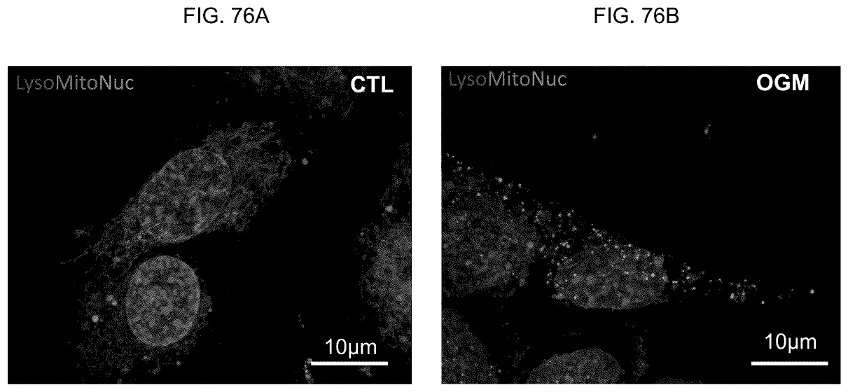

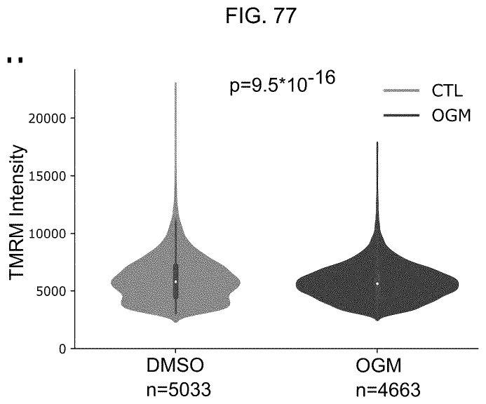

Lipid peroxidation associated with ferroptosis is known to disrupt mitochondrial membranes, resulting in smaller mitochondria. OGM2-treated U87 cells exhibited punctate mitochondria, when stained with vital mitochondrial stain MitoTracker, with decreased mitochondrial membrane potential, as measured by TMRM (Tetramethylrhodamine, methyl ester) staining, without discernable effect on lysosomes. See FIG. 76 and FIG. 77. See also Appendix E, which is a gene set enrichment analysis applied to significantly differentially expressed genes (SDEG) from each cell type. Significantly enriched terms shared by 2 or more cell types are shown.

Transmission electron microscopy (TEM) of U87 cells after 12- and 24-hours of OGM2 treatment demonstrated smaller mitochondria with an increased incidence of ruptured membranes (FIG. 78). Notably. OGM2-treated U87 cells did not exhibit the distended ER seen in the ER stress response, nor mitochondrial membrane blebbing seen in apoptosis (FIG. 78). Moreover, OGM2 treatment did not increase caspase-3 cleavage (FIG. 59B). Lastly, consistent with the known synergy between small molecule ferroptosis inducers and ionizing radiation, OGM2 and ionizing radiation demonstrated exceptionally strong synergistic induction of lipid peroxidation in U87 and U138 cells with a coefficient of drug interaction (CDI)<0.06 (CDI <1 indicates synergism. See FIG. 79. These data suggest that OGM2 treatment induces GBM cell death through ferroptosis.

### Example 33: GPR68 Inhibition Induces Ferroptosis Via ATF4-Dependent Mechanism

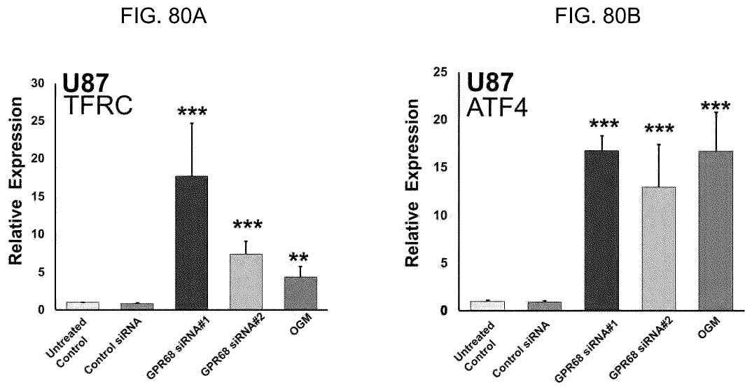

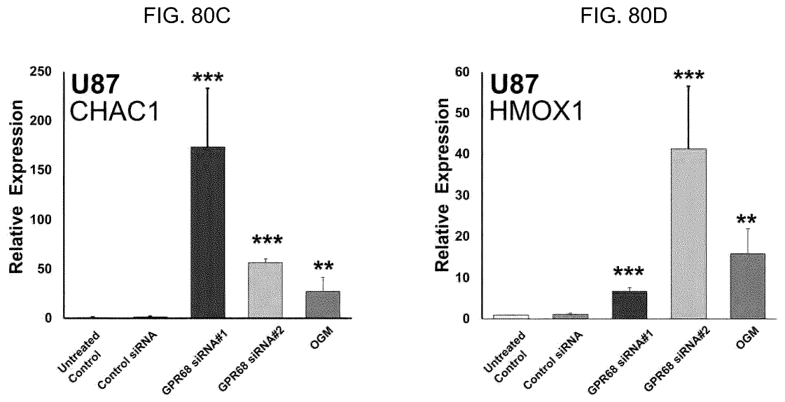

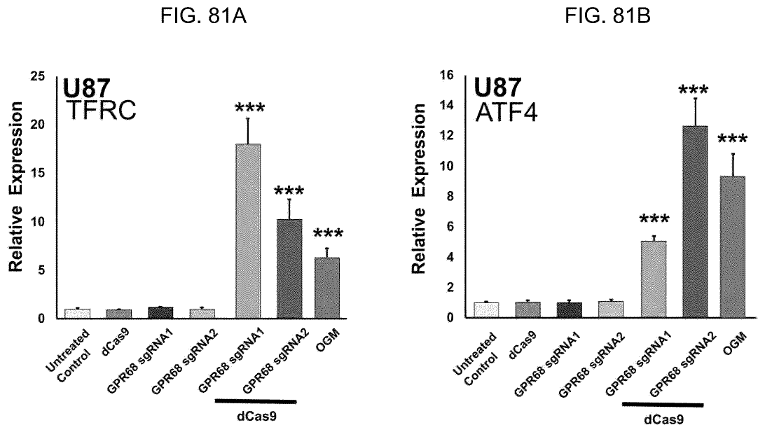

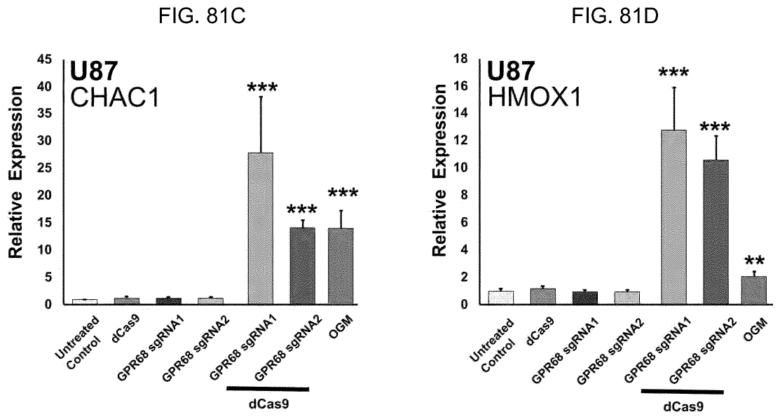

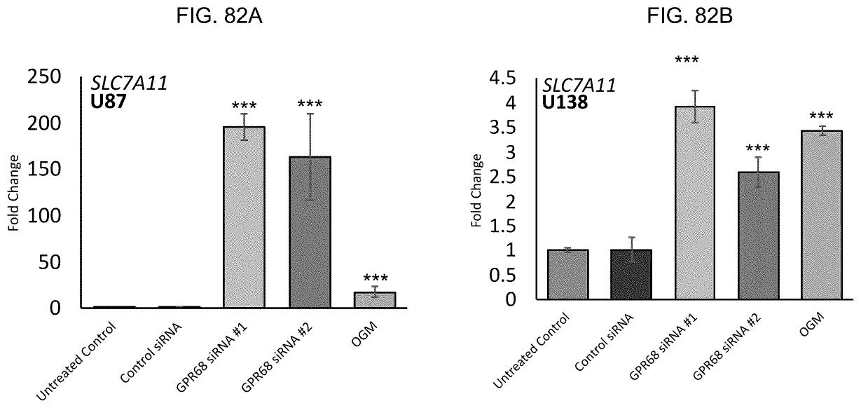

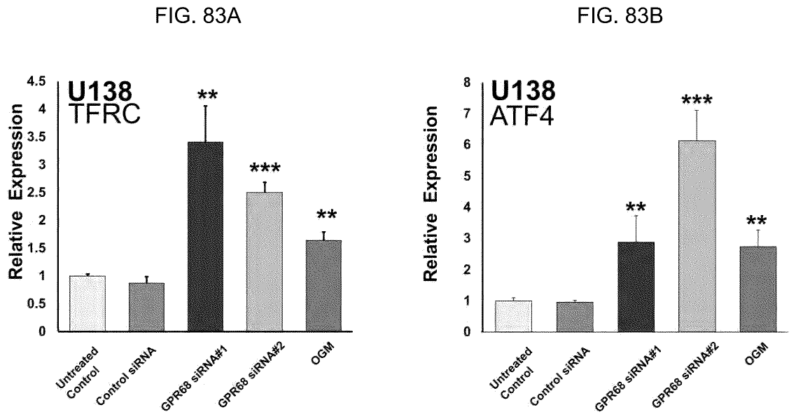

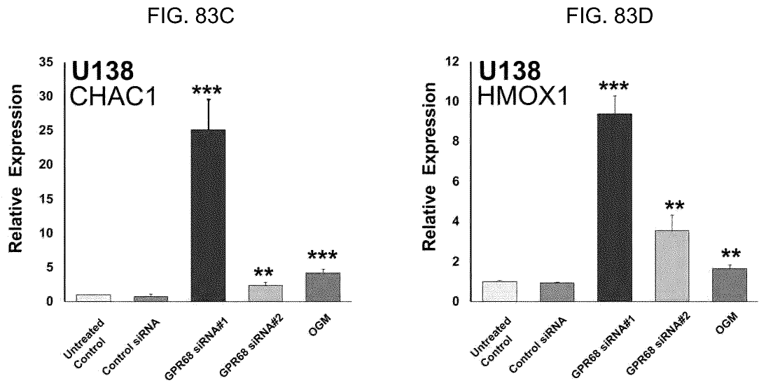

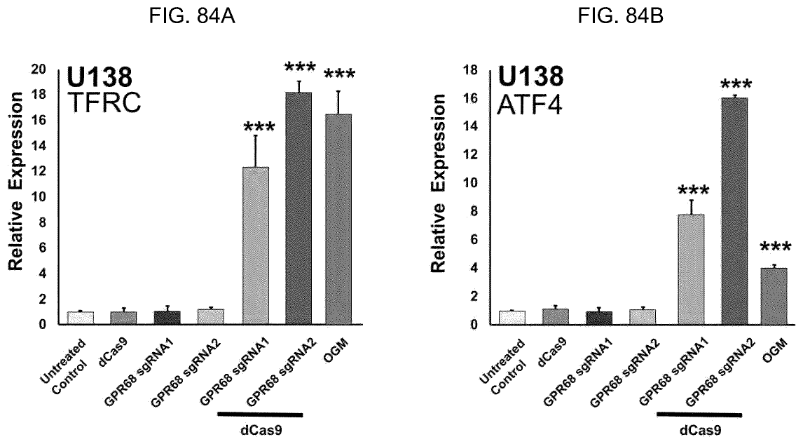

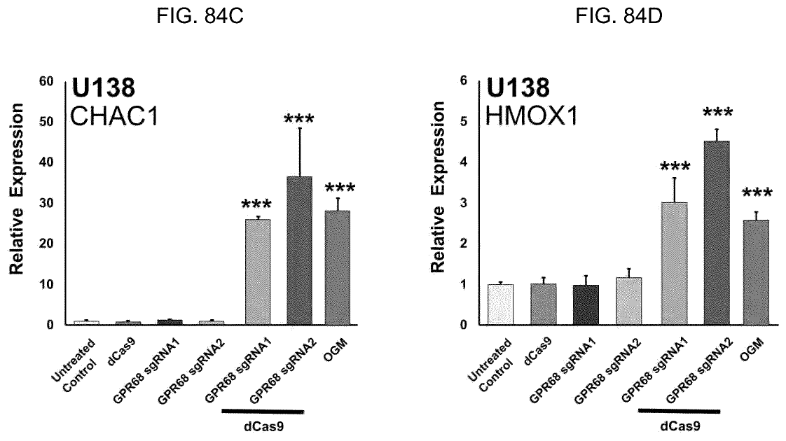

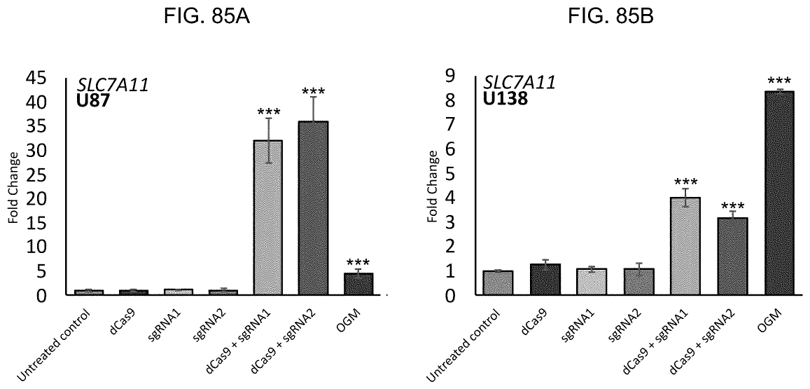

We confirmed that OGM2-induced increased transcription of TFRC, HMOX1, ATF4 and ATF4 targets CHAC1 and SLC7A11 via qPCR (FIG. 79). To confirm that the effects of OGM2 on GBM cells are due to GPR68 inhibition, we assessed biomarkers of ferroptosis after GPR68 knockdown. In both U87 and U138 cells, siRNA-mediated knock-down of GPR68 significantly increased the expression of the ferroptosis markers 7FRC. A7T4 with transcriptional targets CHAC1 and SLC7A11; and increased HMOX1 expression, an indicator of oxidative stress. FIG. 80, FIG. 81, and FIG. 82. Similar results were obtained with CRISPRi-mediated knock-down of GPR68. FIG. 83, FIG. 84, and FIG. 85.

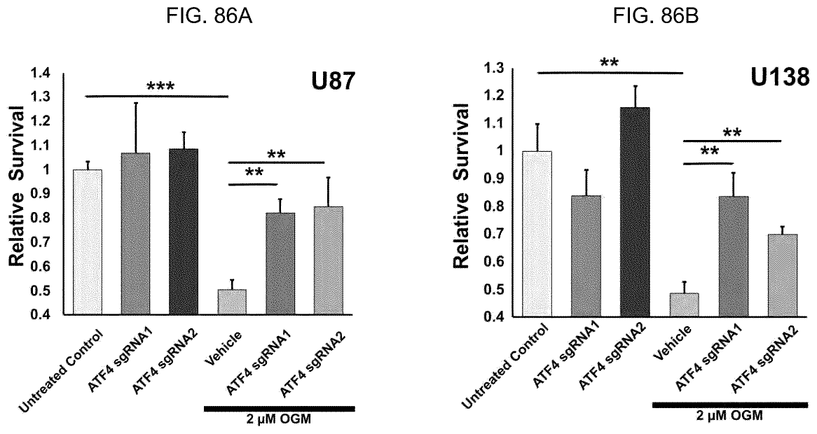

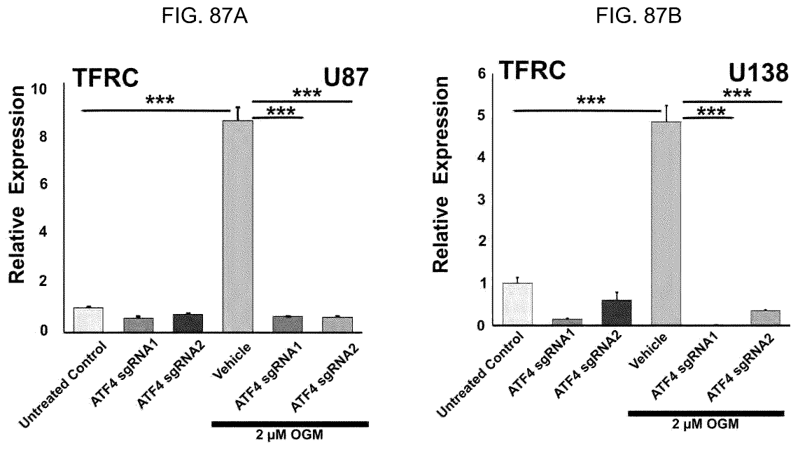

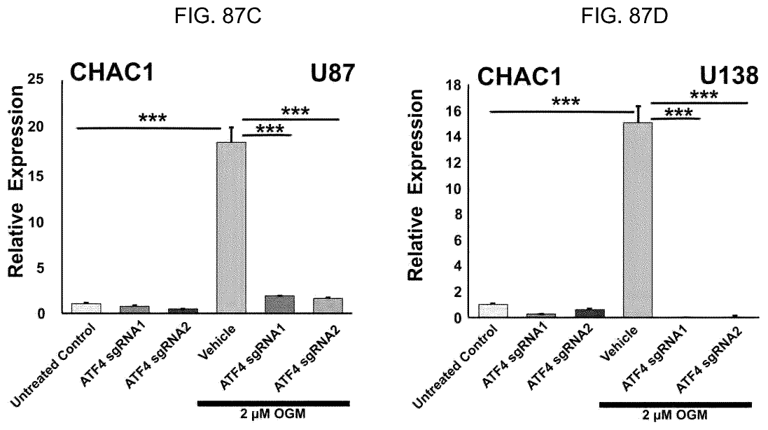

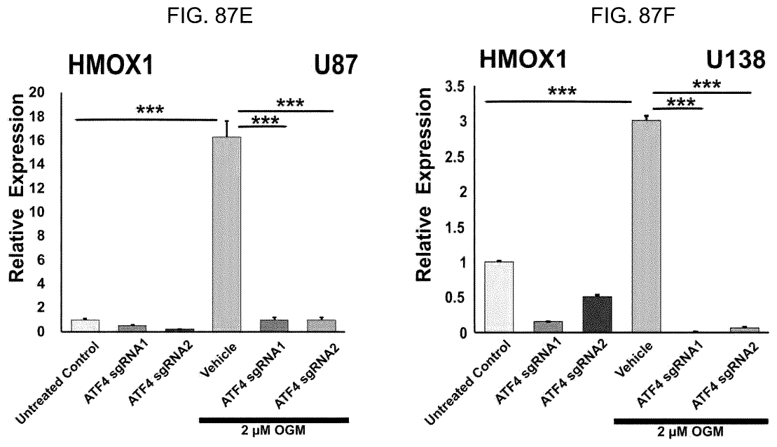

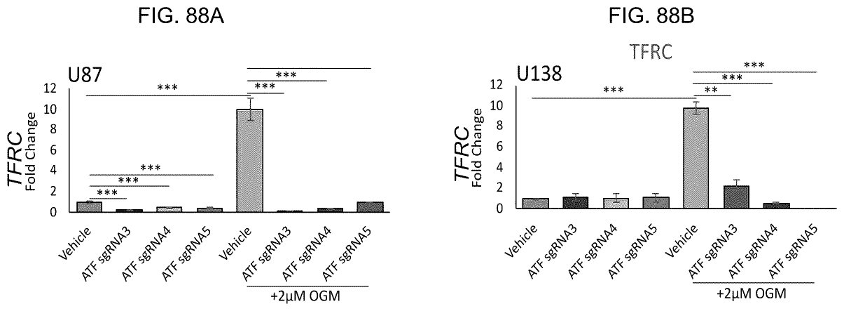

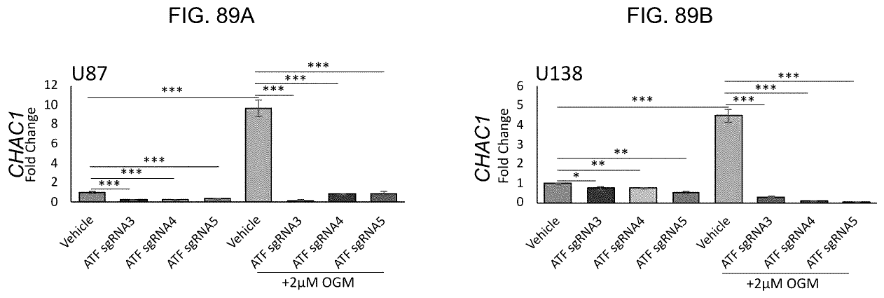

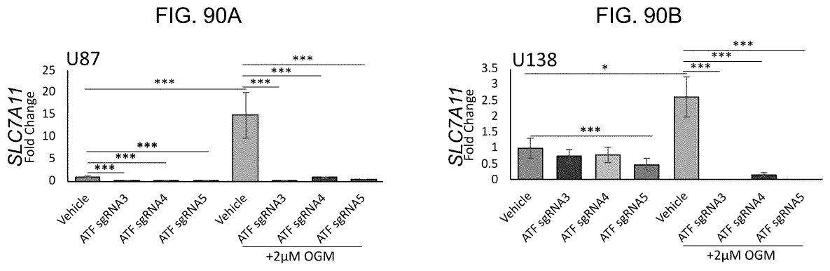

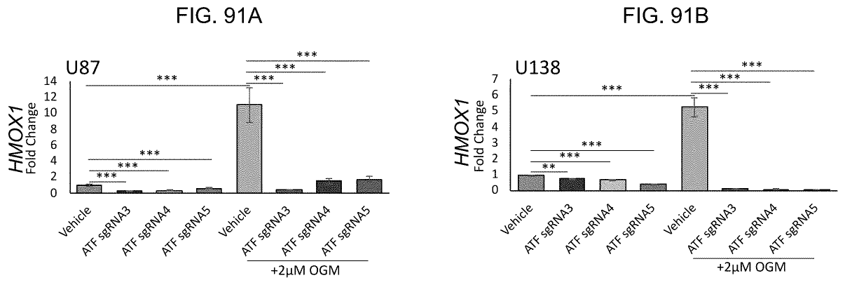

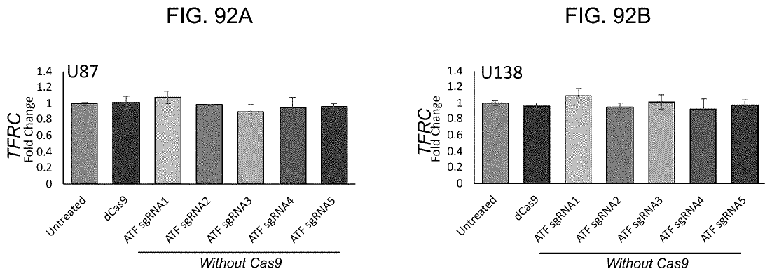

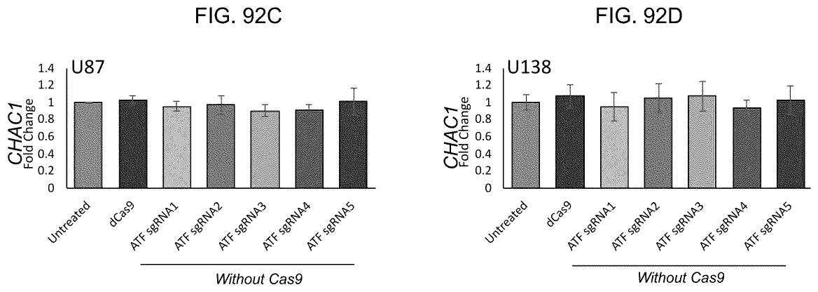

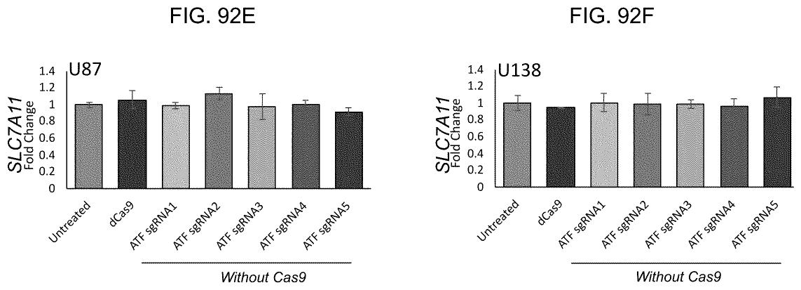

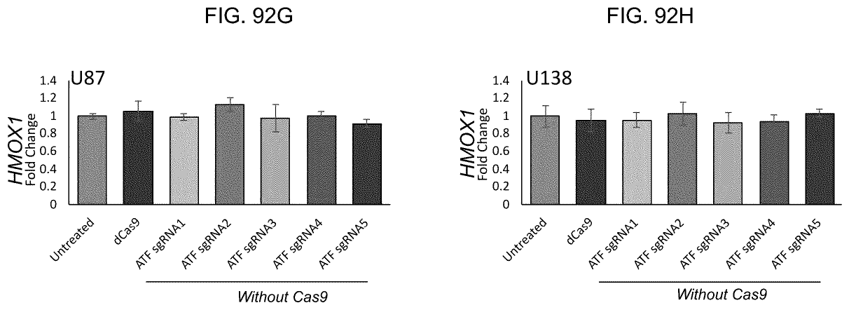

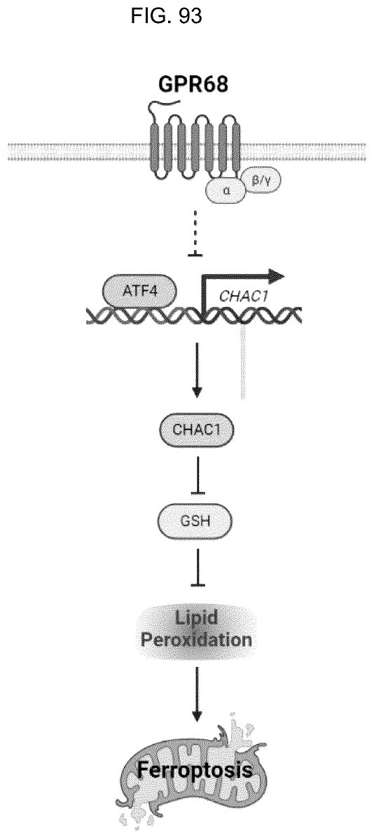

Because loss of GPR68 signaling induced expression of ATF4, whether the ATF4 was required for ferroptosis induction by OGM2 was tested. Knock down of ATF4 significantly attenuated much of the effects of OGM2 on U87 and U138 cells, including the induction of cell death (FIG. 86), the ferroptosis markers TFRC (FIG. 87A, FIG. 87B, and FIG. 88), CHAC1 (FIG. 87C and FIG. 87D, and FIG. 89), and SLC7A11 (FIG. 90), and the oxidative stress marker HMOX1 (FIG. 87E and FIG. 87F and FIG. 91). Cas9 alone and sgRNA alone controls had no effect on any of these genes. See also the negative controls in FIG. 92. Taken together, our results indicate that inhibition of the extracellular acid-induced signaling by GPR68 induces ferroptosis via ATF4-mediated transcription of downstream targets such as CHAC1. Acidic extracellular milieu activates GPR68, which suppresses ATF4 transcription. OGM-mediated GPR68 inhibition induces ATF4 expression, which then increases CHAC1, leading to depletion of glutathione. This ultimately causes accumulation of toxic lipid peroxides, which triggers ferroptosis. See FIG. 93.

### Example 34: Activation of Immunogenic Cell Death

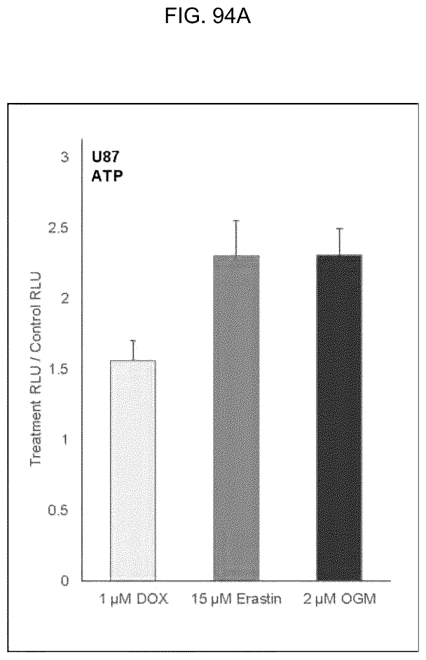

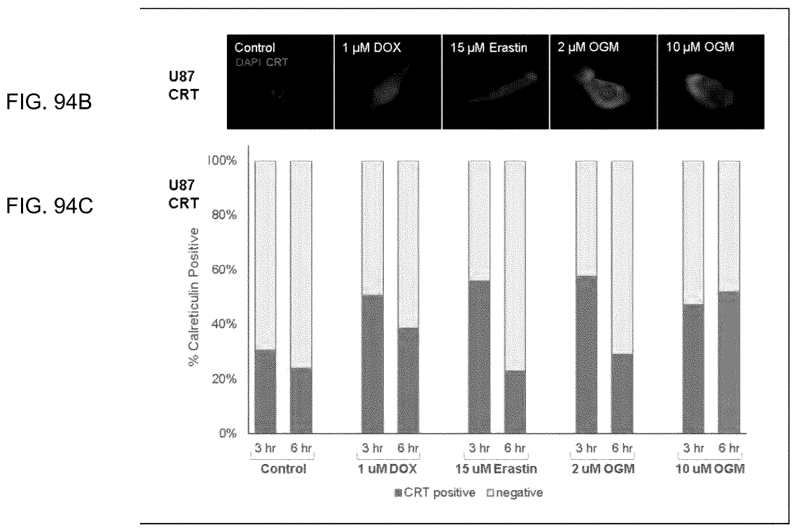

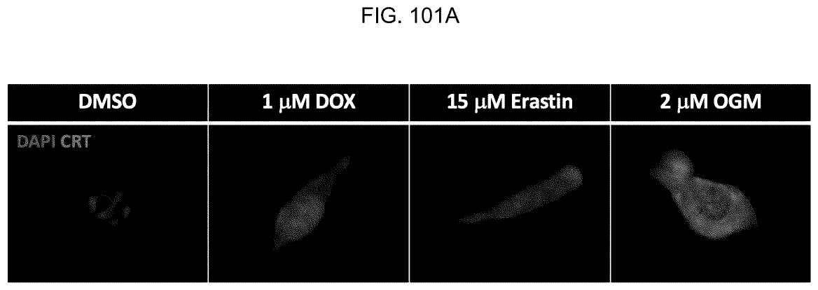

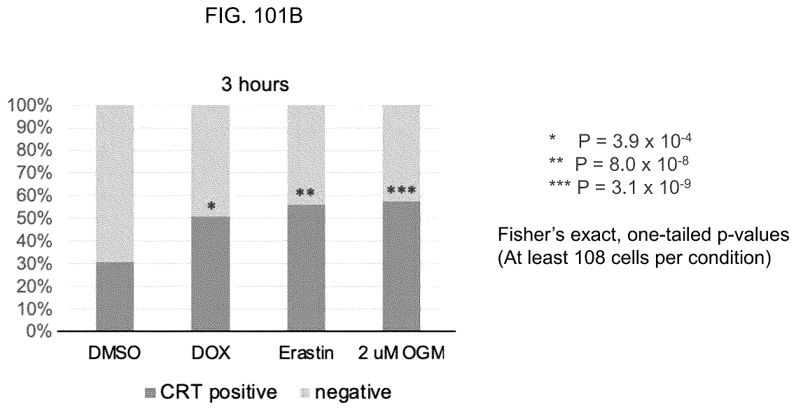

OGM2 treatment of 87 cells increases makers of immunogenic cell death (ICD, such as release of ATP into the extracellular environment (FIG. 101A-94A) and calreticulin (CRT) externalization (FIG. 94B and FIG. 94C). Extracellular ATP content was assessed using the Enliten Atp Assay System (PROMEGA) (FIG. 94A). Calreticulin (CRT) externalization 3- and 6-hours post-treatment (see FIG. 94B and FIG. 94C) in U87 cells was assessed using immunofluorescence analysis using polyclonal antibody against calreticulin (Invitrogen™). Immunogenic cell death (ICD) is defined by expression or release of damage-associated molecular patterns (DAMPs), including extracellular ATP release and calreticulin externalization, which stimulate antitumor immune response, which in turn contributes to long-lasting protective antitumor immunity. Anti-tumor immunity describes the immune system-mediated tumor-cell killing, and elimination of lysed tumor cells followed by resolution of the immune response and re-establishment of normal tissue architecture and homeostasis. Productive anti-tumor immunity may include antigen uptake and processing by dendritic cells (DCs) in the tumor, trafficking of activated DCs to the tumor-draining lymph nodes where they prime anti-tumor T cells, trafficking of anti-tumor T cells to tumors and infiltration of the T cells into the tumor tissue, and re-activation of anti-tumor T cells in the tumor.

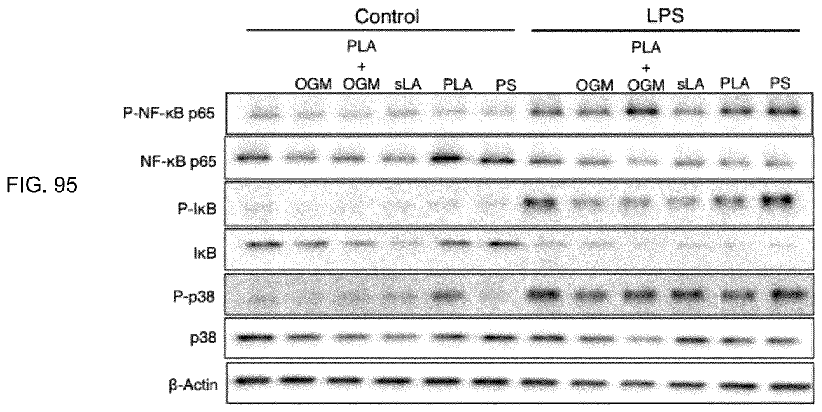

Additionally. GPR68 inhibitor blocks macrophage immunosuppression induced by extracellular lactate (FIG. 95). Therefore, GPR68 inhibitors represent an attractive approach to enhance cancer immunotherapies, including check-point inhibitors, cancer vaccines and CAR-T therapies.

The therapeutic use of GPR68 inhibitors as adjunctive therapy to boost the effects of immunotherapy are as follows. Prior to the initiation of cancer immunotherapy, cancer patients may receive therapeutic agents that inhibit GPR68 activity or knock-down GPR68 expression. After sufficient period to induce cancer cell killing, the patient optionally can receive traditional cancer immunotherapy treatments, including check point inhibitors, such as PD-1, PD-L1 and CTLA4 antagonists, or tumor cell-killing CAR (chimeric antigen receptor)-T cells. Treatment with GPR68 inhibitors can continue during the course of the cancer immunotherapy, which may include the duration during which check point inhibitors and CAR-T cells are functioning in the patient's body (up to few months). Additionally, therapeutic agents to block GPR68 may be used in conjunction with tumor vaccine treatments to boost their therapeutic efficacy. Specifically, GPR68 inhibitors may be administered prior to the initiation of the tumor vaccination to “prime” the anti-cancer immune cells and/or delivered after vaccination for a period of time, for example a month, to boost anti-cancer immune response. Cancer cell killing by GPR68 inhibition will result in enhanced immunological memory against the treated tumors. This will result in lasting protection against residual cancers or cancer recurrence in patients treated with GPR68 inhibitors.

### Example 35: GPR68 Inhibition and Radiation Synergize G2/M Arrest

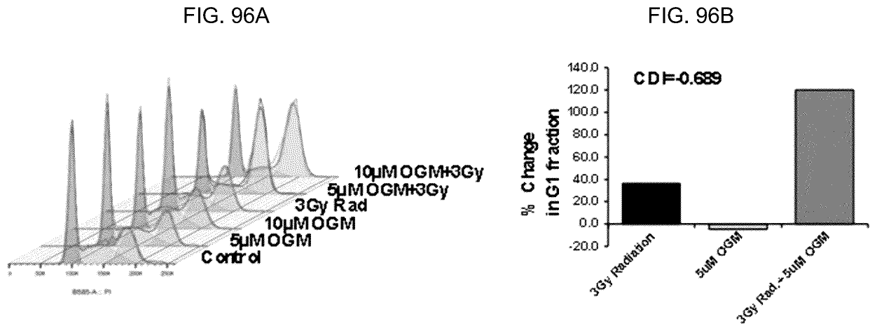

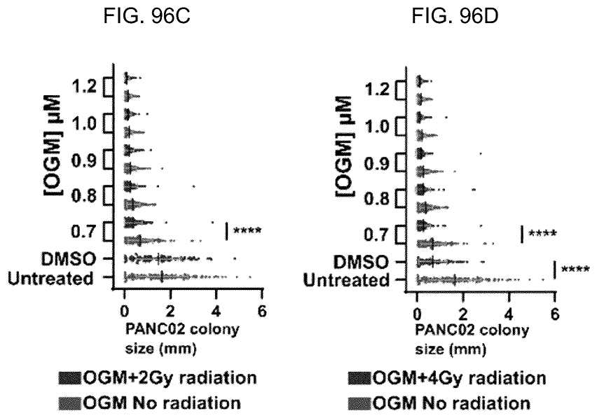

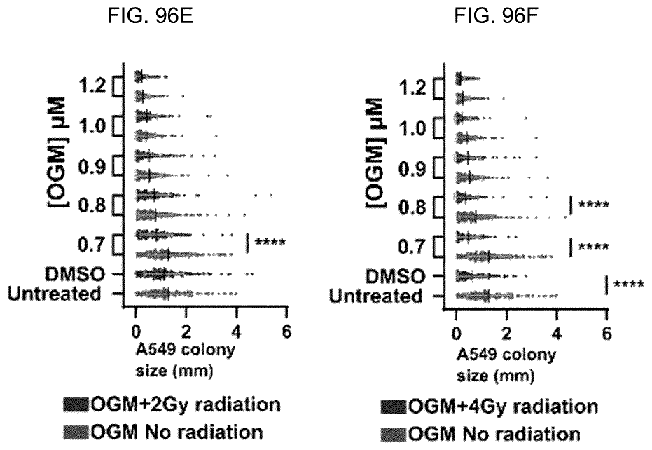

Both radiation and ferroptosis are known to induce G2/M arrest. Therefore, cells were treated with OGM2 and/or 3 Gy radiation. Media was then replaced, and cells were maintained for 24 hours. Cells were then trypsinized, pelleted, and fixed overnight in 70% Methanol at 4° C. Samples were then washed twice with cold PBS before being incubated with 10 mg/ml RNase A and 500 μg/ml propidium iodide in PBS in the dark for 30 minutes at room temperature. Cell cycle progression was determined by Flow cytometry using a LSR II from BD. FIG. 96A presents a cell cycle analysis of OGM2 treatment and irradiated Panc02 cells. FIG. 96B presents the quantitation of 120% increase in the G2 phase fraction from radiation in combination with OGM (FIG. 96A) demonstrating synergy with a coefficient of drug interaction (CDI) of 0.689. These data suggest OGM2 sensitizes cancer to radiation induced G2/M arrest.

### Example 36: OGM2 Synergizes with Radiation in Lung and Pancreatic Cancer

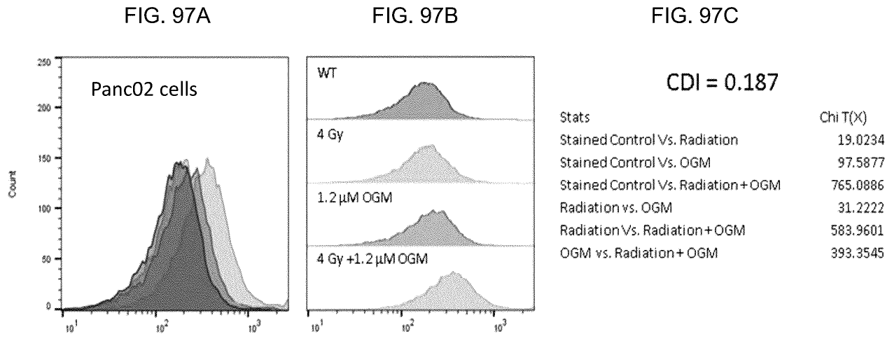

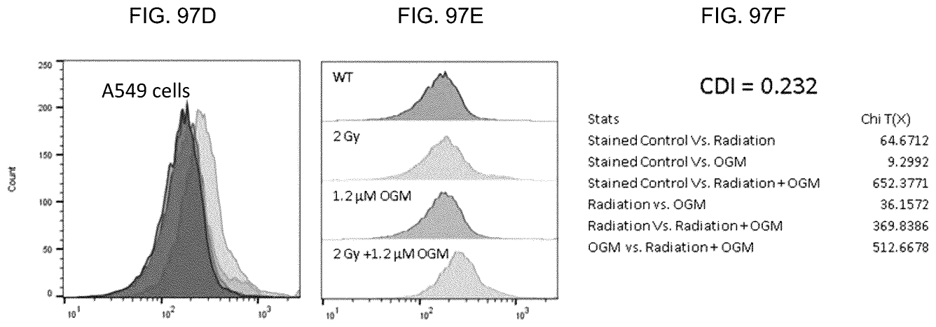

Radiation, like OGM2, induces lipid peroxidation in addition to DNA damage. OGM was tested for its ability to sensitize lung and pancreatic cancers to radiation induced lipid peroxidation and ferroptosis. Cells were incubated at 37° C. in 5% CO₂ in 30 mls DMEM with DMSO, OGM2, or Erastin for 3 days. Then 2.5 μM Liperfluo in 3 ml fresh media was added and cells were incubated at 37° C. in 5% CO₂ for 1 hour. Cells were then trypsinized, pelleted, and resuspended in cell sorting media (1% BSA and 1 mM EDTA in PBS pH 7.4). A BD LSR II was used to record Ten thousand events and the data processed with FlowJo v10.8. OGM2 and ionizing radiation demonstrated very strong synergy (CDI <0.232) for inducing lipid peroxidation in A549 lung cancer, Panc02 pancreatic cancer. All treatments were highly significant with Chi-squared >4 indicating a p<0.01. See FIG. 97. The results show that OGM and radiation synergistically cause lipid peroxidation in both lung and pancreatic cancer cells.

### Example 37: OGM2 Induces Key Ferroptosis Markers in Patient-Derived GMB Cell Lines

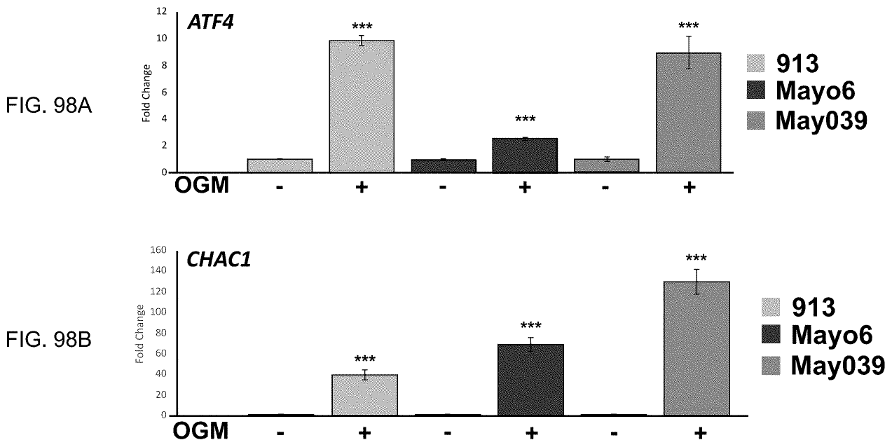

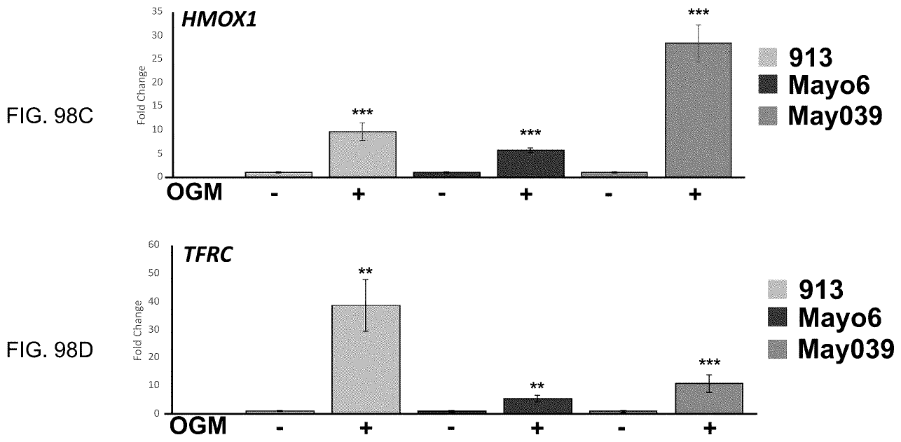

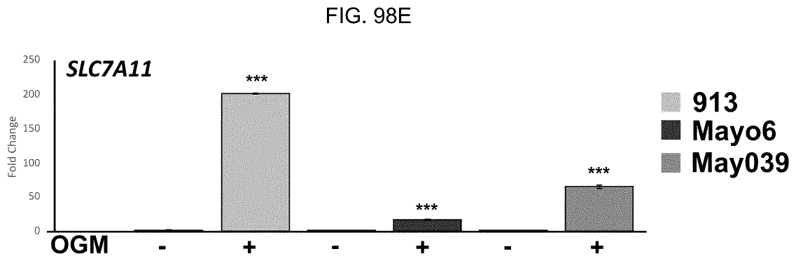

Separate biological replicates of pdx and neurosphere cells treated with OGM for 3 days were generated. RNA was isolated, cDNA generated and qPCR was conducted on select key markers of ferroptosis (ATF4. CHAC1, HMOX1, TFRC, and SLC7A11) that were seen as dysregulated in RNAseq. See FIG. 98. The results show that that OGM treatment reproducibly generates the same transcriptional response by increasing expression of key markers of ferroptosis.

### Example 38: OGM Induces Ferroptosis Through Upregulation of ATF4

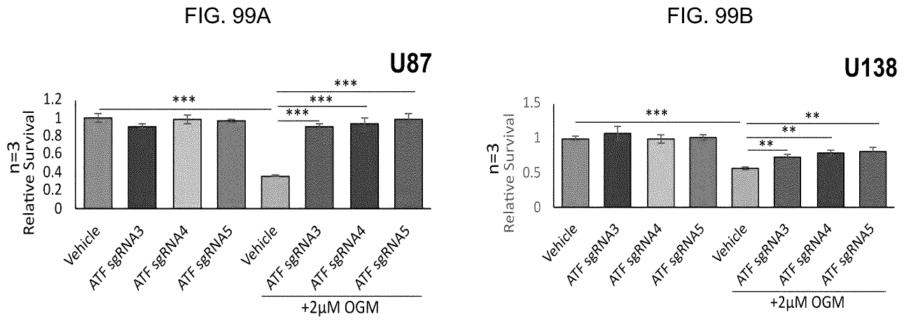

We further confirmed ATF4 inhibition rescues OGM induced ferroptosis in both U87 (FIG. 99A) and U138 cells (FIG. 106B-99B). Cells were reverse transfected on a 12-well cell culture dish (CELLTREAT Scientific Products™) in DMEM with high glucose, GlutaMAX, HEPES, Penicillin-Streptomycin and 10% FBS with 2.5 μgs dCas9 per well. The next day, fresh medium with or without 2 μM OGM was added to the wells and the cells were transfected with 12.5 pmol sgRNA using lipofectamine RNAiMAX. Three days later, cell survival was assessed by lysing the cells with 1× Passive lysis buffer and quantitation with Cell titer glow. Alternatively, total RNA was collected after three days for cDNA generation and qRT-PCR. Cells were reverse transfected on a 12-well cell culture dish (CELLTREAT Scientific Products™) in DMEM with high glucose, GlutaMAX. HEPES. Penicillin-Streptomycin and 10% FBS with 2.5 μgs dCas9 per well. The next day, fresh medium with or without 2 μM OGM was added to the wells and the cells were transfected with 12.5 pmol sgRNA using lipofectamine RNAiMAX. Three days later, cell survival was assessed by lysing the cells with 1× Passive lysis buffer and quantitation with Cell titer glow. Alternatively, total RNA was collected after three days for cDNA generation and qRT-PCR. FIG. 99A shows that CRISPRi knock-down of ATF4, with additional sgRNAs, prevented OGM-induced cell death in U87 cells, while guide RNAs or dCas9 alone had no effect on survival. FIG. 99B shows that knock-down of ATF4 prevented OGM-induced cell death in U138 cells. Additional ATF4 targeting CRISPRi successfully reduced ATF4 expression even in the setting of OGM-induced expression in U87 cells in U138 cells. The results show that OGM2 induced ferroptosis in an ATF4 dependent manner. See Example 42.

Chem1-GPR68 cells were stained with calcium indicator dye, and then exposed to 34 dyn/cm² (3.4 Pa) of force, generating a robust calcium response that could be inhibited by a 5-minute pre-incubation with 3 μM OGM2. The study suggests that ATF4 loss, either due to mutation or gene expression regulation, is a potential mechanism for resistance to tumor killing by GPR68 inhibition (see FIG. 33-32) and that expression of ATF4, either through gene expression regulation, gene therapy or other methods that enhance expression will overcome this potential resistance. Of note, one example of a method to enhance expression of ATF4 gene is using CRISPRa with sgRNA targeting the ATF4 gene, including with the s RNAs with the following sequences:

ATF4 sgRNA1:
(SEQ ID NO: 24)
5′-GAUGUCCCCCUUCGACCAGU-3′,
ATF4 sgRNA2:
(SEQ ID NO: 25)
5′-GCGGUGCUUUGCUGGAAUCG-3′,
ATF4 sgRNA3:
(SEQ ID NO: 26)
5′-CCACCAACACCUCGCUGCUC-3′,
ATF4 sgRNA4:
(SEQ ID NO: 27)
5′-AGCUCAUUUCGGUCAUGUUG-3′,
and
ATF4 sgRNA5:
(SEQ ID NO: 28)
5′-AAUGAGCUUCCUGAGCAGCG-3′.

### Example 39: Effects on Prostate Cancer

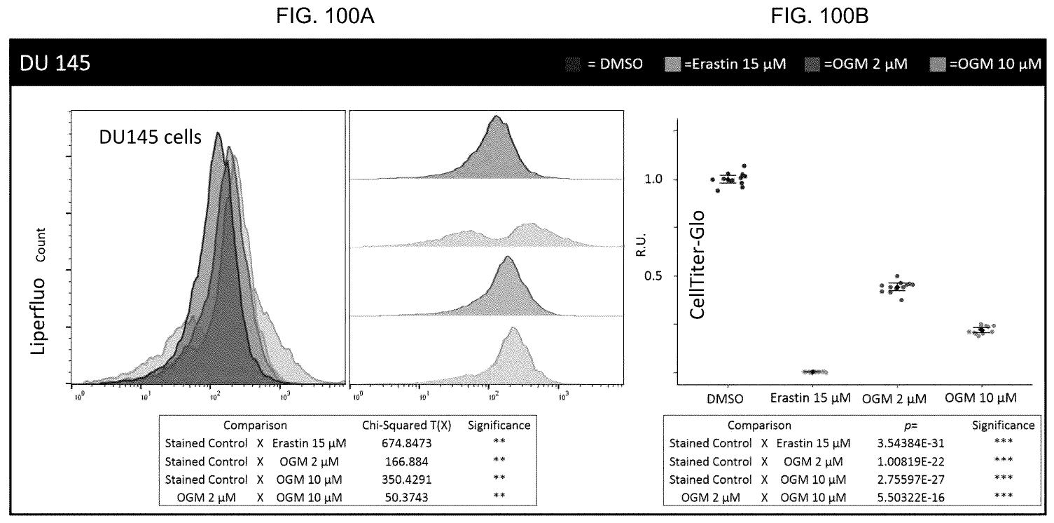

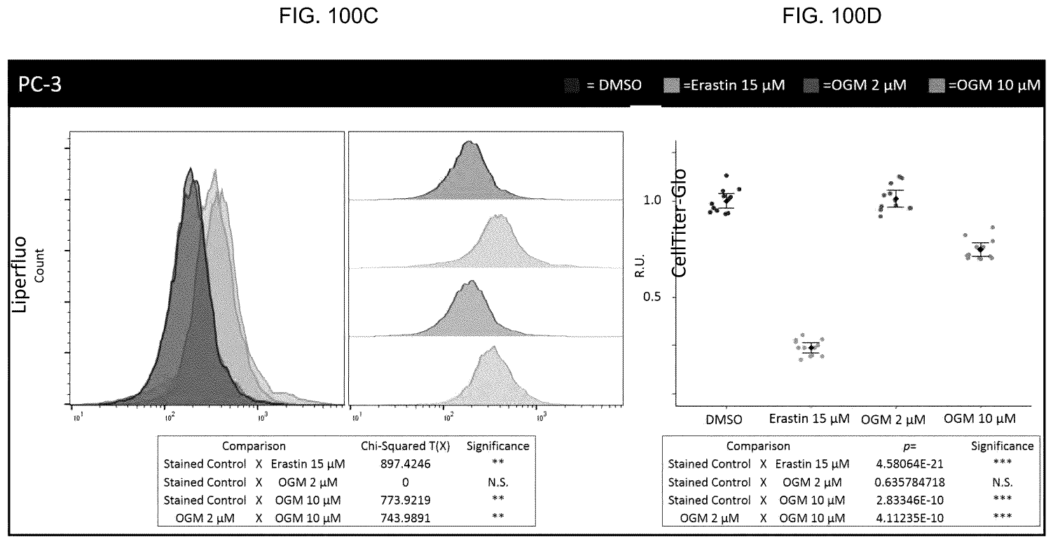

OGM2 kills prostate cancer cells by inducing ferroptosis (FIG. 100). FIG. 100A through FIG. 100D show that OGM2 significantly increased lipid peroxidation, the canonical marker of ferroptosis, and reduce viability in DU145 and PC3 human prostate cancer cells. OGM2 induces ferroptosis in DU145 cells (FIG. 100A) and PC3 cells (FIG. 100C) based on Liperfluo™ lipid peroxidation assay. Erastin, a cystine-glutamate antiporter system X_{c}⁻ inhibitor, is a positive control ferroptosis inducer. Chi square >4 is considered significantly different with p<0.01. OGM2 reduces viability of DU145 cells (FIG. 100B) and PC3 cells (FIG. 100D) based on CellTiter-Glo assay2.

### Example 40: Effects on Variety of Cancer Cell Types

OGM2 kills wide variety of cancer cell types. Cell sensitivity can be classified in to Very Sensitive (LC₅₀<2 μM), Sensitive (LC50=2 to 10 μM), Mildly Sensitive (LC₅₀>10 μM), and Refractory (no killing at 10 μM). The listed established cell were incubated with OGM2 at 2 μM or 10 μM for 72 hours under standard cell culture conditions, and relative cell number was assessed using Cell Titer-Glo Luminescent Cell Viability Assay (Promega™) following the manufacturer's instructions. Luminescence was determined using a Cytation 5 reader and Gen5 software package (BioTek™). See Table 11, below.

TABLE 11
Differential sensitivity of various cancer cell types to killing by OGM2.

| | | | % Survival | | | |
|---|---|---|---|---|---|---|
| | | | 2 μM | 10 μM | LC₅₀ | |
| Species | Origins | Cell Line | OGM | OGM | (μM) | Class |
| Human | Acute monocytic | THP-1 | 33.0 | 9.4 | <2 | Very Sensitive |
| | leukemia | | | | | |
| Human | Acute monocytic | MOLM-14 | 65.3 | 15.3 | 2-10 | Sensitive |
| | leukemia | | | | | |
| Human | B-cell lymphoma | REC-1 | 0.7 | 0.0 | <2 | Very Sensitive |
| Human | Breast adenocarcinoma | MCF7 | 50.6 | 25.2 | 2-10 | Sensitive |
| Human | Breast adenocarcinoma | MDA-MB-231 | 70.1 | 48.1 | 2-10 | Sensitive |
| Human | Breast adenocarcinoma | MDA-MB-436 | 52.6 | 35.7 | 2-10 | Sensitive |
| Human | Breast cancer | T-47D | 76.4 | 61.7 | >10 | Mildly |
| | | | | | | Sensitive |
| Human | Cervical cancer | HeLa | 91.5 | 68.0 | >10 | Mildly |
| | | | | | | Sensitive |
| Human | Chronic myelogenous | K-562 | 56.7 | 37.0 | 2-10 | Sensitive |
| | leukemia | | | | | |
| Human | Colon adenocarcinoma | SW480 | 39.8 | 25.0 | <2 | Very Sensitive |
| Human | Colorectal | HCT 116 | 44.5 | 32.7 | <2 | Very Sensitive |
| | adenocarcinoma | | | | | |
| Human | Colorectal | RKO | 92.4 | 57.6 | >10 | Mildly |
| | adenocarcinoma | | | | | Sensitive |
| Human | Colorectal | HT-29 | 102.8 | 103.9 | No | Refractory |
| | adenocarcinoma | | | | kill | |
| Human | Fibrosarcoma | HT-1080 | 64.2 | 21.4 | 2-10 | Sensitive |
| Human | Glioblastoma | U-138 MG | 40.3 | 35.0 | <2 | Very Sensitive |
| Human | Glioblastoma | U-87 MG | 21.3 | 10.1 | <2 | Very Sensitive |
| Mouse | Glioblastoma | GL261Luc | 1.8 | 0.1 | <2 | Very Sensitive |
| Mouse | Glioblastoma | KR-158-luc | 11.4 | 4.7 | <2 | Very Sensitive |
| Human | Hepatocellular cancer | Hep G2 | 66.8 | 45.7 | 2-10 | Sensitive |
| Human | Lung Adenocarcinoma | A549 | 33.6 | 18.0 | <2 | Very Sensitive |
| Human | Lung cancer, Large cell | NCI-H460 | 51.7 | 26.5 | 2-10 | Sensitive |
| Human | Lung cancer, small cell | NCI-H82 | 102.6 | 59.9 | >10 | Mildly |
| | | | | | | Sensitive |
| Human | Lung cancer, squamous | RWGT2 | 39.9 | 37.1 | <2 | Very Sensitive |
| | cell | | | | | |
| Human | Medulloblastoma | Daoy | 9.0 | 10.5 | <2 | Very Sensitive |
| Human | Melanoma | WM-115 | 92.9 | 40.7 | 2-10 | Sensitive |
| Human | Pancreatic | BxPC-3 | 81.5 | 83.8 | >10 | Mildly |
| | adenocarcinoma | | | | | Sensitive |
| Mouse | Pancreatic | Panc02 | 72.5 | 40.5 | 2-10 | Sensitive |
| | adenocarcinoma | | | | | |
| Human | Prostate adenocarcinoma | DU 145 | 44.2 | 22.2 | <2 | Very Sensitive |
| Human | Prostate adenocarcinoma | PC-3 | 101.3 | 74.5 | >10 | Mildly |
| | | | | | | Sensitive |

These results in indicate that OGM2 can kill wide variety of cancer cell types, including Acute Monocytic Leukemia (Sensitive to Very Sensitive). B-cell Lymphoma (Very Sensitive), Breast Cancer (Mildly Sensitive to Sensitive), Cervical Cancer (Mildly Sensitive), Chronic Myelogenous Leukemia (Sensitive), Colon Cancer (Refractory to Very Sensitive), Fibrosarcoma (Sensitive), Glioblastoma (Very Sensitive), Hepatocellular Cancer (Sensitive), Lung Cancer (Mildly Sensitive to Very Sensitive). Medulloblastoma (Very Sensitive), Melanoma (Sensitive), Pancreatic Cancer (Mildly Sensitive to Sensitive). Prostate Cancer (Mildly Sensitive to Very Sensitive).

### Example 41: Predicting Therapeutic Response of Cancers to GPR68 Inhibition

Cancer cells, which are very sensitive to killing by OGM2 (LCs <2 μM), with reported mRNA expression of GPR68 and the related proton sensing receptor GPR4. LC₅₀'s (concentrations causing 50% lethality) were determined using CellTiter-Glo (Promega™, G7570). Cells were plated in 12-well plates (CELLTREAT Scientific Products, 229112) in 1 ml of DMEM with high glucose, GlutaMAX, HEPES, or RPMI 1640 with Penicillin-Streptomycin (ThermoFisher™, 10564029, 11875119 and 15140122) and 10% FBS. Cells were incubated overnight at 37° C. in 5% CO₂. Then 1 mL of fresh media was added with DMSO, OGM, or positive control (Erastin; APExBIO Technology, LLC, B1524) and cells incubated for 3 days. On the third day, all media was removed, cells were rinsed with 1×PBS, PBS was removed, and 200 μL of 1× Passive Lysis Buffer (Promega™, E1941) was added to each well. Plates were then rocked on a Lab-Net Model 35 Platform Rocker at 9 RPM for 15 minutes at room temperature. Subsequently, 20 μL of lysate was added to 100 μL CellTiter-Glo™ reagent on a LUMITRAC 200 plate (Greiner™ bio-one, 82050-726). Plates were briefly spun down and read on a GloMax-Multi Detection System (Promega™).

Predicted expression levels were determined using the publicly available mRNA database of the human protein atlas (proteinatlas.org). Alternatively, expression levels of GPR68 and GPR4 are determined by qRT-PCR. Total RNA was isolated using the RNeasy™ Plus Mini Kit (Qiagen™) following the manufacturer's instructions, cDNAs were generated using High-Capacity cDNA Reverse Transcription Kit with RNase Inhibitor (ThermoFisher™, 4374966). Samples were run on a Quant Studio 5 real-time PCR system (Applied Biosystems™) with TaqMan™ Universal Master Mix II, with UNG (ThermoFisher™, 4440042). Primers used were GAPDH: Hs02786624g1, GPR68: Hs00268858s1, GPR4: Hs0027099sa (ThermoFisher™).

Human cancer cells are listed below, showing that those with high sensitivity to OGM2 have high GPR68:GPR4 expression ratio. See Table 12, below. Given high degrees of sequence and functional similarities between the proton-sensing receptors GPR68 and GPR4, we hypothesize that there is some degree of redundancy between GPR68 and GPR4. Thus, lower the relative expression of GPR4 relative to GPR68, the higher likelihood that selective inhibition or knockdown of GPR68 w % ill result in tumor cell killing. In such cases, where GPR4 expression is higher, lowering the ratio of GPR68:GPR4 expression, a combination treatment involving inhibition of both GPR68 and GPR4 will kill “OGM-insensitive” cancer cells.

TABLE 12
Human Cancer Cell Data

| | | | | | | RNA | | |
|---|---|---|---|---|---|---|---|---|
| | | % Survival | | | | Expression | | |
| | Cell | 2 μM | 10 μM | LC₅₀ | | (nTPM) | | GPR68:GRP4 |
| Origin | Line | OGM | OGM | (μM) | Class | GPR68 | GPR4 | Ratio |
| Acute monocytic | THP-1 | 33.0 | 9.4 | <2 | Very | 0.9 | 0.0 | infinite |
| leukemia | | | | | Sensitive | | | |
| B-cell lymphoma | REC-1 | 0.7 | 0.0 | <2 | Very | 0.5 | 0.1 | 5 |
| | | | | | Sensitive | | | |
| Colorectal | SW480 | 39.8 | 25.0 | <2 | Very | 0.1 | 0.0 | infinite |
| adenocarcinoma | | | | | Sensitive | | | |
| Colorectal | HCT | 44.5 | 32.7 | <2 | Very | 0.2 | 0.1 | 2 |
| adenocarcinoma | 116 | | | | Sensitive | | | |
| Glioblastoma | U-138 | 40.3 | 35.0 | <2 | Very | 6.1 | 0.1 | 61 |
| | MG | | | | Sensitive | | | |
| Glioblastoma | U-87 | 21.3 | 10.1 | <2 | Very | 27.6 | 0.0 | infinite |
| | MG | | | | Sensitive | | | |
| Lung | A549 | 33.6 | 18.0 | <2 | Very | 0.1 | 0.0 | infinite |
| Adenocarcinoma | | | | | Sensitive | | | |
| Medulloblastoma | Daoy | 9.0 | 10.5 | <2 | Very | 3.1 | 0.1 | 31 |
| | | | | | Sensitive | | | |
| Prostate | DU | 44.2 | 22.2 | <2 | Very | 0.5 | 0.1 | 5 |
| adenocarcinoma | 145 | | | | Sensitive | | | |

Cancer cells, which are minimally sensitive to killing by OGM2 (LC₅₀>10 μM), with reported mRNA expression of GPR68 and GPR4. Cancer cells with low sensitivity to OGM2 have lower GPR68:GPR4 expression ratio, suggesting GPR4 may compensate for inhibited GPR68 (see Table 13 and Table 14, below).

TABLE 13
Human Cancer Cell Data

| | | % Survival | | | | RNA Expression | | |
|---|---|---|---|---|---|---|---|---|
| | Cell | 2 μM | 10 μM | LC₅₀ | | (nTPM) | | 68:4 |
| Origins | Line | OGM | OGM | (μM) | Class | GPR68 | GPR4 | Ratio |
| Cervical cancer | HeLa | 91.5 | 68.0 | >10 | Low | 0.0 | 0.1 | 0 |
| | | | | | Sensitive | | | |
| Colorectal | RKO | 92.4 | 57.6 | >10 | Low | 0.3 | 0.4 | 0.75 |
| adenocarcinoma | | | | | Sensitive | | | |
| Lung cancer, small | NCI- | 102.6 | 59.9 | >10 | Low | 0.3 | 0.2 | 1.5 |
| cell | H82 | | | | Sensitive | | | |
| Prostate | PC-3 | 101.3 | 74.5 | >10 | Low | 0.6 | 8.4 | 0.071 |
| adenocarcinoma | | | | | Sensitive | | | |

Sensitivity of prostate cancer cells to OGM2 can be predicted based on the relative expression of GPR68 and GPR4 in cancer cells. Higher GPR68:GPR4 expression ratio predicts high sensitivity to killing by GPR68 inhibition. By contrast, lower GPR68:GPR4 expression ratio predicts relative resistance to killing by GPR68 inhibition.

TABLE 14
Human Cancer Cell Data.

| | | % Survival | | | | RNA Expression | | |
|---|---|---|---|---|---|---|---|---|
| | Cell | 2 μM | 10 μM | LC₅₀ | | (nTPM) | | GPR68:GPR4 |
| Origins | Line | OGM | OGM | (μM) | Class | GPR68 | GPR4 | Ratio |
| Prostate | DU | 44.2 | 22.2 | <2 | Very | 0.5 | 0.1 | 5 |
| adenocarcinoma | 145 | | | | Sensitive | | | |
| Prostate | PC-3 | 101.3 | 74.5 | >10 | Low | 0.6 | 8.4 | 0.071 |
| adenocarcinoma | | | | | Sensitive | | | |

The cells listed in the tables above were incubated with OGM2 at 2 μM or 10 μM for 72 hours under standard cell culture conditions, and relative cell number was assessed using Cell Titer-Glo Luminescent Cell Viability Assay (Promega™) following the manufacturer's instructions. Luminescence was determined using a Cytation 5 reader and Gen5 software package (BioTek™). RNA expression of GPR68 and GPR4 in the corresponding cancer cell lines are listed, according to the publicly available The Human Protein Atlas version 22.0 and Ensembl version 103.38 (see proteinatlas.org). The consensus normalized expression value (“nTPM”=normalize transcript per million) was calculated as the maximum nTPM value for each gene in the two data sources.

In summary, in cases where the ratio is 2 or higher in the individual tumor sample, and hence lower relative expression of GPR4, this predicts therapeutic efficacy of GPR68 knock-down. Thus, treatment can involve GPR68 knock-down alone. In cases where the ratio is lower than 2, and hence higher relative expression of GPR4, this predicts that a combination treatment involving inhibition of both GPR68 and GPR4 to kill “OGM-insensitive” cancer cells is preferred.

Therefore, in certain embodiments, the invention comprises a method of predicting the sensitivity of individual cancers to killing by GPR68 inhibition. This method involves obtaining a fresh or frozen tumor sample from a subject at the time of diagnostic tissue biopsy, surgical excision, bone marrow biopsy or peripheral blood draw. mRNA is isolated from the tumor sample using methods known to the person of skill. Then, cDNAs are generated from the mRNA so that the normalized expressions of both GPR68 and GPR4 in the tumor sample can be determined by real-time CPR. The ratio of the normalized expression of GPR68 relative to the normalized expression of GPR4 in tumor samples provides a method of predicting the sensitivity of the particular tumor to killing by GPR68 inhibition. A ratio of 2:1 (GPR68:GPR4) or higher in individual tumor samples indicates increased sensitivity of the tumor to GPR68 inhibition, and hence predicts therapeutic efficacy of the therapeutic agents. In cases where, the ratio is lower than 2:1 (GPR68:GPR4), and hence higher relative expression of GPR4 exists, killing by GPR68 inhibition alone is reduced. Therefore, a combination treatment involving inhibition of both GPR68 and GPR4 should be employed to kill “OGM-insensitive” cancer cells.

### Example 42: Cancer Immunotherapy

Immunogenic cell death (ICD) is a form of cell death resulting in a regulated activation of the immune response. Two hallmarks of immunogenic cell death (ICD) are Externalization of Calreticulin (CRT), a damage-associated molecular pattern (DAMP) and ATP secretion. Upon induction of ICD, CRT, which is normally located in the lumen of the endoplasmic reticulum, becomes translated to the dying cell's surface, where it functions as a damage-associated molecular pattern (DAMP), triggering immune response, including phagocytosis by antigen presenting cells. Additionally. ATP released during ICD has variety of immune stimulatory effects, including recruitment of immune/inflammatory cells.

For the CRT externalization assay, U87 cells were seeded on 96-well plate, then treated with DMSO, 1 mM Doxorubicin (DOX), 15 mM Erastin and 2 mM OGM2 (shown as OGM in the figures). At 3 hours, cells were fixed (4% paraformaldehyde) without permeabilization, and immunostained for extracellular CRT with anti-calreticulin polyclonal antibody (Invitrogen™). Nuclei were stained with DAPI. Cells labelled with CRT were counted automatically with Lionheart™ automated microscope and % of CRT+ cells calculated. At least 10⁸ cells per condition.

For the ATP secretion assay, U87 cells were seeded on 96-well plate, then treated with DMSO, 1 mM Doxorubicin (DOX), 15 mM Erastin and 2 mM OGM2 (OGM). At 6 hours, extracellular ATP levels were measured using the ENLITEN ATP Assay (Promega™). Experiments were done in quadruplicates.

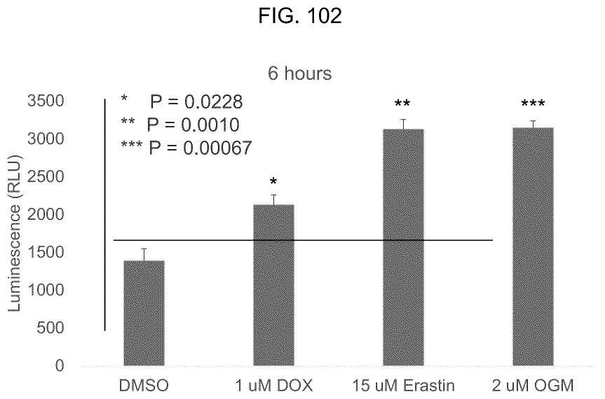

Treatment of U87 glioblastoma cells with OGM2 (shown as OGM in FIG. 101 and FIG. 102) results in significant externalization of CRT at 3 hours of treatment (FIG. 101), and secretion of ATP at 6 hours (FIG. 102). Results for treatment with 1 mM Doxorubicin and 15 mM erastin, a ferroptosis inducer indicate that inhibition of GPR68 results in immunogenic cell death, which activate lasting anti-tumor immune response. Thus, compounds such as OGM2, co-administered with cancer immunotherapies, boost the efficacy of cancer immunotherapies. Moreover, compounds such as OGM2, by itself or co-administered with cancer vaccines, can result in lasting anti-tumor immunological memory.

The therapeutic use of GPR68 inhibitors as adjunctive therapy to boost the effects of immunotherapy is as follows. Prior to or with the initiation of cancer immunotherapy, cancer patients receive therapeutic agents that inhibit GPR68 activity or knock down GPR68. After sufficient period to induce cancer cell killing, the patient receives traditional cancer immunotherapy treatments, including for example, check point inhibitors, such as PD-1. PD-L1 and CTLA4 antagonists, or tumor cell-killing CAR (chimeric antigen receptor)-T cells. Treatment with GPR68 inhibitors optionally continues during the course of the cancer immunotherapy treatment, optionally including the entire period of time during which check point inhibitors and/or CAR-T cells are functioning in the patient's body (up to few months).

### REFERENCES

All references listed below and throughout the specification are hereby incorporated by reference in their entirety.
1. Adams et al., 2006. Early, H+-V-ATPase-dependent proton flux is necessary for consistent left-right patterning of non-mammalian vertebrates. Development 133, 1657-1671. doi:10.1242dev.02341.
2. Ahn et al., 2003. Desensitization, internalization, and signaling functions of beta-arrestins demonstrated by RNA interference. Proc Natl Acad Sci USA 100, 1740-1744. doi:10.1073/pnas.262789099.
3. Aoki et al., 2013. Proton-sensing ovarian cancer G protein-coupled receptor 1 on dendritic cells is required for airway responses in a murine asthma model. PLoS One. November 11; 8(11): e79985. doi: 10.1371/journal.pone.0079985.
4. Asadi et al., 2022. Caspase-3: Structure, function, and biotechnological aspects. Biotechnol Appl Biochem. 69:4. 1633-1645.
5. Baebler et al., 2018. A novel OG 1 (GPR68) inhibitor attenuates inflammation in a murine model of acute colitis. 2018 European Crohn's & Colitis Organization Meeting Abstract P087.
6. Bailey et al., 2012. Targeting the Metabolic Microenvironment of Tumors. Adv Pharmacol. 65, 63-107.
7. Bertrand et al., 2012. Developmental pathways in colon cancer: Crosstalk between WNT, BMP, Hedgehog and Notch. Cell Cycle. 11, 4344-4351.
8. Bhujwalla et al., 2001. The physiological environment in cancer vascularization, invasion and metastasis. Novartis Found. Symp. 240, 23-38; discussion 38.
9. Boedtkjer et al., 2020. The acidic tumor microenvironment as a driver of cancer. Annu Rev Physiol. 82, 103-126.
10. Boonpiyathad et al., 2019. Immunologic Mechanisms in Asthma. Semin Immunol. 2019 December; 46:101333. doi: 10.1016/j.smim.2019.101333.
11. Brandes et al., 2008. MGMT promoter methylation status can predict the incidence and outcome of pseudoprogression after concomitant radiochemotherapy in newly diagnosed glioblastoma patients. Journal of Clinical Oncology. 26, 2192-2197.
12. Cao et al., 2016. Mechanisms of ferroptosis. Cell Mol Life Sci. 73, 2195-2209.
13. Cao et al., 2009 The correlation and prognostic significance of MGMT promoter methylation and MGMT protein in glioblastomas. Neurosurgery. 65, 866-875.
14. Carroll et al., 2014. R PheWAS: data analysis and plotting tools for phenome-wide association studies in the R environment. Bioinformatics 30, 2375-2376. doi:10.1093/bioinformatics/btu197.
15. Castellone et al., R. D., 2011. Inhibition of tumor cell migration and metastasis by the proton-sensing GPR4 receptor. Cancer Lett. 312. 197-208. doi:10.1016/j.canlet.2011.08.013.
16. Cerami et al., 2012. The cBio cancer genomics portal: an open platform for exploring multidimensional cancer genomics data. Cancer Discov. 2, 401-404. doi:10.1158/2159-8290.CD-12-0095.
17. Chatterjee et al., 2019. Targeting the crosstalks of Wnt pathway with Hedgehog and Notch for cancer therapy. Pharmacol Res. 142, 251-261.
18. Chen et al., 2021. Characteristics and Biomarkers of Ferroptosis. Front Cell Dev Biol. 9, 637162.
19. Chen et al., 2009. Small molecule-mediated disruption of Wnt-dependent signaling in tissue regeneration and cancer. Nat. Chem. Biol. 5, 100-107. doi:10.1038/nchembio. 137.
20. Chen et al., 2017. CHAC1 degradation of glutathione enhances cystine-starvation-induced necroptosis and ferroptosis in human triple negative breast cancer cells via the GCN2-eIF2α-ATF4 pathway. Oncotarget. 8, 114588-114602.
21. Chen et al., 2009. Expression and function of pro ton-sensing G-protein-coupied receptors in inflammatory pain. Molecular Pain 5:39.
22. Cheng et al., 2010. Luciferase Reporter Assay System for Deciphering GPCR Pathways. Curr. Chem. Genomics 4, 84-91. doi:10.2174/1875397301004010084.
23. Chiu et al., 2020. Asparagine Synthetase in Cancer: Beyond Acute Lymphoblastic Leukemia. Front Oncol. 9, 1480 (2020).
24. Choi et al., 1996. Heme Oxygenase-1: Function, Regulation, and Implication of a Novel Stress-inducible Protein in Oxidant-induced Lung Injury. Am J Respir Cell Mol Biol. 15, 9-19.
25. Coakley et al., 2003. Abnormal surface liquid pH regulation by cultured cystic fibrosis bronchial epithelium. Proc Natl Acad Sci USA.; 100(26):16083-8. Epub 2003 Dec. 10.
26. Crawford et al, 2015. Human CHAC1 Protein Degrades Glutathione. and mRNA Induction Is Regulated by the Transcription Factors ATF4 and ATF3 and a Bipartite ATF/CRE Regulatory Element. J Biol Chem. 290(25):15878-15891.
27. Cui et al., 2017. Advances in multicellular spheroids formation. Journal Royal Society Interface. 14(127): 20160877.
28. de Valliere et al., 2015. G Protein-coupled pH-sensing Receptor OGR1 Is a Regulator of Intestinal Inflammation. Inflammatory Bowel Dis 21: 1269-1281.
29. de Vallière et al., 2016. Hypoxia Positively Regulates the Expression of pH-Sensing G-Protein-Coupled Receptor OGR1 (GPR68). Cell Mol Gastroenterol Hepatol. 2(6): 796-810.
30. Denny et al., 2013. Systematic comparison of phenome-wide association study of electronic medical record data and genome-wide association study data. Nat. Biotechnol. 31, 1102-1110. doi:10.1038/nbt.2749.
31. Denny et al., 2016. Phenome-Wide Association Studies as a Tool to Advance Precision Medicine. Annu. Rev. Genomics Hum. Genet. 17, 353-373. doi:10.1146/annurev-genom-090314-024956.
32. Dixon et al., 2014. Pharmacological inhibition of cystine-glutamate exchange induces endoplasmic reticulum stress and ferroptosis. Elife. 3, e02523.
33. Doan et al., 2018. Identification of radiation responsive genes and transcriptome profiling via complete RNA sequencing in a stable radioresistant U87 glioblastoma model. Oncotarget. 9:34, 23532-235429.
34. D'Souza et al., 2016. OGR1/GPR68 Modulates the Severity of Experimental Autoimmune Encephalomyelitis and Regulates Nitric Oxide Production by Macrophages. PLoS ONE 11(2): e0148439.
35. Faletti et al., 2021. LSD1-directed therapy affects glioblastoma tumorigenicity by deregulating the protective ATF4-dependent integrated stress response. Sci Transl Med. 13, 7036.
36. Gao et al., 2013. Integrative analysis of complex cancer genomics and clinical profiles using the cBioPortal. Sci. Signal. 6, p11. doi:10.1126/scisignal.2004088.
37. Gayan et al., 2017. Inherent aggressive character of invasive and non-invasive cells dictates the in vitro migration pattern of multicellular spheroid. Sci. Rep. 7, 11527. doi:10.1038/s41598-017-10078-7.
38. Ge et al., 2018. iDEP: an integrated web application for differential expression and pathway analysis of RNA-Seq data. BMC Bioinformatics 19:1, 1-24.
39. Glunde et al., 2003. Extracellular acidification alters lysosomal trafficking in human breast cancer cells. Neoplasia 5, 533-545.
40. Hamano et al., 2020. Transcriptional Activation of Chac1 and Other Atf4-Target Genes Induced by Extracellular 1-Serine Depletion is negated with Glycine Consumption in Hepa1-6 Hepatocarcinoma Cells. Nutrients. 12, 1-11.
41. Hanahan et al., 2011. Hallmarks of Cancer: The Next Generation. Cell: 144: 646-674.
42. Hao et al., 2013. Selective Small Molecule Targeting β-Catenin Function Discovered by In Vivo Chemical Genetic Screen. Cell Rep. 4:5, 898-904.
43. Hao et al., 2010. In vivo structure—Activity relationship study of dorsomorphin analogues identifies selective VEGF and BMP inhibitors. ACS Chem Biol. 5, 245-253.
44. Hegi et al., 2005. MGMT Gene Silencing and Benefit from Temozolomide in Glioblastoma. New England Journal of Medicine. 352, 997-1003.
45. Helmlinger et al., 1997. Interstitial pH and p02 gradients in solid tumors in vivo: high-resolution measurements reveal a lack of correlation. Nat Med. 3, 177-182.
46. Hill et al., 2001. pH, hypoxia and metastasis. Novartis Found. Symp. 240, 154-65; discussion 165.
47. Homma et al., 2022. Methionine Deprivation Reveals the Pivotal Roles of Cell Cycle Progression in Ferroptosis That Is Induced by Cysteine Starvation. Cells. 11:10, 1603.
48. Horman et al., 2017. Functional profiling of microtumors to identify cancer associated fibroblast-derived drug targets. Oncotarget 8, 99913-99930. doi:10.18632/oncotarget.21915.
49. Houessinon et al., 2016. Metallothionein-1 as a biomarker of altered redox metabolism in hepatocellular carcinoma cells exposed to sorafenib. Mol Cancer. 15, 1-10.
50. Hu et al., 2016. Living near a Major Road in Beijing: Association with Lower Lung Function, Airway Acidification, and Chronic Cough. Chin Med J 129:2184-90.
51. Huang et al., 2008. Extracellular Acidification Elicits Spatially and Temporally Distinct Ca2+ Signals. Current Biology. 18, 781-785.
52. Huang et al., 2007. DAVID Bioinformatics Resources: Expanded annotation database and novel algorithms to better extract biology from large gene lists. Nucleic Acids Res. 35, W169-75.
53. Hunt et al., 2018. Gastric Aspiration and Its Role in Airway Inflammation. Open Respir Med J. 2018; 12:1-10. Published January 23. doi:10.2174/1874306401812010001.
54. Hunter et al., 2006. Does the tumor microenvironment influence radiation-induced apoptosis? Apoptosis 10:11, 1727-1735.
55. Jiang et al., 2017. EGLN1/c-Myc Induced Lymphoid-Specific Helicase Inhibits Ferroptosis through Lipid Metabolic Gene Expression Changes. Theranostics. 7, 3293-3305.
56. Jiang et al., 2021. Ferroptosis: mechanisms, biology and role in disease. Nature Reviews Molecular Cell Biology. 22:4, 266-282.
57. Jung et al., 2019. Apical-basal polarity inhibits epithelial-mesenchymal transition and tumour metastasis by PAR-complex-mediated SNAI1 degradation. Nat. Cell Biol. 21, 359-371. doi:10.1038/s41556-019-0291-8.
58. Justus et al., 2013. Acidic tumor microenvironment and pH-sensing G protein-coupled receptors. Front. Physiol. 4, 354. doi:10.3389/fphys.2013.00354.
59. Kato et al., 2013. Acidic extracellular microenvironment and cancer. Cancer Cell Int. 13, 1-8.
60. Ketelut-Cameiro et al., 2022. Apoptosis, Pyroptosis, and Necroptosis-Oh My! The Many Ways a Cell Can Die. J Mol Biol. 434:4, 167378.
61. Kim et al., 2013. TopHat2: accurate alignment of transcriptomes in the presence of insertions, deletions and gene fusions. Genome Biol. 14:4, R36.
62. Kodric et al., 2007. An investigation of airway acidification in asthma using induced sputum: a study of feasibility and correlation. Am. J. Respir. Crit. Care Med 175, 905-910.
63. Kondo et al., 2017. Extracellular Acidic pH Activates the Sterol Regulatory Element-Binding Protein 2 to Promote Tumor Progression. Cell Rep. 18, 2228-2242.
64. Krieger et al., 2016. Increased bone density in mice lacking the proton receptor OGR1. Kidney International 89:565-573.
65. Kuba et al., 2005. A crucial role of angiotensin converting enzyme 2 (ACE2) in SARS coronavirus-induced lung injury. Nat Med. 11(8):875-9.
66. Kwak et al., 2013. Live image profiling of neural crest lineages in zebrafish transgenic lines. Mol. Cells 35, 255-260. doi:10.1007/s10059-013-0001-5.
67. Lange et al., 2008. ATF4 is an oxidative stress-inducible, prodeath transcription factor in neurons in vitro and in vivo. J Exp Med. 205, 1227-1242.
68. Lauko et al., 2022. Cancer cell heterogeneity & plasticity in glioblastoma and brain tumors. Semin Cancer Biol. 82, 162-175.
69. Lee et al., 2006. Tumor stem cells derived from glioblastomas cultured in bFGF and EGF more closely mirror the phenotype and genotype of primary tumors than do serum-cultured cell lines. Cancer Cell. 9, 391-403.
70. Lei et al., 2020. The role of ferroptosis in ionizing radiation-induced cell death and tumor suppression. Cell Research 30:2, 146-162.
71. Li et al., 2019. Carbonic anhydrase 9 confers resistance to ferroptosis/apoptosis in malignant mesothelioma under hypoxia. Redox biology. 26, 101297.
72. Li et al., 2020. Ferroptosis: past, present and future. Cell Death & Disease 11:2, 1-13.
73. Li et al., 2012. Identification and characterization of distinct C-terminal domains of the human hydroxycarboxylic acid receptor-2 that are essential for receptor export, constitutive activity, desensitization, and internalization. Mol. Pharmacol. 82, 1150-1161. doi:10.1124/mol. 112.081307.
74. Liu et al., 2013. Regulator of G-protein signaling 2 inhibits acid-induced mucin5AC hypersecretion in human airway epithelial cells. Respir Physiol Neurobiol. 185(2):265-71.
75. Lomelino et al., 2017. Asparagine synthetase: Function, structure, and role in disease. J Biol Chem. 292, 19952-19958.
76. Lopez-Lazaro, 2008. The Warburg effect: why and how do cancer cells activate glycolysis in the presence of oxygen? Anti-Cancer Agents in Medicinal Chemistry 8 (3): 305-12.
77. Louis et al., 2016. The 2016 World Health Organization Classification of Tumors of the Central Nervous System: a summary. Acta Neuropathol. 131, 803-820.
78. Love et al., 2014. Moderated estimation of fold change and dispersion for RNA-seq data with DESeq2. Genome Biol. 15, 550.
79. Lu et al., 2021. MiR-27a-3p Promotes Non-Small Cell Lung Cancer Through SLC7A11-Mediated-Ferroptosis. Frontiers in oncology. 11, 759346.
80. Ludwig et al., 2013. The Na+/H+-exchanger (NHEa) generates pH nanodomains at focal adhesions. J. Cell. Physiol. 228, 1351-1358. doi:10.1002/jcp.24293.
81. Ludwig et al., 2003. Proton-sensing G-protein-coupled receptors. Nature 425:6953, 93-98.
82. Luttrell et al., 2002. The role of beta-arrestins in the termination and transduction of G-protein-coupled receptor signals. J. Cell Sci. 115, 455-465.
83. Mahon, 2011. pHluorin2: an enhanced, ratiometric, pH-sensitive green florescent protein. Advances in Bioscience and Biotechnology. 02, 132-137.
84. Marik, 2001. Aspiration pneumonitis and aspiration pneumonia. N. Engl. J. Med. 344(9):665-671.
85. Marino et al., 2012. Autophagy is a protective mechanism for human melanoma cells under acidic stress. J Biol Chem. 287(36):30664-76.
86. Martinez-Zaguilan et al., 1993. Vacuolar-type H(+)-ATPases are functionally expressed in plasma membranes of human tumor cells. Am. J. Physiol. 265, C1015-29. doi:10.1152/ajpcell.1993.265.4.C1015.
87. Martinez-Zaguilán et al., 1998. Distinct regulation of pH in and [Ca2+] in human melanoma cells with different metastatic potential. J. Cell. Physiol. 176, 196-205. doi:10.1002/(SICI)1097-4652(199807)176:1<196::AID-JCP21>3.0.CO; 2-4.
88. Martinez-Zaguilán et al., 1996. Acidic pH enhances the invasive behavior of human melanoma cells. Clin. Exp. Metastasis 14, 176-186.
89. Matthews et al., 2011. Anti-tumour/metastasis effects of the potassium-sparing diuretic amiloride: an orally active anti-cancer drug waiting for its call-of-duty? Int. J. Cancer 129, 2051-2061. doi:10.1002/ijc.26156.
90. Matute-Bello et al., 2008. Animal models of acute lung injury. Am J Physiol Lung Cell Mol Physiol. 295(3):L379-L399. doi:10.1152/ajplung.00010.2008.
91. McLean et al., 2000. Malignant gliomas display altered pH regulation by NHE1 compared with nontransformed astrocytes. Am J Physiol, Cell Physiol 278, C676-88. doi:10.1152/ajpcell.2000.278.4.C676.
92. Metheny et al., 2006. Tracheobronchial aspiration of gastric contents in critically ill tube-fed patients: frequency, outcomes, and risk factors. Crit Care Med.
93. Minoux et al., 2010. Molecular mechanisms of cranial neural crest cell migration and patterning in craniofacial development. Development. 137, 2605-2621.
94. Miraglia et al., 2005. Na+/H+ exchanger activity is increased in doxorubicin-resistant human colon cancer cells and its modulation modifies the sensitivity of the cells to doxorubicin. Int. J. Cancer 115, 924-929. doi:10.1002/ijc.20959.
95. Mochimaru et al., 2015. Extracellular acidification activates ovarian cancer G-protein-coupled receptor 1 and GPR4 homologs of zebra fish. Biochem. Biophys. Res. Commun. 457, 493-499. doi:10.1016/j.bbrc.2014.12.105.
96. Monteiro et al., 2017. The Role of Hypoxia in Glioblastoma Invasion. Cells 6(4): 45.
97. Morgenbesser et al., 1994. p53-dependent apoptosis produced by Rb-deficiency in the developing mouse lens. Nature. 371, 72-74.
98. Nassios et al., 2019. Expression of proton-sensing G-protein-coupled receptors in selected skin tumors. Exp. Dermatol. 28, 66-71. doi:10.1111/exd.13809.
99. Neri et al., 2011. Interfering with pH regulation in tumours as a therapeutic strategy. Nature Reviews Drug Discovery 10:767-777.
100. Nguyen et al., 2018. Molecular Markers of Therapy-Resistant Glioblastoma and Potential Strategy to Combat Resistance. International Journal of Molecular Sciences. 19, 1765.
101. Nishisho et al., 2011. The a3 isoform vacuolar type H⁺-ATPase promotes distant metastasis in the mouse B16 melanoma cells. Mol. Cancer Res. 9, 845-855. doi:10.1158/1541-7786.MCR-10-0449.
102. Oppitz et al., 2007. Non-malignant migration of B16 mouse melanoma cells in the neural crest and invasive growth in the eye cup of the chick embryo. Melanoma Res. 17, 17-30. doi:10.1097/CMR.0b013e3280114f49.
103. Paganetti et al., 1988. Glioblastoma infiltration into central nervous system tissue in vitro: involvement of a metalloprotease. J Cell Bio. 107(6 Pt 1):2281-91.
104. Parks et al., 2013. Disrupting proton dynamics and energy metabolism for cancer therapy. Nat. Rev. Cancer 13, 611-623. doi:10.1038/nrc3579.
105. Patel et al., 2014. Single-cell RNA-seq highlights intratumoral heterogeneity in primary glioblastoma. Science. 344, 1396-1401.
106. Peppicelli et al., 2014. Contribution of acidic melanoma cells undergoing epithelial-to-mesenchymal transition to aggressiveness of non-acidic melanoma cells. Clin. Exp. Metastasis 31, 423-433. doi:10.1007/s10585-014-9637-6.
107. Poltorack et al., 2022. Understanding the role of cysteine in ferroptosis: progress & paradoxes. FEBS J. 289, 374-385.
108. Ren et al., 2011. Effects of ovarian cancer G protein coupled receptor 1 on the proliferation, migration, and adhesion of human ovarian cancer cells. Chin. Med. J. 124, 1327-1332.
109. Riemann et al., 2015. Acidic environment activates inflammatory programs in fibroblasts via a cAMP-MAPK pathway. Biochim. Biophys. Acta 1853, 299-307. doi:10.1016/j.bbamcr.2014.11.022.
110. Robas et al., 2005. Identification of orphan G protein-coupled receptor ligands using FLIPR assays. Methods Mol Biol. 306, 17-26.
111. Rocha et al., 2020. Neural crest development: insights from the zebrafish. Developmental Dynamics. 249, 88-111.
112. Rofstad et al., 2006. Acidic extracellular pH promotes experimental metastasis of human melanoma cells in athymic nude mice. Cancer Res. 66, 6699-6707. doi:10.1158/0008-5472.CAN-06-0983.
113. Rohani et al., 2019. Acidification of Tumor at Stromal Boundaries Drives Transcriptome Alterations Associated with Aggressive Phenotypes. Cancer Res. 79, 1952-1966.
114. Roma-Rodrigues et al., 2019. Targeting Tumor Microenvironment for Cancer Therapy. International Journal of Molecular Sciences 20, 840.
115. Röttinger et al., 1982. Radioresistance secondary to low ph in human glial cells and Chinese hamster ovary cells. Int J Radiat Oncol Biol Phys. 8, 1309-1314.
116. Rowe et al., 2021. The evolution and mechanism of GPCR proton sensing. Journal of Biological Chemistry. 296.
117. Ruzicka et al., 2015. ZFIN, The zebrafish model organism database: Updates and new directions. Genesis. 53, 498-509.
118. Ryter et al., 2002. Heme oxygenase-1: molecular mechanisms of gene expression in oxygen-related stress. Antioxid Redox Signal. 4, 625-32.
119. Ryter et al., 2005. Heme oxygenase-1: redox regulation of a stress protein in lung and cell culture models. Antioxid Redox Signal. 7, 80-91.
120. Sanderlin et al., 2019. Pharmacological inhibition of GPR4 remediates intestinal inflammation in a mouse colitis model. Eur J Pharmacol. 852, 218-230.
121. Sato et al., 2018. The ferroptosis inducer erastin irreversibly inhibits system xc- and synergizes with cisplatin to increase cisplatin's cytotoxicity in cancer cells. Scientific Reports 2018 8:1, 8, 1-9.
122. Saxena et al., 2012. The GPCR OGR1 (GPR68) mediates diverse signalling and contraction of airway smooth muscle in response to small reductions in extracellular pH. Br J Pharmacol. 166(3):981-90.
123. Schneider et al., 2009. The Na+/H+ exchanger NHE1 is required for directional migration stimulated via PDGFR-alpha in the primary cilium. J. Cell Biol. 185, 163-176. doi:10.1083/jcb.200806019.
124. Schriek et al., 2005. Human SK-Mel 28 melanoma cells resume neural crest cell migration after transplantation into the chick embryo. Melanoma Res. 15, 225-234.
125. Sennoune et al., 2004. Vacuolar H+-ATPase in human breast cancer cells with distinct metastatic potential: distribution and functional activity. Am J Physiol, Cell Physiol 286, C1443-52. doi:10.1152/ajpcell.00407.2003.
126. Sente et al., 2018. Molecular mechanism of modulating arrestin conformation by GPCR phosphorylation. Nat. Struct. Mol. Biol. 25, 538-545. doi:10.1038/s41594-018-0071-3.
127. Shimizu et al., 2007. Exhaled breath marker in asthma patients with gastroesophageal reflux disease. J Clin Biochem Nutr. 41(3); 147-153. doi:10.3164/jcbn.2007020.
128. Schindelin et al., 2012. Fiji: an open-source platform for biological-image analysis. Nature Methods 2012 9:7, 9, 676-682.
129. Shi et al., 2009. The role of cellular oxidative stress in regulating glycolysis energy metabolism in hepatoma cells. Mol Cancer. 8, 1-15.
130. Singh et al., 2007. Ovarian cancer G protein-coupled receptor 1, a new metastasis suppressor gene in prostate cancer. J Natl Cancer Inst 99, 1313-1327. doi:10.1093/jnci/djm107.
131. Sinnberg et al., 2018. Wnt-signaling enhances neural crest migration of melanoma cells and induces an invasive phenotype. Mol. Cancer 17, 59. doi:10.1186/s12943-018-0773-5.
132. Schilling, 1997. Genetic analysis of craniofacial development in the vertebrate embryo. Bioessays. 19, 459-468.
133. Shalini et al., 2015. Old, new and emerging functions of caspases. Cell Death Differ. 22, 526-539 (2015).
134. Shi et al., 2021. Multifaceted mechanisms mediating cystine starvation-induced ferroptosis. Nat Commun. 12, 1 4792.
135. Son et al., 2017. Pneumonitis and pneumonia after aspiration. J Dent Anesth Pain Med. 17(1):1-12. doi: 10.17245/jdapm.2017.17.1.1
136. Stawicki et al., 2014. The zebrafish merovingian mutant reveals a role for pH regulation in hair cell toxicity and function. DMM Disease Models and Mechanisms. 7, 847-856.
137. Stewart et al., 2006. Zebrafish foxd3 is selectively required for neural crest specification, migration and survival. Dev. Biol. 292, 174-188. doi:10.1016/j.ydbio.2005.12.035.
138. Stock et al., 2005. Migration of human melanoma cells depends on extracellular pH and Na+/H+ exchange. J Physiol (Lond) 567, 225-238. doi:10.1113/jphysiol.2005.088344.
139. Stock et al., 2007. pH nanoenvironment at the surface of single melanoma cells. Cell. Physiol. Biochem. 20, 679-686. doi:10.1159/000107550.
140. Stupp et al., 2005. Radiotherapy plus concomitant and adjuvant temozolomide for glioblastoma. N Engl J Med. 352, 987-996.
141. Stitwe et al., 2007. pH dependence of melanoma cell migration: protons extruded by NHE1 dominate protons of the bulk solution. J Physiol (Lond) 585, 351-36). doi:10.1113/jphysiol.2007.145185.
142. Sun et al., 2016. Metallothionein-1G facilitates sorafenib resistance through inhibition of ferroptosis. Hepatology. 64, 488-500.
143. Suresh et al., 2019. Hypoxia-Inducible Factor (HIF)-1α Promotes Inflammation and Injury Following Aspiration-Induced Lung Injury in Mice. Shock. 52(6):612-621.
144. Sutoo et al., 2020. Adaptation to chronic acidic extracellular pH elicits a sustained increase in lung cancer cell invasion and metastasis. Clin Exp Metastasis. 37, 133-144.
145. Takebe et al., 2015. Targeting Notch, Hedgehog, and Wnt pathways in cancer stem cells: clinical update. Nat Rev Clin Oncol. 12, 445-464.
146. Tang et al., 2021, Ferroptosis Becomes Immunogenic: implications for anticancer treatments. Oncoimmunology 10(1), e1862949.
147. Tomura et al., 2010. Proton-sensing G-protein-coupled receptors and their physiological roles. Folia Pharmacologica Japonica. 135, 240-244.
148. Vainshtein et al., 2002. A high-throughput, nonisotopic, competitive binding assay for kinases using nonselective inhibitor probes (ED-NSIP). J Biomol Screen. 7, 507-514.
149. Vandenberg et al., 2011. V-ATPase-dependent ectodermal voltage and pH regionalization are required for craniofacial morphogenesis. Dev. Dyn. 240, 1889-1904. doi:10.1002/dvdy.22685.
150. Vander Heiden et al., 2009. Understanding the Warburg effect: the metabolic requirements of cell proliferation. Science 324, 1029-1033. doi:10.1126/science.1160809.
151. Velcicky et al., 2017. Development of Selective, Orally Active GPR4 Antagonists with Modulatory Effects on Nociception, Inflammation, and Angiogenesis. J Med Chem. 60, 3672-3683.
152. Wang et al., 2019. Artesunate activates the ATF4-CHOP-CHAC1 pathway and affects ferroptosis in Burkitt's Lymphoma. Biochem Biophys Res Commun. 519, 533-539.
153. Wang et al., 2020. GPR68 Is a Neuroprotective Proton Receptor in Brain Ischemia. Stroke. 51, 3690-3700.
154. Warburg, On the origin of cancer cells. Science 123 (3191): 309-14.
155. Webb et al., 2011. Dysregulated pH: a perfect storm for cancer progression. Nat. Rev. Cancer 11, 671-677. doi:10.1038/nrc3110.
156. Wei et al., 2018. Coincidence Detection of Membrane Stretch and Extracellular pH by the Proton-Sensing Receptor OGR1 (GPR68). Curr. Biol. 28, 3815-3823.e4. doi:10.1016/j.cub.2018.10.046.
157. Wei et al., 2017. Functional expression of calcium-permeable canonical transient receptor potential 4-containing channels promotes migration of medulloblastoma cells. J Physiol (Lond) 595, 5525-5544. doi:10.1113/JP274659.
158. Weiß et al., 2017. Proton-sensing G protein-coupled receptors as regulators of cell proliferation and migration during tumor growth and wound healing. Exp. Dermatol. 26, 127-132. doi:10.1111/exd.13209.
159. Westerfield. 2000. The zebrafish book: a guide for the laboratory use of zebrafish, 4th ed. University of Oregon Press, Eugene, OR.
160. White, 1994. P53, guardian of Rb. Nature. 371, 21-22.
161. Wiley et al., 2018. GPR68, a proton-sensing GPCR, mediates interaction of cancer-associated fibroblasts and cancer cells. FASEB J. 32, 1170-1183. doi:10.1096/fj.201700834R.
162. Wiley et al., 2019. GPR68: an emerging drug target in cancer. Int. J. Mol. Sci. 20. doi:10.3390/ijms20030559.
163. Williams et al., 2015. An in vivo chemical genetic screen identifies phosphodiesterase 4 as a pharmacological target for hedgehog signaling inhibition. Cell Rep. 11, 43-50. doi:10.1016/j.celrep.2015.03.001.
164. Wils et al., 2018. Epigenetic regulation of the Hedgehog and Wnt pathways in cancer. Crit Rev Oncol Hematol. 121, 23-44.
165. Wojtkowlak et al., 2012. Chronic autophagy is a cellular adaptation to tumor acidic pH microenvironments, Cancer Res, 1 5; 72(16):3938-47.
166. Worsley et al., 2022. The acidic tumour microenvironment: Manipulating the immune response to elicit escape. Hum Immunol. 83, 399-408.
167. Xu et al., 2022. Sevoflurane Induces Ferroptosis of Glioma Cells Through Activating the ATF4-CHAC1 Pathway. Front Oncol. 12, 859621.
168. Xu et al., 2018. GPR68 senses flow and is essential for vascular physiology. Cell 173, 762-775.e16. doi:10.1016/j.cell.2018.03.076.
169. Ye et al., 2020. Radiation-Induced Lipid Peroxidation Triggers Ferroptosis and Synergizes with Ferroptosis Inducers. ACS Chem Biol. 15:2, 469-484.
170. Yi et al., 2020. Oncogenic activation of PI3K-AKT-mTOR signaling suppresses ferroptosis via SREBP-mediated lipogenesis. Proc Natl Acad Sci USA. 117, 31189-31197.
171. Yu et al, 2008. Dorsomorphin inhibits BMP signals required for embryogenesis and iron metabolism. Nat. Chem. Biol. 4, 33-41. doi:10.1038/nchembio.2007.54.
172. Zanoni et al., 2016. 3D tumor spheroid models for in vitro therapeutic screening: a systematic approach to enhance the biological relevance of data obtained. Scientific Reports. 6, 1-11.
173. Zhang et al., 2018. The p53 Pathway in Glioblastoma. Cancers (Basel). 10:9, 297.
174. Zhao et al., 2020. Onco. Targets Ther. 13:5429-5441.
175. Zhou et al., 2017. Identification of Phosphorylation Codes for Arrestin Recruitment by G Protein-Coupled Receptors. Cell 170, 457-469.e13. doi:10.1016/j.cell.2017.07.002.
176. Zhou et al., 2020. FerrDb: a manually curated resource for regulators and markers of ferroptosis and ferroptosis-disease associations. Database: the journal of biological databases and curation. BAAA021.
177. Zhu et al., 2008. Development of a universal high-throughput calcium assay for G-protein-coupled receptors with promiscuous G-protein Gal5/16. Acta Pharmacologica Sinica 29:4, 507-516.
178. Zhu et al., 2016. Proton-sensing GPCR-YAP Signalling Promotes Cancer-associated Fibroblast Activation of Mesenchymal Stem Cells. Int. J. Biol. Sci. 12, 389-396. doi:10.7150/ijbs.13688.
179. Kepp et al., Oncoimmunology 2014; 3(9): e955691

### APPENDIX E

Lengthy table referenced here
US20240327364A1-20241003-T00001
Please refer to the end of the specification for access instructions.

### APPENDIX D

Lengthy table referenced here
US20240327364A1-20241003-T00002
Please refer to the end of the specification for access instructions.

### APPENDIX B

Lengthy table referenced here
US20240327364A1-20241003-T00003
Please refer to the end of the specification for access instructions.

### APPENDIX A

Lengthy table referenced here
US20240327364A1-20241003-T00004
Please refer to the end of the specification for access instructions.

### APPENDIX C

Lengthy table referenced here
US20240327364A1-20241003-T00005
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240327364A1). An electronic copy of the table will also be available from the USPTO upon request and payment of the fee set forth in 37 CFR 1.19(b)(3).

1. A method of inducing ferroptosis for treatment in a subject in need thereof, comprising administering to the subject a therapeutic GPR68 inhibiting 1,2-dihydro-3′H-spiro[indole-3,2′-[1,3,4]thiadiazole]-2-one agent of Formula I or a salt thereof: 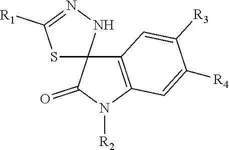
wherein R₁ is an optionally substituted 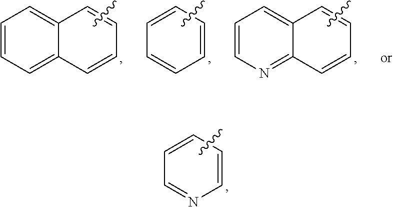
wherein the substitution is selected from —H, —CH₃, —CH₂CH₃, —OCH₃, —Br, —F, and —CF₃; wherein R₂ is —H or —CH₃; and wherein R₃ and R₄ independently are —H, —CH₃, —CH₂CH₃, —OCH₃, —CN, —F, —COOCH₃, —COOH, —SO₂NH₂.

2. The method of claim 1 wherein the subject is suffering from cancer or an acute lung injury.

3. The method of claim 2 wherein the subject is suffering from cancer.

4. The method of claim 3 wherein the cancer is a lymphoma, a leukemia, a germ cell tumor, a blastoma, a sarcoma, a blood cancer, a skin cancer, a breast cancer, a cervical cancer, an ovarian cancer, a breast cancer, a prostate cancer, a kidney cancer, a lung cancer, a pancreatic cancer, a liver cancer, a colon or colorectal cancer, and a brain cancer.

5. The method of claim 3 wherein the cancer is glioblastoma multiforme, medulloblastoma, fibrosarcoma, monocytic leukemia, B-cell lymphoma, chronic myelogeous leukemia, neuroendocrine prostate cancer, lung, colon, breast, pancreatic, and melanoma.

6. The method of claim 3 further comprising administering a cancer chemotherapeutic agent to the subject.

7. The method of claim 6 wherein the cancer chemotherapeutic agent is temozolomide or doxorubicin.

8. The method of claim 6 wherein co-administration of the therapeutic GPR68 inhibiting 1,2-dihydro-3′H-spiro[indole-3,2′-[1,3,4]thiadiazole]-2-one agent of Formula I and a cancer chemotherapeutic agent synergistically induces ferroptosis, immunogenic cell death, or both in cancer cells in the context of acidic tumor microenvironment.

9. The method of claim 3 further comprising administering radiation therapy to the subject.

10. The method of claim 9 wherein co-administration of the therapeutic GPR68 inhibiting 1,2-dihydro-3′H-spiro[indole-3,2′-[1,3,4]thiadiazole]-2-one agent of Formula I and radiation therapy to the subject synergistically induces ferroptosis, immunogenic cell death, or both in cancer cells in the context of acidic tumor microenvironment.

11. The method of claim 3 further comprising administering an ATF4 activating agent to the subject to overcome therapeutic resistance.

12. The method of claim 3 further comprising administering a cancer immunotherapy agent to the subject.

13. The method of claim 12 wherein the cancer immunotherapy agent is selected from the group consisting of ipilimumab, pembrolizumab, nivolumab, and atezolizumab.

14. The method of claim 3 wherein co-administration of the therapeutic GPR68 inhibiting 1,2-dihydro-3′H-spiro[indole-3,2′-[1,3,4]thiadiazole]-2-one agent of Formula I and a cancer immunotherapy agent to the subject promotes anti-cancer immunity.

15. The method of claim 2 wherein the subject is suffering from an acute lung injury.

16. The method of claim 15 wherein the acute lung injury is caused by bacterial infection, viral infection, inhalation injury, trauma, or mechanical ventilation-induced barotrauma.

17. The method of claim 15 wherein the subject is suffering from acute respiratory distress syndrome.

18. The method of claim 15 wherein the subject is at risk of developing acute respiratory distress syndrome.

19. The method of claim 1 wherein the therapeutic GPR68 inhibiting 1,2-dihydro-3′H-spiro[indole-3,2′-[1,3,4]thiadiazole]-2-one agent of Formula I or a salt thereof is OGM2.

20. A method of claim 1 wherein the therapeutic GPR68 inhibiting 1,2-dihydro-3′H-spiro[indole-3,2′-[1,3,4]thiadiazole]-2-one agent of Formula I or a salt thereof is OGM17.

21. A method of claim 1 wherein the therapeutic GPR68 inhibiting 1,2-dihydro-3′H-spiro[indole-3,2′-[1,3,4]thiadiazole]-2-one agent of Formula I or a salt thereof is OGM24.

22. A method of claim 1 wherein the therapeutic GPR68 inhibiting 1,2-dihydro-3′H-spiro[indole-3,2′-[1,3,4]thiadiazole]-2-one agent of Formula I or a salt thereof is OGM74.

23. A method of inducing ferroptosis for treatment in a subject in need thereof, comprising
administering to the subject a therapeutic amount of a therapeutic DNA or RNA molecule, wherein the DNA or RNA molecule comprises a sequence that silences, degrades or modulates GPR68-coding RNA.

24. The method of claim 23, wherein the administering the DNA or RNA molecule comprises a method selected from the group consisting of:
(a) administering the DNA or RNA molecule in a lipid nanoparticle formulation by intravenous injection;
(b) administering the DNA or RNA molecule in a polymeric nanoparticle formulation by inhalation;
(c) administering the DNA or RNA molecule in a viral vector formula by intramuscular injection;
(d) administering the DNA or RNA molecule in a conjugate formulation by topical application;
(e) administering the DNA or RNA molecule in a prodrug formulation by oral administration; and
(f) administering the DNA or RNA molecule in a nanoparticle formulation comprising a targeting ligand by subcutaneous injection.

25. The method of claim 23, wherein the DNA or RNA molecule is an siRNA or a microRNA.

26. A method of predicting the sensitivity of individual cancers to killing by GPR68 inhibition, comprising the steps of:
(a) obtaining a fresh or frozen tumor sample from a patient at the time of diagnostic tissue biopsy, surgical excision, bone marrow biopsy or peripheral blood draw;
(b) isolating mRNA from the tumor sample;
(c) generating cDNAs from the mRNA;
(d) determining the normalized expressions of GPR68 and GPR4 in the tumor sample by performing real-time qPCR;
(e) determining the ratio of the normalized expression of GPR68 relative to the normalized expression of GPR4 in the tumor sample,
wherein a ratio of 2:1 GPR68:GPR4 or higher in an individual tumor sample indicates increased sensitivity of the tumor to killing by GPR68 inhibition, and
wherein a ratio of less than 2:1 a ratio of 2:1 GPR68:GPR4 in an individual tumor sample indicates a lack of increased sensitivity of the tumor to killing by GPR68 inhibition.

27. The method of claim 26 wherein a ratio of 2:1 GPR68:GPR4 or higher in the individual tumor sample predicts therapeutic efficacy of the therapeutic agents.

28. A method of enhancing the therapeutic efficacy of cancer immunotherapy in a subject in need thereof, comprising the steps of:
(a) administering to the subject a therapeutic amount of a therapeutic DNA or RNA molecule, wherein the DNA or RNA molecule comprises a sequence that silences, degrades or modulates GPR68-coding RNA; and
(b) initiating cancer immunotherapy for the subject after or during the performance of (a).

29. The method of claim 28, wherein the cancer immunotherapy comprises administration of a checkpoint inhibitor or a tumor cell-killing chimeric antigen receptor T cells.

30. A method of enhancing immunological memory against tumors, comprising the steps of:
(a) administering to the subject a therapeutic amount of a therapeutic DNA or RNA molecule, wherein the DNA or RNA molecule comprises a sequence that silences, degrades or modulates GPR68-coding RNA; and
(b) administering to the subject a tumor vaccine.


SEQUENCE LISTING (28 sequences)

SEQ ID NO: 1 - protein, 16 aa, synthetic construct
  REGION 1..16: note = Synthetic: Rhodopsin
  MOD_RES 7: note = PHOSPHORYLATION
  MOD_RES 9: note = PHOSPHORYLATION
GDDASATVSKTETSQV

SEQ ID NO: 2 - protein, 17 aa, synthetic construct
  REGION 1..17: note = Synthetic: Vasopressin2
  MOD_RES 7: note = PHOSPHORYLATION
  MOD_RES 9: note = PHOSPHORYLATION
  MOD_RES 10: note = PHOSPHORYLATION
  MOD_RES 13: note = PHOSPHORYLATION
SLGQDESCTTASSSLAK

SEQ ID NO: 3 - protein, 17 aa, synthetic construct
  REGION 1..17: note = Synthetic: GPR68
  MOD_RES 7: note = PHOSPHORYLATION
  MOD_RES 10: note = PHOSPHORYLATION
PLGPEASGKSGAQGEEE

SEQ ID NO: 4 - protein, 5 aa, synthetic construct
  REGION 1..5: note = Synthetic: rs1745752
PLGPX

SEQ ID NO: 5 - DNA, 30 bp, synthetic construct
  misc_feature 1..30: note = Synthetic: Fig. 43 - target sequence of the zebrafish GPR68 morpholino
  source 1..30: mol_type = other DNA
gactctgaacatgtggttggaaaaatgaac

SEQ ID NO: 6 - DNA, 36 bp, synthetic construct
  misc_feature 1..36: note = Synthetic: Fig. 44 - target sequence of the zebrafish gpr68 sgRNA
  source 1..36: mol_type = other DNA
tcggaaggggtagaccaccgccaggtatcggtcaat

SEQ ID NO: 7 - DNA, 36 bp, synthetic construct
  misc_feature 1..36: note = Synthetic: Fig. 44 - target sequence of the zebrafish gpr68 sgRNA
  source 1..36: mol_type = other DNA
attgaccgatacctggcggtggtctaccccttccga

SEQ ID NO: 8 - DNA, 25 bp, synthetic construct
  misc_feature 1..25: note = Synthetic: OGR1 morpholino
  source 1..25: mol_type = other DNA
tttttccaaccacatgttcagagtc

SEQ ID NO: 9 - DNA, 25 bp, synthetic construct
  misc_feature 1..25: note = Synthetic: Mismatch morpholino
  source 1..25: mol_type = other DNA
cctcttacctcagttacaatttata

SEQ ID NO: 10 - DNA, 22 bp, synthetic construct
  misc_feature 1..22: note = Synthetic: GPR4
  source 1..22: mol_type = other DNA
ccctcctgtcataattccatcc

SEQ ID NO: 11 - DNA, 22 bp, synthetic construct
  misc_feature 1..22: note = Synthetic: GPR4
  source 1..22: mol_type = other DNA
tggtctacagggaagagatgag

SEQ ID NO: 12 - DNA, 20 bp, synthetic construct
  misc_feature 1..20: note = Synthetic: GPR65
  source 1..20: mol_type = other DNA
tggctgttgtctaccctttg

SEQ ID NO: 13 - DNA, 22 bp, synthetic construct
  misc_feature 1..22: note = Synthetic: GPR65
  source 1..22: mol_type = other DNA
ccacaacatgacagcattgaag

SEQ ID NO: 14 - DNA, 20 bp, synthetic construct
  misc_feature 1..20: note = Synthetic: GPR68
  source 1..20: mol_type = other DNA
gtttgaaggcggcagaaatg

SEQ ID NO: 15 - DNA, 20 bp, synthetic construct
  misc_feature 1..20: note = Synthetic: GPR68
  source 1..20: mol_type = other DNA
gtggaatgaggaggcatgaa

SEQ ID NO: 16 - DNA, 20 bp, synthetic construct
  misc_feature 1..20: note = Synthetic: GPR132
  source 1..20: mol_type = other DNA
ttcaggagcatcaagcagag

SEQ ID NO: 17 - DNA, 22 bp, synthetic construct
  misc_feature 1..22: note = Synthetic: GPR132
  source 1..22: mol_type = other DNA
cgaagcagactaggaagatgac

SEQ ID NO: 18 - DNA, 20 bp, synthetic construct
  misc_feature 1..20: note = Synthetic: GAPDH
  source 1..20: mol_type = other DNA
gtttgaaggcggcagaaatg

SEQ ID NO: 19 - DNA, 20 bp, synthetic construct
  misc_feature 1..20: note = Synthetic: GAPDH
  source 1..20: mol_type = other DNA
gtggaatgaggaggcatgaa

SEQ ID NO: 20 - RNA, 20 nt, synthetic construct
  misc_feature 1..20: note = Synthetic: GPR68 sgRNA1
  source 1..20: mol_type = other RNA
accgccatcctgtttataga

SEQ ID NO: 21 - RNA, 20 nt, synthetic construct
  misc_feature 1..20: note = Synthetic: GPR68 sgRNA2
  source 1..20: mol_type = other RNA
gaaggggccacactcctcat

SEQ ID NO: 22 - RNA, 20 nt, synthetic construct
  misc_feature 1..20: note = Synthetic: GPR68 sgRNA3
  source 1..20: mol_type = other RNA
ccataccatccaccagacgc

SEQ ID NO: 23 - RNA, 20 nt, synthetic construct
  misc_feature 1..20: note = Synthetic: GPR68 sgRNA4
  source 1..20: mol_type = other RNA
gccccttcaggcccaaagat

SEQ ID NO: 24 - RNA, 20 nt, synthetic construct
  misc_feature 1..20: note = Synthetic: ATF4 sgRNA1
  source 1..20: mol_type = other RNA
gatgtcccccttcgaccagt

SEQ ID NO: 25 - RNA, 20 nt, synthetic construct
  misc_feature 1..20: note = Synthetic: ATF4 sgRNA2
  source 1..20: mol_type = other RNA
gcggtgctttgctggaatcg

SEQ ID NO: 26 - RNA, 20 nt, synthetic construct
  misc_feature 1..20: note = Synthetic: ATF4 sgRNA3
  source 1..20: mol_type = other RNA
ccaccaacacctcgctgctc

SEQ ID NO: 27 - RNA, 20 nt, synthetic construct
  misc_feature 1..20: note = Synthetic: ATF4 sgRNA4
  source 1..20: mol_type = other RNA
agctcatttcggtcatgttg

SEQ ID NO: 28 - RNA, 20 nt, synthetic construct
  misc_feature 1..20: note = Synthetic: ATF4 sgRNA5
  source 1..20: mol_type = other RNA
aatgagcttcctgagcagcg